(19) United States
(12) Patent Application Publication
(10) Pub. No.: US 2020/0365808 A1
(43) Pub. Date: Nov. 19, 2020

(54) ORGANIC ELECTROLUMINESCENSE DEVICE AND ELECTRONIC APPARATUS

(71) Applicant: IDEMITSU KOSAN CO.,LTD., Tokyo (JP)

(72) Inventors: Yuki NAKANO, Sodegaura-shi (JP); Taro YAMAKI, Sodegaura-shi (JP); Satomi TASAKI, Sodegaura-shi (JP); Tomoki KATO, Sodegaura-shi (JP)

(73) Assignee: IDEMITSU KOSAN CO.,LTD., Tokyo (JP)

(21) Appl. No.: 16/872,627

(22) Filed: May 12, 2020

(30) Foreign Application Priority Data
May 14, 2019 (JP) 2019-091546

(51) Int. Cl. H01L 51/00; C09K 11/06; C09K 11/02; C07C 15/28; C07D 487/22

(52) U.S. Cl. CPC H01L 51/0058; C09K 11/06; C09K 11/02; C07C 15/28; C07D 487/22; H01L 51/5012; H01L 51/0072; C09K 2211/1022; C09K 2211/1014; C09K 2211/1007; H01L 51/0061

(57) ABSTRACT

An organic electroluminescence device, comprising: a cathode; an anode; and at least one organic layer disposed between the cathode and the anode, wherein the emitting layer comprises a compound represented by the following formula (1) and a compound represented by the following formula (A1).

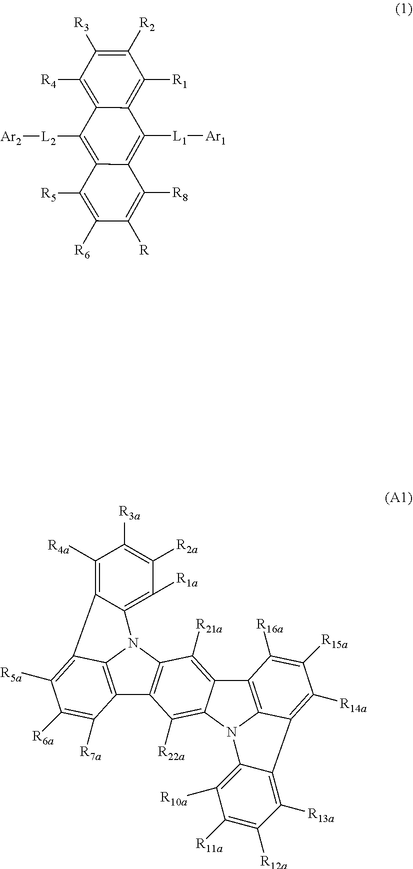

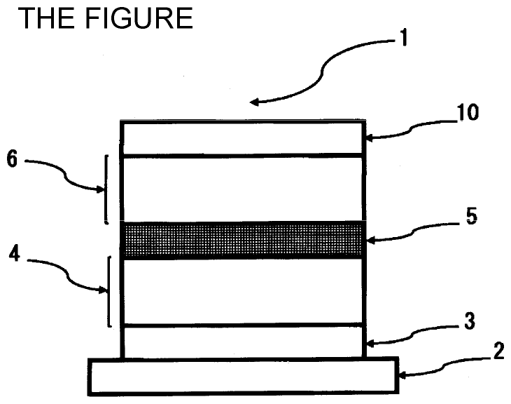

### TECHNICAL FIELD

The invention relates to an organic electroluminescence device and an electronic apparatus using the same.

### BACKGROUND ART

When voltage is applied to an organic electroluminescence device (hereinafter, referred to as an organic EL device in several cases), holes and electrons are injected into an emitting layer from an anode and a cathode, respectively. Then, thus injected holes and electrons are recombined in the emitting layer, and excitons are formed therein.

Patent Documents 1 to 4 disclose the use of an anthracene compound having a specific structure as a host material of an emitting layer of an organic EL device.

### RELATED ART DOCUMENTS

### Patent Documents

[Patent Document 1] WO2010/099534
[Patent Document 2] WO2010/135395
[Patent Document 3] WO2011/028216
[Patent Document 4] WO2010/071362

### SUMMARY OF THE INVENTION

It is an object of the invention to provide an organic electroluminescence device having a low CIEy value and a long lifetime.

It is another object of the invention to provide an electronic apparatus using the organic electroluminescence device.

According to the invention, an organic electroluminescence device according to the first aspect, an electronic apparatus according to the first aspect, a compound according to the second aspect, a material for an organic electroluminescence device according to the second aspect, an organic electroluminescence device according to the second aspect, and an electronic apparatus according to the second aspect are provided.

1. An organic electroluminescence device according to the first aspect, comprising: a cathode; an anode; and an emitting layer disposed between the cathode and the anode, wherein the emitting layer comprises a compound represented by the following formula (1) and a compound represented by the following formula (A1),

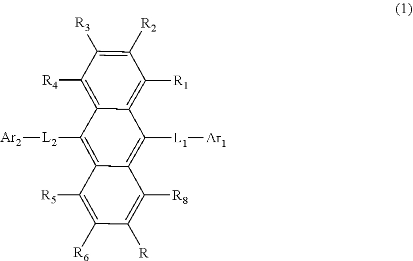

wherein in the formula (1),

at least one of R₁ to R₈ is a group represented by the following formula (1-2);

-L₃-Ar₃ (1-2)

L₁ to L₃ are independently

a single bond,

a substituted or unsubstituted phenylene group,

a substituted or unsubstituted naphthylene group,

a substituted or unsubstituted biphenylene group,

a substituted or unsubstituted terphenylene group,

a substituted or unsubstituted anthrylene group, or

a substituted or unsubstituted phenanthrylene group;

Ar₁ to Ar₃ are independently

a substituted or unsubstituted phenyl group,

a substituted or unsubstituted naphthyl group,

a substituted or unsubstituted biphenyl group,

a substituted or unsubstituted terphenyl group,

a substituted or unsubstituted anthryl group, or

a substituted or unsubstituted phenanthryl group;

when each of L₁, L₂, L₃, Ar₁, Ar₂ and Ar₃ has a substituent, the substituent is

an alkyl group including 1 to 50 carbon atoms,

a cycloalkyl group including 3 to 50 ring carbon atoms, or

an alkylsilyl group including 1 to 50 carbon atoms;

R₁ to R₈ which are not the group represented by the formula (1-2) are independently

a hydrogen atom,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄), or

—S—(R₉₀₅);

R₉₁₀ to R₉₀₅ are independently

a hydrogen atom,

a substituted or unsubstituted alkyl group including 1 to 50 carbon atoms,

a substituted or unsubstituted cycloalkyl group including 3 to 50 ring carbon atoms, or

a substituted or unsubstituted aryl group including 6 to 50 ring carbon atoms;

when two or more of each of R₉₀₁ to R₉₀₅ are present, the two or more of each of R₉₀₁ to R₉₀₅ may be the same or different;

at least one of R₁ to R₈ which are not the group represented by the formula (1-2) is a deuterium atom; and

adjacent two or more among R₁ to R₄ do not form a ring by bonding with each other, and adjacent two or more among R₅ to R₈ do not form a ring by bonding with each other,

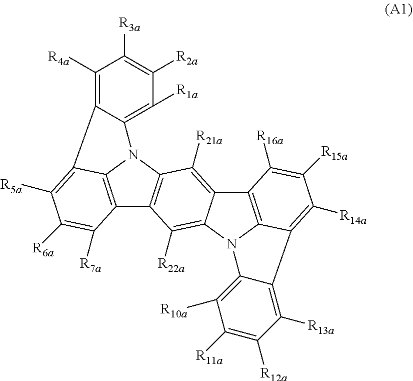

wherein in the formula (A1),

one or more sets of adjacent two or more among R₁ₐ to R₇ₐ and R₁₀ₐ to R₁₆ₐ form a substituted or unsubstituted, saturated or unsaturated ring by bonding with each other, or do not form a substituted or unsubstituted, saturated or unsaturated ring;

R₁ₐ to R₇ₐ and R₁₀ₐ to R₁₆ₐ which do not form the substituted or unsubstituted, saturated or unsaturated ring, and R₂₁ₐ and R₂₂ₐ are independently a hydrogen atom or a substituent;

the substituent is

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms;

R₉₀₁ to R₉₀₇ are independently

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same or different.

2. An electronic apparatus according to the first aspect, comprising the organic electroluminescence device according to 1.
3. A compound according to the second aspect, which is represented by the following formula (1):

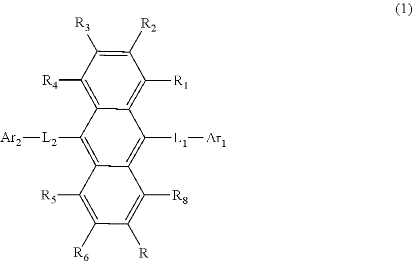

wherein in the formula (1),

at least one of R₁ to R₈ is a group represented by the following formula (1-2);

-L₃-Ar₃ (1-2)

L₁ to L₃ are independently

a single bond,

a substituted or unsubstituted phenylene group,

a substituted or unsubstituted naphthylene group,

a substituted or unsubstituted biphenylene group,

a substituted or unsubstituted terphenylene group,

a substituted or unsubstituted anthrylene group, or

a substituted or unsubstituted phenanthrylene group;

Ar₁ to Ar₃ are independently

a substituted or unsubstituted phenyl group,

a substituted or unsubstituted naphthyl group,

a substituted or unsubstituted biphenyl group,

a substituted or unsubstituted terphenyl group,

a substituted or unsubstituted anthryl group, or

a substituted or unsubstituted phenanthryl group.

when each of L₁, L₂, L₃, Ar₁, Ar₂ and Ar₃ has a substituent, the substituent is

an alkyl group including 1 to 50 carbon atoms,

a cycloalkyl group including 3 to 50 ring carbon atoms, or

an alkylsilyl group including 1 to 50 carbon atoms.

R₁ to R₈ which are not the group represented by the formula (1-2) are independently

a hydrogen atom,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄), or

—S—(R₉₀₅);

R₉₀₁ to R₉₀₅ are independently

a hydrogen atom,

a substituted or unsubstituted alkyl group including 1 to 50 carbon atoms,

a substituted or unsubstituted cycloalkyl group including 3 to 50 ring carbon atoms, or

a substituted or unsubstituted aryl group including 6 to 50 ring carbon atoms;

when two or more of each of R₉₀₁ to R₉₀₅ are present, the two or more of each of R₉₀₁ to R₉₀₅ may be the same or different;

at least one of R₁ to R₈ which are not the group represented by the formula (1-2) is a deuterium atom; and

adjacent two or more among R₁ to R₄ do not form a ring by bonding with each other, and

adjacent two or more among R₅ to R₈ do not form a ring by bonding with each other.

4. A material for an organic electroluminescence device according to the second aspect, comprising the compound represented by the formula (1) according to 3.
5. The organic electroluminescence device according to the second aspect, comprising: a cathode; an anode; and at least one organic layer disposed between the cathode and the anode, wherein at least one layer of the at least one organic layer comprises the compound according to 3.
6. An electronic apparatus according to the second aspect, comprising the organic electroluminescence device according to 5.

According to the invention, an organic electroluminescence device having a low CIEy value and a long lifetime can be provided.

### BRIEF DESCRIPTION OF THE DRAWINGS

The FIGURE is a diagram showing a schematic configuration of an aspect of an organic EL device according to an aspect of the invention.

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinazoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinazoline ring is not included in the number of ring atoms of the quinazoline ring. Therefore, the number of ring atoms of a quinazoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the ZZ group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group.

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkynyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

### “Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

Unsubstituted Aryl Group (Specific Example Group G1A):

a phenyl group,

a p-biphenyl group,

a m-biphenyl group,

an o-biphenyl group,

a p-terphenyl-4-yl group,

a p-terphenyl-3-yl group,

a p-terphenyl-2-yl group,

a m-terphenyl-4-yl group,

a m-terphenyl-3-yl group,

a m-terphenyl-2-yl group,

an o-terphenyl-4-yl group,

an o-terphenyl-3-yl group,

an o-terphenyl-2-yl group,

a 1-naphthyl group,

a 2-naphthyl group,

an anthryl group,

a benzanthryl group,

a phenanthryl group,

a benzophenanthryl group,

a phenalenyl group,

a pyrenyl group,

a chrysenyl group,

a benzochrysenyl group,

a triphenylenyl group,

a benzotriphenylenyl group,

a tetracenyl group,

a pentacenyl group,

a fluorenyl group,

a 9,9′-spirobifluorenyl group,

a benzofluorenyl group,

a dibenzofluorenyl group,

a fluoranthenyl group,

a benzofluoranthenyl group,

a perylenyl group, and

a monovalent aryl group derived by removing one hydrogen atom from the ring structures represented by any of the following general formulas (TEMP-1) to (TEMP-15).

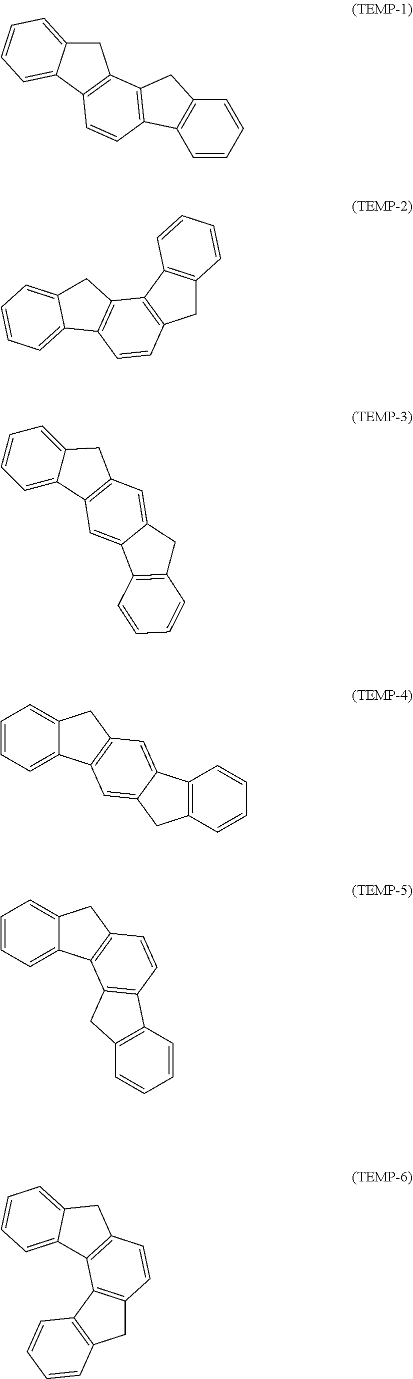

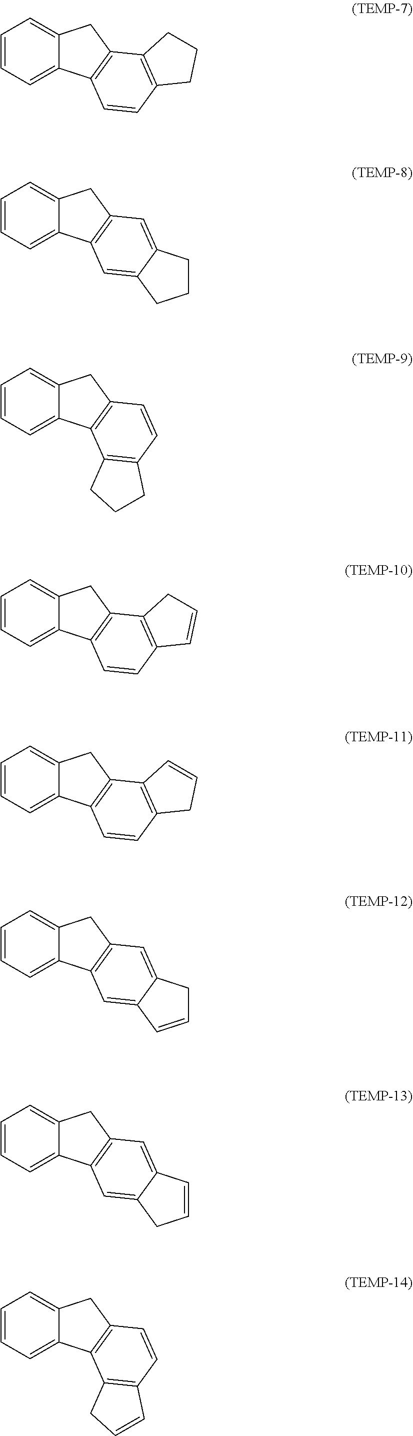

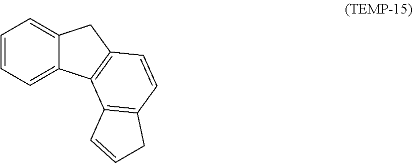

Substituted Aryl Group (Specific Example Group G1B):

an o-tolyl group,

a m-tolyl group,

a p-tolyl group,

a p-xylyl group,

a m-xylyl group,

an o-xylyl group,

a p-isopropylphenyl group,

a m-isopropylphenyl group,

an o-isopropylphenyl group,

a p-t-butylphenyl group,

a m-t-butylphenyl group,

an o-t-butylphenyl group,

a 3,4,5-trimethylphenyl group,

a 9,9-dimethylfluorenyl group,

a 9,9-diphenylfluorenyl group,

a 9,9-bis(4-methylphenyl)fluorenyl group,

a 9,9-bis(4-isopropylphenyl)fluorenyl group,

a 9,9-bis(4-t-butylphenyl)fluorenyl group,

a cyanophenyl group,

a triphenylsilylphenyl group,

a trimethylsilylphenyl group,

a phenylnaphthyl group,

a naphthylphenyl group, and

a group in which one or more hydrogen atoms of a monovalent group derived from the ring structures represented by any of the general formulas (TEMP-1) to (TEMP-15) are substituted by a substituent.

“Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”.). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

Unsubstituted Heterocyclic Group Containing a Nitrogen Atom (Specific Example Group G2A1):

a pyrrolyl group,

an imidazolyl group,

a pyrazolyl group,

a triazolyl group,

a tetrazolyl group,

an oxazolyl group,

an isoxazolyl group,

an oxadiazolyl group,

a thiazolyl group,

an isothiazolyl group,

a thiadiazolyl group,

a pyridyl group,

a pyridazinyl group,

a pyrimidinyl group,

a pyrazinyl group,

a triazinyl group,

an indolyl group,

an isoindolyl group,

an indolizinyl group,

a quinolizinyl group,

a quinolyl group,

an isoquinolyl group,

a cinnolyl group,

a phthalazinyl group,

a quinazolinyl group,

a quinoxalinyl group,

a benzimidazolyl group,

an indazolyl group,

a phenanthrolinyl group,

a phenanthridinyl group,

an acridinyl group,

a phenazinyl group,

a carbazolyl group,

a benzocarbazolyl group,

a morpholino group,

a phenoxazinyl group,

a phenothiazinyl group,

an azacarbazolyl group, and

a diazacarbazolyl group.

Unsubstituted Heterocyclic Group Containing an Oxygen Atom (Specific Example Group G2A2):

a furyl group,

an oxazolyl group,

an isoxazolyl group,

an oxadiazolyl group,

a xanthenyl group,

a benzofuranyl group,

an isobenzofuranyl group,

a dibenzofuranyl group,

a naphthobenzofuranyl group,

a benzoxazolyl group,

a benzisoxazolyl group,

a phenoxazinyl group,

a morpholino group,

a dinaphthofuranyl group,

an azadibenzofuranyl group,

a diazadibenzofuranyl group,

an azanaphthobenzofuranyl group, and

a diazanaphthobenzofuranyl group.

Unsubstituted Heterocyclic Group Containing a Sulfur Atom (Specific Example Group G2A3):

a thienyl group,

a thiazolyl group,

an isothiazolyl group,

a thiadiazolyl group,

a benzothiophenyl group (benzothienyl group),

an isobenzothiophenyl group (isobenzothienyl group),

a dibenzothiophenyl group (dibenzothienyl group),

a naphthobenzothiophenyl group (naphthobenzothienyl group),

a benzothiazolyl group,

a benzisothiazolyl group,

a phenothiazinyl group,

a dinaphthothiophenyl group (dinaphthothienyl group),

an azadibenzothiophenyl group (azadibenzothienyl group),

a diazadibenzothiophenyl group (diazadibenzothienyl group),

an azanaphthobenzothiophenyl group (azanaphthobenzothienyl group), and

a diazanaphthobenzothiophenyl group (diazanaphthobenzothienyl group).

Monovalent Heterocyclic Group Derived by Removing One Hydrogen Atom from the Ring Structures Represented by any of the Following General Formulas (TEMP-16) to (TEMP-33) (Specific Example Group G2A4):

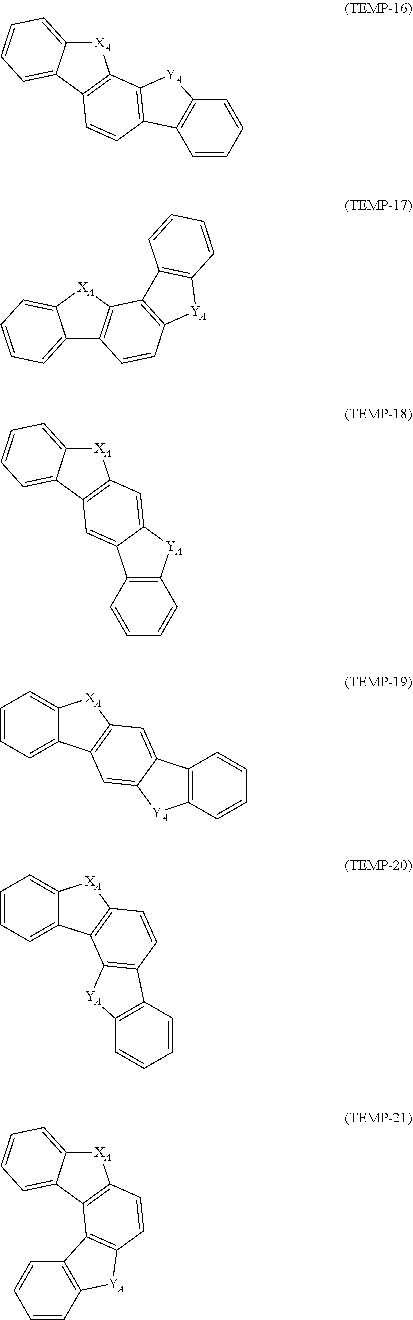

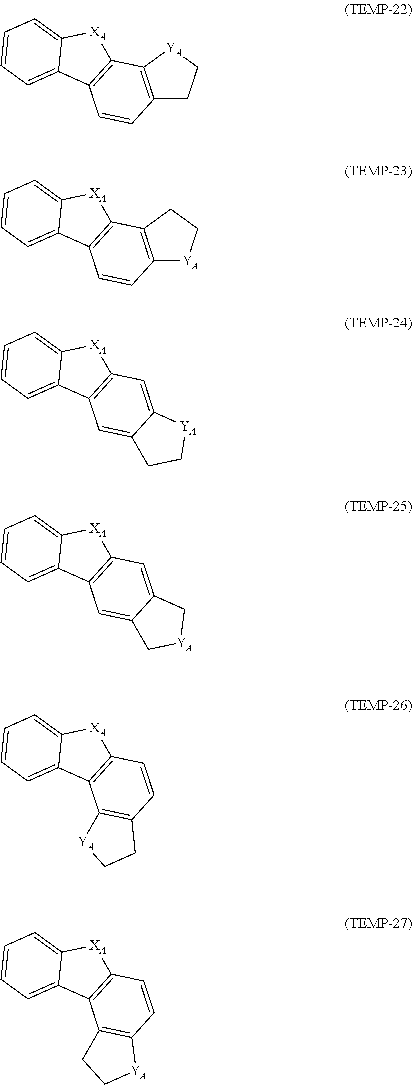

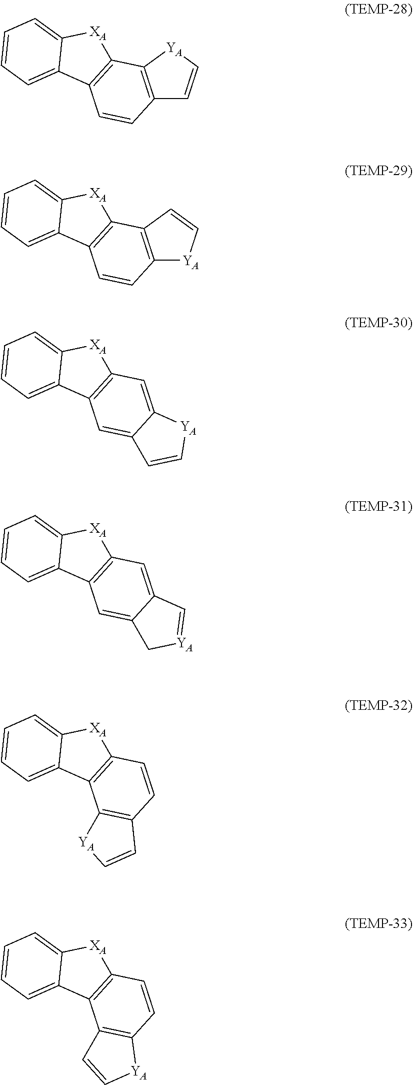

In the general formulas (TEMP-16) to (TEMP-33), X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.

Substituted Heterocyclic Group Containing a Nitrogen Atom (Specific Example Group G2B1):

a (9-phenyl)carbazolyl group,

a (9-biphenylyl)carbazolyl group,

a (9-phenyl)phenylcarbazolyl group,

a (9-naphthyl)carbazolyl group,

a diphenylcarbazol-9-yl group,

a phenylcarbazol-9-yl group,

a methylbenzimidazolyl group,

an ethylbenzimidazolyl group,

a phenyltriazinyl group,

a biphenylyltriazinyl group,

a diphenyltriazinyl group,

a phenylquinazolinyl group, and

a biphenylylquinazolinyl group.

Substituted Heterocyclic Group Containing an Oxygen Atom (Specific Example Group G2B2):

a phenyldibenzofuranyl group,

a methyldibenzofuranyl group,

a t-butyldibenzofuranyl group, and

a monovalent residue of spiro[9H-xanthene-9,9′-[9H]fluorene].

Substituted Heterocyclic Group Containing a Sulfur Atom (Specific Example Group G2B3):

a phenyldibenzothiophenyl group,

a methyldibenzothiophenyl group,

a t-butyldibenzothiophenyl group, and

a monovalent residue of spiro[9H-thioxanthene-9,9′-[9H]fluorene].

Group in which One or More Hydrogen Atoms of the Monovalent Heterocyclic Group Derived from the Ring Structures Represented by any of the Following General Formulas (TEMP-16) to (TEMP-33) are Substituted by a Substituent (Specific Example Group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

“Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an“alkyl group substituted by a substituent”.). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

Unsubstituted Alkyl Group (Specific Example Group G3A):

a methyl group,

an ethyl group,

a n-propyl group,

an isopropyl group,

a n-butyl group,

an isobutyl group,

a s-butyl group, and

a t-butyl group.

Substituted Alkyl Group (Specific Example Group G3B):

a heptafluoropropyl group (including isomers),

a pentafluoroethyl group,

a 2,2,2-trifluoroethyl group, and

a trifluoromethyl group.

“Substituted or Unsubstituted Alkenyl Group”

Specific examples of the “substituted or unsubstituted alkenyl group” described in this specification (specific example group G4) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group G4B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

Unsubstituted Alkenyl Group (Specific Example Group G4A):

a vinyl group,

an allyl group,

a 1-butenyl group,

a 2-butenyl group, and

a 3-butenyl group.

Substituted Alkenyl Group (Specific Example Group G4B):

a 1,3-butanedienyl group,

a 1-methylvinyl group,

a 1-methylallyl group,

a 1,1-dimethylallyl group,

a 2-methylally group, and

a 1,2-dimethylallyl group.

“Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”.). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

Unsubstituted Alkynyl Group (Specific Example Group G5A):

an ethynyl group.

“Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”.). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

Unsubstituted Cycloalkyl Group (Specific Example Group G6A):

a cyclopropyl group,

a cyclobutyl group,

a cyclopentyl group,

a cyclohexyl group,

a 1-adamantyl group,

a 2-adamantyl group,

a 1-norbornyl group, and

a 2-norbornyl group.

Substituted Cycloalkyl Group (Specific Example Group G6B):

a 4-methylcyclohexyl group.

“Group Represented by —Si (R₉₀₁)(R₉₀₂)(R₉₀₃)”

Specific examples of the group represented by —Si(R₉₀₀)(R₉₀₂)(R₉₀₃) described in this specification (specific example group G7) include:

—Si(G1)(G1)(G1),

—Si(G1)(G2)(G2),

—Si(G1)(G1)(G2),

—Si(G2)(G2)(G2),

—Si(G3)(G3)(G3), and

—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

“Group Represented by —O—(R₉₀₄)”

Specific examples of the group represented by —O—(R₉₀₄) in this specification (specific example group G8) include:

—O(G1),

—O(G2),

—O(G3), and

—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —S—(R₉₀₅)”

Specific examples of the group represented by —S—(R₉₀₅) in this specification (specific example group G9) include:

—S(G1),

—S(G2),

—S(G3), and

—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —N(R₉₀₆)(R₉₀₇)”

Specific examples of the group represented by —N(R₉₀₆)(R₉₀₇) in this specification (specific example group G10) include:

—N(G1)(G1),

—N(G2)(G2),

—N(G1)(G2),

—N(G3)(G3), and

—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

“Halogen Atom”

Specific examples of the “halogen atom” described in this specification (specific example group G11) include a fluorine atom, a chlorine atom, a bromine atom, an iodine atom, and the like.

“Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

“Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

“Substituted or Unsubstituted Alkoxy Group”

Specific examples of the “substituted or unsubstituted alkoxy group” described in this specification include a group represented by —O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

“Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

“Substituted or Unsubstituted Aryloxy Group”

Specific examples of the “substituted or unsubstituted aryloxy group” described in this specification include a group represented by —O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

“Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

“Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

“Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

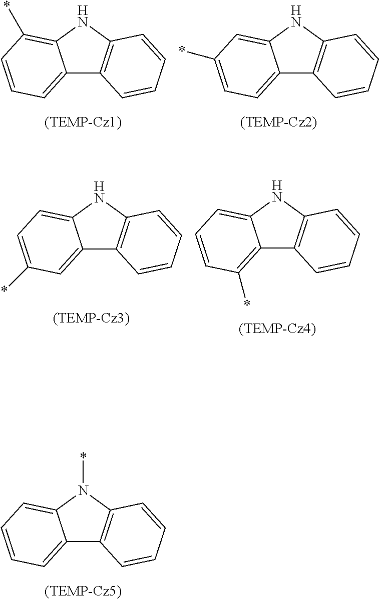

In this specification, the (9-phenyl)carbazolyl group is specifically any of the following groups, unless otherwise specified in this specification.

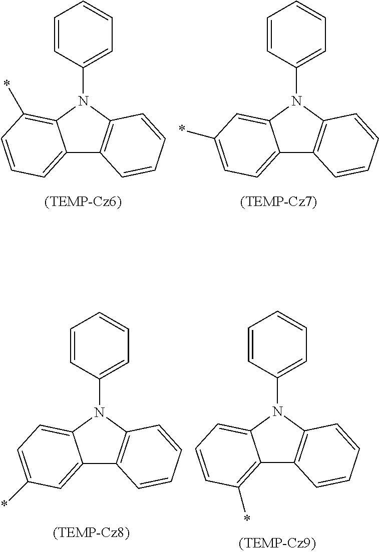

In the general formulas (TEMP-Cz1) to (TEMP-Cz9), * represents a bonding position.

In this specification, the dibenzofuranyl group and the dibenzothiophenyl group are specifically any of the following groups, unless otherwise specified in this specification.

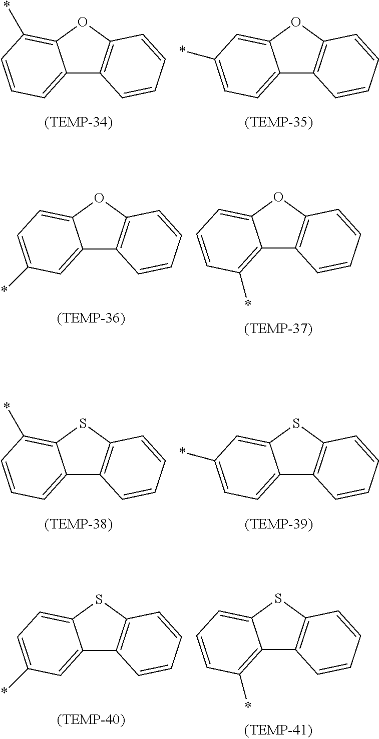

In the general formulas (TEMP-34) to (TEMP-41), * represents a bonding position.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

“Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

“Substituted or Unsubstituted Divalent Heterocyclic Group”

The “substituted or unsubstituted divalent heterocyclic group” described in this specification is a divalent group derived by removing one hydrogen atom on the heterocyclic ring of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocyclic ring of the “substituted or unsubstituted heterocyclic group” described in the specific example group G2, and the like.

“Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

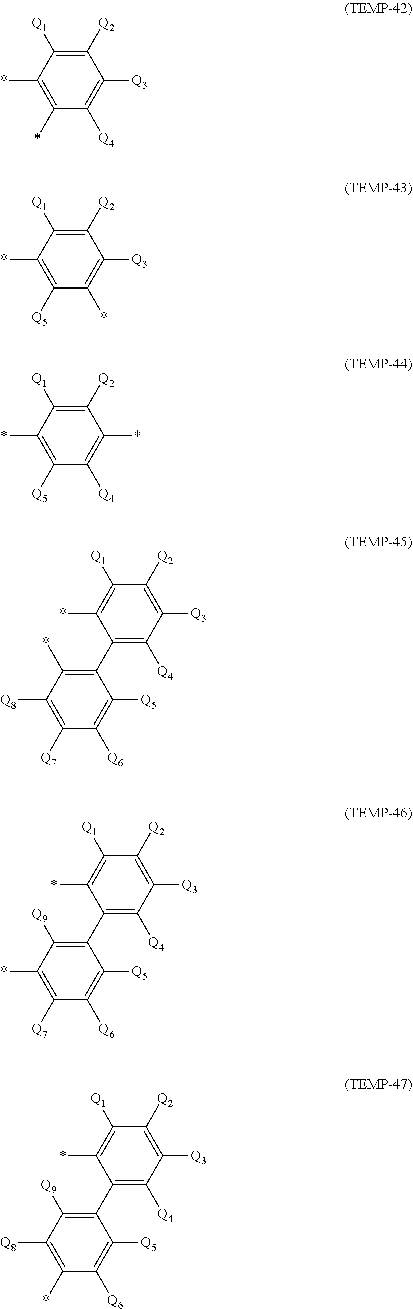

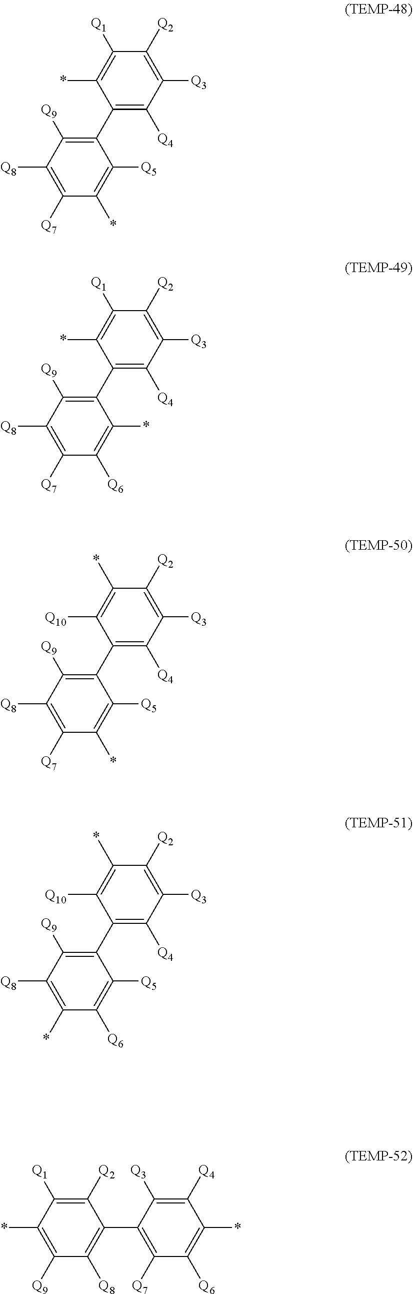

In the general formulas (TEMP-42) to (TEMP-52), Q₁ to Q₁₀ are independently a hydrogen atom or a substituent.

In the general formulas (TEMP-42) to (TEMP-52), * represents a bonding position.

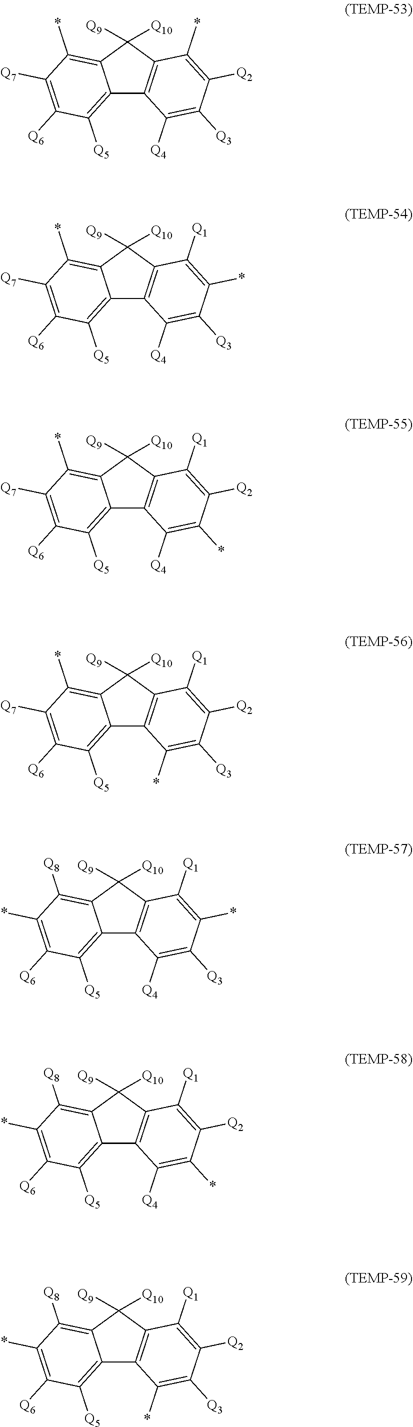

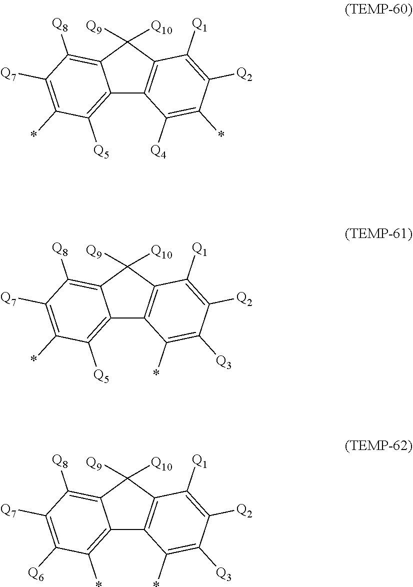

In the general formulas (TEMP-53) to (TEMP-62), Q₁ to Q₁₀ are independently a hydrogen atom or a substituent.

Q₉ and Q₁₀ may be bonded with each other via a single bond to form a ring.

In the general formulas (TEMP-53) to (TEMP-62), * represents a bonding position.

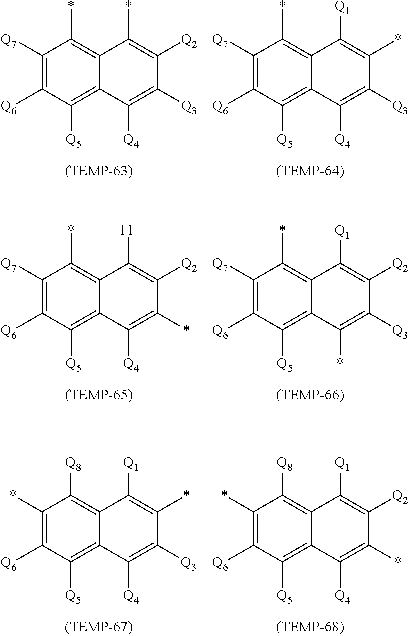

In the general formulas (TEMP-63) to (TEMP-68), Q₁ to Q₈ are independently a hydrogen atom or a substituent.

In the general formulas (TEMP-63) to (TEMP-68), * represents a bonding position.

The substituted or unsubstituted divalent heterocyclic group described in this specification is preferably any group of the following general formulas (TEMP-69) to (TEMP-102), unless otherwise specified in this specification.

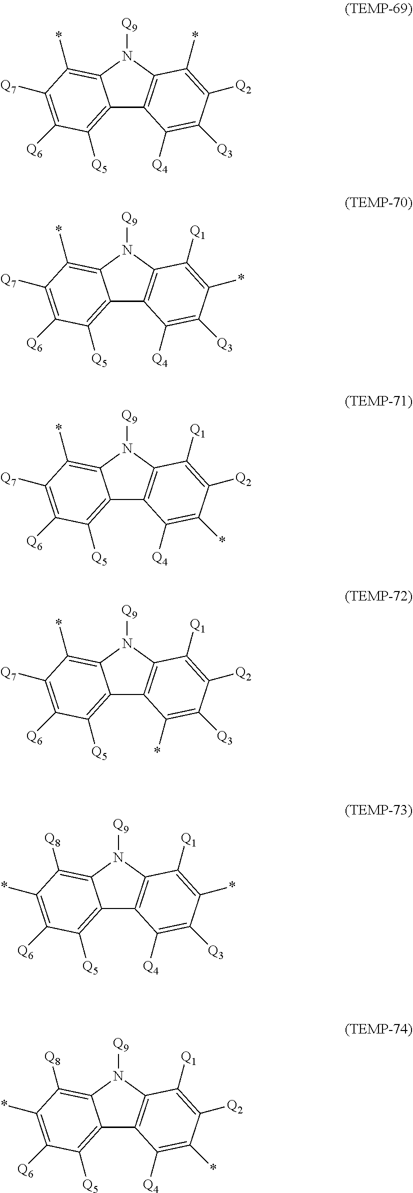

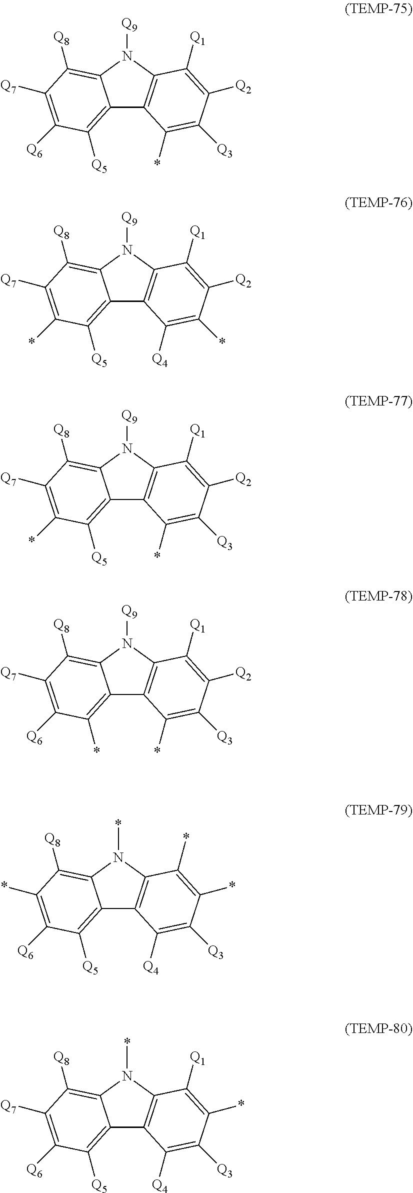

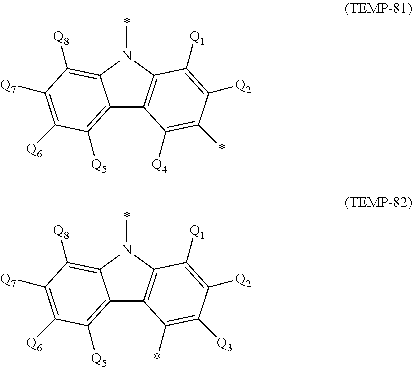

In the general formulas (TEMP-69) to (TEMP-82), Q₁ to Q₉ are independently a hydrogen atom or a substituent.

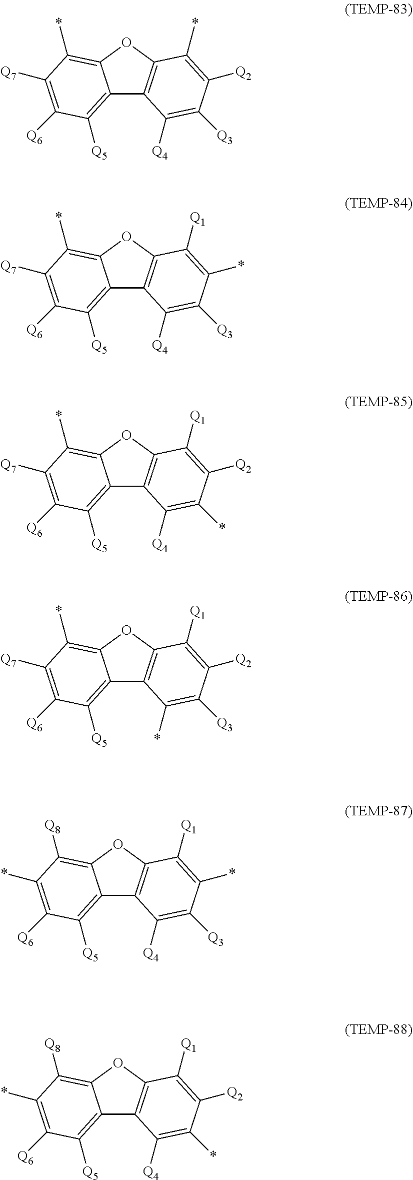

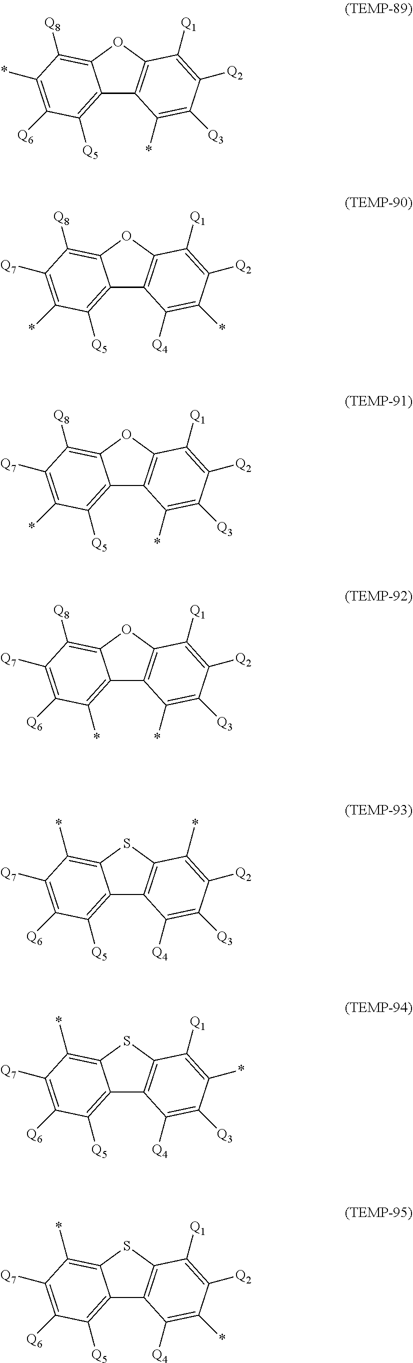

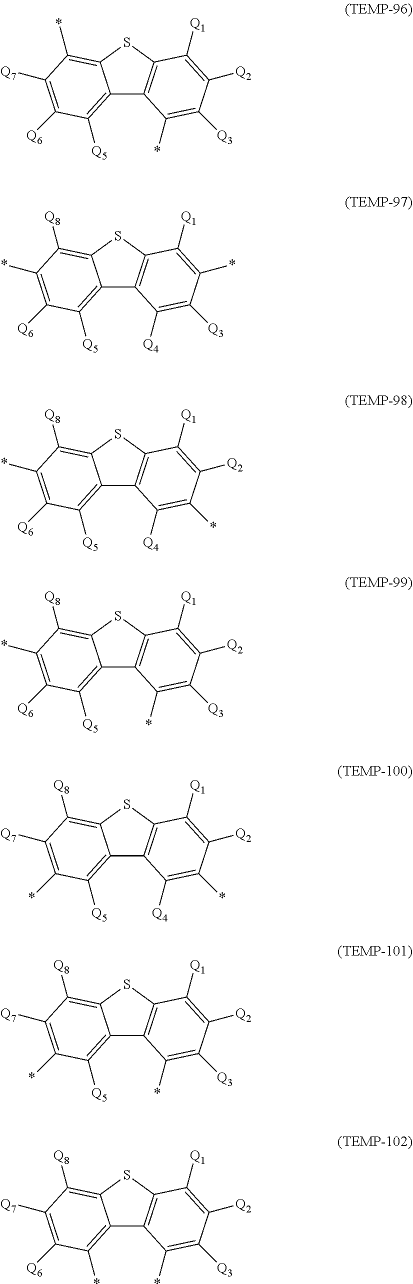

In the general formulas (TEMP-83) to (TEMP-102), Q₁ to Q₈ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

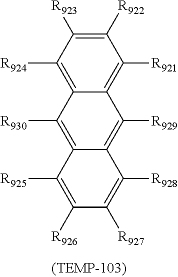

For example, in the case where “one or more sets of adjacent two or more among R₉₂₁ to R₉₃₀ form a ring by bonding with each other”, the one sets of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₆, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆ form a ring Q_{B} by bonding with each other, the anthracene compound represented by the general formula (TEMP-103) is represented by the following general formula (TEMP-104).

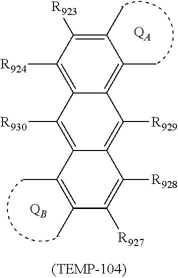

The case where the “pair of adjacent two or more” form a ring includes not only the case where the pair of adjacent “two” is bonded with as in the above-mentioned examples, but also the case where the pair of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

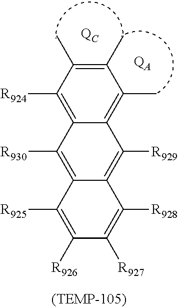

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may forma saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TMEP-104) is a benzene ring, the ring Q_{A} is a monocycle. When the ring Q_{A} of the general formula (TMEP-104) is a naphthalene ring, the ring Q_{A} is a fused ring.

The “unsaturated ring” means an aromatic hydrocarbon ring or an aromatic heterocyclic ring. The “saturated ring” means an aliphatic hydrocarbon ring, or a non-aromatic heterocyclic ring.

Specific examples of the aromatic hydrocarbon ring include a structure in which the group listed as a specific example in the specific example group G1 is terminated by a hydrogen atom.

Specific examples of the aromatic heterocyclic ring include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary elements in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary elements. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary element” is preferably at least one element selected from the group consisting of a carbon element, a nitrogen element, an oxygen element, and a sulfur element, unless otherwise specified in this specification. In the arbitrary element (for example, a carbon element or a nitrogen element), a bond which does not form a ring may be terminated with a hydrogen atom or the like, or may be substituted with “arbitrary substituent” described below. When an arbitrary element other than a carbon element is contained, the ring formed is a heterocyclic ring.

The number of “one or more arbitrary element(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less elements which is at least one kind selected from a carbon elements, a nitrogen element, an oxygen element, and a sulfur element.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:

an unsubstituted alkyl group including 1 to 50 carbon atoms,

an unsubstituted alkenyl group including 2 to 50 carbon atoms,

an unsubstituted alkynyl group including 2 to 50 carbon atoms,

an unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

an unsubstituted aryl group including 6 to 50 ring carbon atoms, and

an unsubstituted heterocyclic group including 5 to 50 ring atoms,

wherein, R₉₀₁ to R₉₀₇ are independently

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present, the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of:

an alkyl group including 1 to 50 carbon atoms,

an aryl group including 6 to 50 ring carbon atoms, and

a heterocyclic group including 5 to 50 ring atoms.

In one embodiment, the substituent in the case of “substituted or unsubstituted” is a group selected from the group consisting of:

an alkyl group including 1 to 18 carbon atoms,

an aryl group including 6 to 18 ring carbon atoms, and

a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [The Organic Electroluminescence Device According to the First Aspect]

The organic electroluminescence device according to the first aspect of the invention comprises:

a cathode;

an anode; and

an emitting layer disposed between the cathode and the anode,

wherein the emitting layer comprises a compound represented by the following formula (1) and a compound represented by the following formula (A1).

The compound represented by the formula (1) and the compound represented by the formula (1A) will be described later.

The organic EL device according to an aspect of the invention exhibits high device performance by having the above-mentioned configuration. Specifically, the organic EL device that simultaneously satisfies both properties of a low CIEy value and a long lifetime can be provided.

Further, according to the first aspect of the organic EL device, by using the compound represented by the formula (1) and the compound represented by the formula (A1) with each other in the emitting layer of the organic EL device in combination, a method for improving the performance of the organic EL device can also be provided. According to the second aspect of the organic EL device, by using the compound represented by the formula (1) in the emitting layer of the organic EL device, a method for improving the performance of the organic EL device can also be provided. Specifically, the method can improve the performance of the organic EL device as compared with the case where a compound having the same structure as the compound represented by the formula (1) except that the compound has only protium atoms as hydrogen atoms (hereinafter, also referred to as a “protium form”) is used as the host material. The case where the protium form is used means the case where substantially only protium form (the ratio of the protium form to the sum of the compound represented by the formula (1) and the protium form is 90 mol % or more, 95 mol % or more, or 99 mol % or more) is used as the host material in the emitting layer.

That is, the performance can be improved by using, as the host material, a compound in which at least one of the protium atom on the anthracene skeleton of the protium form is replaced with a deuterium atom (the compound represented by the formula (1)) instead of the protium form.

The schematic configuration of the organic EL device in one embodiment according to the first and second aspects of the invention will be explained referring to the FIGURE.

The organic EL device 1 according to one embodiment of the invention includes a substrate 2, an anode 3, a cathode 10, an emitting layer 5 as an organic layer, an organic layer 4 between the anode 3 and the emitting layer 5, and an organic layer 6 between the emitting layer 5 and the cathode 10.

Each of the organic layer 4 and the organic layer 6 may be a single layer or may consist of a plurality of layers.

In the organic EL device according to the first aspect, the compound represented by the formula (1) and the compound represented by the formula (A1) are contained in the emitting layer 5 between the anode 3 and the cathode 10. Each of these compounds may be contained singly or in combination of two or more.

In one embodiment, the organic EL device comprises a hole-transporting layer between the anode 3 and the emitting layer 5, i.e. in the organic layer 4.

In one embodiment, the organic EL device comprises an electron-transporting layer between the cathode 10 and the emitting layer 5, i.e. in the organic layer 6.

In the organic EL device according to the second aspect, the compound represented by the formula (1) is contained in the organic layer, i.e., the emitting layer 5, the organic layer 4 between the anode 3 and the emitting layer 5, or the organic layer 6 between the emitting layer 5 and the cathode 10, and is preferably contained in the emitting layer 5. The compound represented by the formula (1) may be contained singly or in combination of two or more.

### <Compound Represented by the Formula (1)>

The emitting layer of the organic EL device according to the first aspect contains a compound represented by the following formula (1). The compound represented by the following formula (1) is characterized in that at least one of hydrogen atoms which is directly bonded with a carbon atom constituting anthracene skeleton is a deuterium atom.

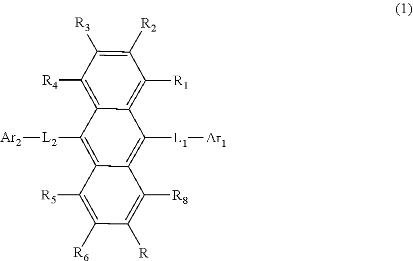

In the formula (1),

at least one of R₁ to R₈ is a group represented by the following formula (1-2);

-L₃-Ar₃ (1-2)

L₁ to L₃ are independently

a single bond,

a substituted or unsubstituted phenylene group,

a substituted or unsubstituted naphthylene group,

a substituted or unsubstituted biphenylene group,

a substituted or unsubstituted terphenylene group,

a substituted or unsubstituted anthrylene group, or

a substituted or unsubstituted phenanthrylene group;

Ar₁ to Ar₃ are independently

a substituted or unsubstituted phenyl group,

a substituted or unsubstituted naphthyl group,

a substituted or unsubstituted biphenyl group,

a substituted or unsubstituted terphenyl group,

a substituted or unsubstituted anthryl group, or

a substituted or unsubstituted phenanthryl group.

when each of L₁, L₂, L₃, Ar₁, Ar₂ and Ar₃ has a substituent, the substituent is

an alkyl group including 1 to 50 carbon atoms,

a cycloalkyl group including 3 to 50 ring carbon atoms, or

an alkylsilyl group including 1 to 50 carbon atoms.

R₁ to R₈ which are not the group represented by the formula (1-2) are independently

a hydrogen atom,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄), or

—S—(R₉₀₅).

R₉₀₁ to R₉₀₅ are independently

a hydrogen atom,

a substituted or unsubstituted alkyl group including 1 to 50 carbon atoms,

a substituted or unsubstituted cycloalkyl group including 3 to 50 ring carbon atoms, or

a substituted or unsubstituted aryl group including 6 to 50 ring carbon atoms;

when two or more of each of R₉₀₁ to R₉₀₅ are present, the two or more of each of R₉₀₁ to R₉₀₅ may be the same or different;

at least one of R₁ to R₈ which are not the group represented by the formula (1-2) is a deuterium atom;

adjacent two or more among R₁ to R₄ do not form a ring by bonding with each other, and adjacent two or more among R₅ to R₈ do not form a ring by bonding with each other.

The compound represented by the formula (1) has at least one deuterium atom bonded with the anthracene skeleton in the formula.

The presence of a deuterium atom in a compound is confirmed by mass spectrometry or ¹H-NMR analysis. The bonding positions of the deuterium atom in the compound is identified by ¹H-NMR analysis. Specifically, it can be confirmed by the following method.

The target compound is subjected to mass spectrometry, and if the molecular weight is increased by 1 compared to the corresponding compound in which all hydrogen atoms are protium atoms, it can be confirmed that the compound contains one deuterium atom. In addition, the number of deuterium atoms in the molecule can be confirmed by the integral value obtained by ¹H-NMR analysis of the target compound, since a deuterium atom gives no signal in ¹H-NMR analysis. In addition, the binding position of the deuterium atom can be identified by subjecting the target compound to ¹H-NMR analysis, and assigning the obtained signals.

In one embodiment, in the formula (1), at least two of R₁ to R which are not the group represented by the formula (1-2) are deuterium atoms.

In one embodiment, in the formula (1), all of R₁ to R₈ which are not the group represented by the formula (1-2) are deuterium atoms.

In one embodiment, in the formula (1), at least one hydrogen atom which one or more selected from the group consisting of L₁ and L₂ have is a deuterium atom.

In one embodiment, in the formula (1), L₁ and L₂ are independently a single bond, a substituted or unsubstituted phenylene group, or a substituted or unsubstituted naphthylene group.

In one embodiment, in the formula (1), at least one hydrogen atom which one or more selected from the group consisting of Ar₁ and Ar₂ have is a deuterium atom.

In one embodiment, in the formula (1), Ar₁ and Ar₂ are independently a substituted or unsubstituted phenyl group, a substituted or unsubstituted naphthyl group, or a substituted or unsubstituted phenanthryl group.

In one embodiment, the emitting layer comprises a compound represented by the formula (1) and a compound having the same structures as the compound represented by the formula (1) except that the compound has only protium atoms as hydrogen atoms (hereinafter also referred to as “protium form”), and the content ratio of the latter to the sum of these compounds is 99 mol % or less. The content of the protium form can be confirmed by mass spectrometry.

In one embodiment, the emitting layer of the organic EL device according to an aspect of the invention comprises the compound represented by the formula (1) and its protium form, and the content ratio of the former to the sum of these compounds is 30 mol % or more, 50 mol % or more, 70 mol % or more, 90 mol % or more, 95 mol % or more, 99 mol % or more, or 100 mol %.

In one embodiment, in the formula (1), one or both of L₁ and L₂ in the formula (1) is single bonds.

In one embodiment, in the formula (1), one of L₁ and L₂ in the formula (1) is a single bond.

In one embodiment, in the formula (1), both of L₁ and L₂ in the formula (1) are single bonds.

In one embodiment, in the formula (1), one or more hydrogen atoms which Ar₁ to Ar₃ have are deuterium atoms.

In one embodiment, in the formula (1), all of hydrogen atoms which Ar₁ to Ar₃ have are deuterium atoms.

In one embodiment, in the formula (1), one or more hydrogen atoms which L₁ and L₂ among L₁ to L₃ have are deuterium atoms.

In one embodiment, in the formula (1), all of hydrogen atoms which L₁ to L₃ have are deuterium atoms.

In one embodiment, in the formula (1), all of hydrogen atoms which Ar₁ to Ar₃ and L₁ to L₃ have are deuterium atoms.

In one embodiment, in the formula (1), one of Ar₁ and Ar₂ is a substituted or unsubstituted 1-naphthyl group and the other is a substituted or unsubstituted 1-naphthyl group or 2-naphthyl group.

In one embodiment, in the formula (1), one of Ar₁ and Ar₂ is a substituted or unsubstituted 1-naphthyl group which do not have a deuterium atom, the other is a substituted or unsubstituted 1-naphthyl group which do not have a deuterium atom, or a substituted or unsubstituted 2-naphthyl group which do not have a deuterium atom.

In one embodiment, in the formula (1), one of Ar₁ and Ar₂ is a substituted or unsubstituted 1-naphthyl group which has one or more deuterium atoms, the other is a substituted or unsubstituted 1-naphthyl group which has one or more deuterium atoms, or a substituted or unsubstituted 2-naphthyl group which has one or more deuterium atoms.

In one embodiment, in the formula (1), Ar₃ is a substituted or unsubstituted phenyl group, a substituted or unsubstituted 1-naphthyl group, or a substituted or unsubstituted 2-naphthyl group.

In one embodiment, in the formula (1), Ar₃ is a substituted or unsubstituted phenyl group which has a deuterium atom, a substituted or unsubstituted 1-naphthyl group which has a deuterium atom, or a substituted or unsubstituted 2-naphthyl group which has a deuterium atom.

In one embodiment, in the formula (1), L₃ is a single bond, and

Ar₃ is a substituted or unsubstituted phenyl group which do not have a deuterium atom, or a substituted or unsubstituted naphthyl group which do not have a deuterium atom.

In one embodiment, in the formula (1), L₃ is a single bond, and

Ar₃ is a substituted or unsubstituted phenyl group which has a deuterium atom, or a substituted or unsubstituted naphthyl group which has a deuterium atom.

Examples of the terphenyl group in Ar₁ to Ar₃ include groups having the following structures:

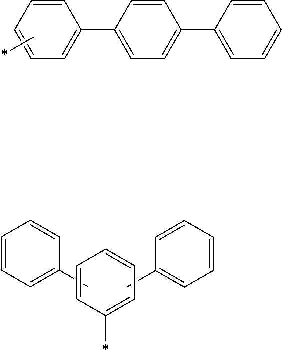

wherein in the formula, * is bonded with any of L₁ to L₃.

The compound represented by the formula (1) within the scope of the invention can be synthesized in accordance with the synthetic methods described in Examples by using known alternative reactions or raw materials tailored to the target compound.

Specific examples of the compound represented by the formula (1) will be described below, but the compound represented by the formula (1) are not limited to these specific examples. “D” in the following specific examples represents a deuterium atom.

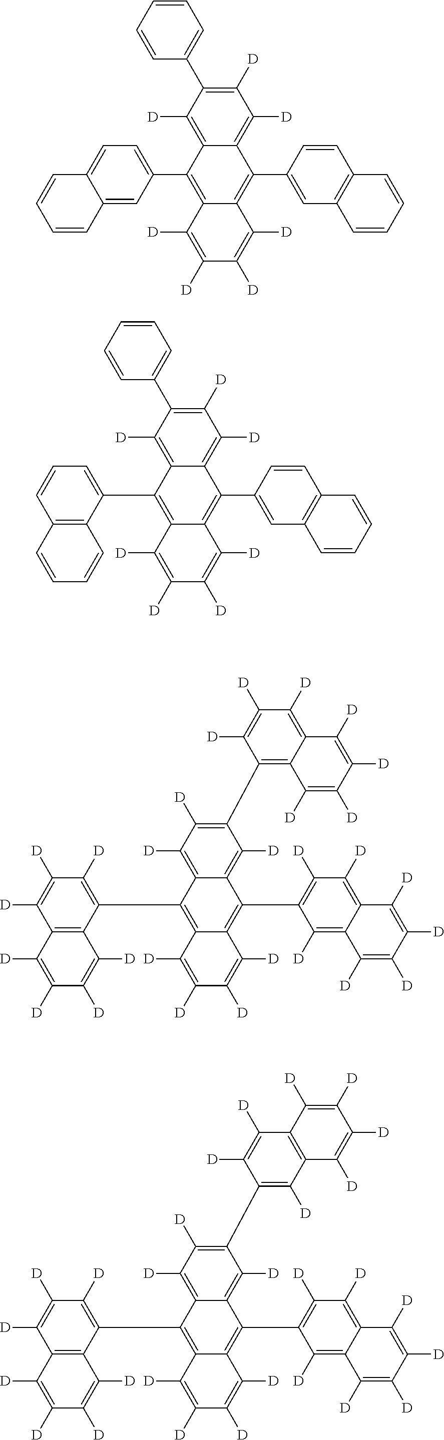

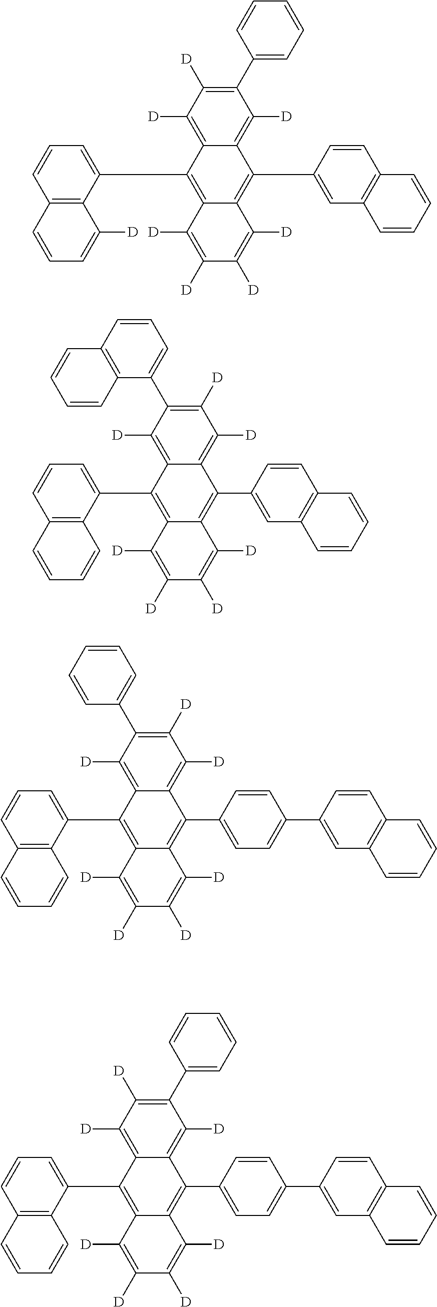

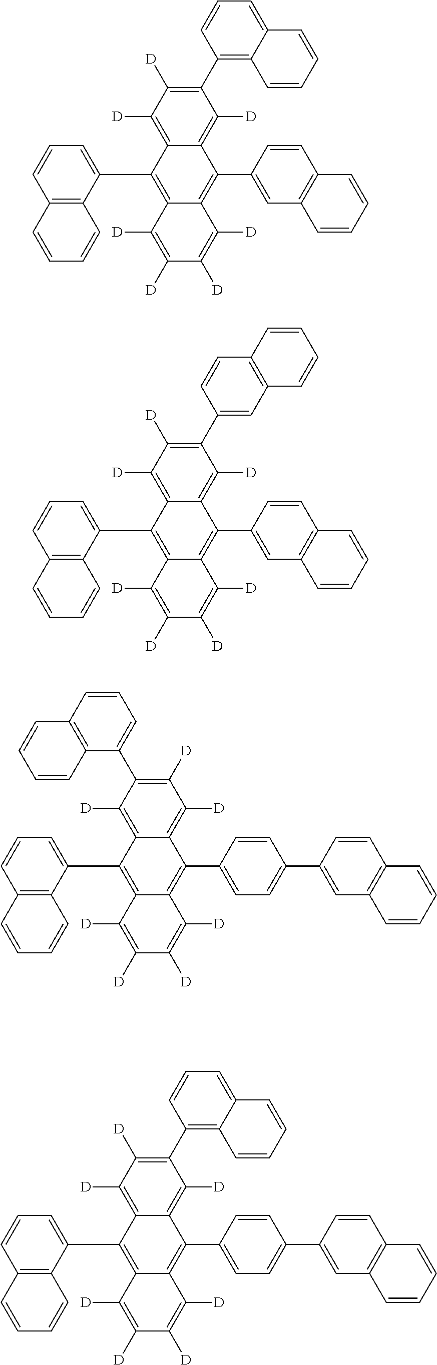 [SMILES: [2H]c1c([2H])c([2H])c2c(-c3cccc4ccccc34)c3c([2H])c([2H])c(-c4cccc5ccccc45)c([2H])c3c(-c3ccc4ccccc4c3)c2c1[2H] ; [2H]c1c([2H])c([2H])c2c(-c3cccc4ccccc34)c3c([2H])c([2H])c(-c4ccc5ccccc5c4)c([2H])c3c(-c3ccc4ccccc4c3)c2c1[2H] ; [2H]c1c([2H])c([2H])c2c(-c3cccc4ccccc34)c3c([2H])c(-c4cccc5ccccc45)c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c(-c4cccc5ccccc45)c([2H])c3c(-c3ccc(-c4ccc5ccccc5c4)cc3)c2c1[2H]]

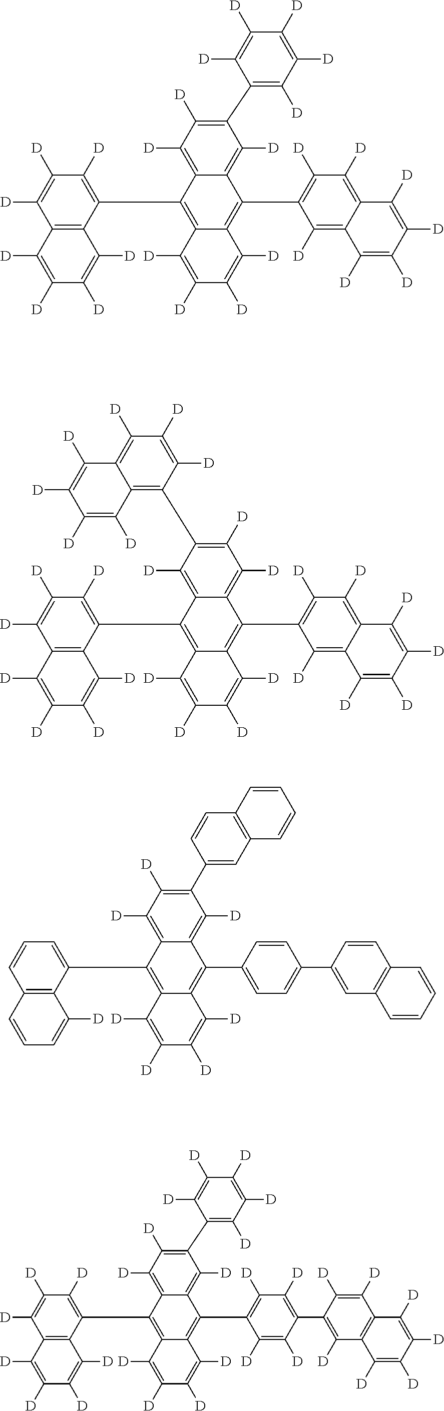

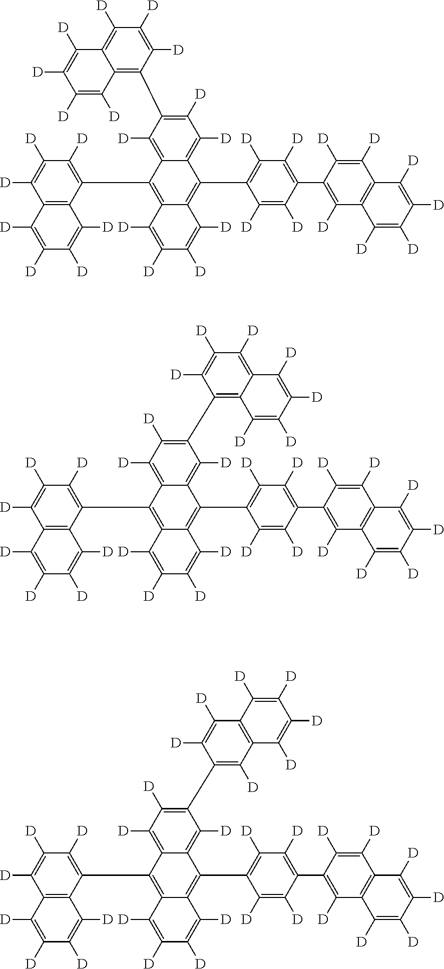

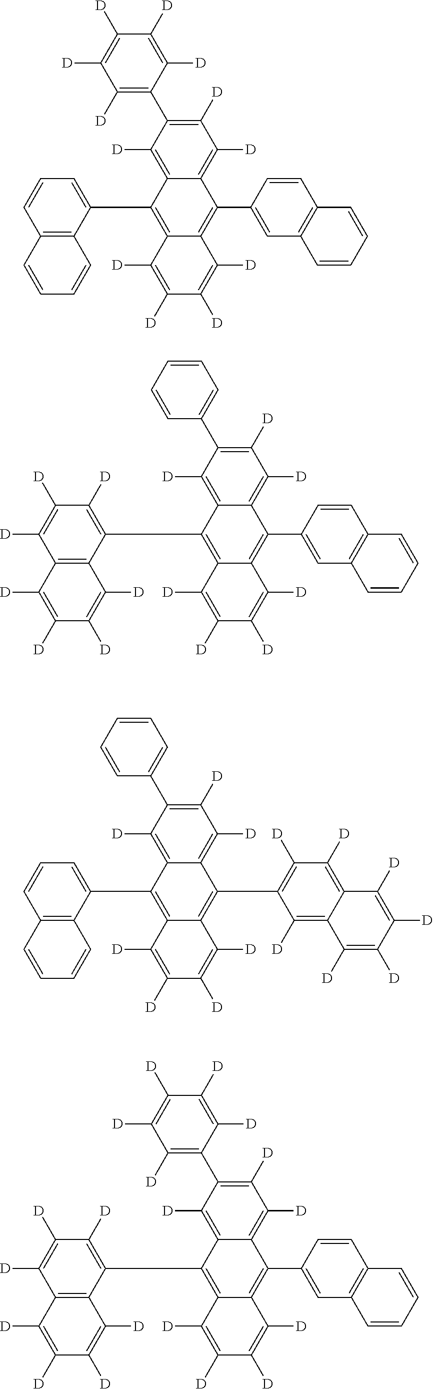

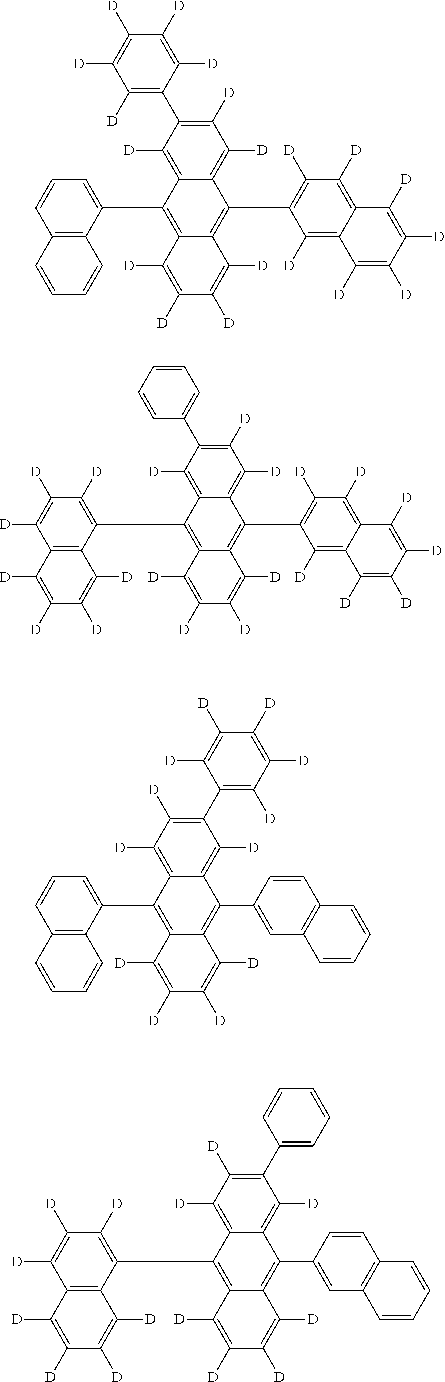

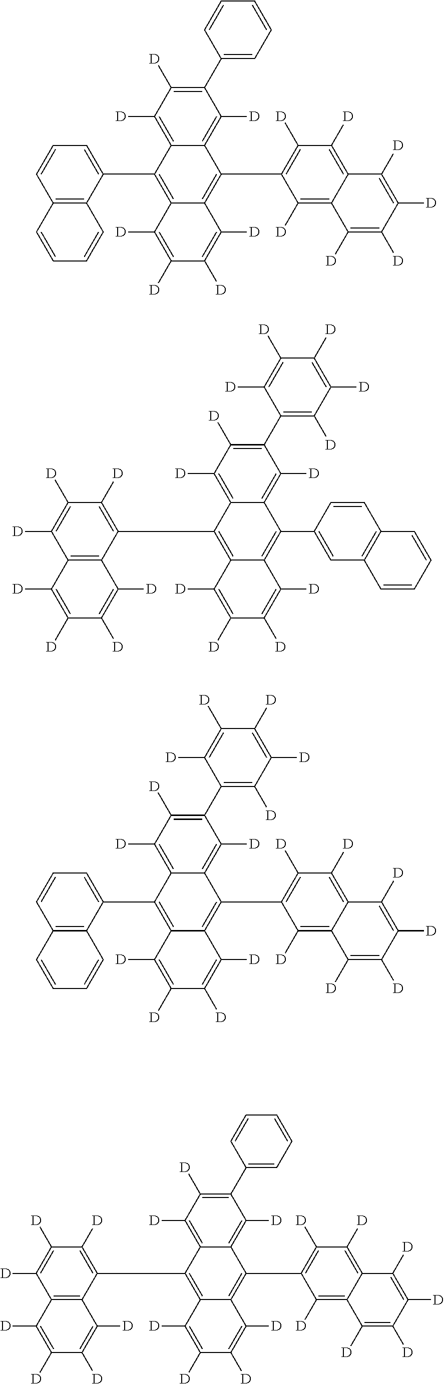

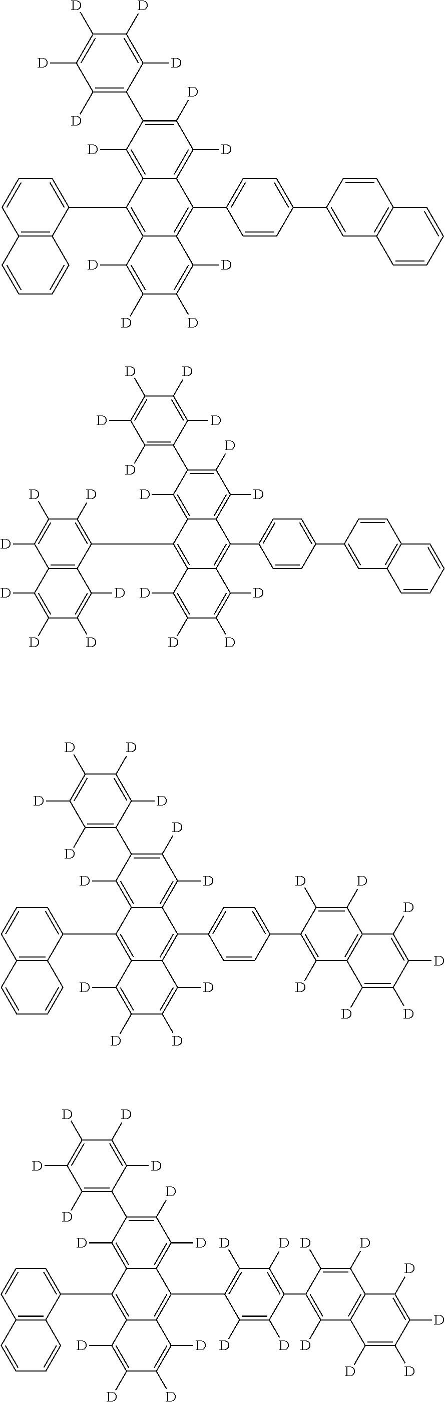

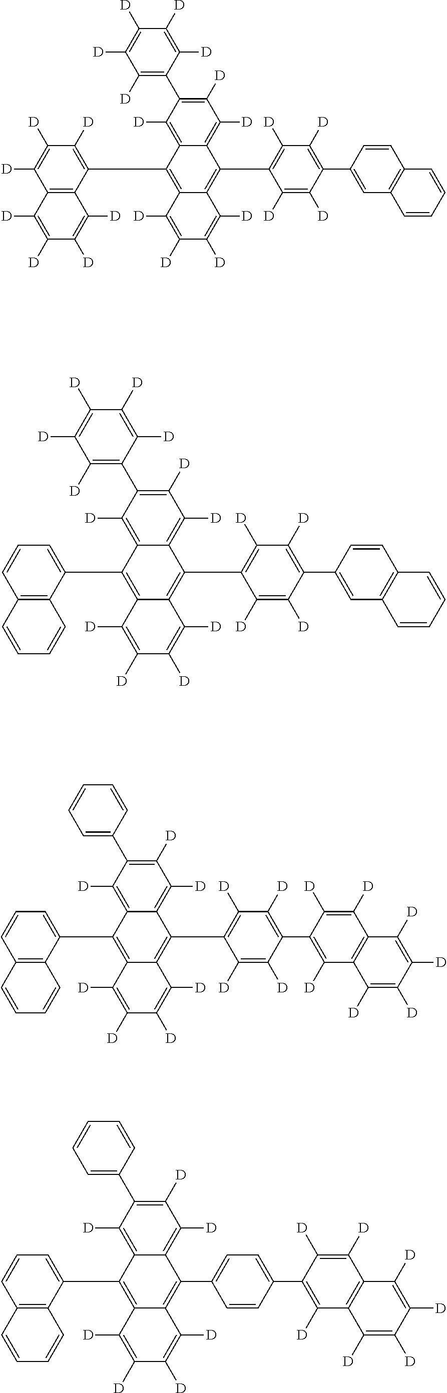

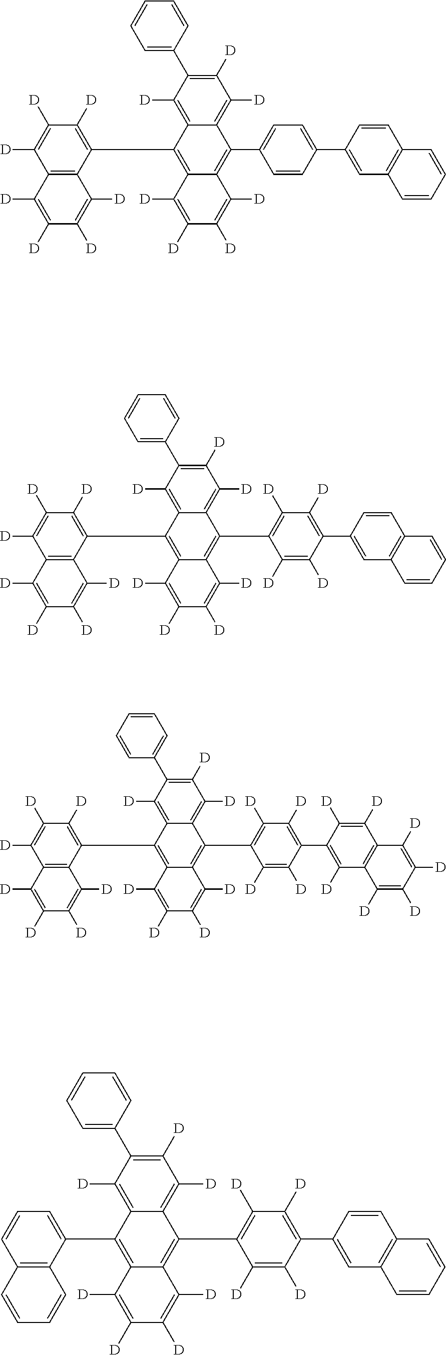

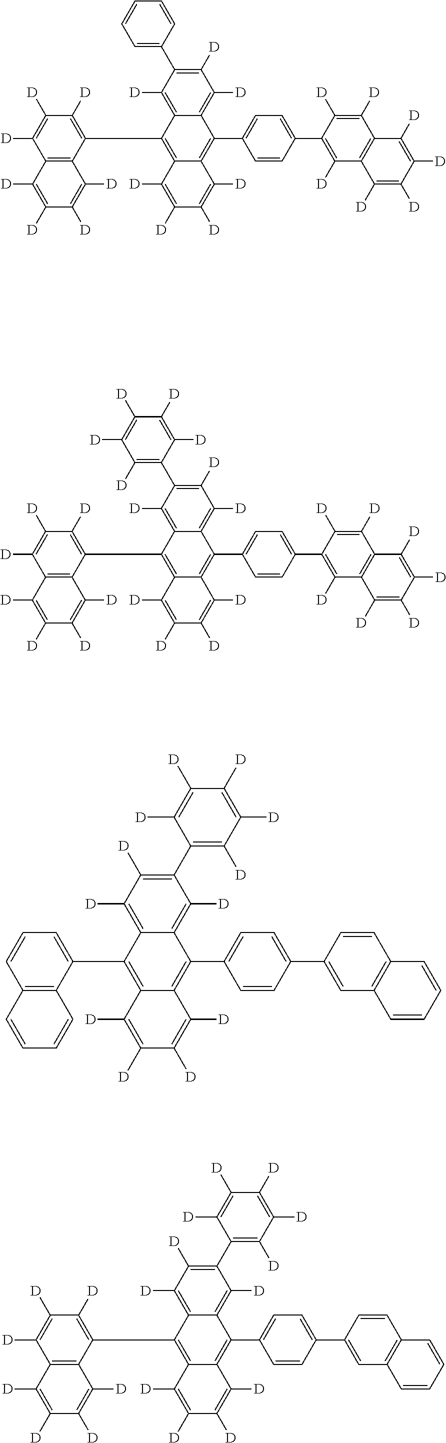

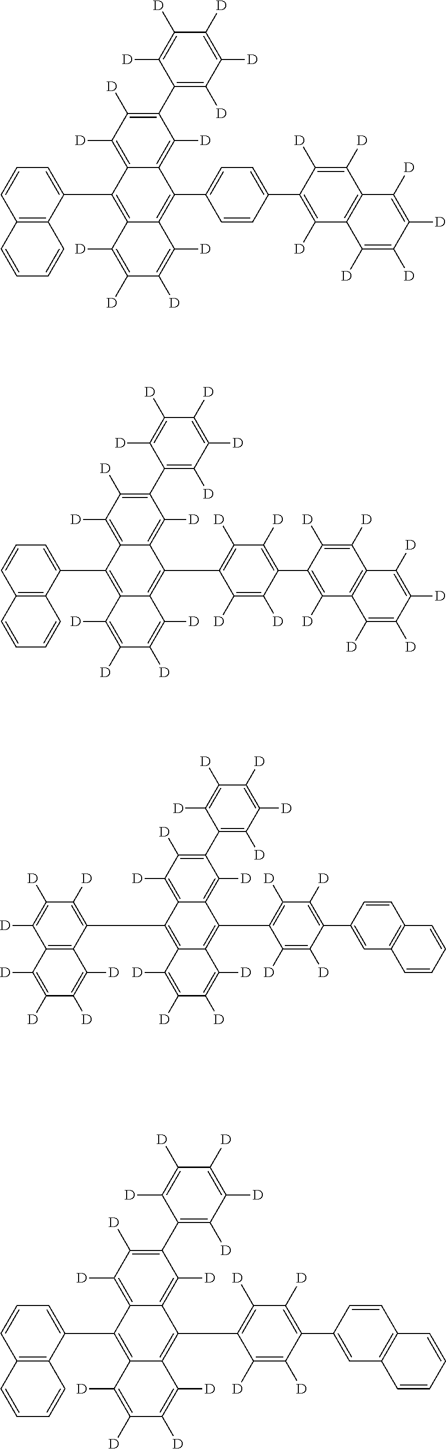

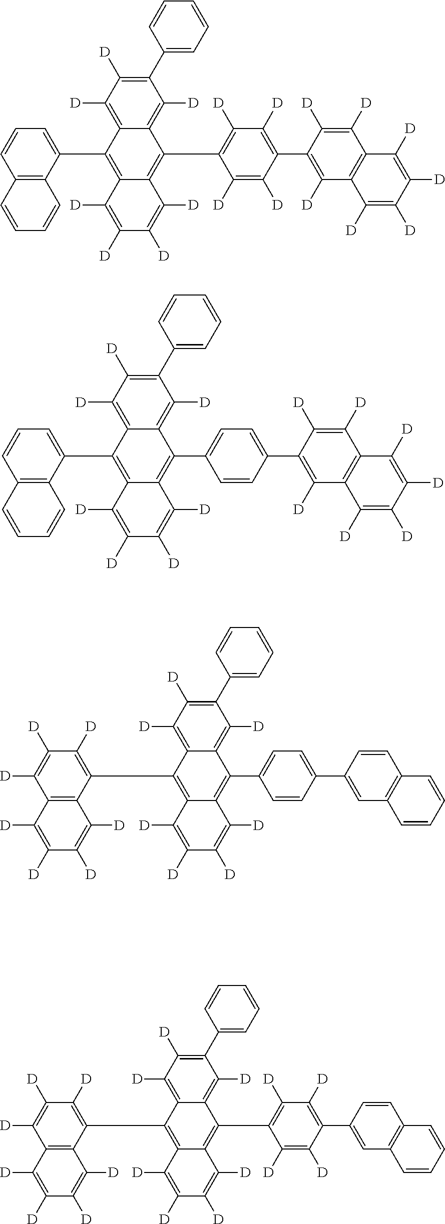

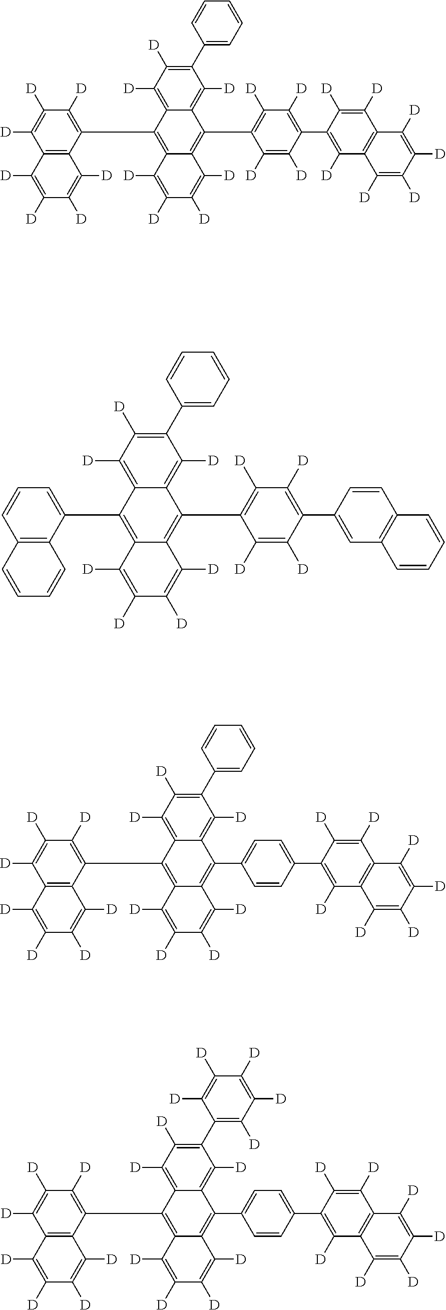

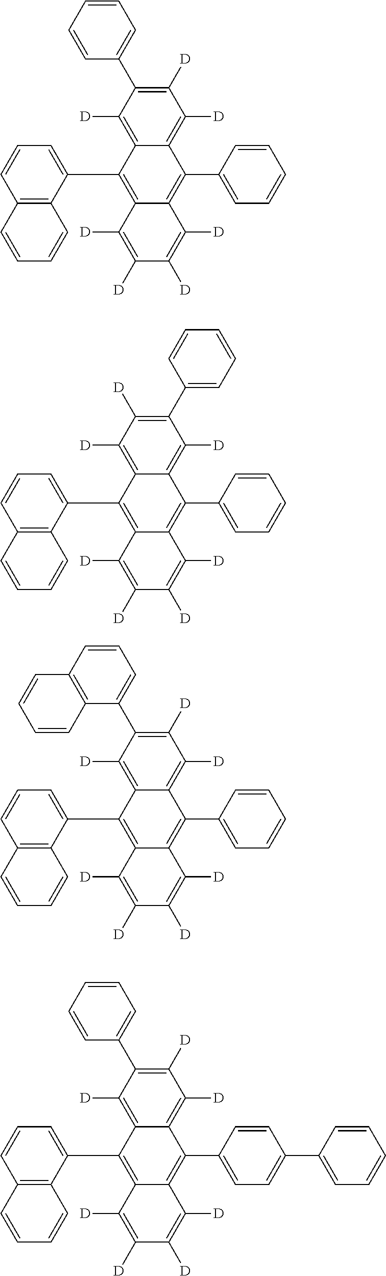

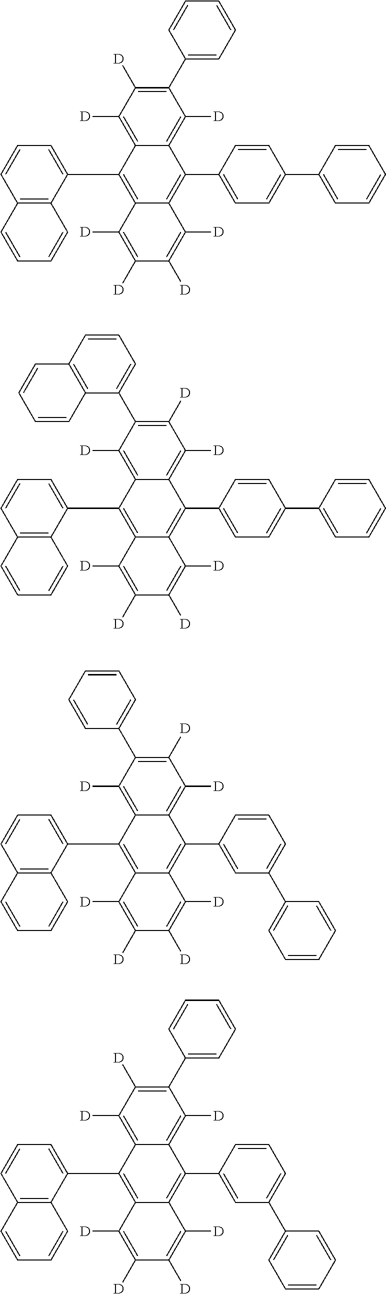

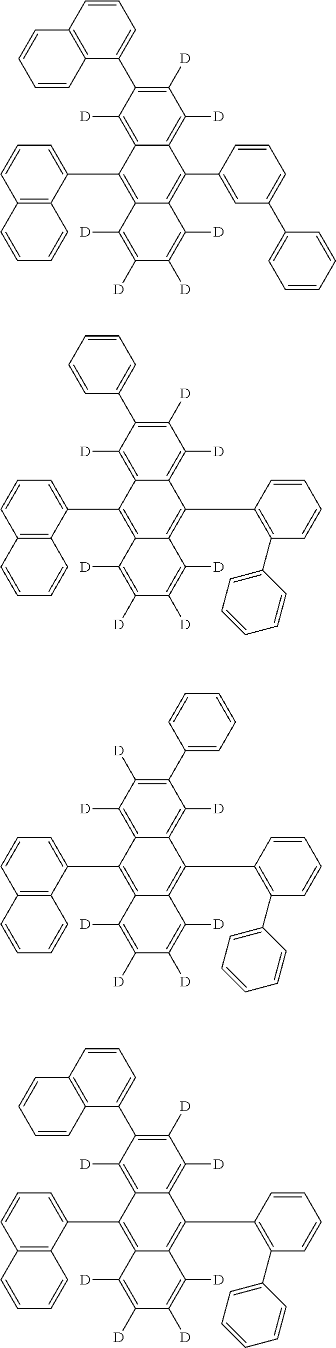

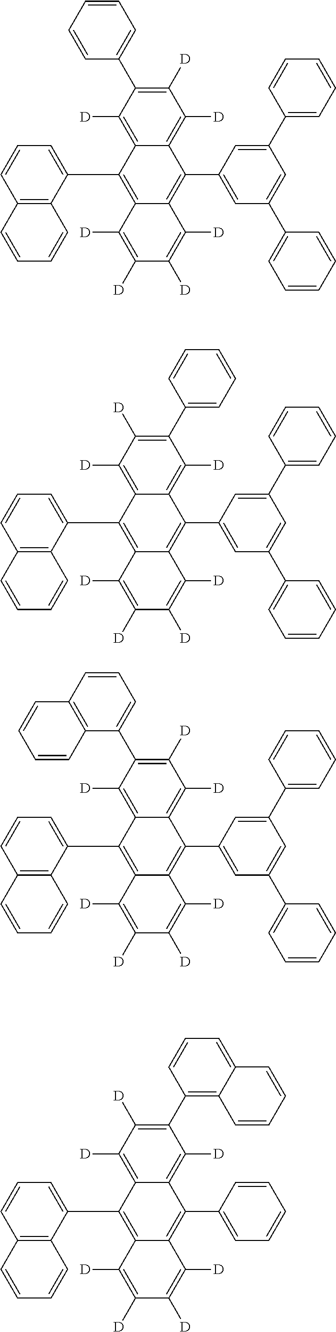

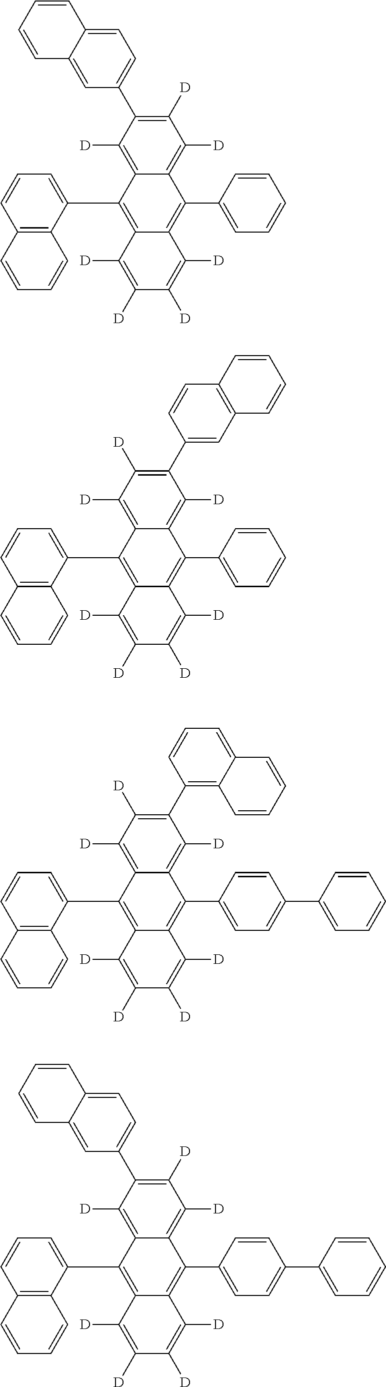 [SMILES: [2H]c1c([2H])c([2H])c2c(-c3cccc4ccccc34)c3c([2H])c(-c4ccc5ccccc5c4)c([2H])c([2H])c3c(-c3ccccc3)c2c1[2H] ; [2H]c1c([2H])c([2H])c2c(-c3cccc4ccccc34)c3c([2H])c([2H])c(-c4ccc5ccccc5c4)c([2H])c3c(-c3ccccc3)c2c1[2H] ; [2H]c1c([2H])c([2H])c2c(-c3cccc4ccccc34)c3c([2H])c([2H])c(-c4cccc5ccccc45)c([2H])c3c(-c3ccc(-c4ccccc4)cc3)c2c1[2H] ; [2H]c1c([2H])c([2H])c2c(-c3cccc4ccccc34)c3c([2H])c(-c4ccc5ccccc5c4)c([2H])c([2H])c3c(-c3ccc(-c4ccccc4)cc3)c2c1[2H]]

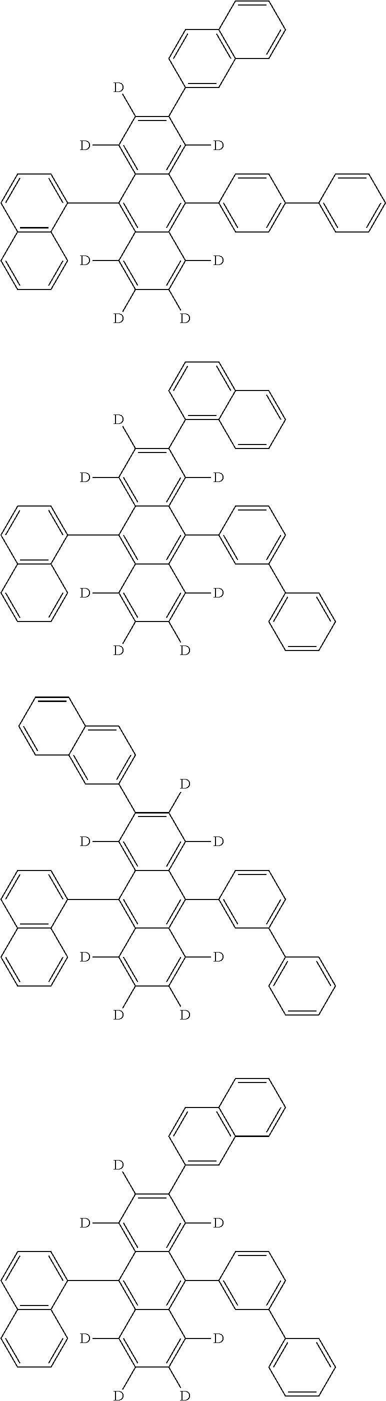

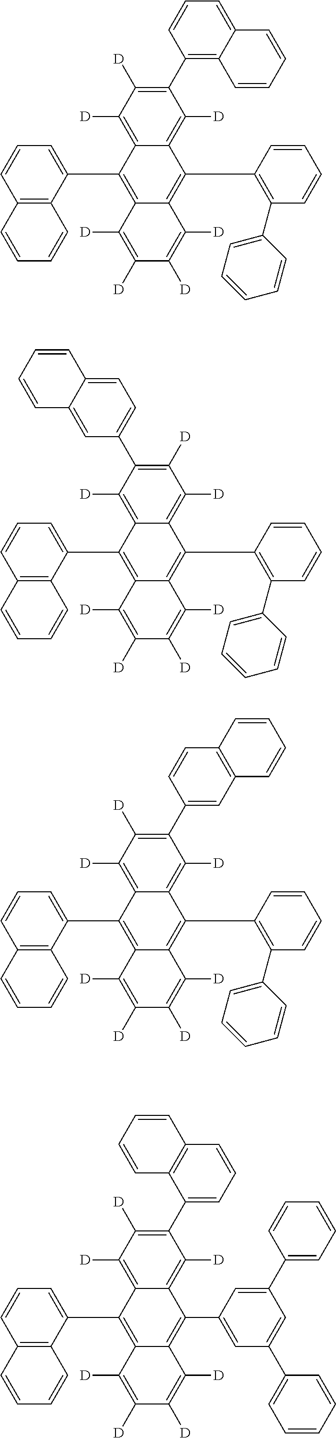

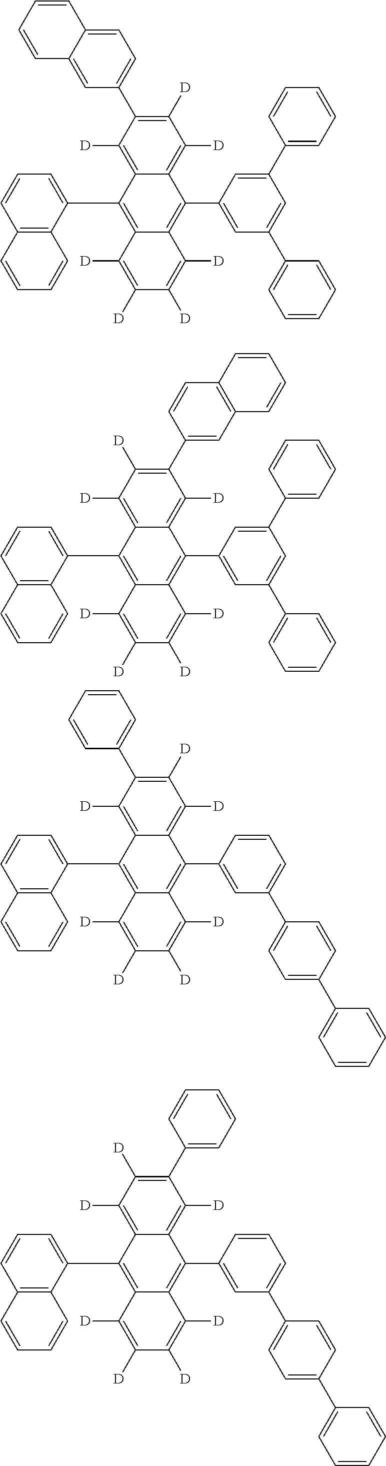

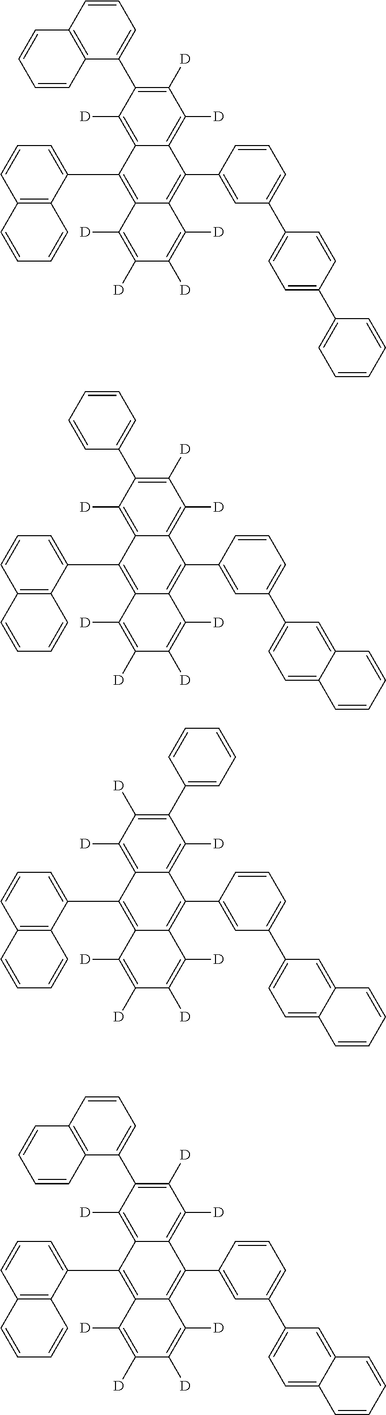 [SMILES: [2H]c1c([2H])c([2H])c2c(-c3cccc4ccccc34)c3c([2H])c(-c4cccc5ccccc45)c([2H])c([2H])c3c(-c3cccc(-c4ccc(-c5ccccc5)cc4)c3)c2c1[2H] ; [2H]c1c([2H])c([2H])c2c(-c3cccc4ccccc34)c3c([2H])c(-c4ccccc4)c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c(-c4ccccc4)c([2H])c3c(-c3cccc(-c4ccc5ccccc5c4)c3)c2c1[2H] ; [2H]c1c([2H])c([2H])c2c(-c3cccc4ccccc34)c3c([2H])c(-c4cccc5ccccc45)c([2H])c([2H])c3c(-c3cccc(-c4ccc5ccccc5c4)c3)c2c1[2H]]

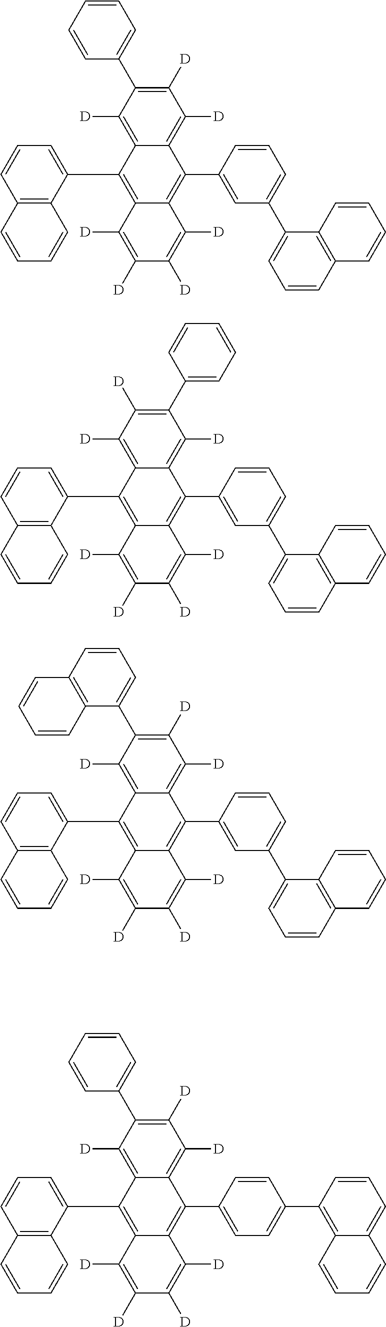

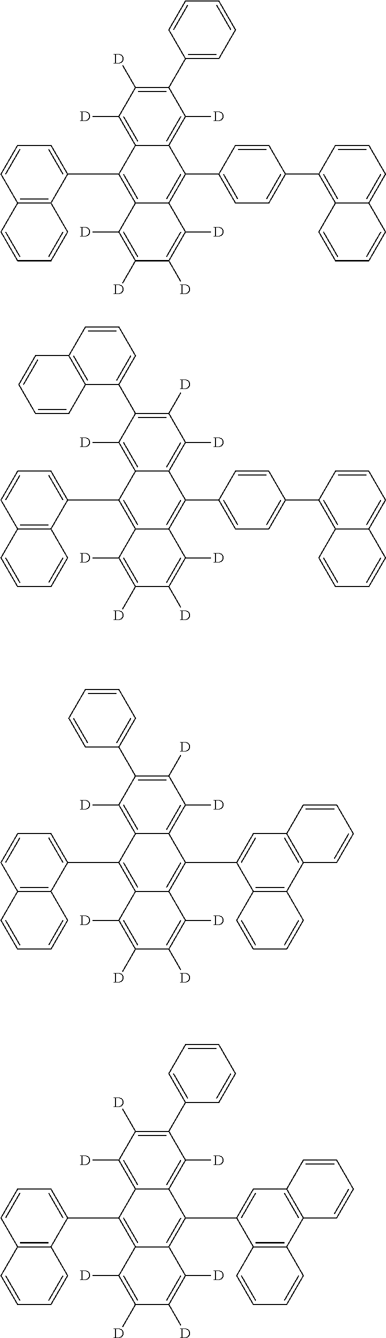

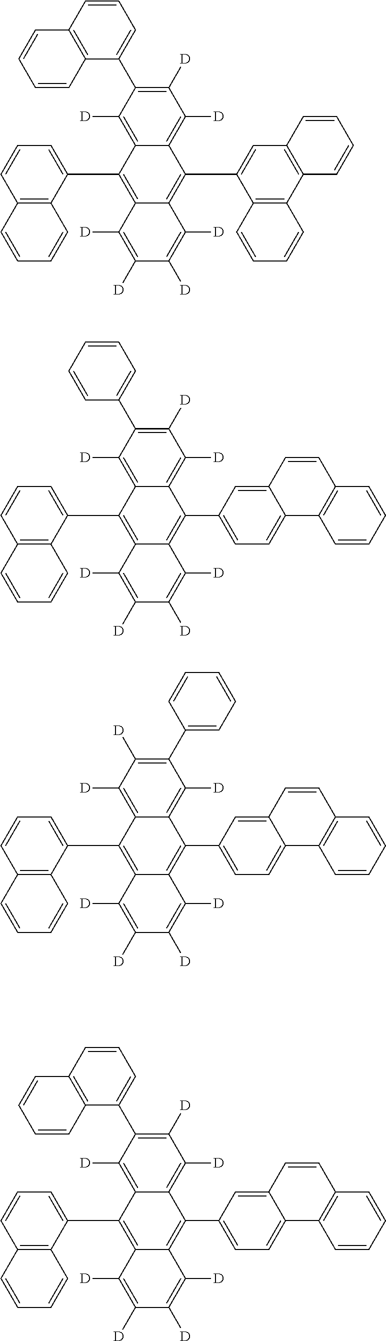

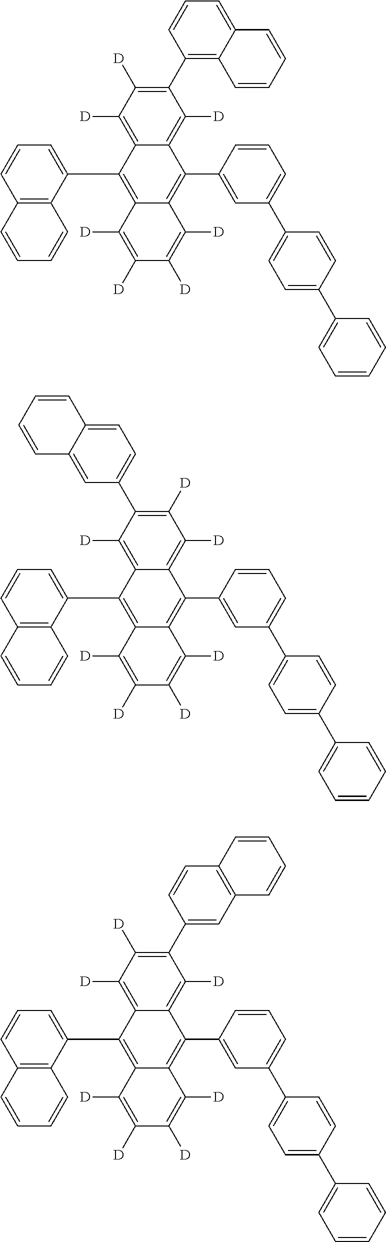 [SMILES: [2H]c1c([2H])c([2H])c2c(-c3cccc4ccccc34)c3c([2H])c([2H])c(-c4cccc5ccccc45)c([2H])c3c(-c3cccc(-c4ccc(-c5ccccc5)cc4)c3)c2c1[2H] ; [2H]c1c([2H])c([2H])c2c(-c3cccc4ccccc34)c3c([2H])c(-c4ccc5ccccc5c4)c([2H])c([2H])c3c(-c3cccc(-c4ccc(-c5ccccc5)cc4)c3)c2c1[2H] ; [2H]c1c([2H])c([2H])c2c(-c3cccc4ccccc34)c3c([2H])c([2H])c(-c4ccc5ccccc5c4)c([2H])c3c(-c3cccc(-c4ccc(-c5ccccc5)cc4)c3)c2c1[2H]]

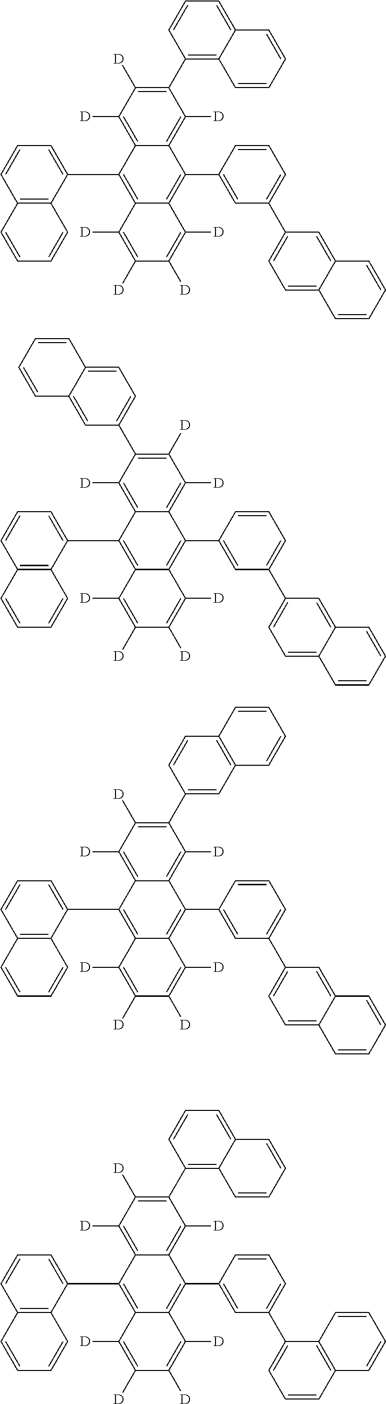 [SMILES: [2H]c1c([2H])c([2H])c2c(-c3cccc4ccccc34)c3c([2H])c([2H])c(-c4cccc5ccccc45)c([2H])c3c(-c3cccc(-c4ccc5ccccc5c4)c3)c2c1[2H] ; [2H]c1c([2H])c([2H])c2c(-c3cccc4ccccc34)c3c([2H])c(-c4ccc5ccccc5c4)c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c(-c4ccc5ccccc5c4)c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c(-c4cccc5ccccc45)c([2H])c3c(-c3cccc(-c4cccc5ccccc45)c3)c2c1[2H]]

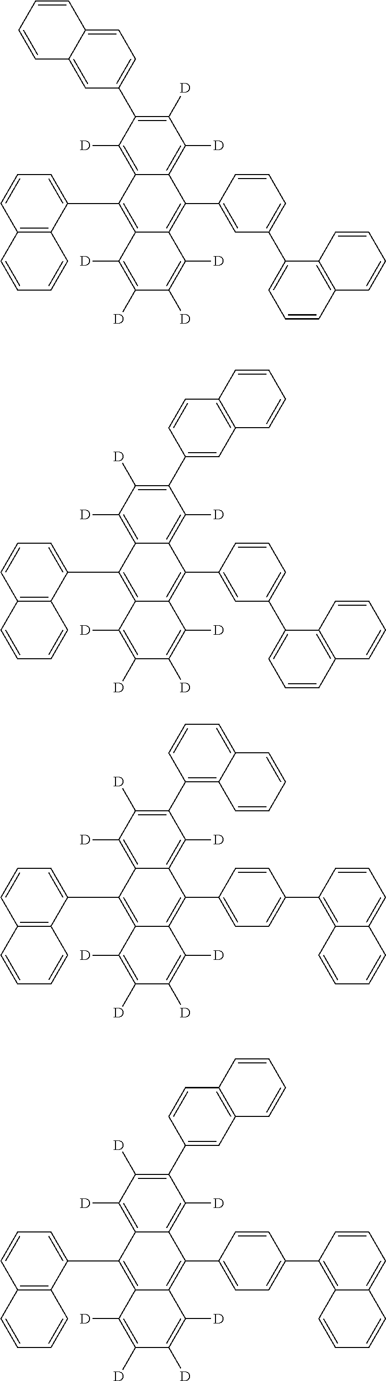

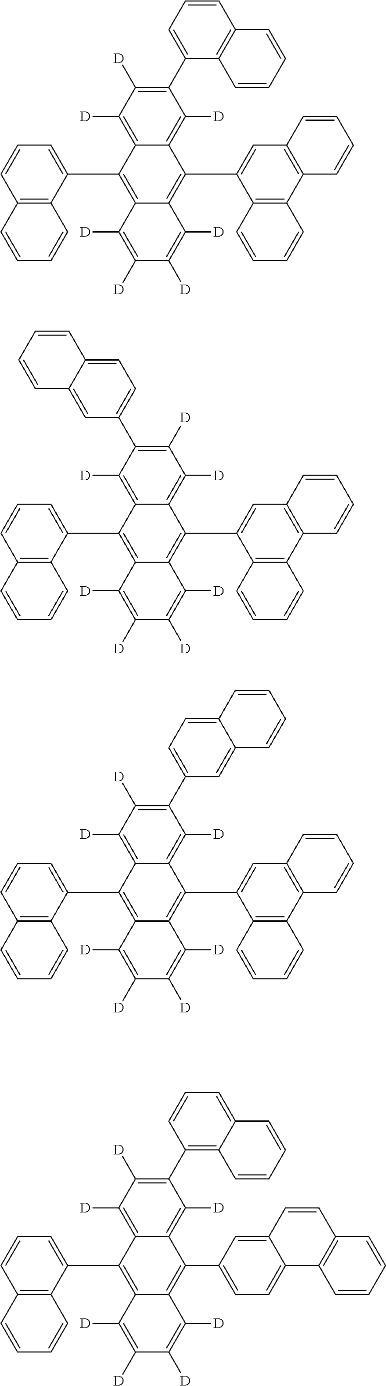 [SMILES: [2H]c1c([2H])c([2H])c2c(-c3cc4ccccc4c4ccccc34)c3c([2H])c(-c4cccc5ccccc45)c([2H])c([2H])c3c(-c3cccc4ccccc34)c2c1[2H] ; [2H]c1c([2H])c([2H])c2c(-c3cc4ccccc4c4ccccc34)c3c([2H])c([2H])c(-c4ccc5ccccc5c4)c([2H])c3c(-c3cccc4ccccc34)c2c1[2H] ; [2H]c1c([2H])c([2H])c2c(-c3cc4ccccc4c4ccccc34)c3c([2H])c(-c4ccc5ccccc5c4)c([2H])c([2H])c3c(-c3cccc4ccccc34)c2c1[2H] ; [2H]c1c([2H])c([2H])c2c(-c3cccc4ccccc34)c3c([2H])c([2H])c(-c4cccc5ccccc45)c([2H])c3c(-c3ccc4c(ccc5ccccc54)c3)c2c1[2H]]

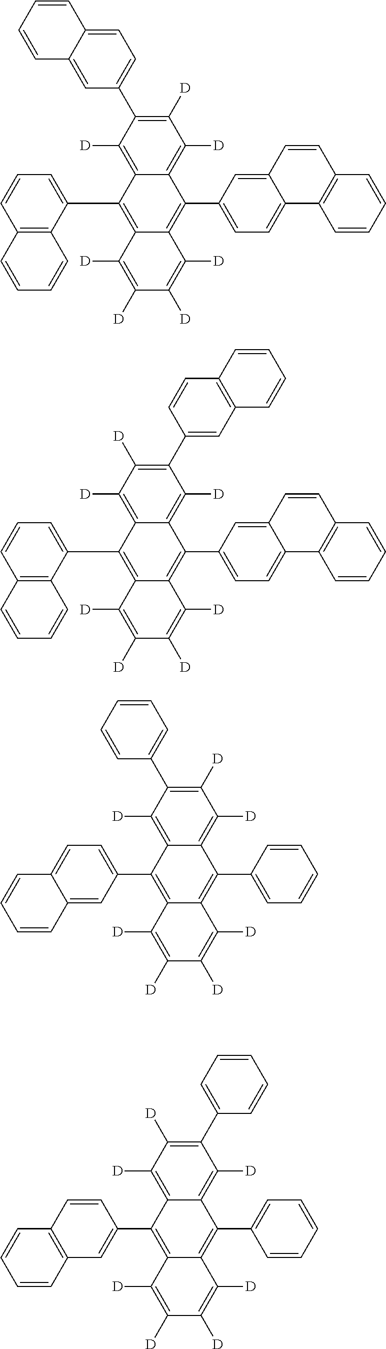

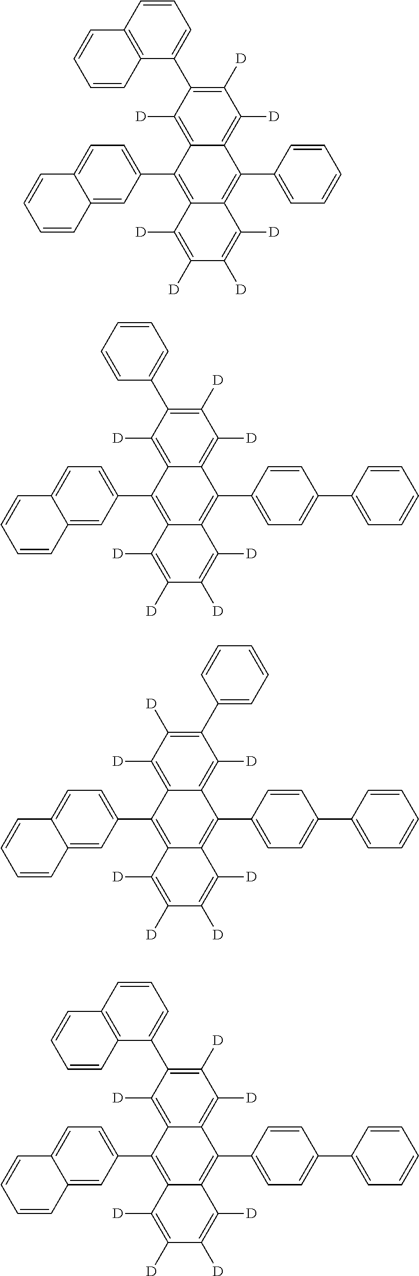

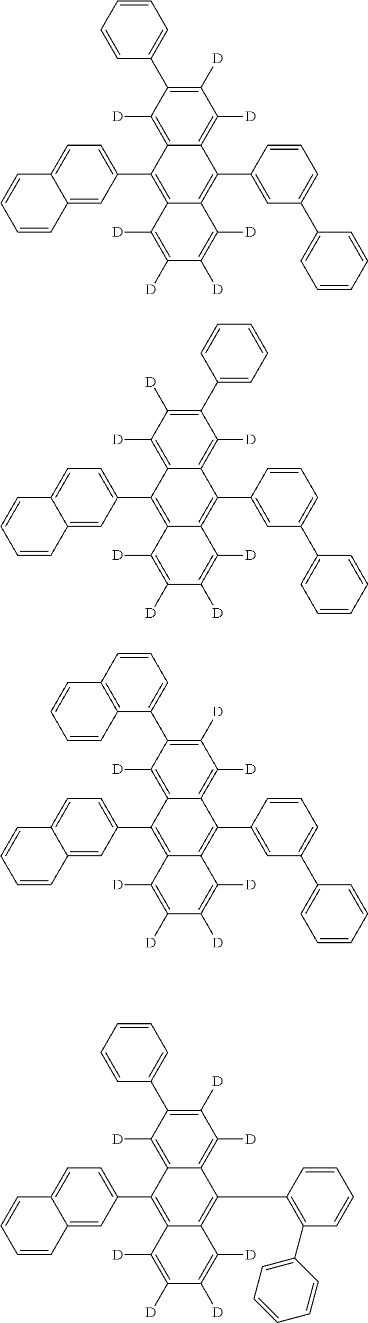

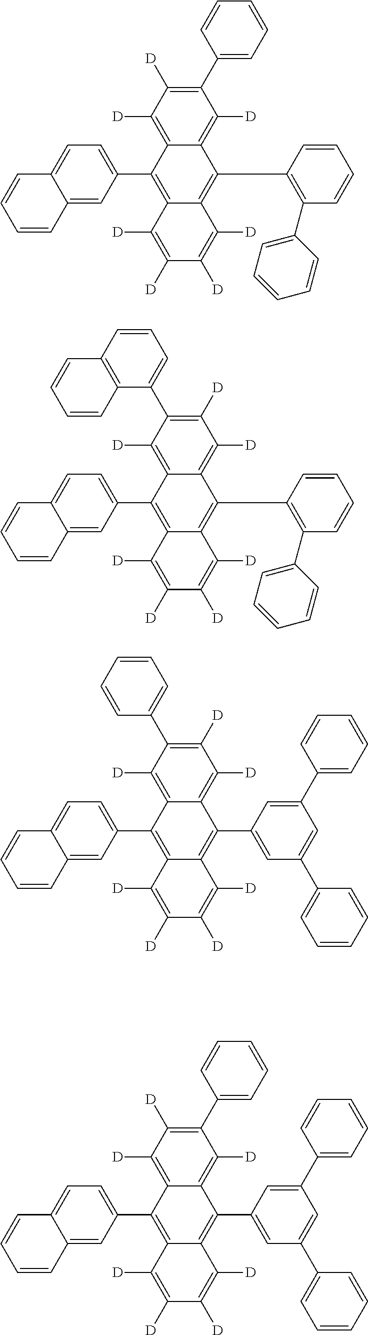

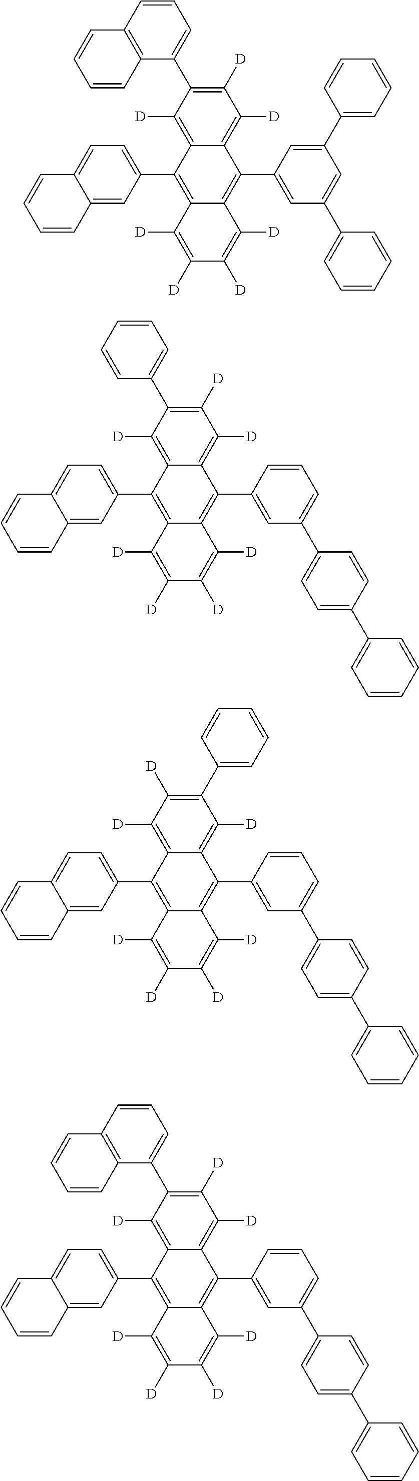

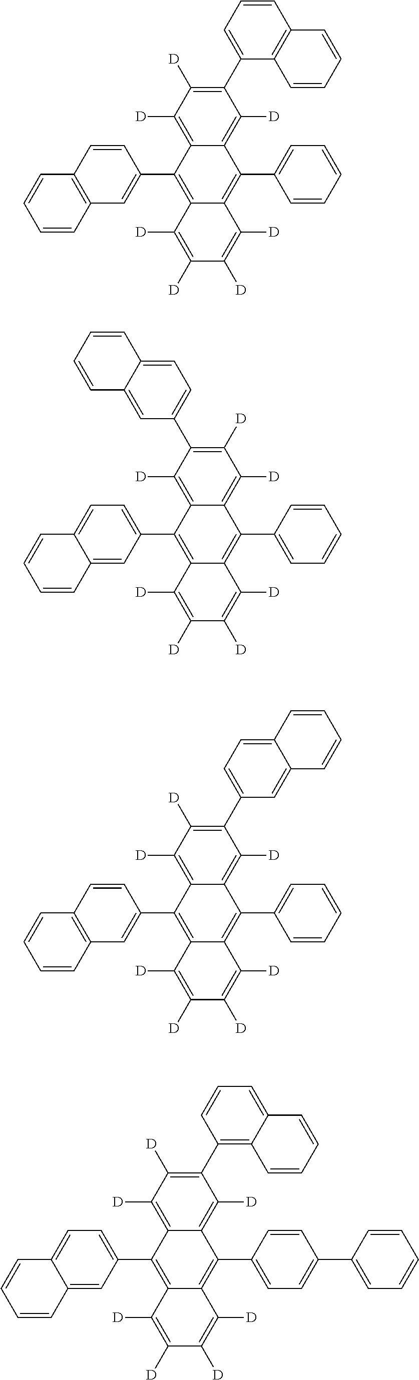 [SMILES: [2H]c1c([2H])c([2H])c2c(-c3ccccc3)c3c([2H])c(-c4cccc5ccccc45)c([2H])c([2H])c3c(-c3ccc4ccccc4c3)c2c1[2H] ; [2H]c1c([2H])c([2H])c2c(-c3ccc4ccccc4c3)c3c([2H])c(-c4ccc5ccccc5c4)c([2H])c([2H])c3c(-c3ccccc3)c2c1[2H] ; [2H]c1c([2H])c([2H])c2c(-c3ccccc3)c3c([2H])c(-c4ccc5ccccc5c4)c([2H])c([2H])c3c(-c3ccc4ccccc4c3)c2c1[2H] ; [2H]c1c([2H])c([2H])c2c(-c3ccc(-c4ccccc4)cc3)c3c([2H])c(-c4cccc5ccccc45)c([2H])c([2H])c3c(-c3ccc4ccccc4c3)c2c1[2H]]

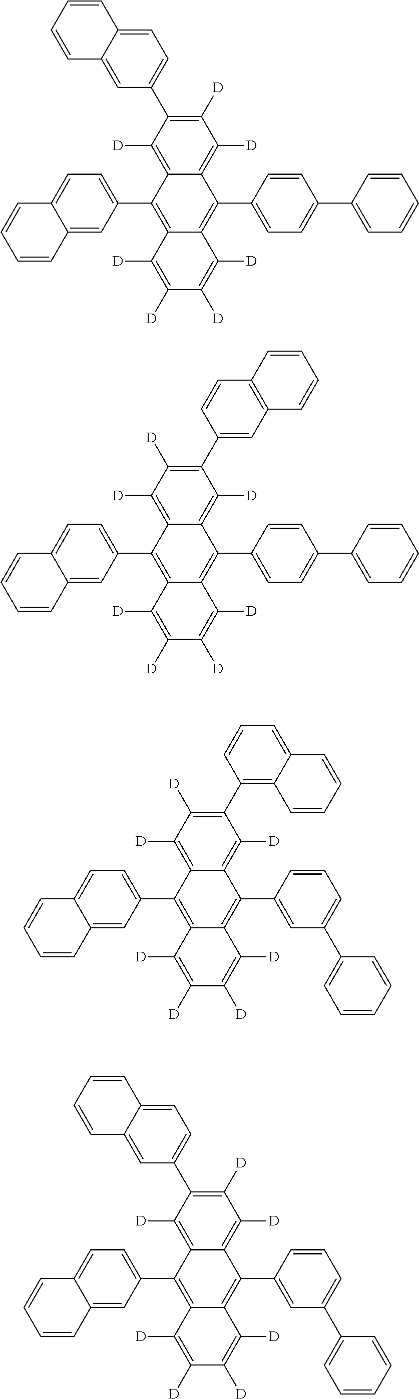 [SMILES: [2H]c1c([2H])c([2H])c2c(-c3ccc4ccccc4c3)c3c([2H])c(-c4ccc5ccccc5c4)c([2H])c([2H])c3c(-c3ccc(-c4ccccc4)cc3)c2c1[2H] ; [2H]c1c([2H])c([2H])c2c(-c3ccc(-c4ccccc4)cc3)c3c([2H])c(-c4ccc5ccccc5c4)c([2H])c([2H])c3c(-c3ccc4ccccc4c3)c2c1[2H] ; [2H]c1c([2H])c([2H])c2c(-c3cccc(-c4ccccc4)c3)c3c([2H])c(-c4cccc5ccccc45)c([2H])c([2H])c3c(-c3ccc4ccccc4c3)c2c1[2H] ; [2H]c1c([2H])c([2H])c2c(-c3ccc4ccccc4c3)c3c([2H])c(-c4ccc5ccccc5c4)c([2H])c([2H])c3c(-c3cccc(-c4ccccc4)c3)c2c1[2H]]

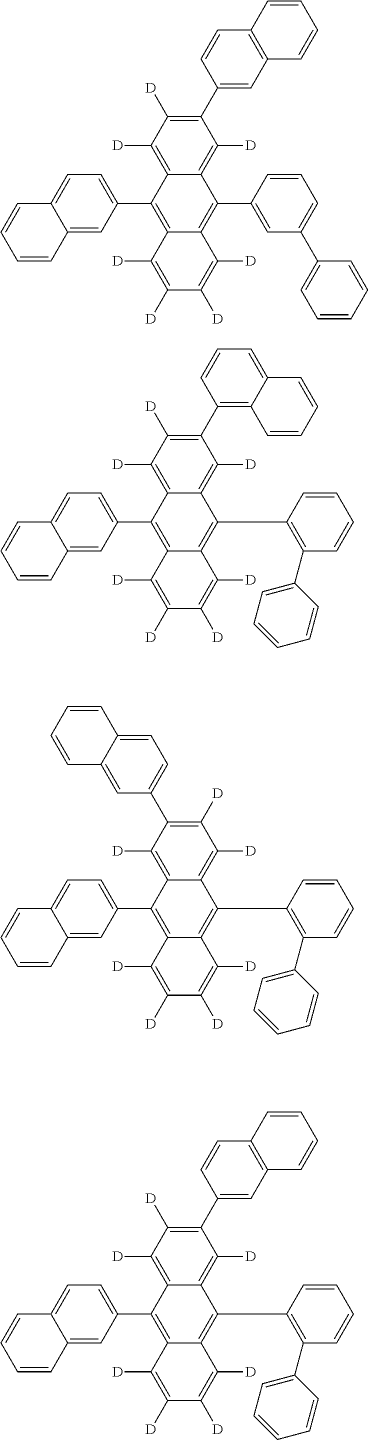

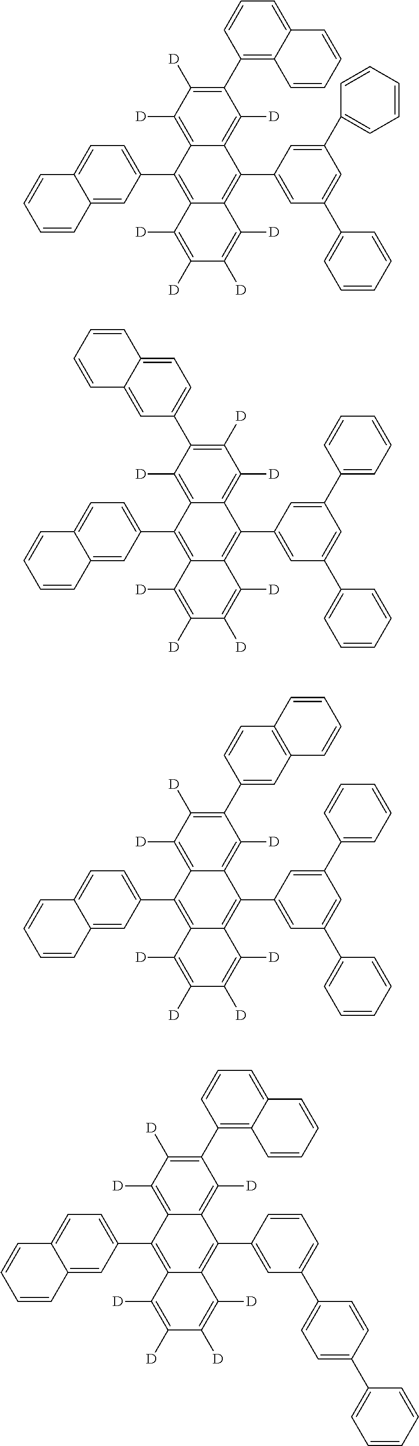

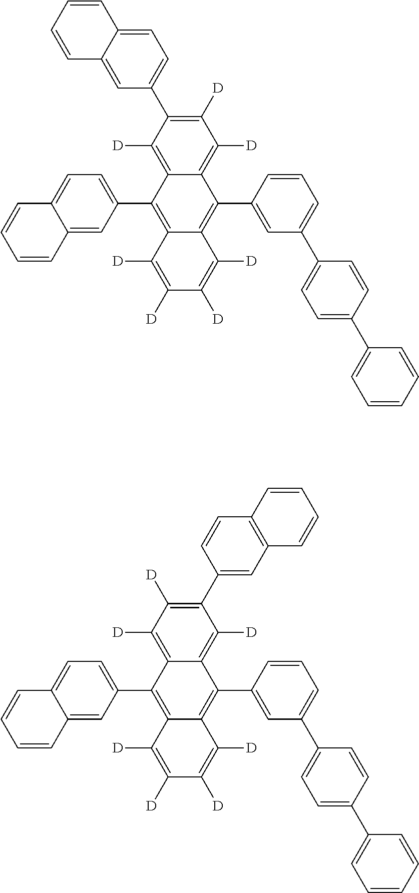 [SMILES: [2H]c1c([2H])c([2H])c2c(-c3ccc4ccccc4c3)c3c([2H])c(-c4ccc5ccccc5c4)c([2H])c([2H])c3c(-c3cccc(-c4ccc(-c5ccccc5)cc4)c3)c2c1[2H] ; [2H]c1c([2H])c([2H])c2c(-c3cccc(-c4ccc(-c5ccccc5)cc4)c3)c3c([2H])c(-c4ccc5ccccc5c4)c([2H])c([2H])c3c(-c3ccc4ccccc4c3)c2c1[2H]]

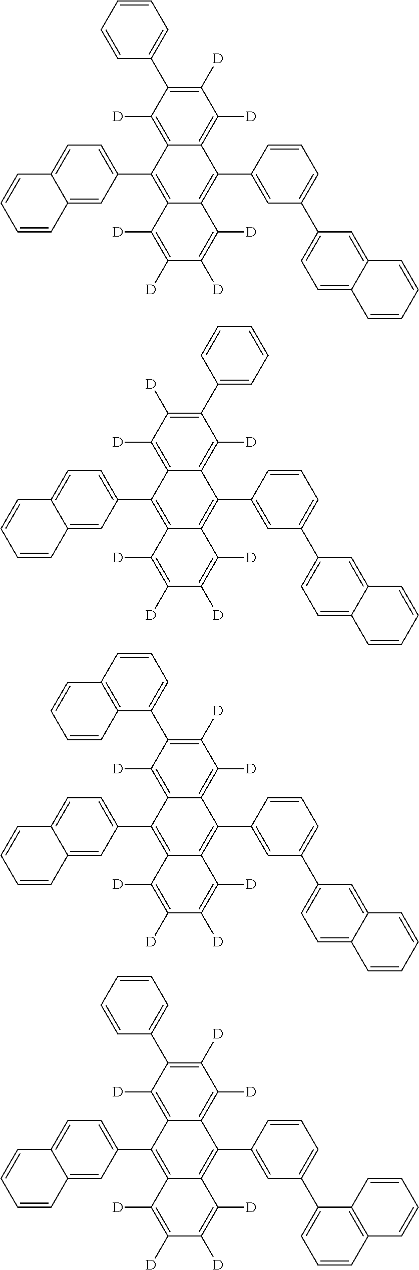

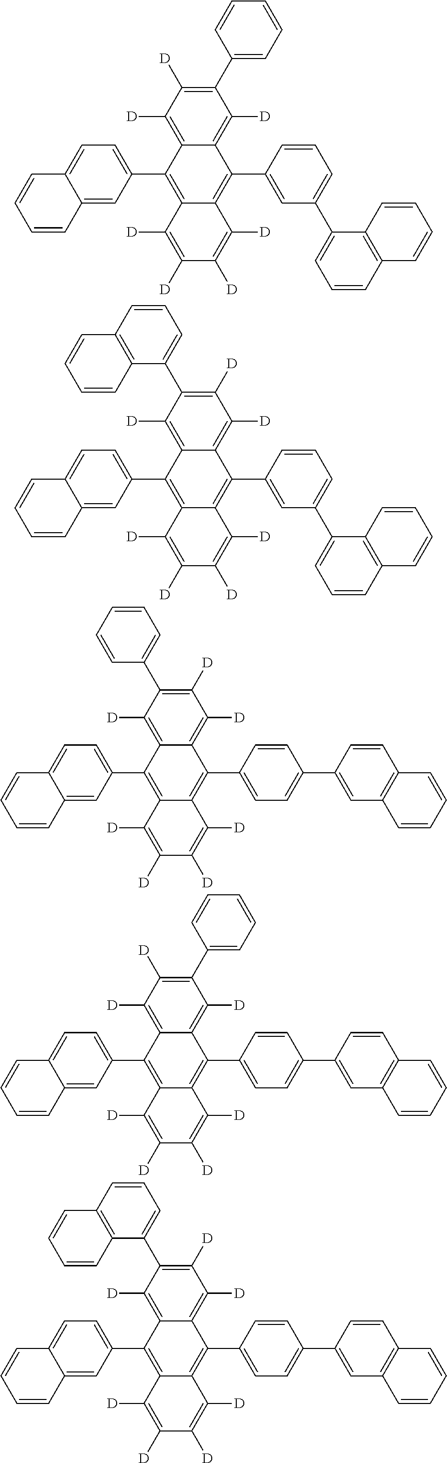

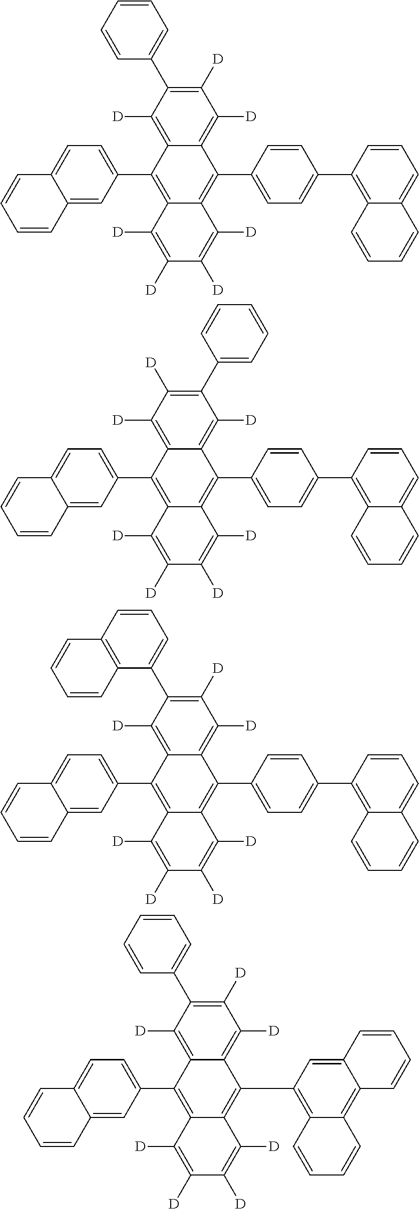

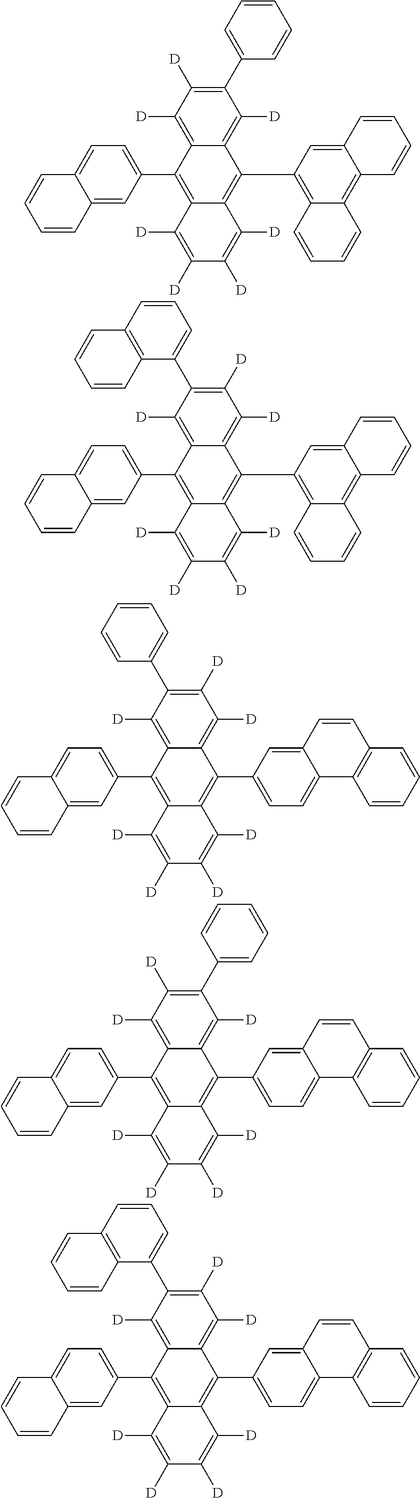

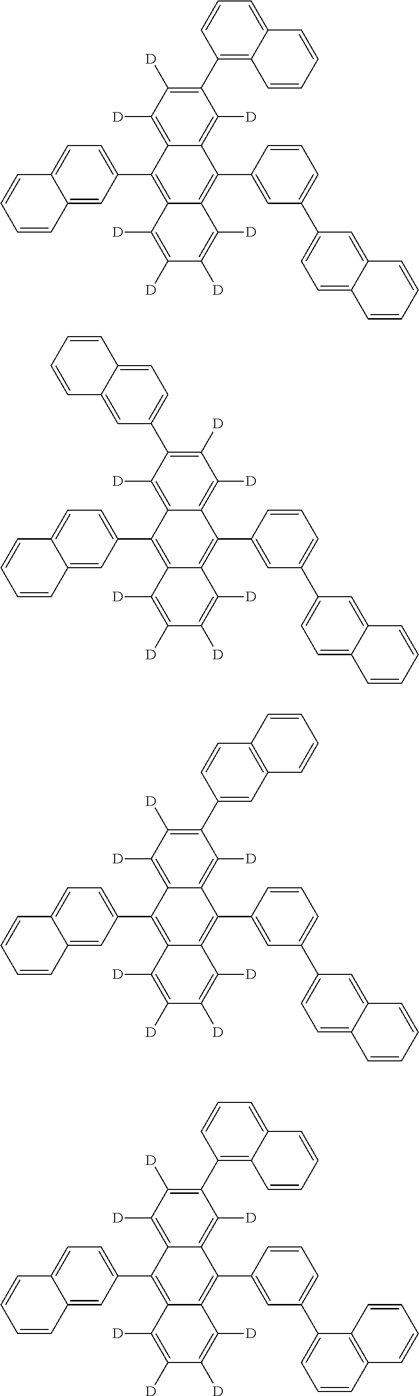 [SMILES: [2H]c1c([2H])c([2H])c2c(-c3cccc(-c4ccc5ccccc5c4)c3)c3c([2H])c(-c4cccc5ccccc45)c([2H])c([2H])c3c(-c3ccc4ccccc4c3)c2c1[2H] ; [2H]c1c([2H])c([2H])c2c(-c3ccc4ccccc4c3)c3c([2H])c(-c4ccc5ccccc5c4)c([2H])c([2H])c3c(-c3cccc(-c4ccc5ccccc5c4)c3)c2c1[2H] ; [2H]c1c([2H])c([2H])c2c(-c3cccc(-c4ccc5ccccc5c4)c3)c3c([2H])c(-c4ccc5ccccc5c4)c([2H])c([2H])c3c(-c3ccc4ccccc4c3)c2c1[2H] ; [2H]c1c([2H])c([2H])c2c(-c3cccc(-c4cccc5ccccc45)c3)c3c([2H])c(-c4cccc5ccccc45)c([2H])c([2H])c3c(-c3ccc4ccccc4c3)c2c1[2H]]

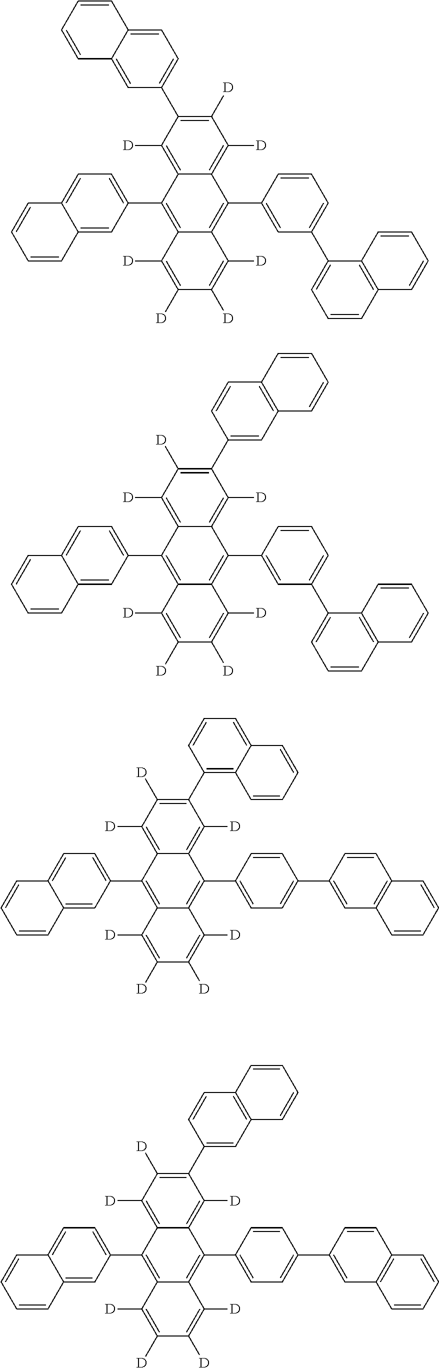

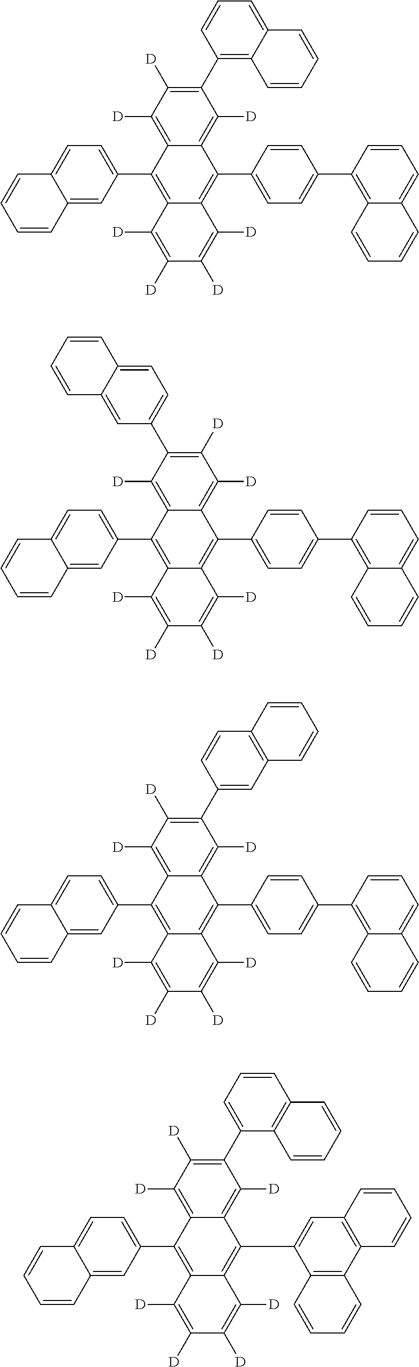 [SMILES: [2H]c1c([2H])c([2H])c2c(-c3ccc(-c4cccc5ccccc45)cc3)c3c([2H])c(-c4cccc5ccccc45)c([2H])c([2H])c3c(-c3ccc4ccccc4c3)c2c1[2H] ; [2H]c1c([2H])c([2H])c2c(-c3ccc4ccccc4c3)c3c([2H])c(-c4ccc5ccccc5c4)c([2H])c([2H])c3c(-c3ccc(-c4cccc5ccccc45)cc3)c2c1[2H] ; [2H]c1c([2H])c([2H])c2c(-c3ccc(-c4cccc5ccccc45)cc3)c3c([2H])c(-c4ccc5ccccc5c4)c([2H])c([2H])c3c(-c3ccc4ccccc4c3)c2c1[2H] ; [2H]c1c([2H])c([2H])c2c(-c3cc4ccccc4c4ccccc34)c3c([2H])c(-c4cccc5ccccc45)c([2H])c([2H])c3c(-c3ccc4ccccc4c3)c2c1[2H]]

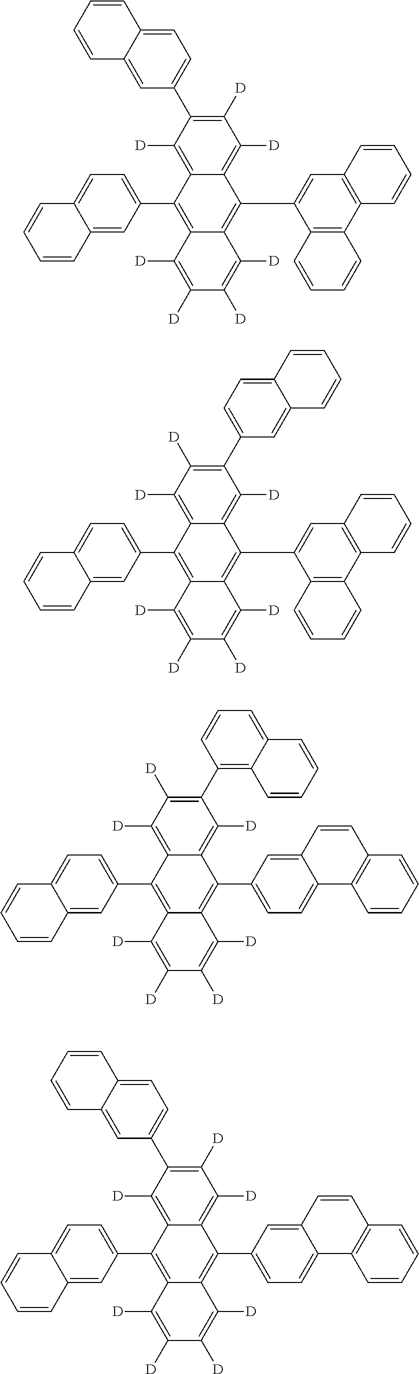

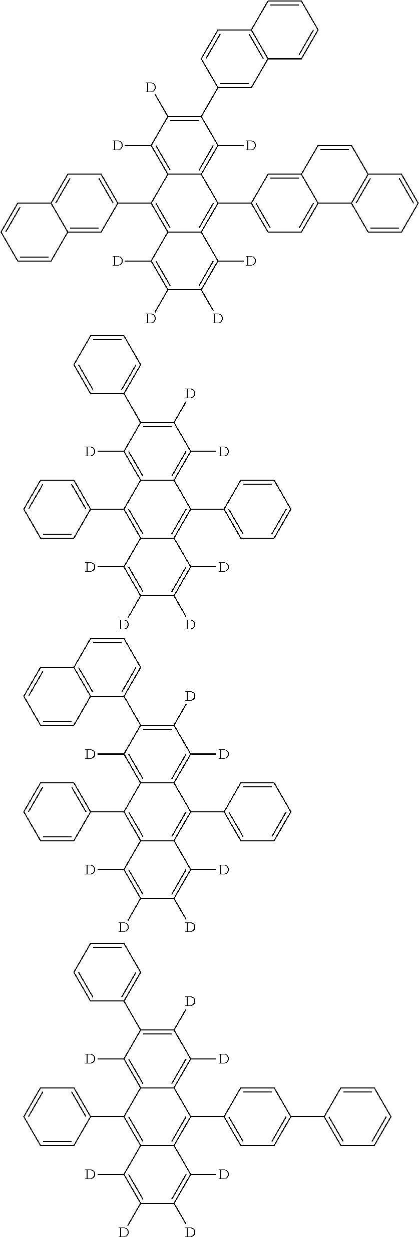

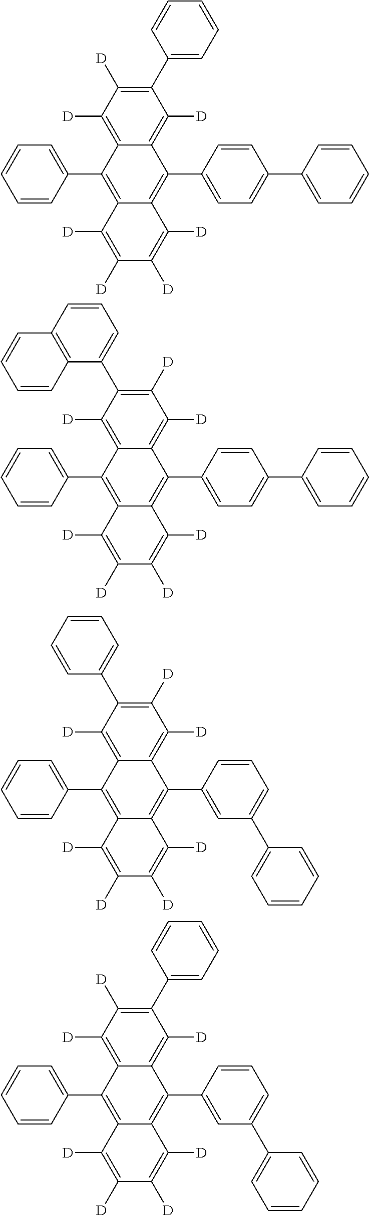

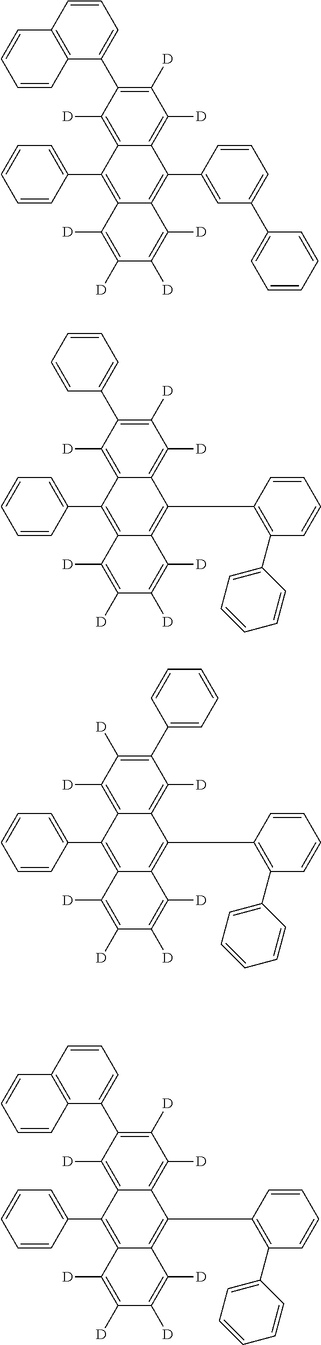

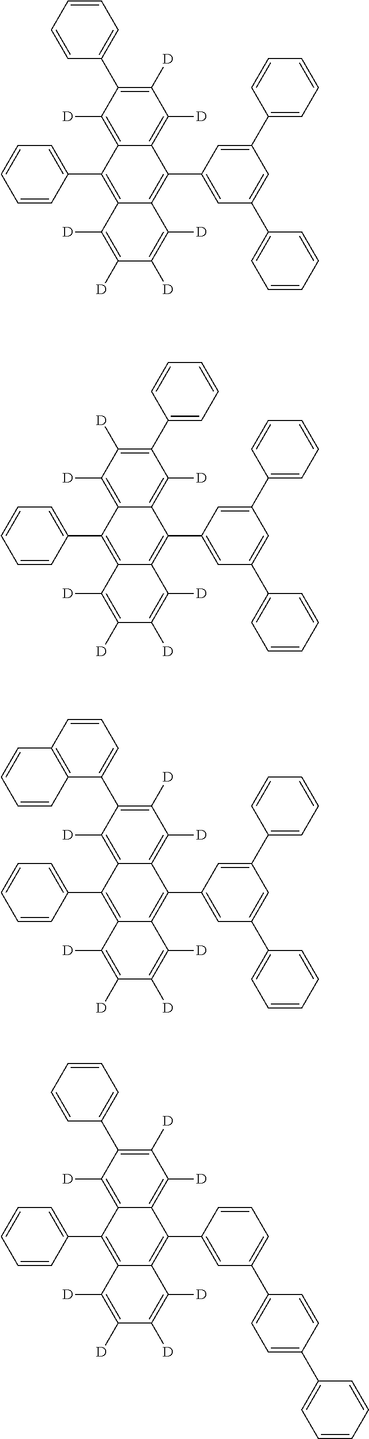 [SMILES: [2H]c1c([2H])c([2H])c2c(-c3ccccc3)c3c([2H])c(-c4ccccc4)c([2H])c([2H])c3c(-c3cc(-c4ccccc4)cc(-c4ccccc4)c3)c2c1[2H] ; [2H]c1c([2H])c([2H])c2c(-c3cc(-c4ccccc4)cc(-c4ccccc4)c3)c3c([2H])c(-c4ccccc4)c([2H])c([2H])c3c(-c3ccccc3)c2c1[2H] ; [2H]c1c([2H])c([2H])c2c(-c3ccccc3)c3c([2H])c(-c4cccc5ccccc45)c([2H])c([2H])c3c(-c3cc(-c4ccccc4)cc(-c4ccccc4)c3)c2c1[2H] ; [2H]c1c([2H])c([2H])c2c(-c3ccccc3)c3c([2H])c(-c4ccccc4)c([2H])c([2H])c3c(-c3cccc(-c4ccc(-c5ccccc5)cc4)c3)c2c1[2H]]

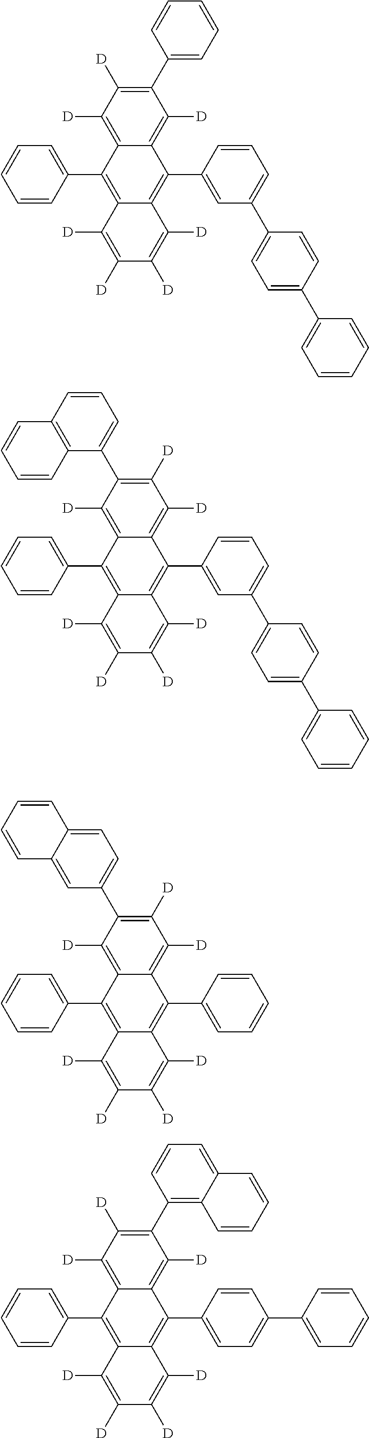

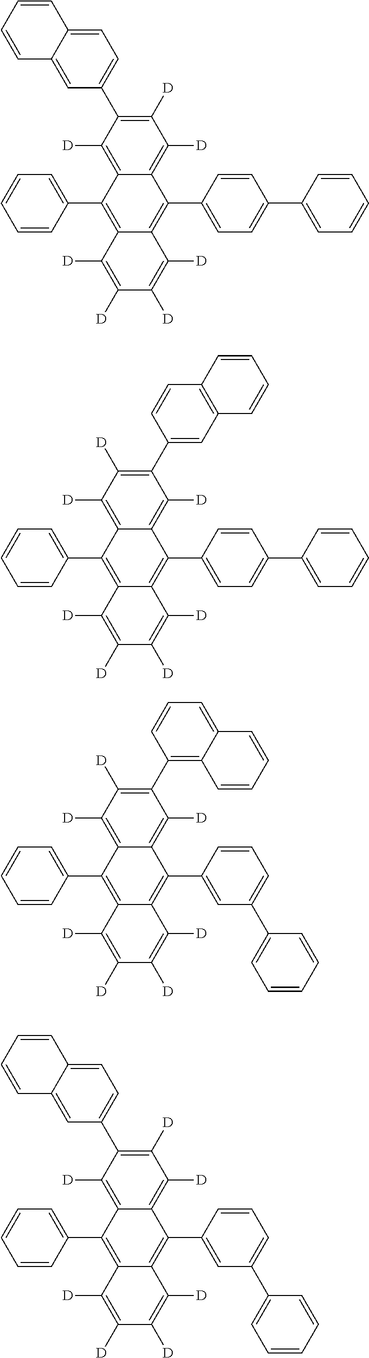

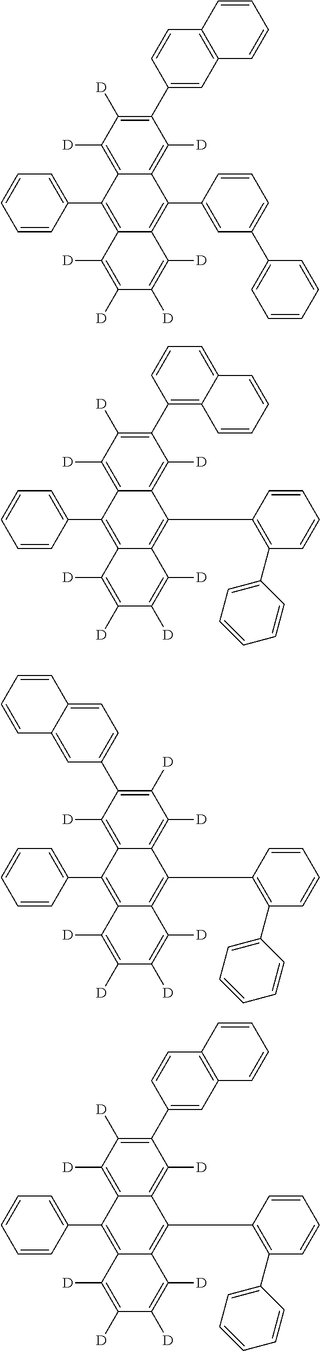

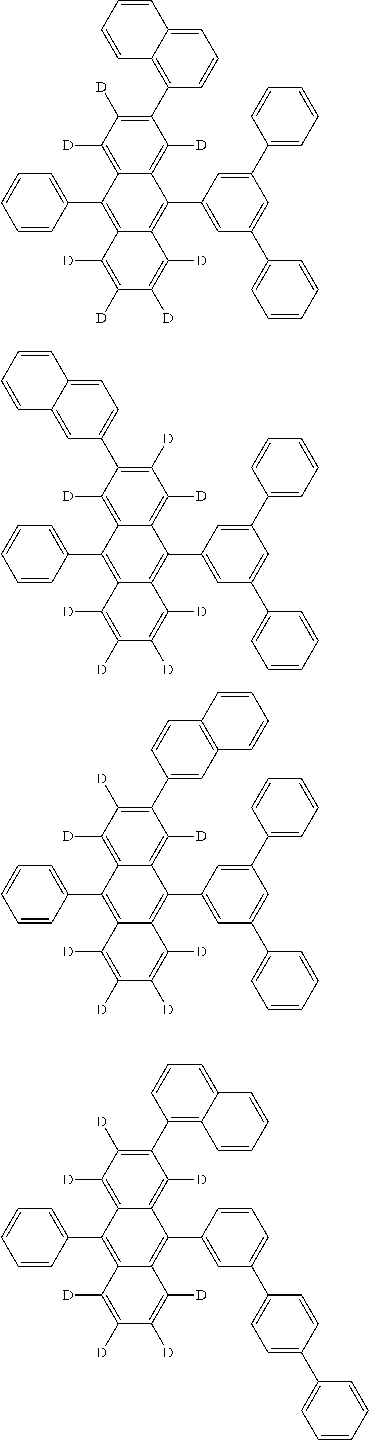

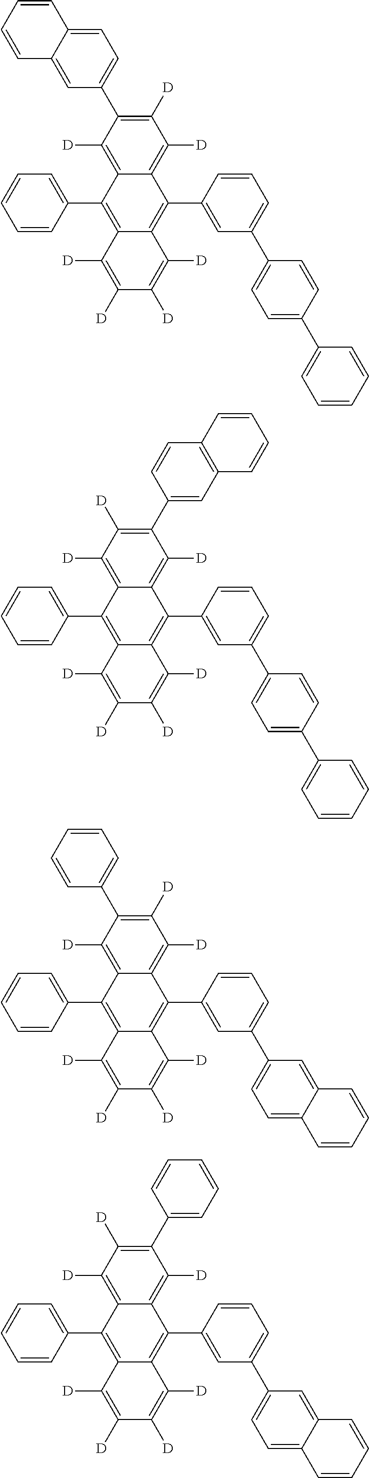

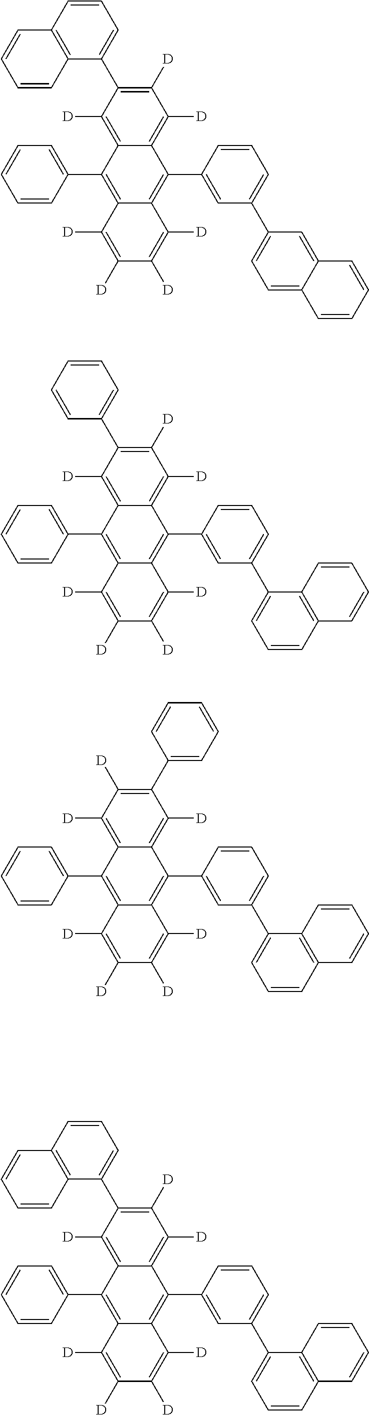 [SMILES: [2H]c1c([2H])c([2H])c2c(-c3ccccc3)c3c([2H])c(-c4cccc5ccccc45)c([2H])c([2H])c3c(-c3cccc(-c4ccc5ccccc5c4)c3)c2c1[2H] ; [2H]c1c([2H])c([2H])c2c(-c3ccccc3)c3c([2H])c(-c4ccccc4)c([2H])c([2H])c3c(-c3cccc(-c4cccc5ccccc45)c3)c2c1[2H] ; [2H]c1c([2H])c([2H])c2c(-c3cccc(-c4cccc5ccccc45)c3)c3c([2H])c(-c4ccccc4)c([2H])c([2H])c3c(-c3ccccc3)c2c1[2H] ; [2H]c1c([2H])c([2H])c2c(-c3ccccc3)c3c([2H])c(-c4cccc5ccccc45)c([2H])c([2H])c3c(-c3cccc(-c4cccc5ccccc45)c3)c2c1[2H]]

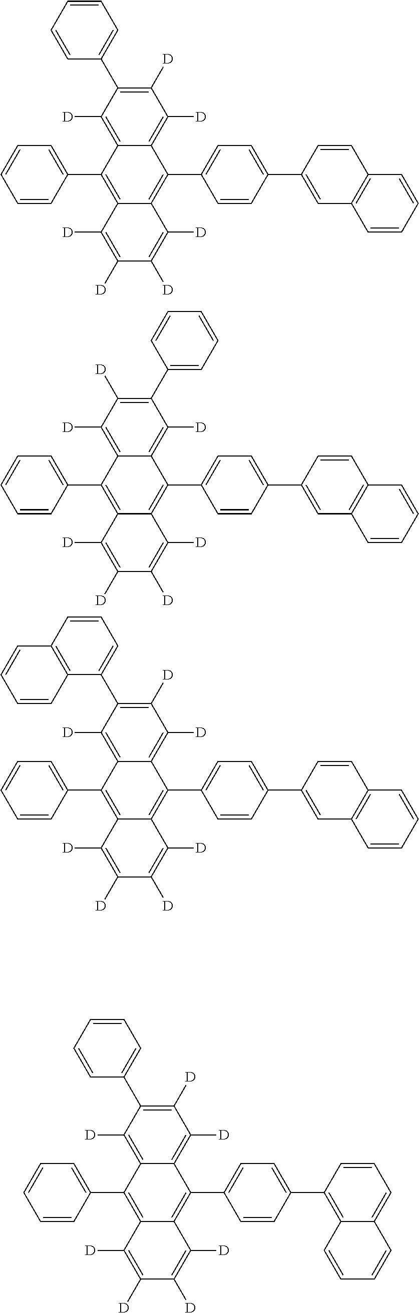

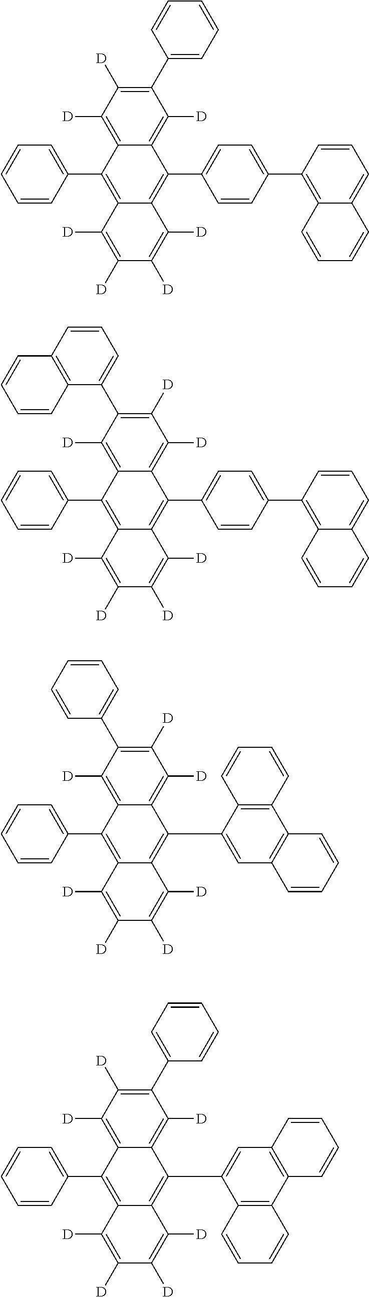

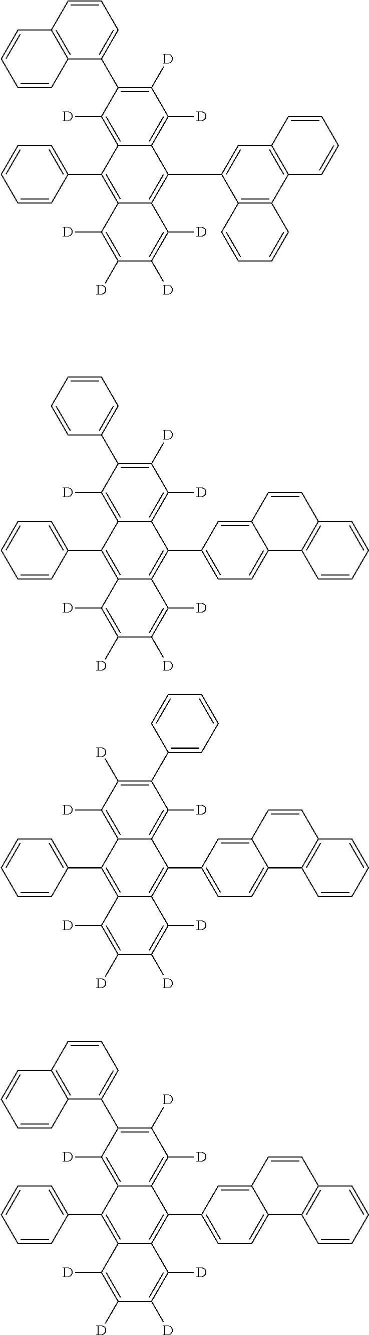

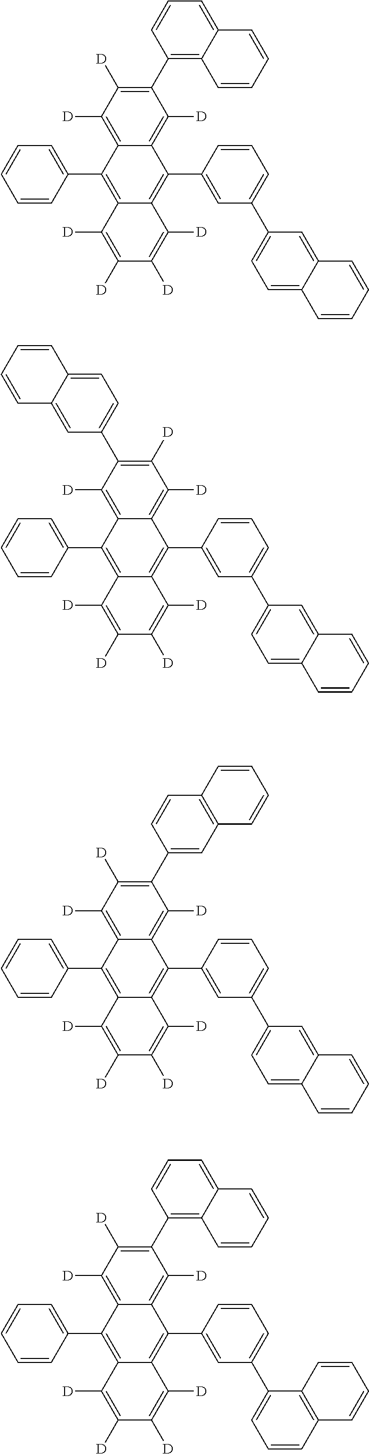 [SMILES: [2H]c1c([2H])c([2H])c2c(-c3cccc(-c4ccc5ccccc5c4)c3)c3c([2H])c(-c4cccc5ccccc45)c([2H])c([2H])c3c(-c3ccccc3)c2c1[2H] ; [2H]c1c([2H])c([2H])c2c(-c3ccccc3)c3c([2H])c(-c4ccc5ccccc5c4)c([2H])c([2H])c3c(-c3cccc(-c4ccc5ccccc5c4)c3)c2c1[2H] ; [2H]c1c([2H])c([2H])c2c(-c3cccc(-c4ccc5ccccc5c4)c3)c3c([2H])c(-c4ccc5ccccc5c4)c([2H])c([2H])c3c(-c3ccccc3)c2c1[2H] ; [2H]c1c([2H])c([2H])c2c(-c3cccc(-c4cccc5ccccc45)c3)c3c([2H])c(-c4cccc5ccccc45)c([2H])c([2H])c3c(-c3ccccc3)c2c1[2H]]

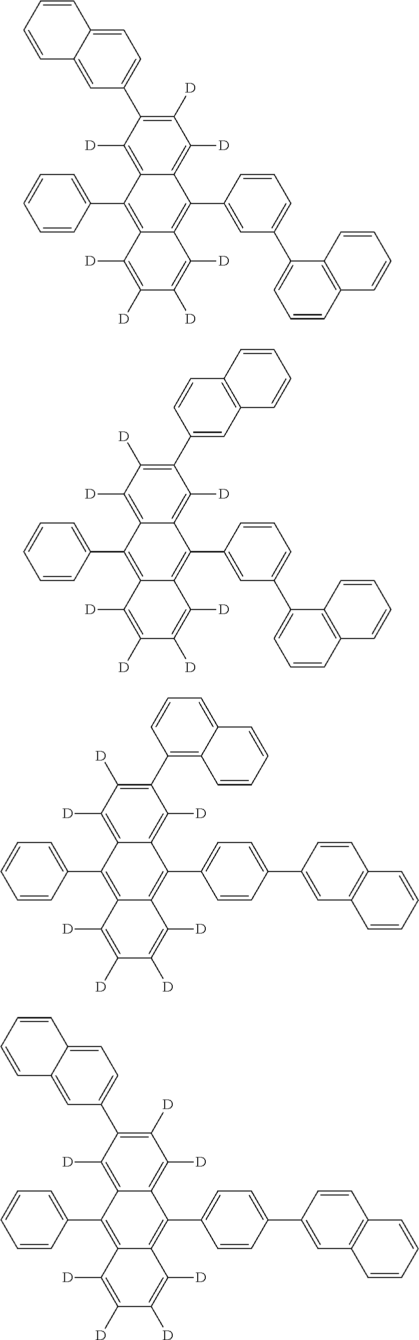

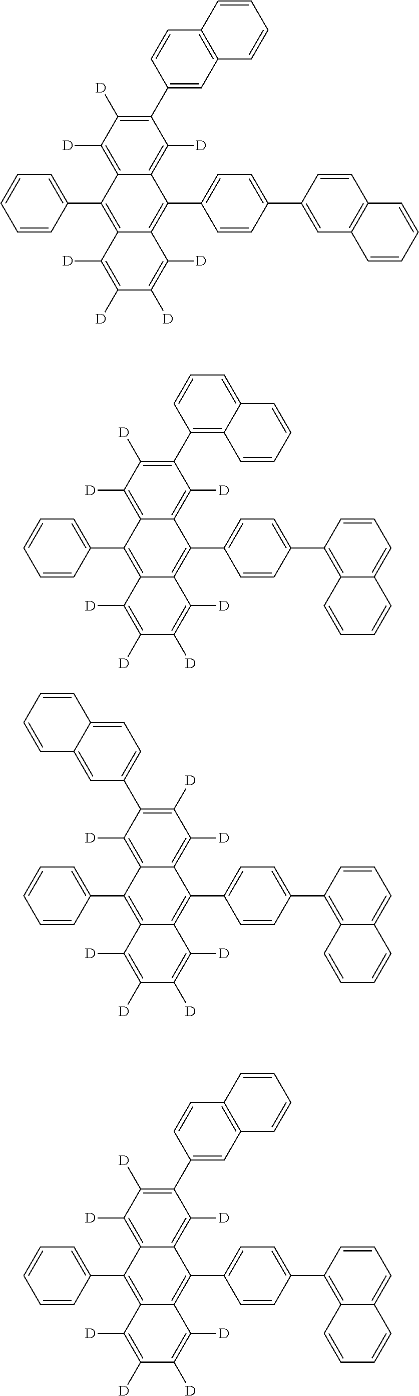 [SMILES: [2H]c1c([2H])c([2H])c2c(-c3ccc(-c4ccc5ccccc5c4)cc3)c3c([2H])c(-c4ccc5ccccc5c4)c([2H])c([2H])c3c(-c3ccccc3)c2c1[2H] ; [2H]c1c([2H])c([2H])c2c(-c3ccc(-c4cccc5ccccc45)cc3)c3c([2H])c(-c4cccc5ccccc45)c([2H])c([2H])c3c(-c3ccccc3)c2c1[2H] ; [2H]c1c([2H])c([2H])c2c(-c3ccccc3)c3c([2H])c(-c4ccc5ccccc5c4)c([2H])c([2H])c3c(-c3ccc(-c4cccc5ccccc45)cc3)c2c1[2H] ; [2H]c1c([2H])c([2H])c2c(-c3ccc(-c4cccc5ccccc45)cc3)c3c([2H])c(-c4ccc5ccccc5c4)c([2H])c([2H])c3c(-c3ccccc3)c2c1[2H]]

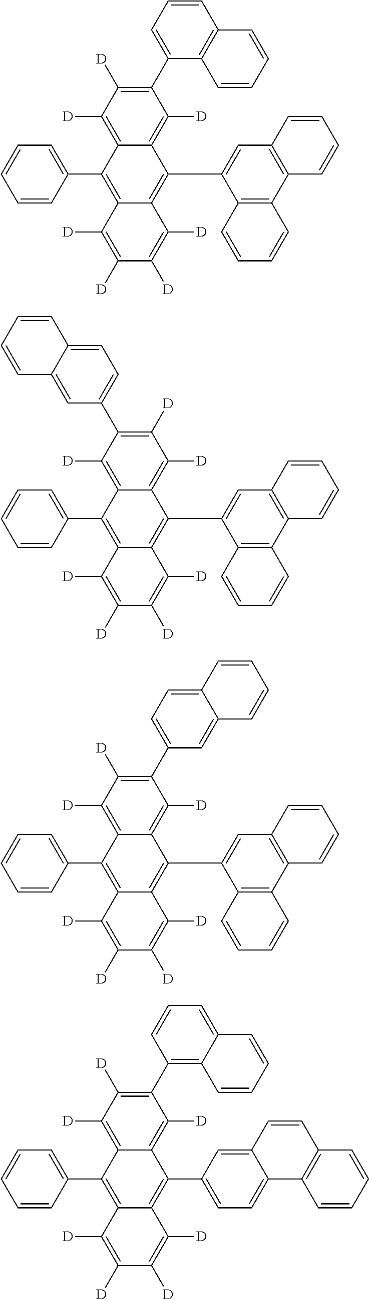

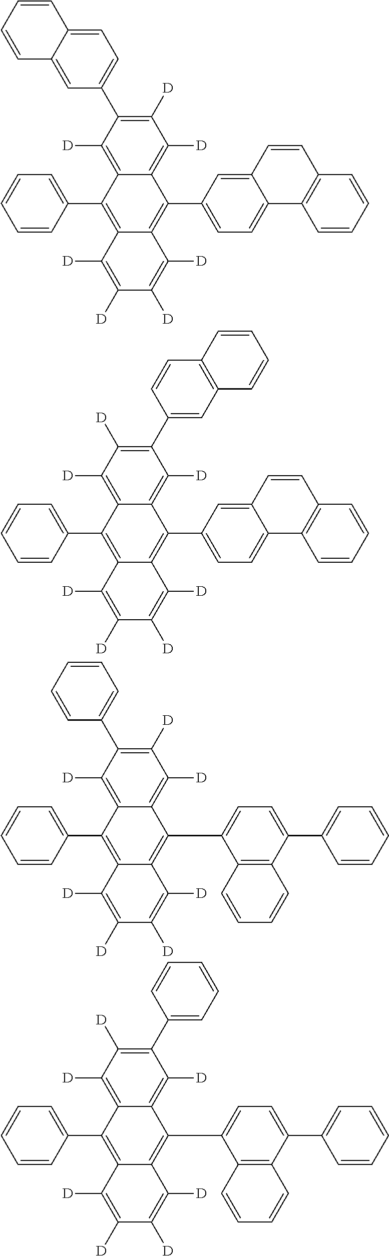

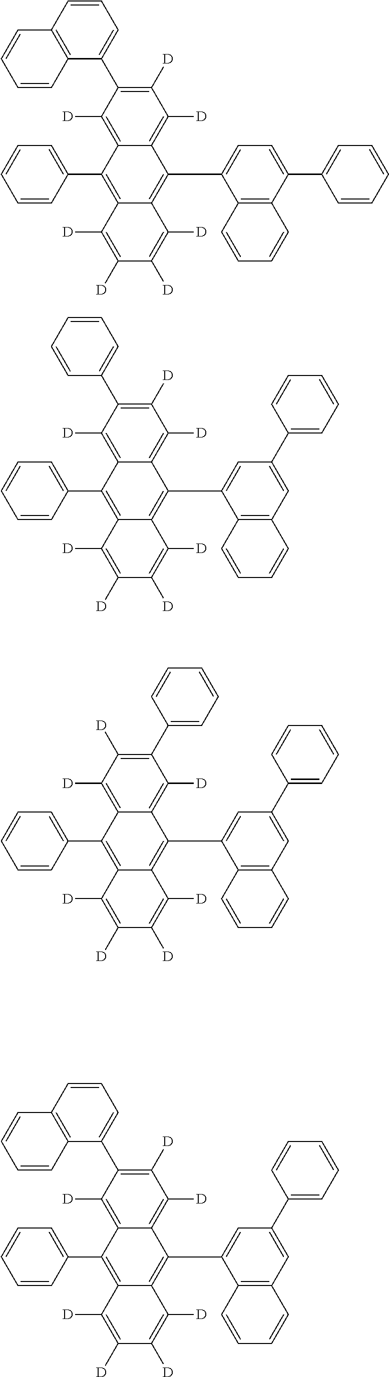

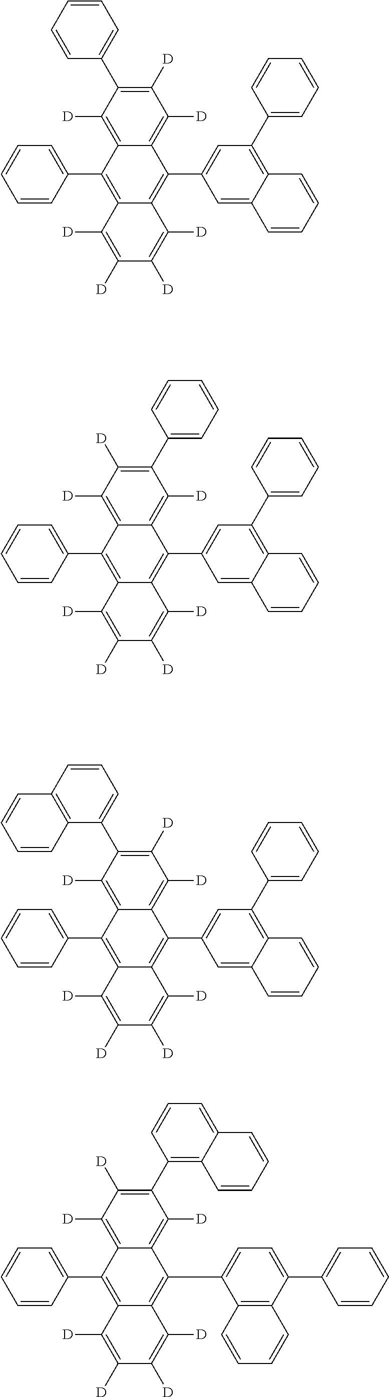

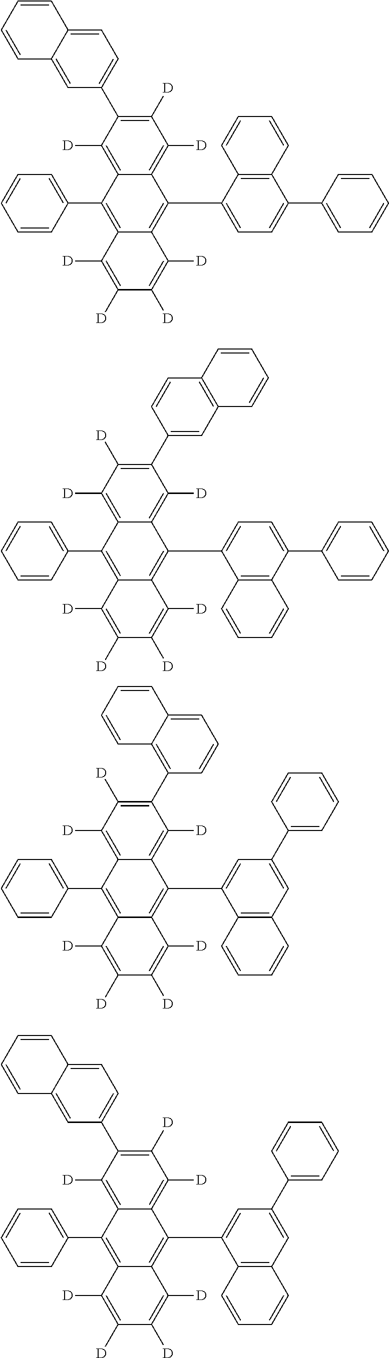

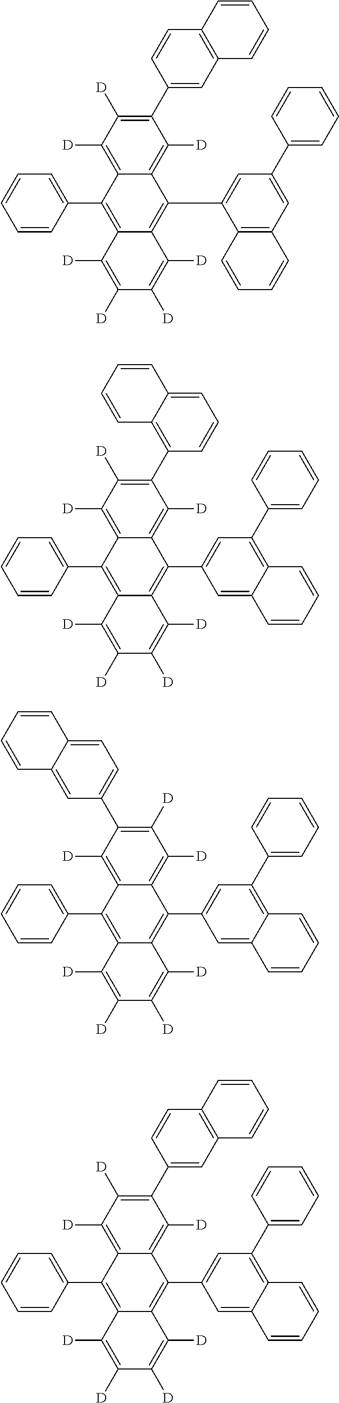 [SMILES: [2H]c1c([2H])c([2H])c2c(-c3cc(-c4ccccc4)cc4ccccc34)c3c([2H])c(-c4ccc5ccccc5c4)c([2H])c([2H])c3c(-c3ccccc3)c2c1[2H] ; [2H]c1c([2H])c([2H])c2c(-c3cc(-c4ccccc4)c4ccccc4c3)c3c([2H])c(-c4cccc5ccccc45)c([2H])c([2H])c3c(-c3ccccc3)c2c1[2H] ; [2H]c1c([2H])c([2H])c2c(-c3ccccc3)c3c([2H])c(-c4ccc5ccccc5c4)c([2H])c([2H])c3c(-c3cc(-c4ccccc4)c4ccccc4c3)c2c1[2H] ; [2H]c1c([2H])c([2H])c2c(-c3cc(-c4ccccc4)c4ccccc4c3)c3c([2H])c(-c4ccc5ccccc5c4)c([2H])c([2H])c3c(-c3ccccc3)c2c1[2H]]

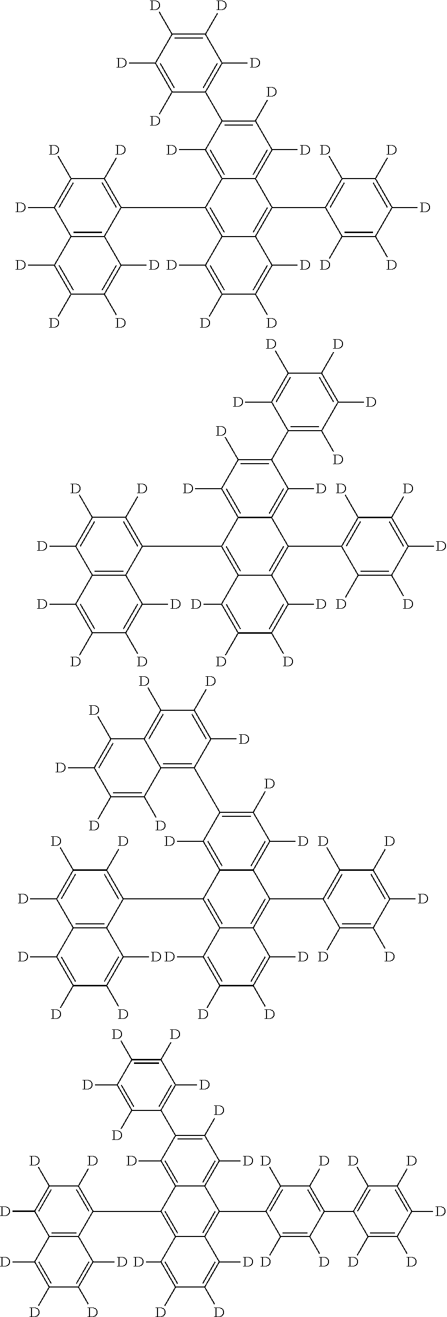

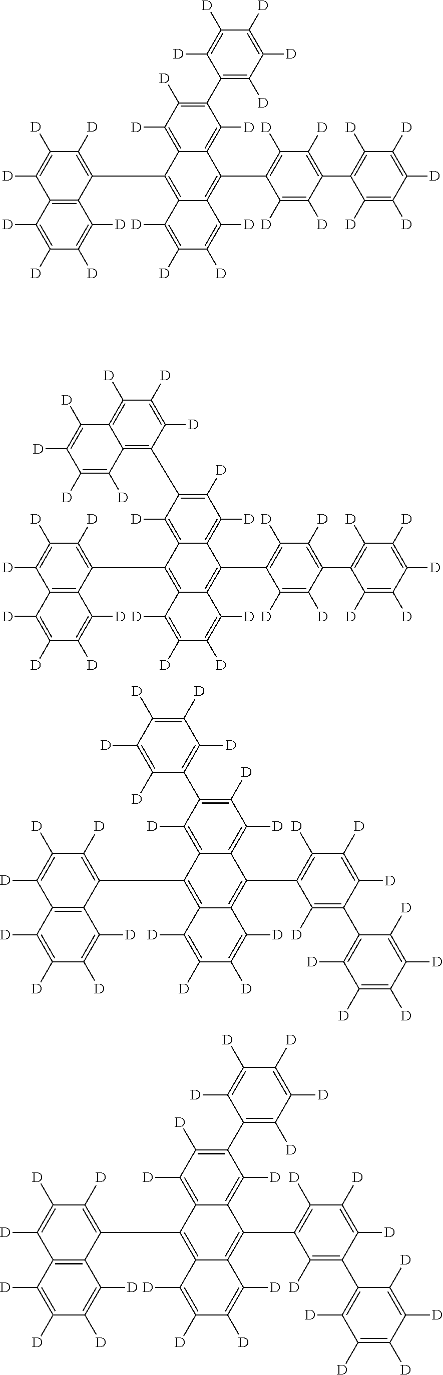

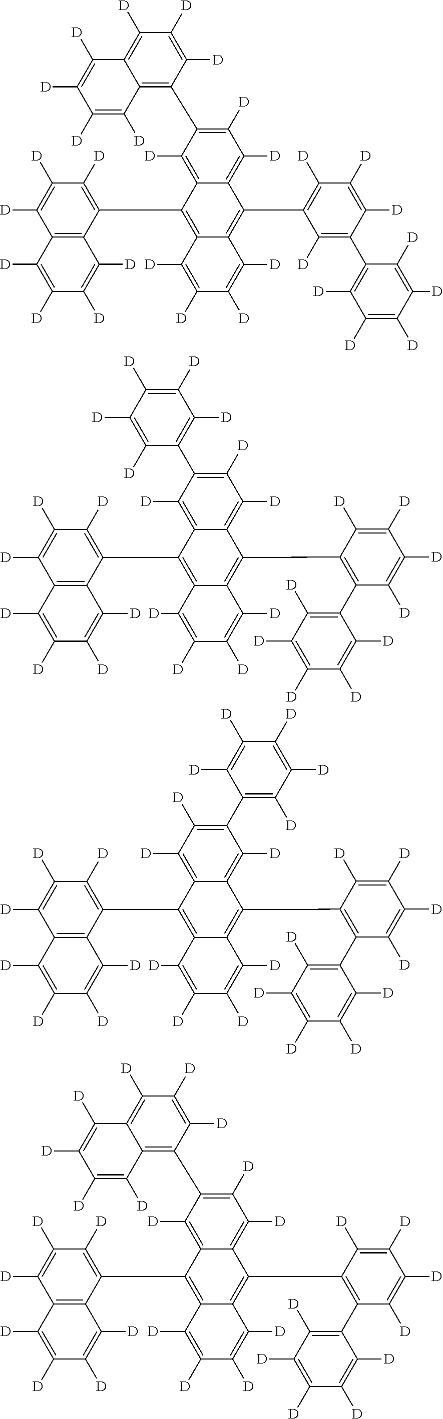

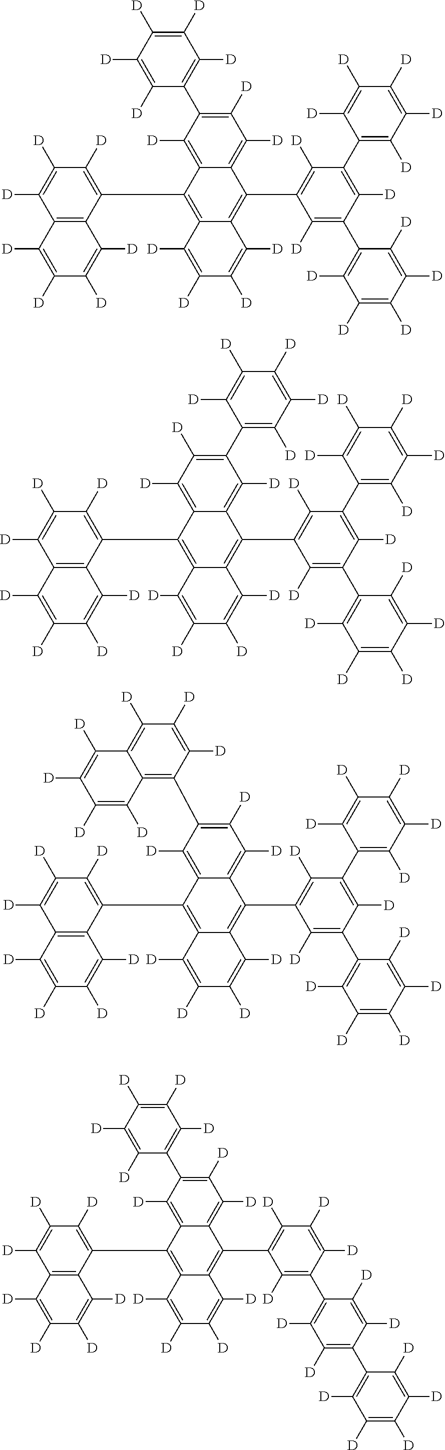

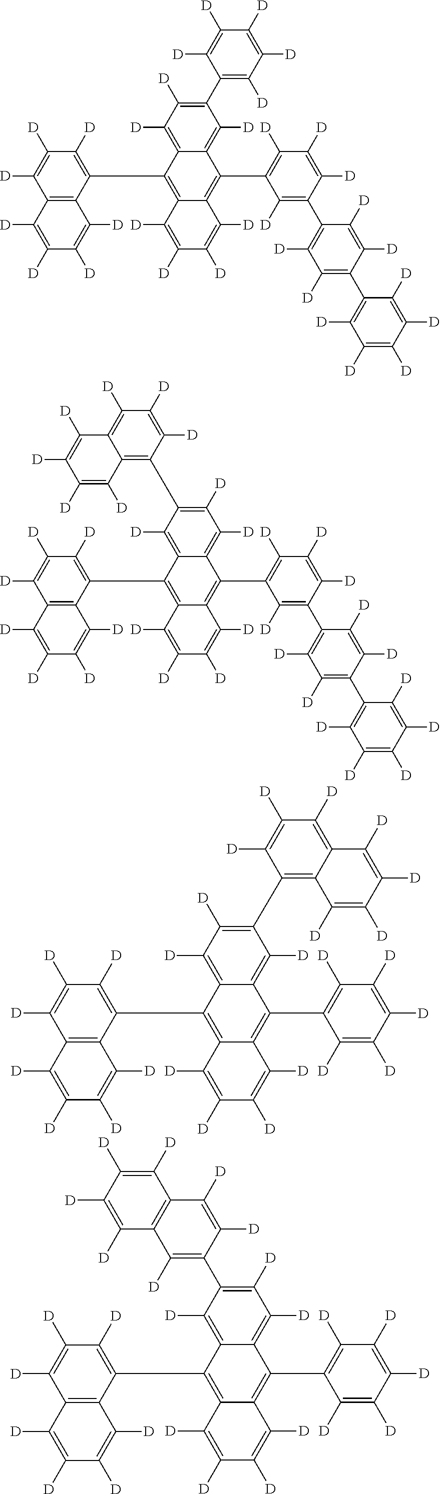

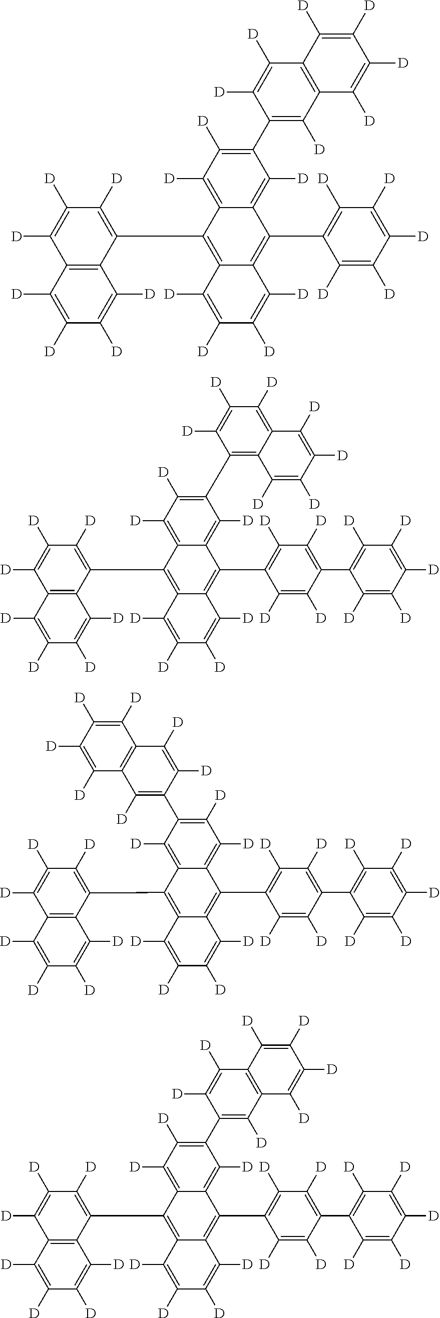

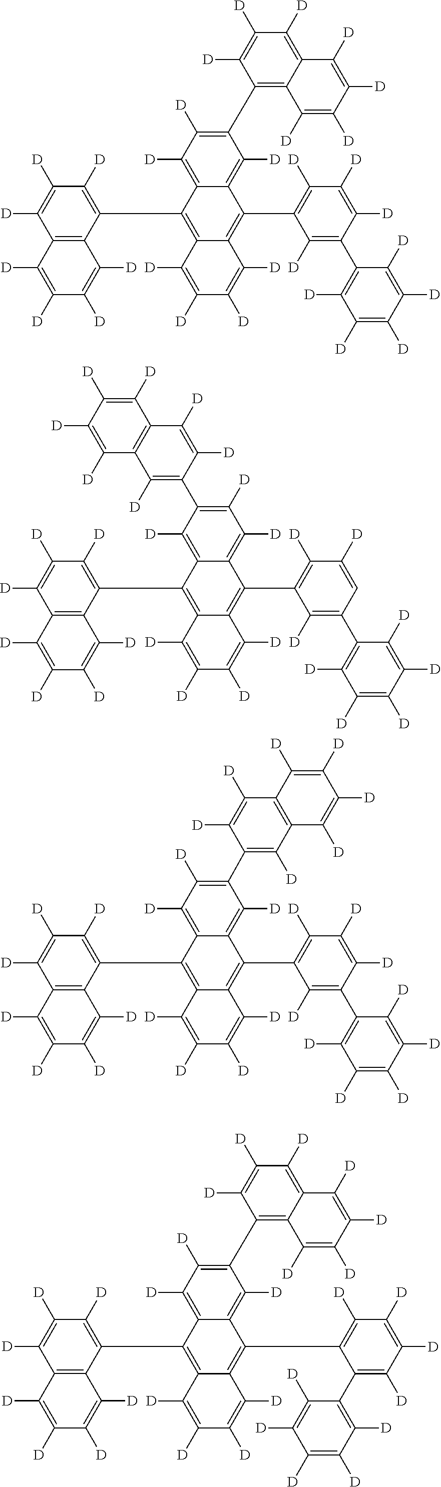

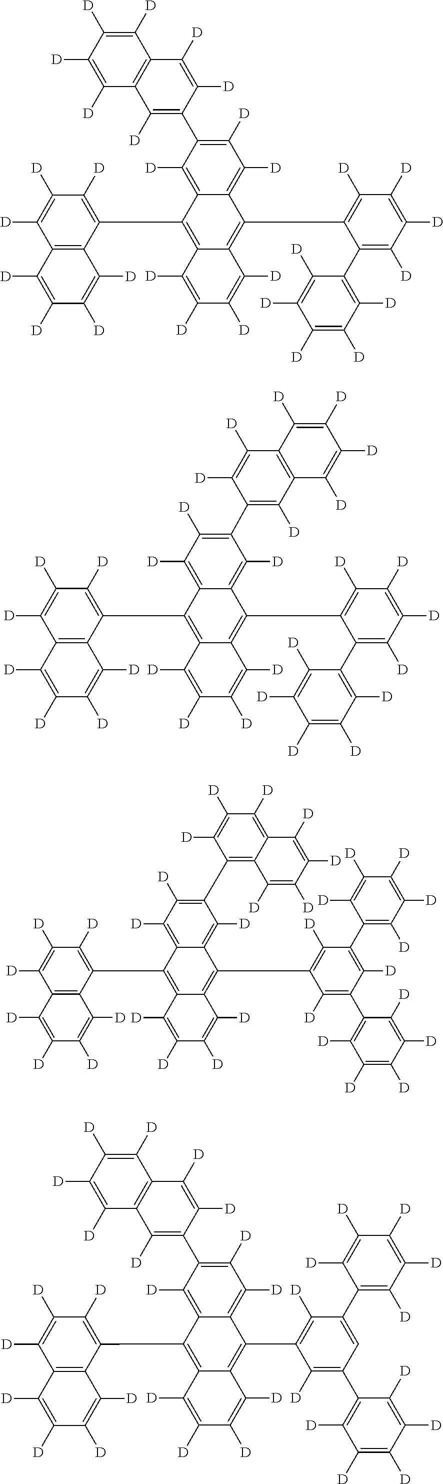

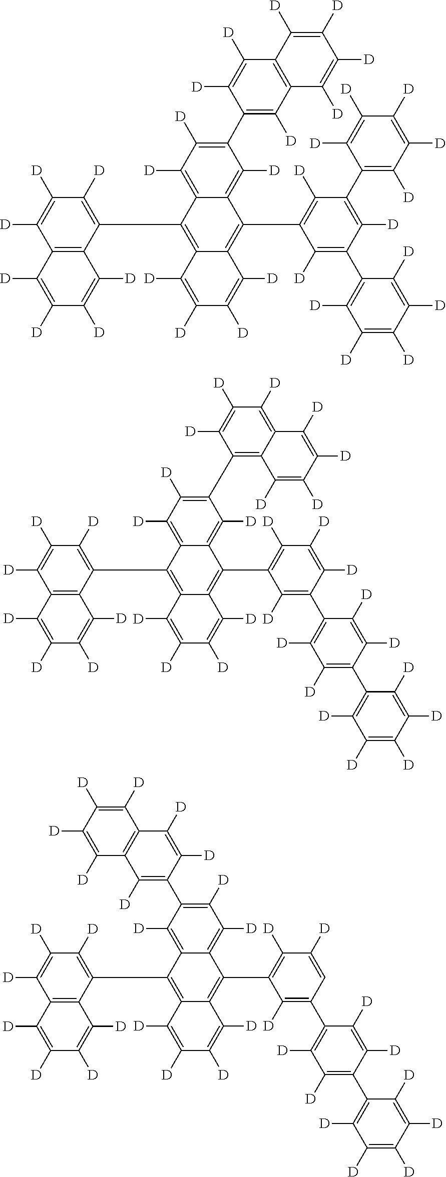

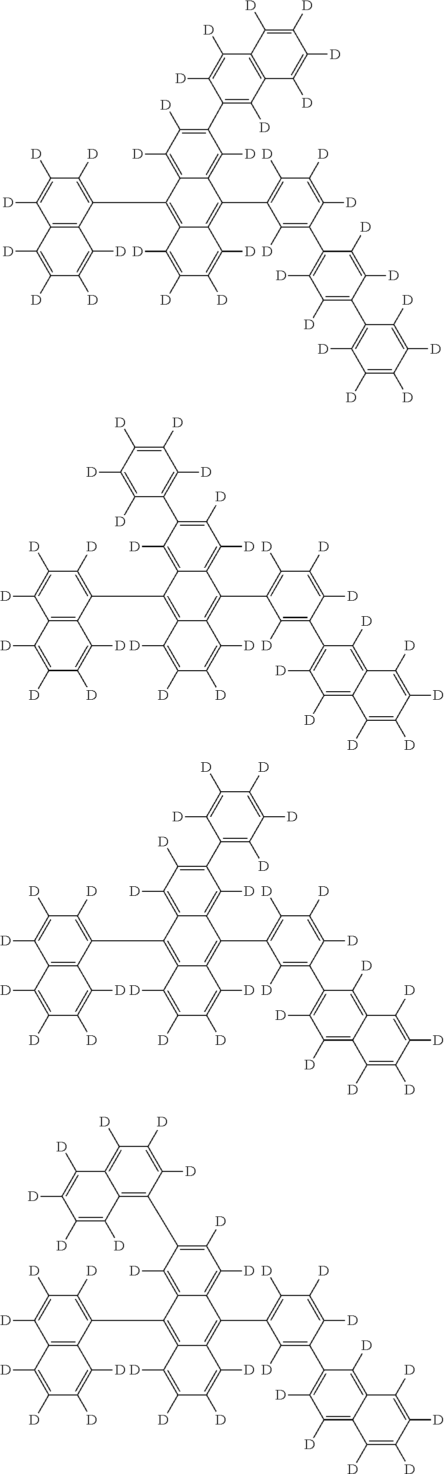

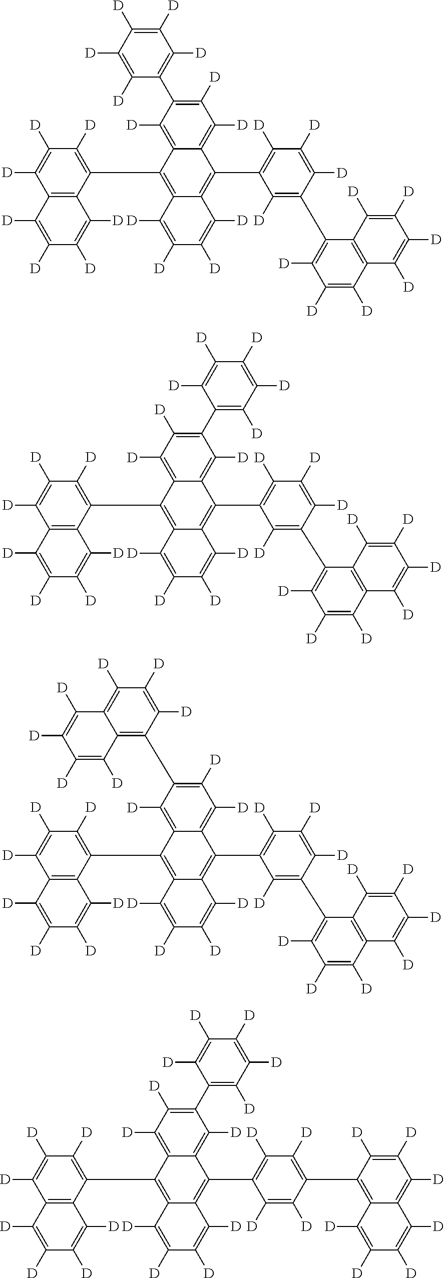

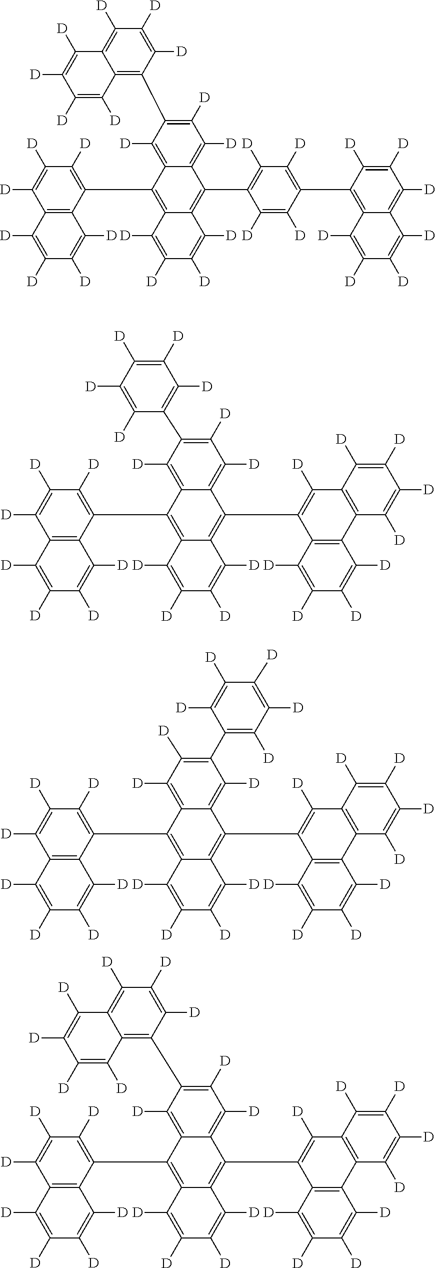

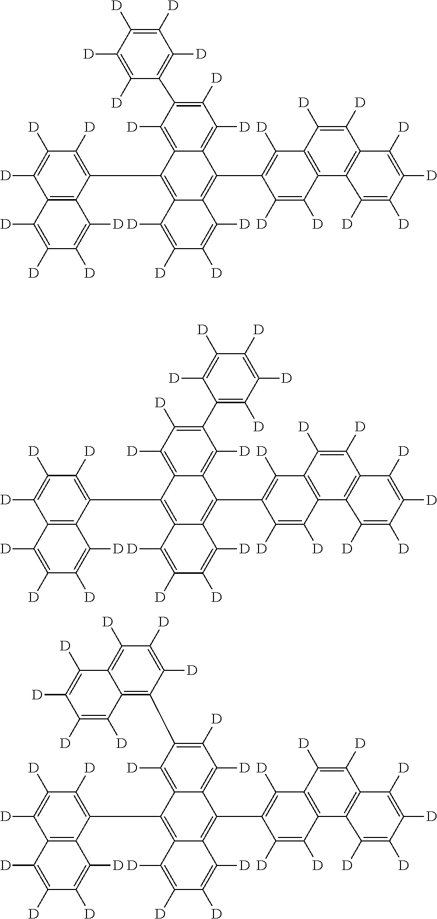

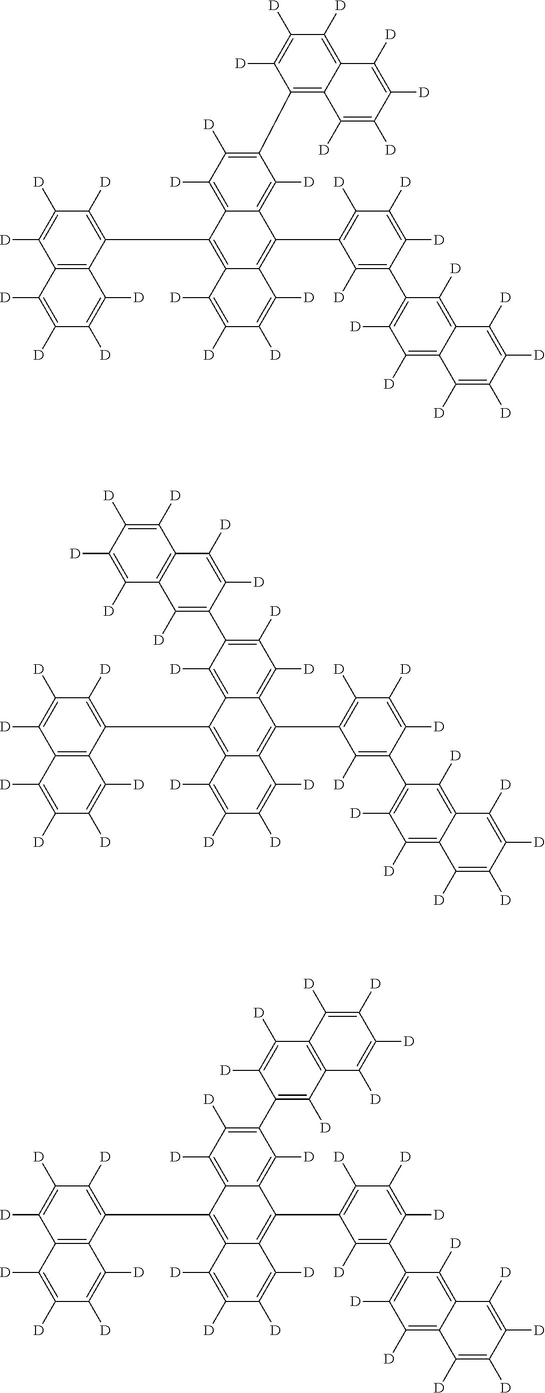

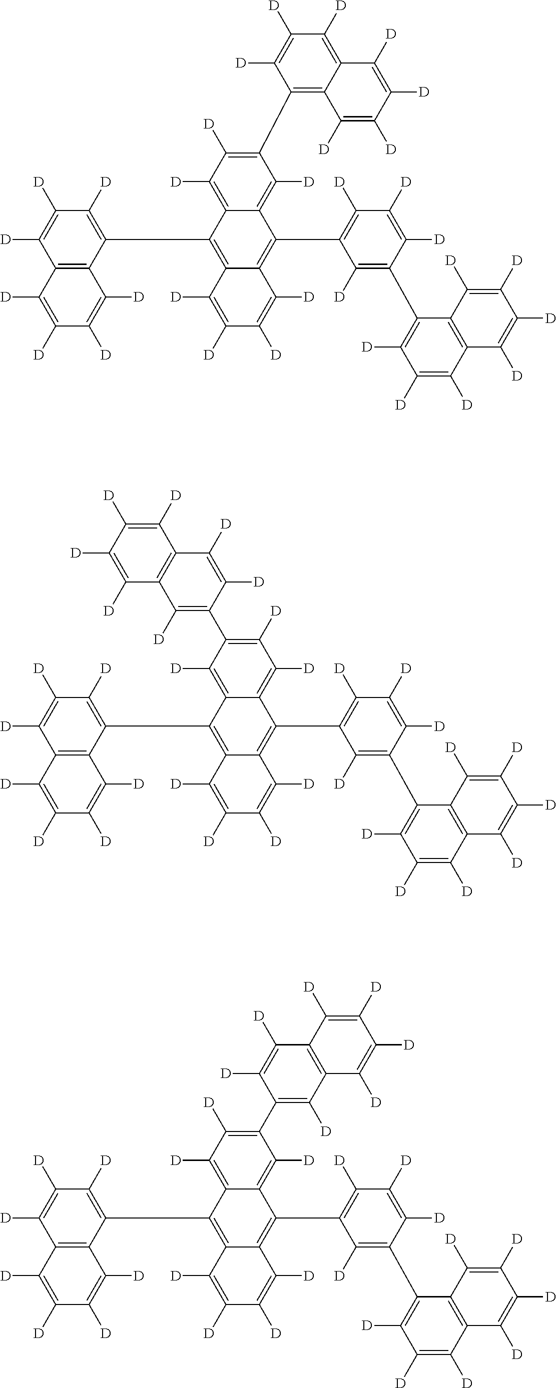

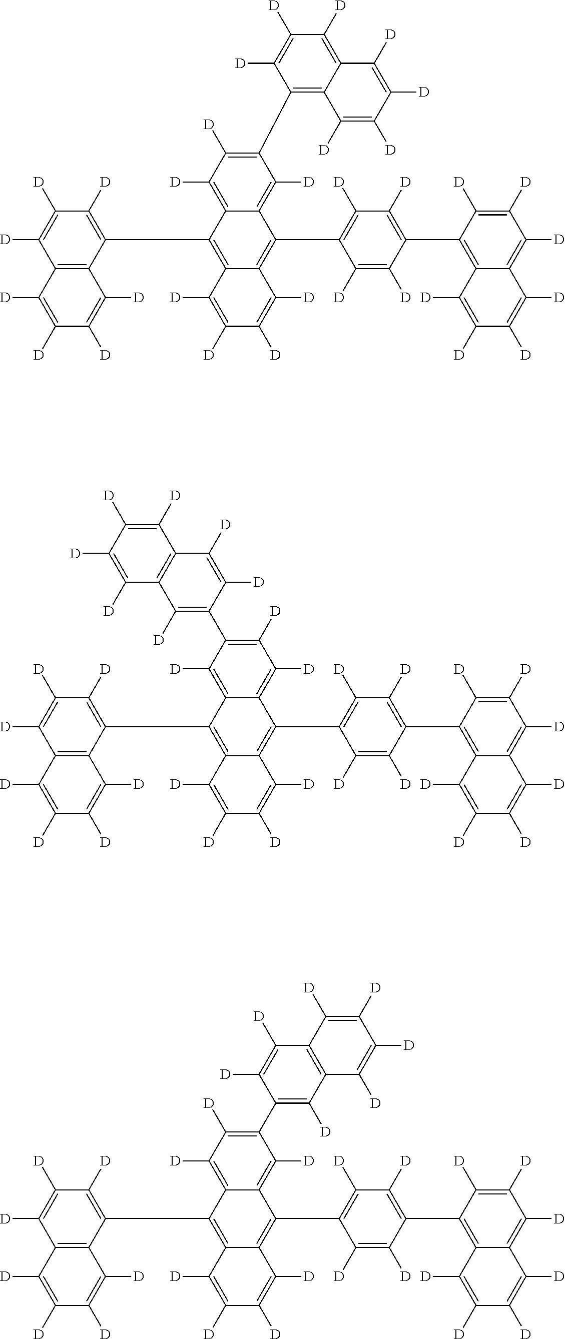

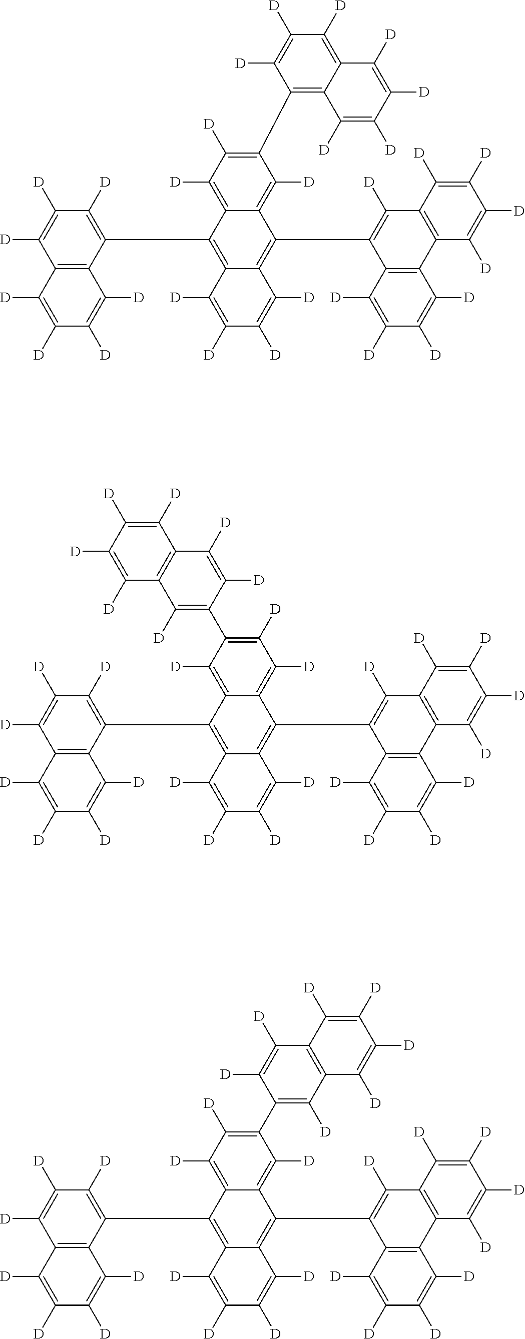

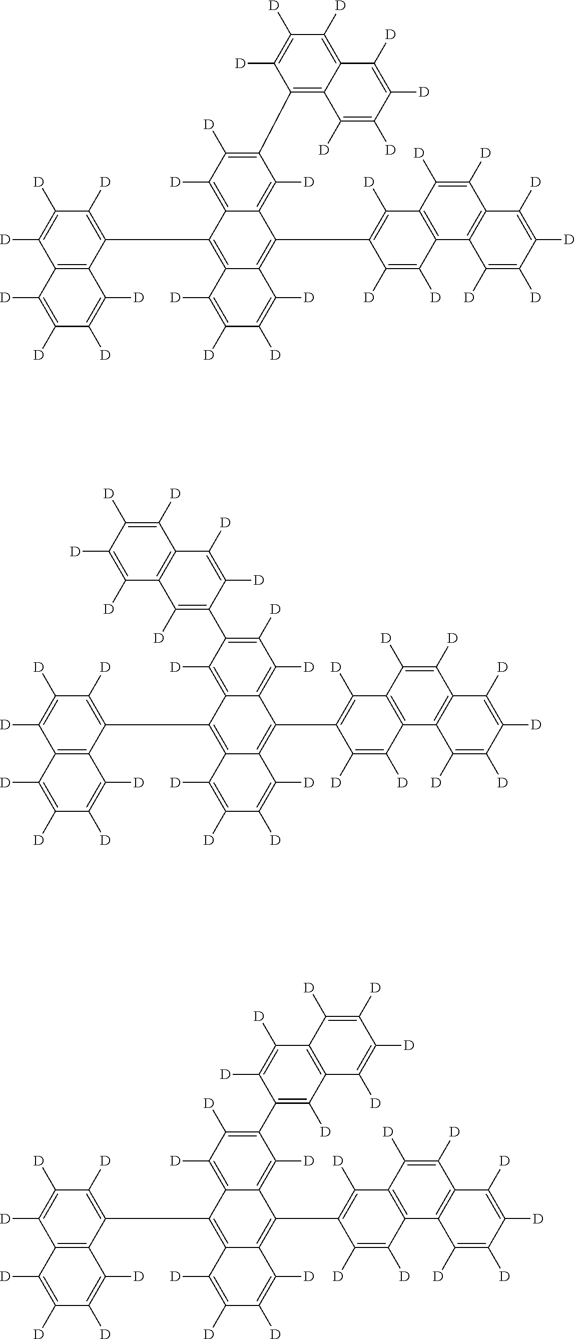

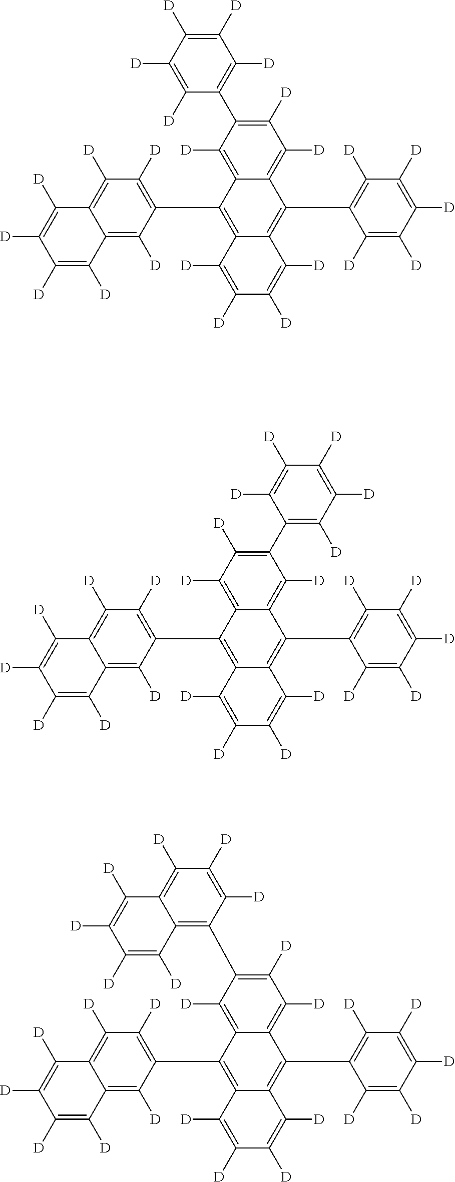

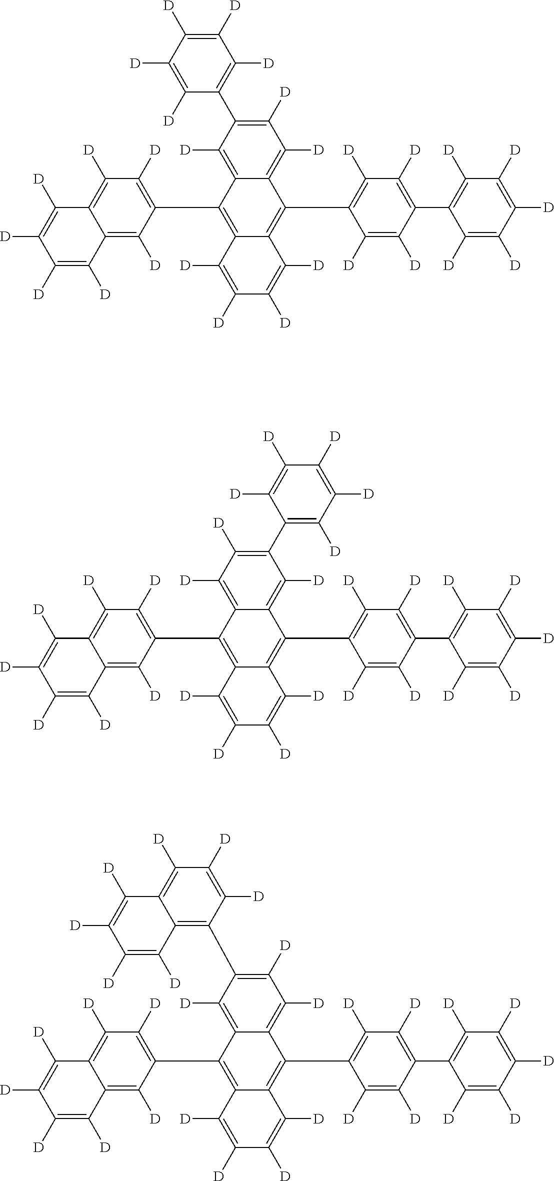

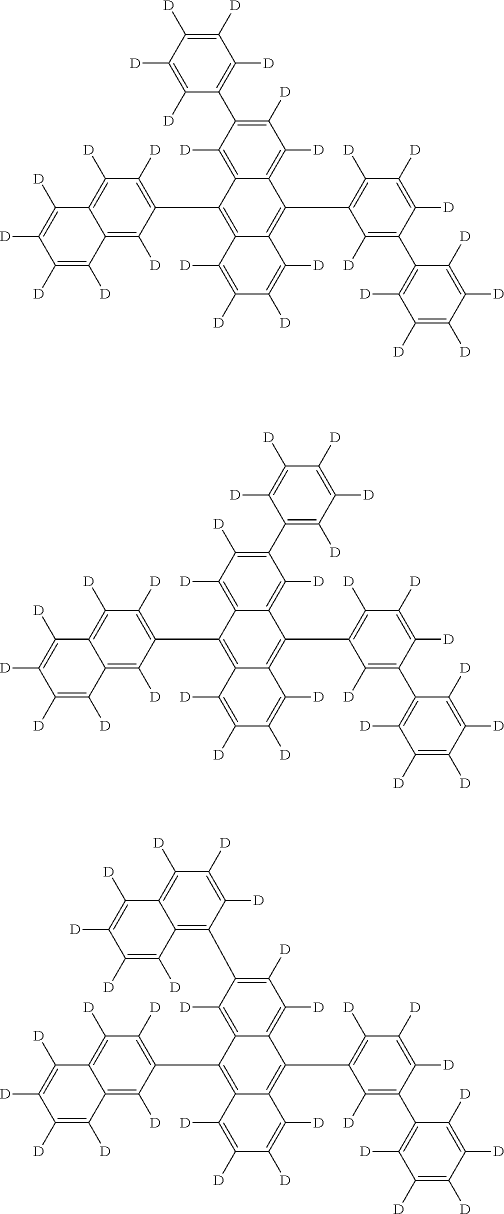

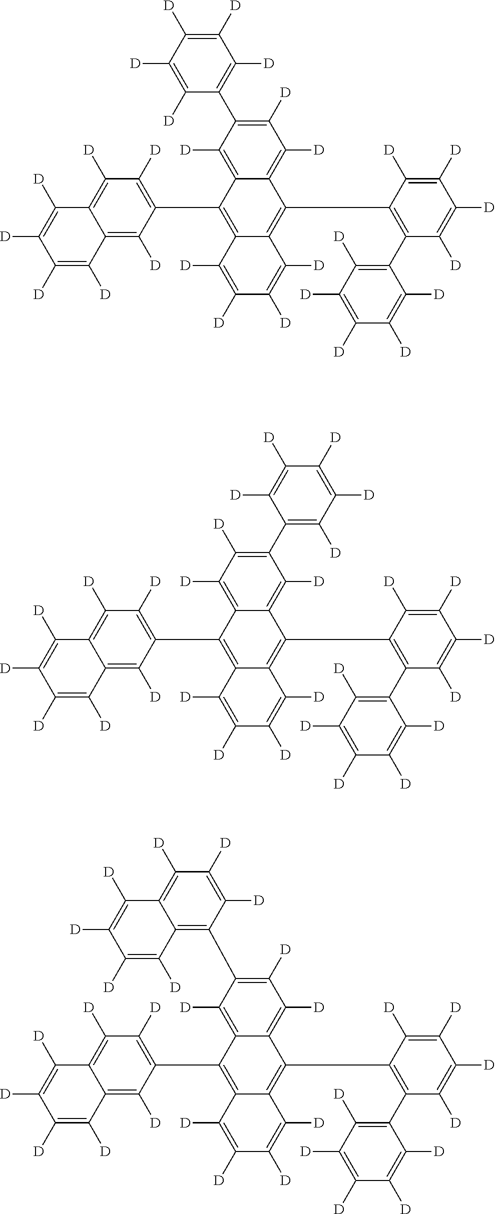

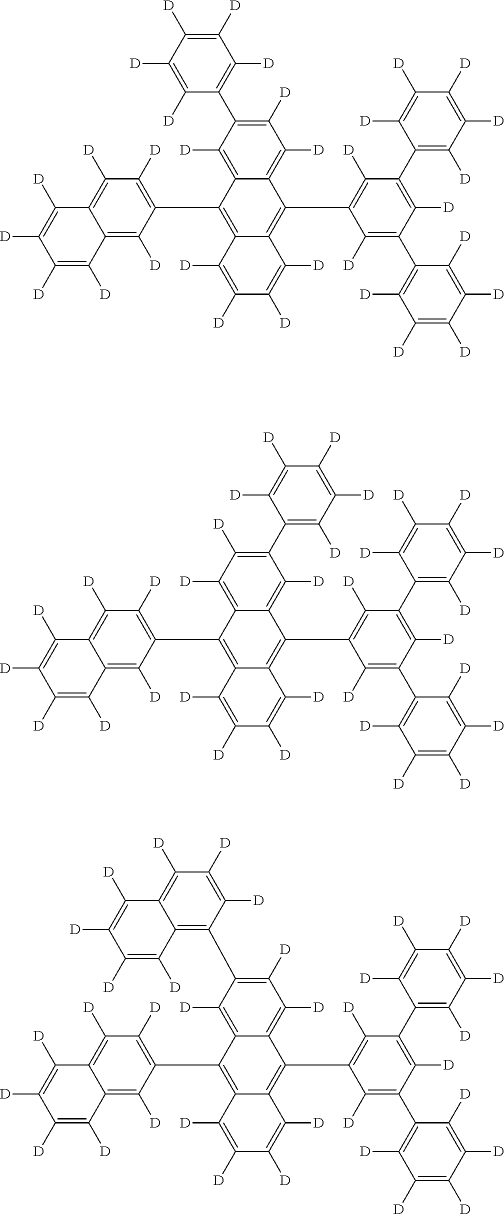

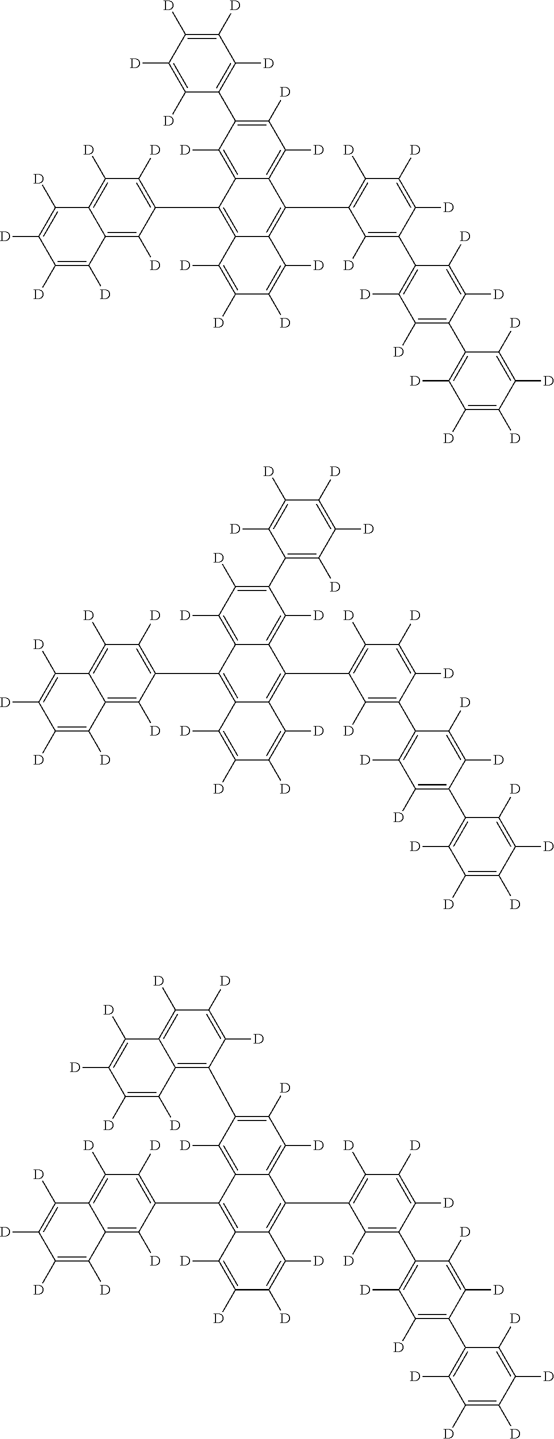

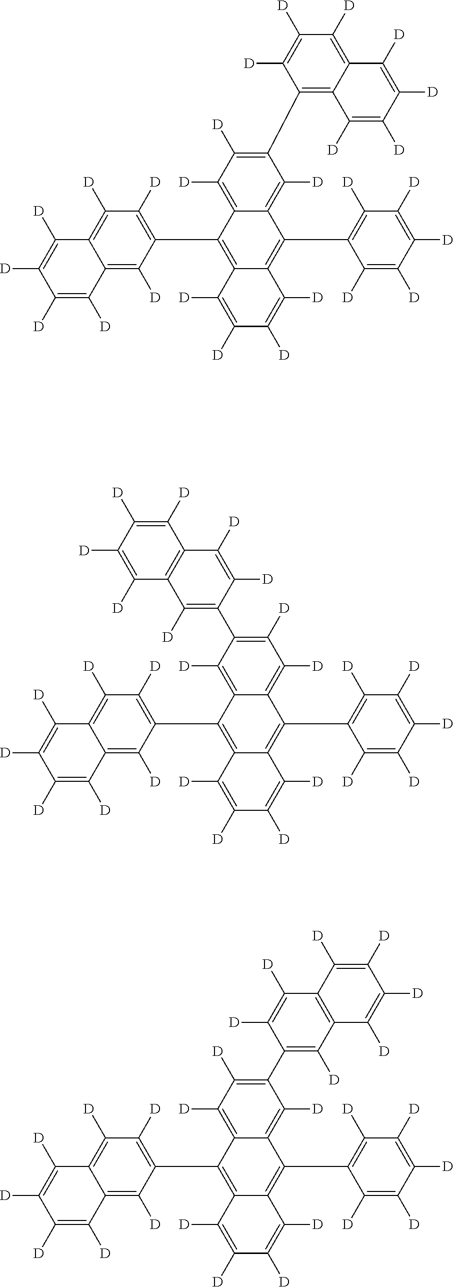

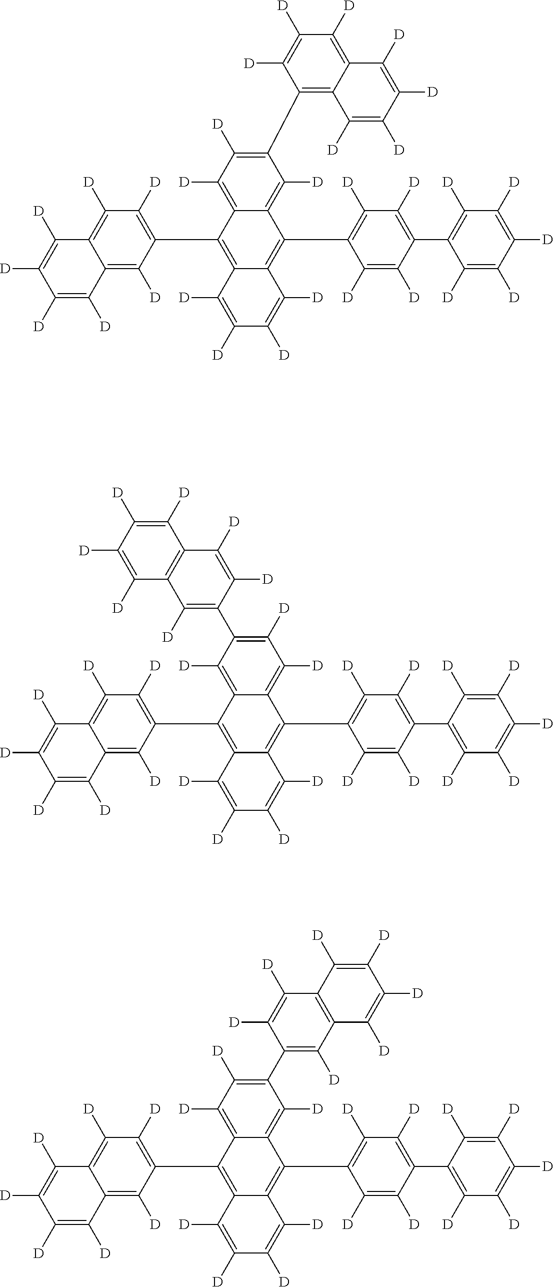

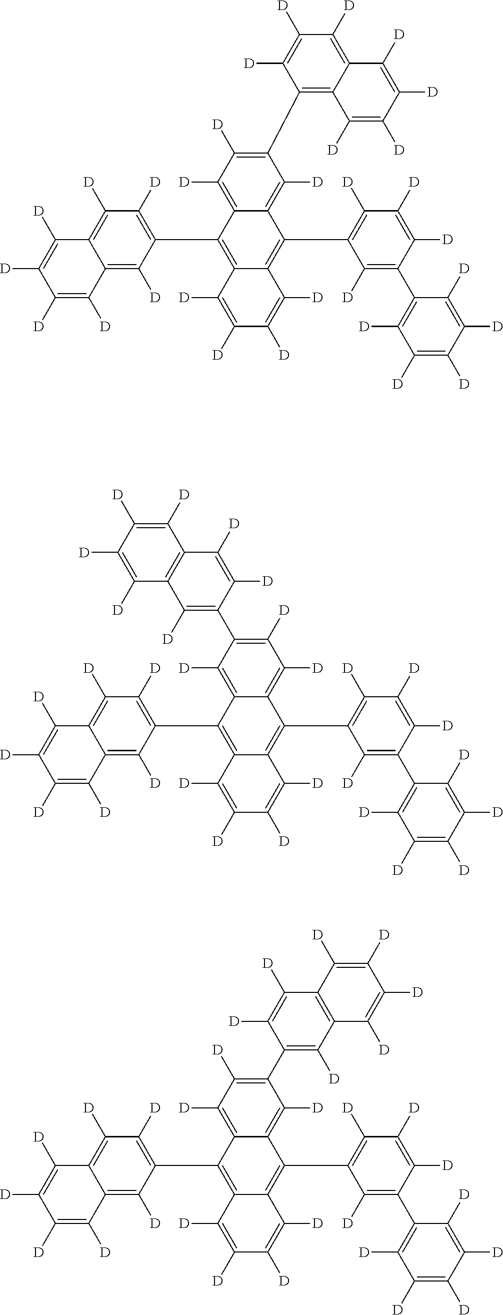

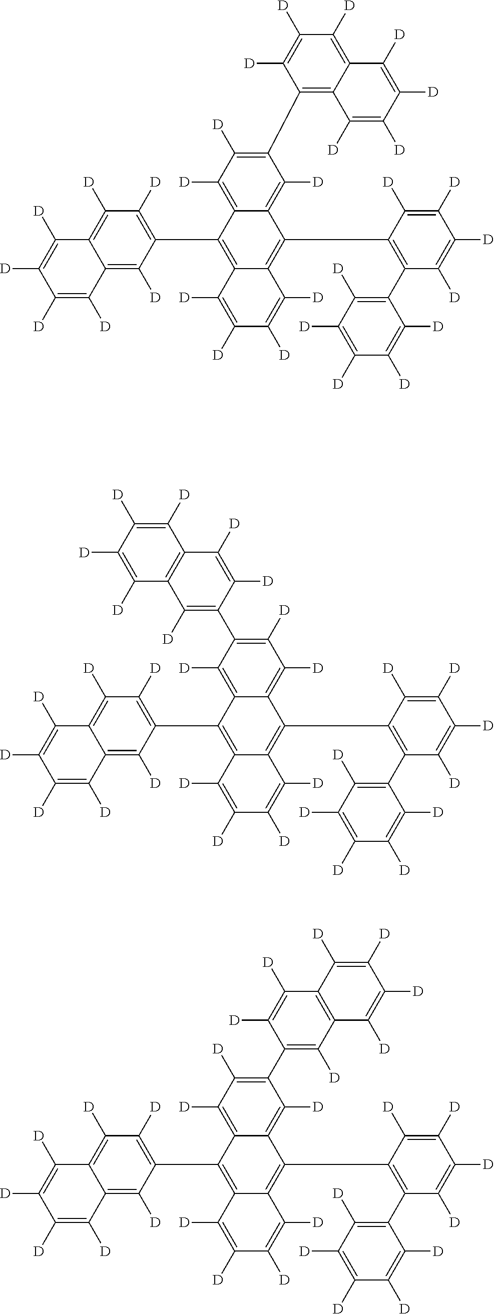

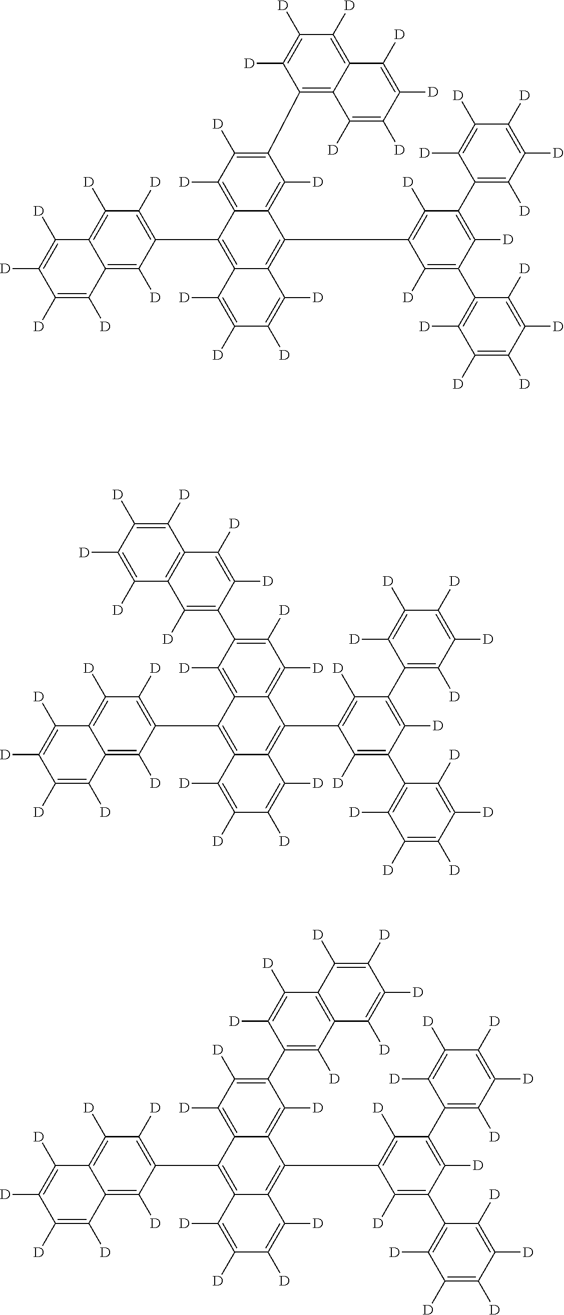

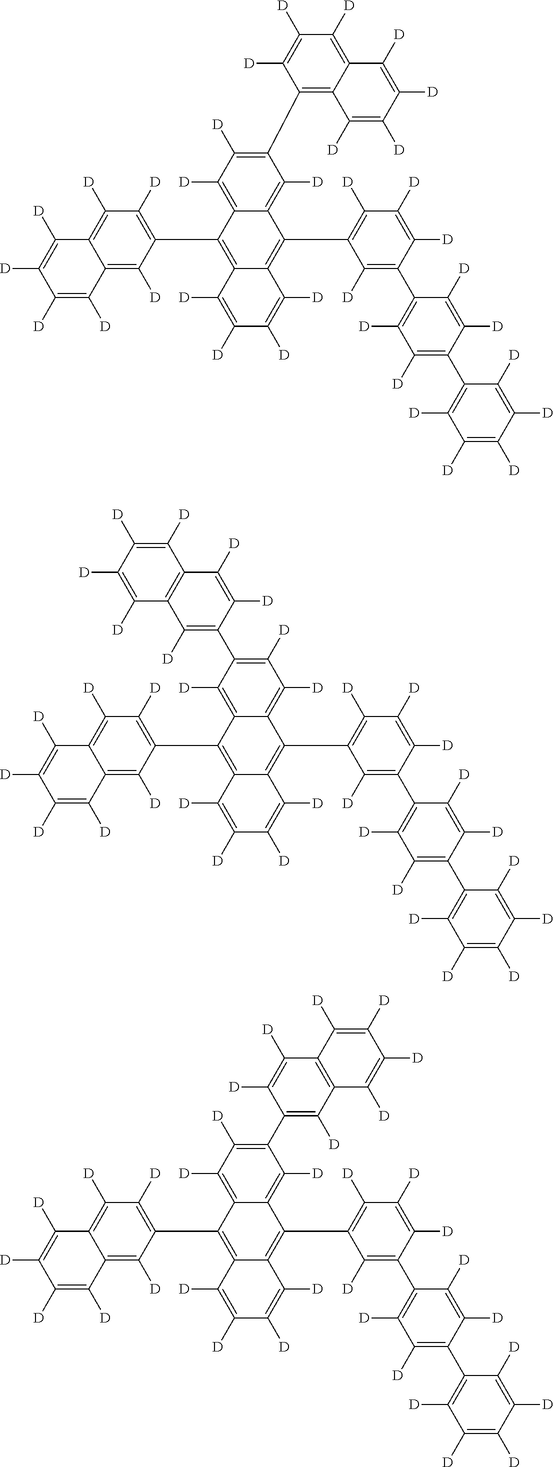

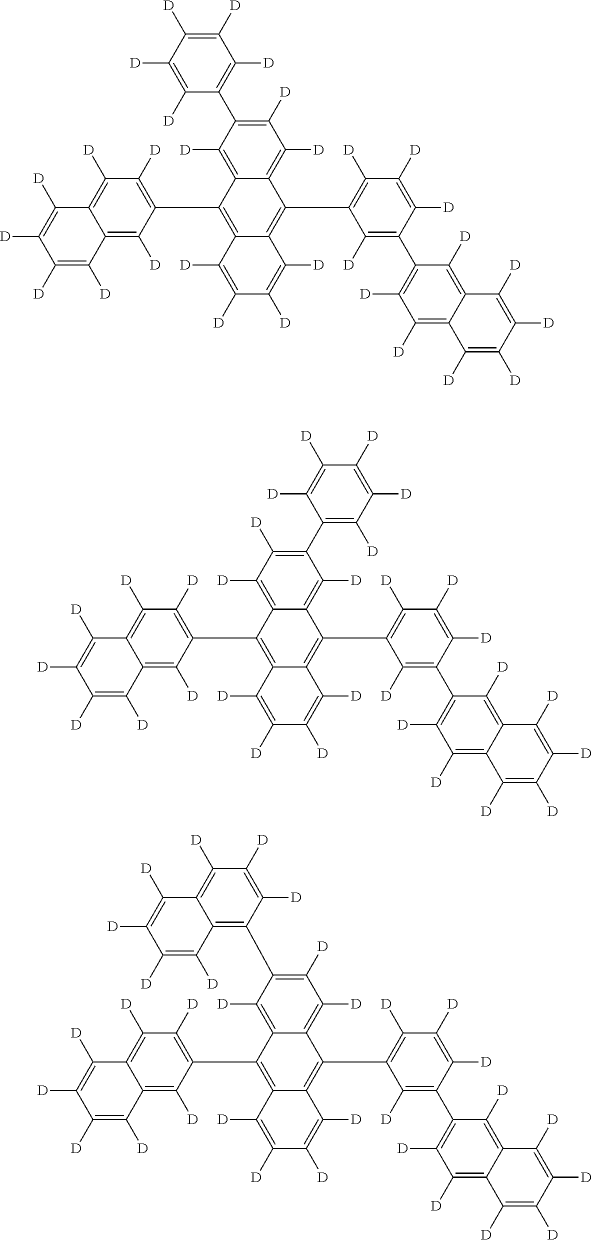

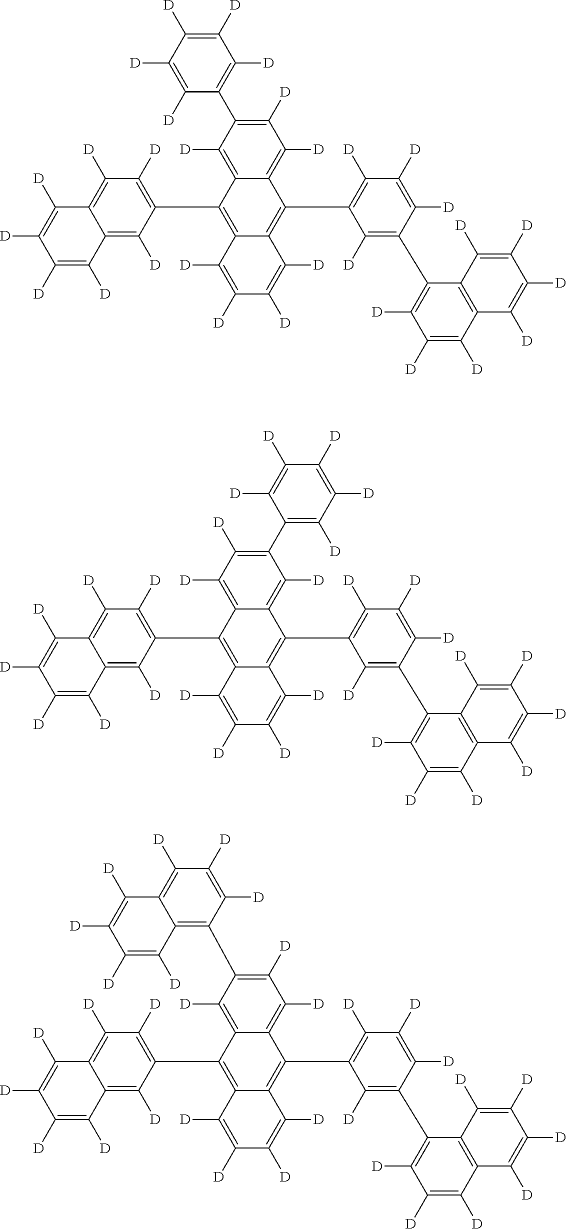

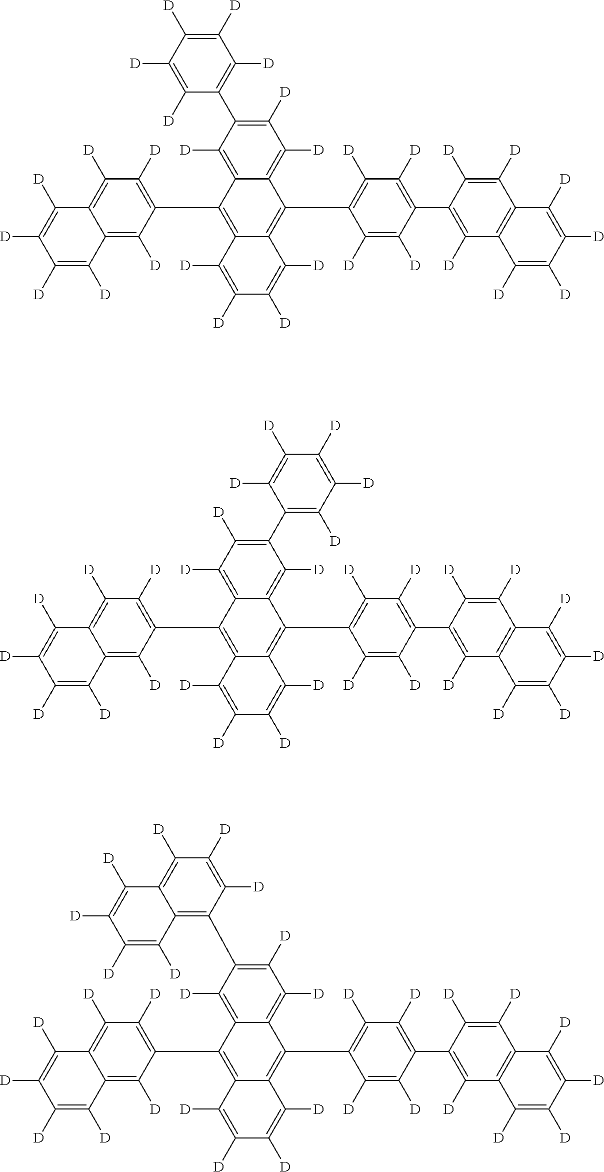

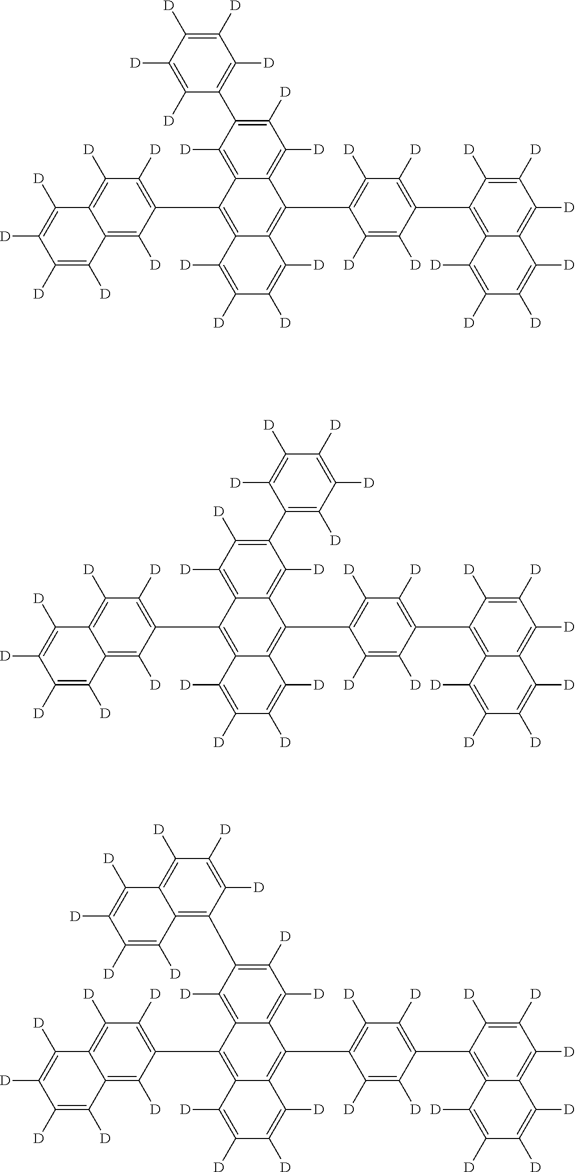

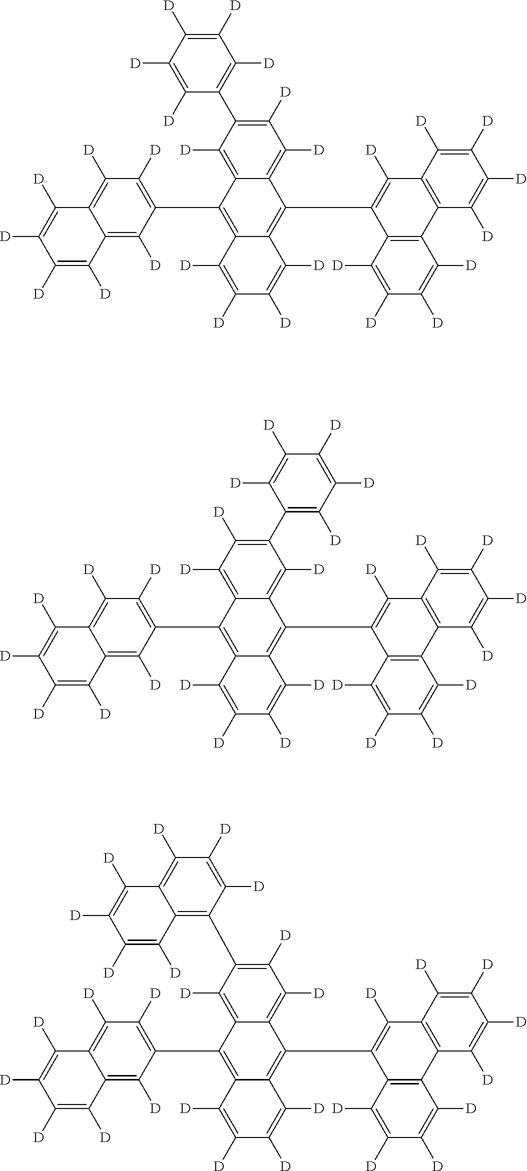

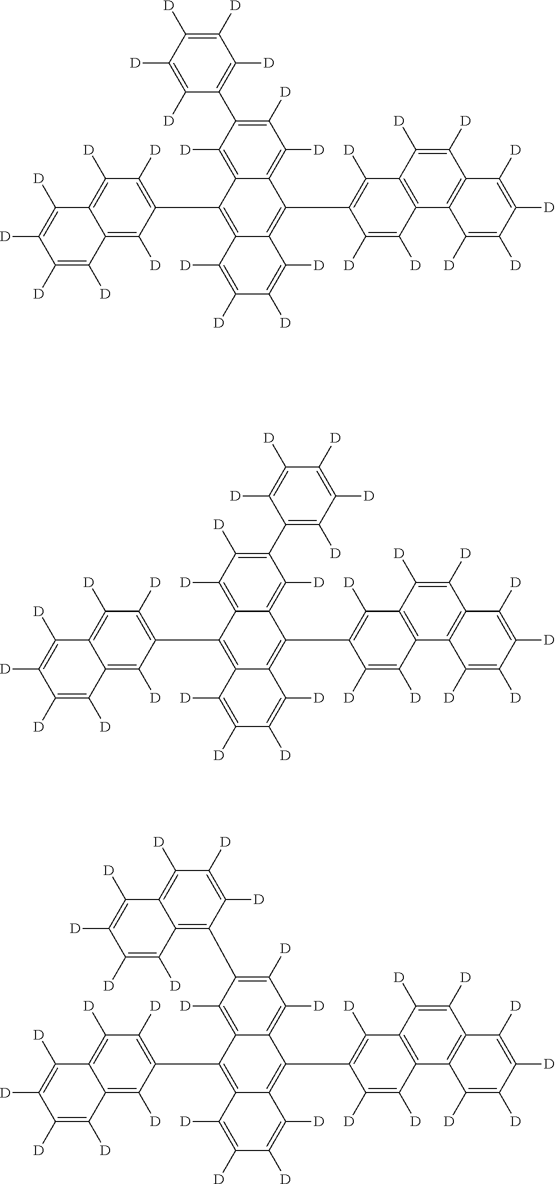

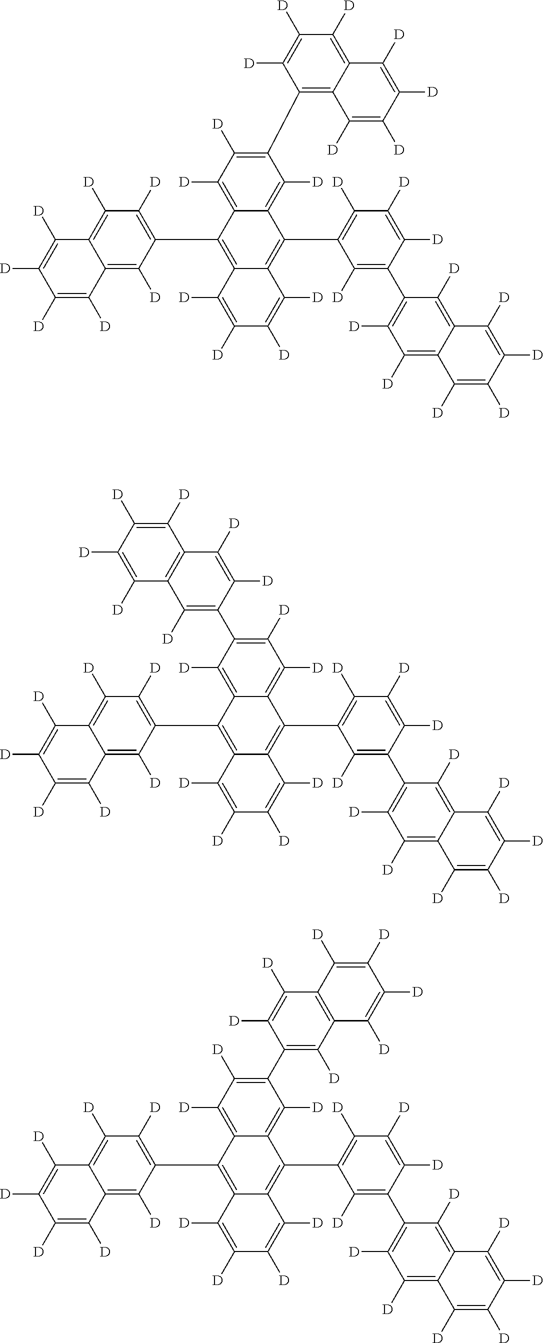

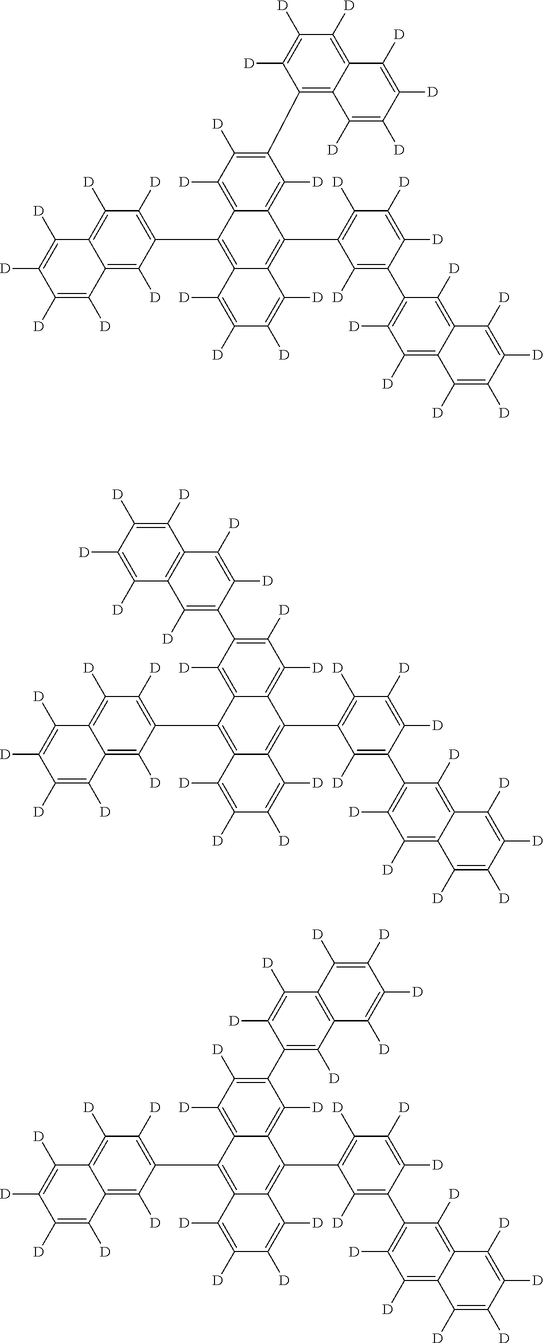

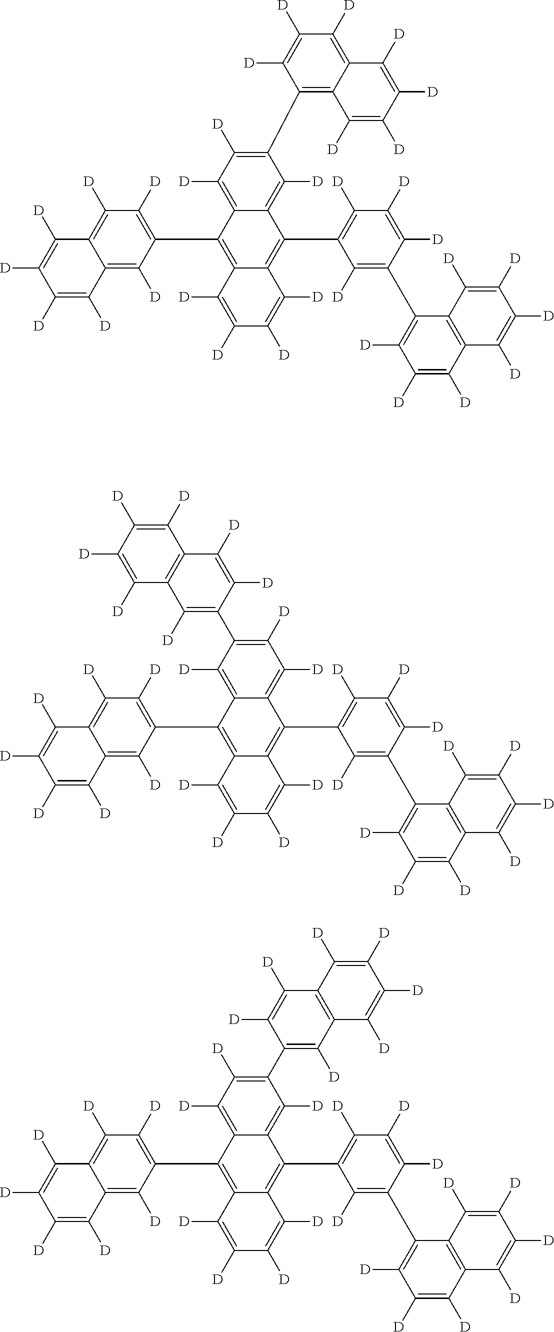

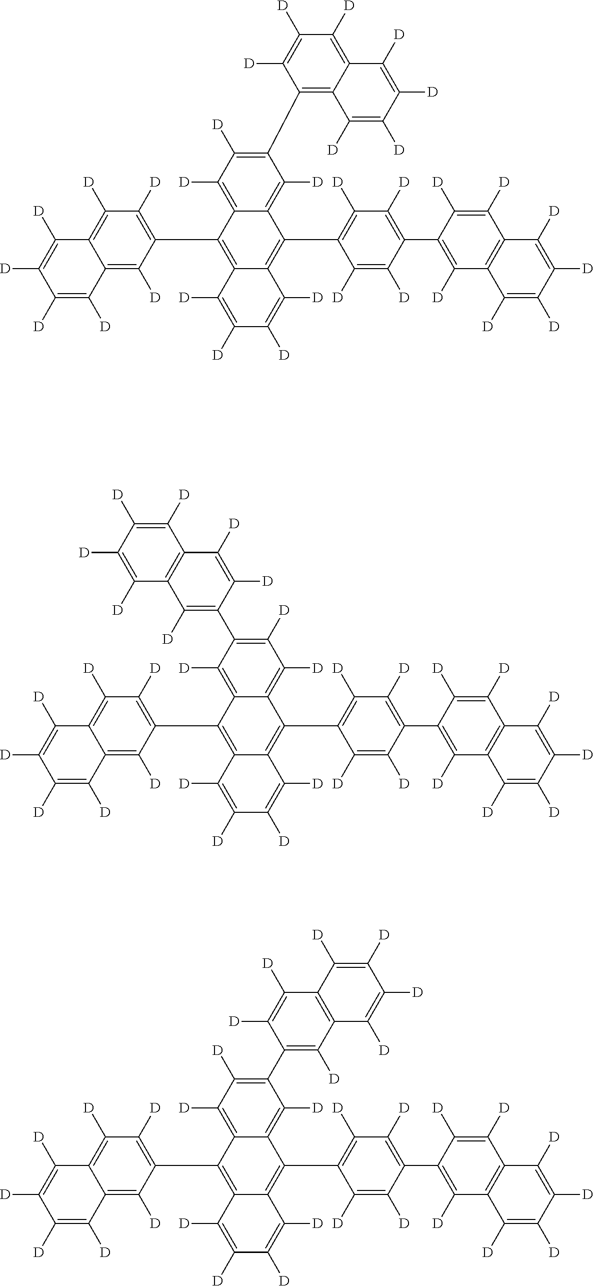

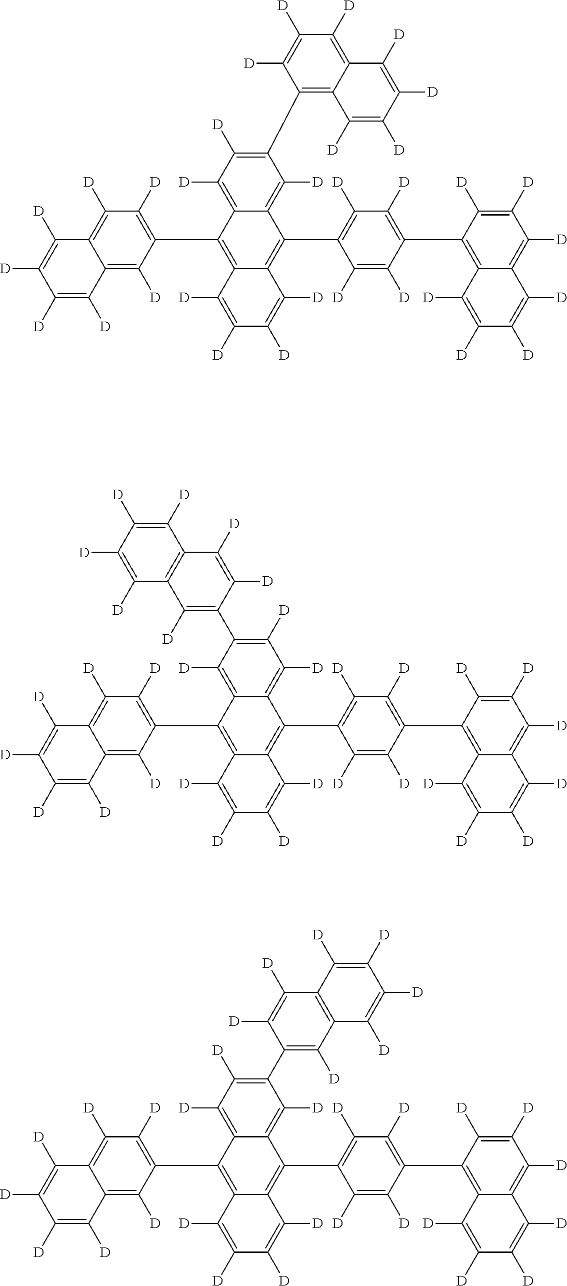

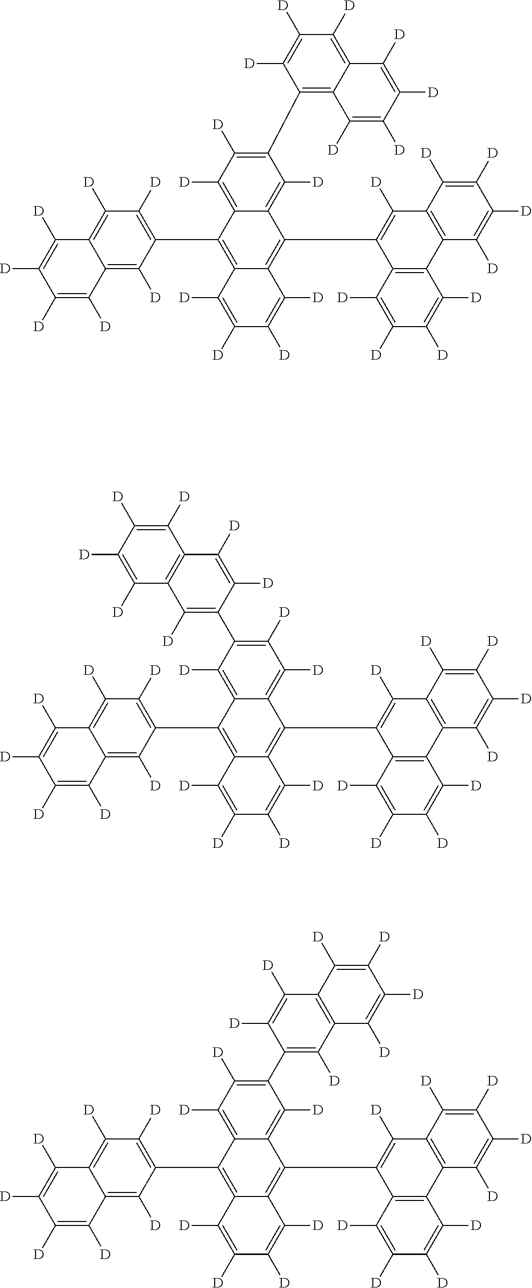

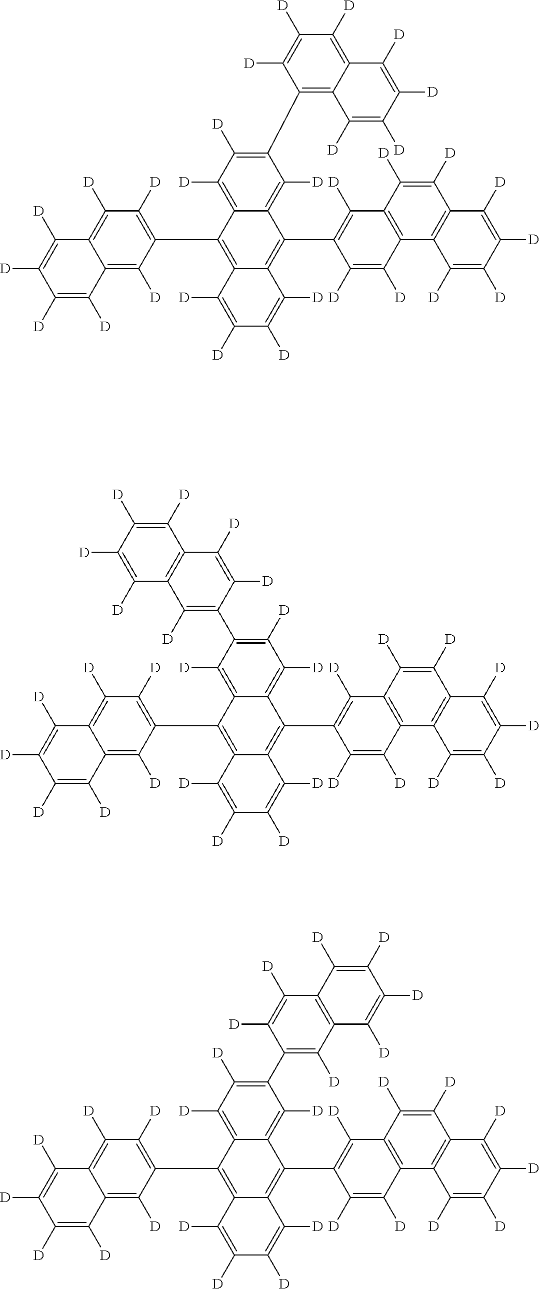

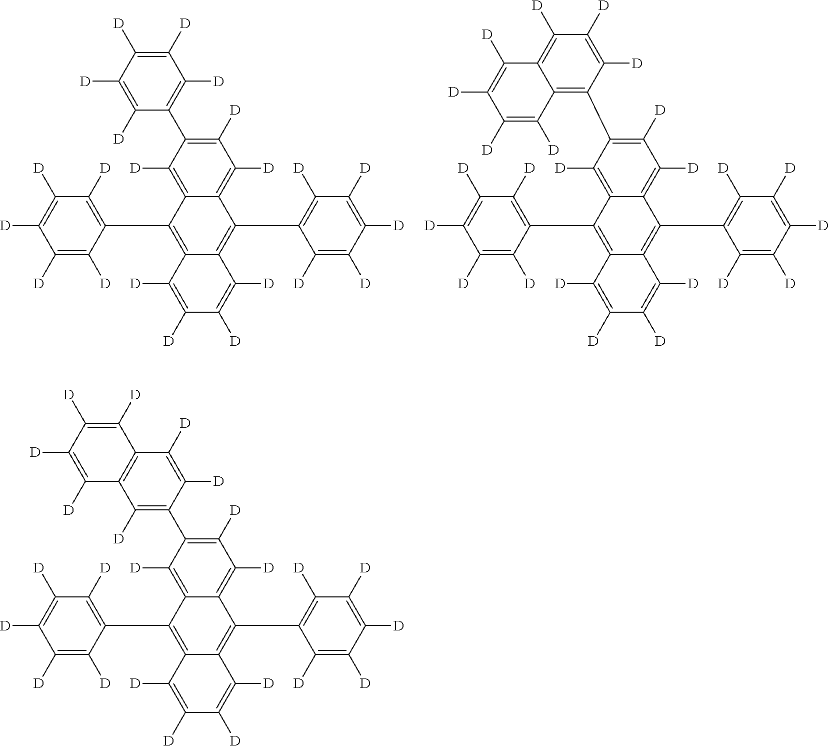

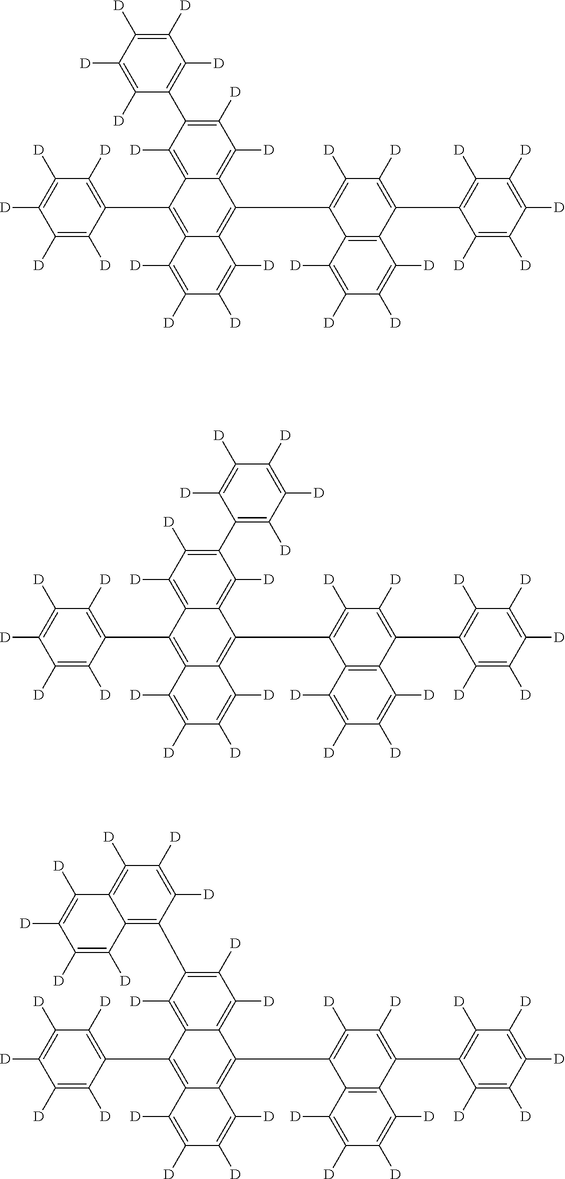

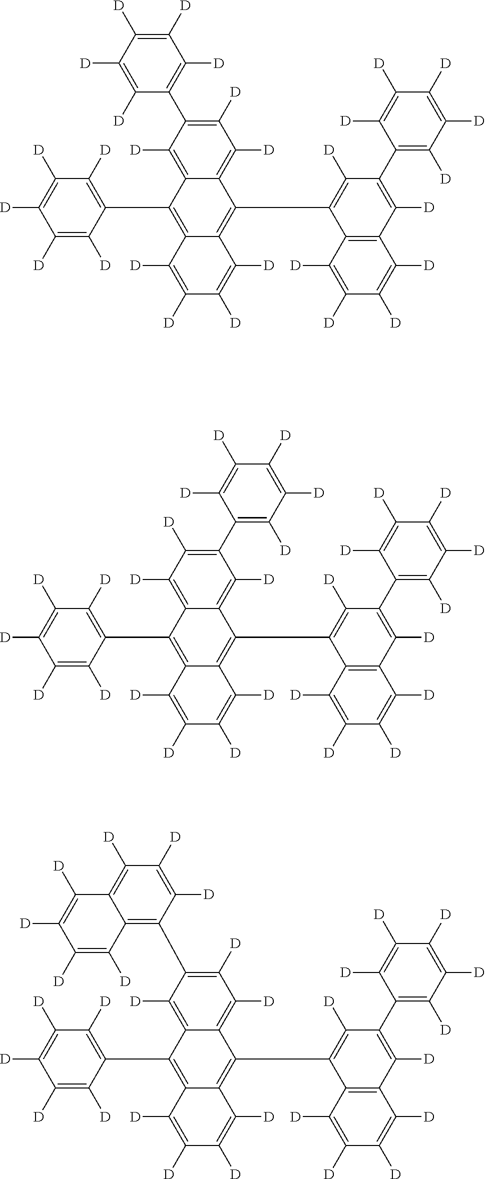

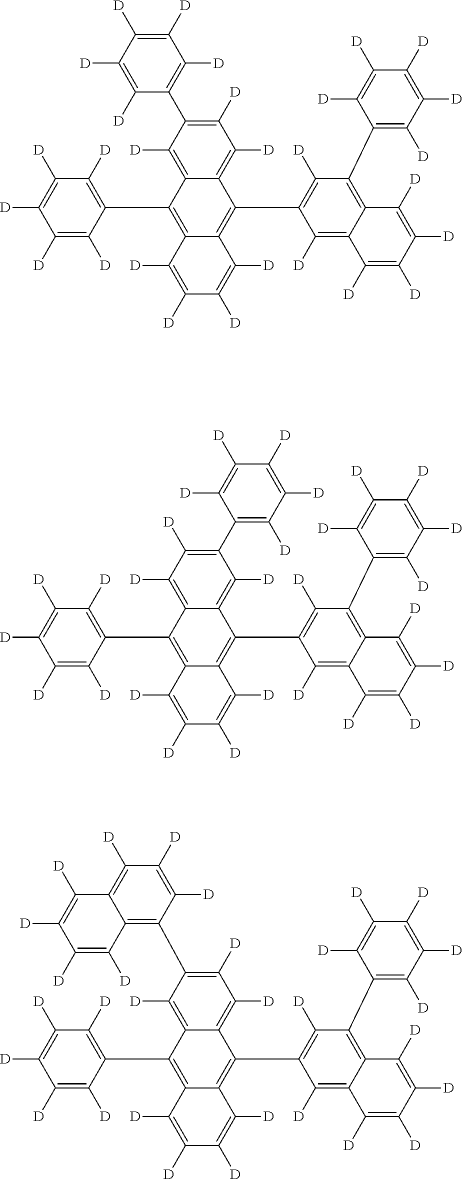

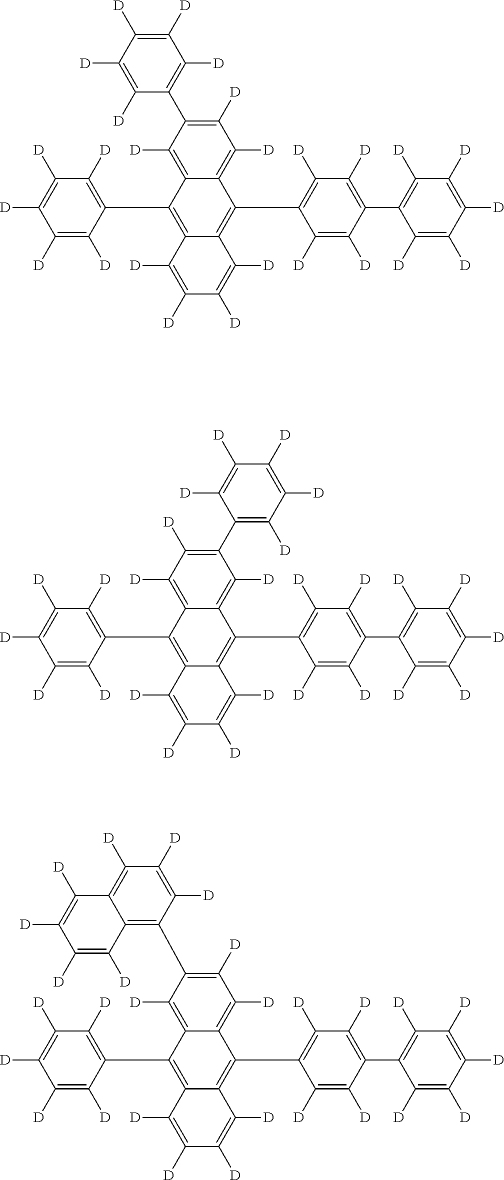

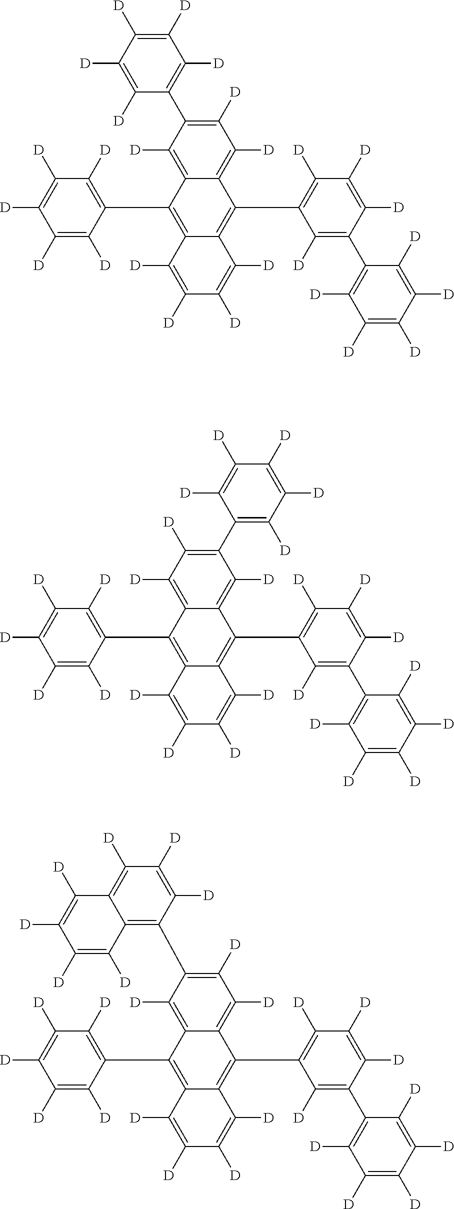

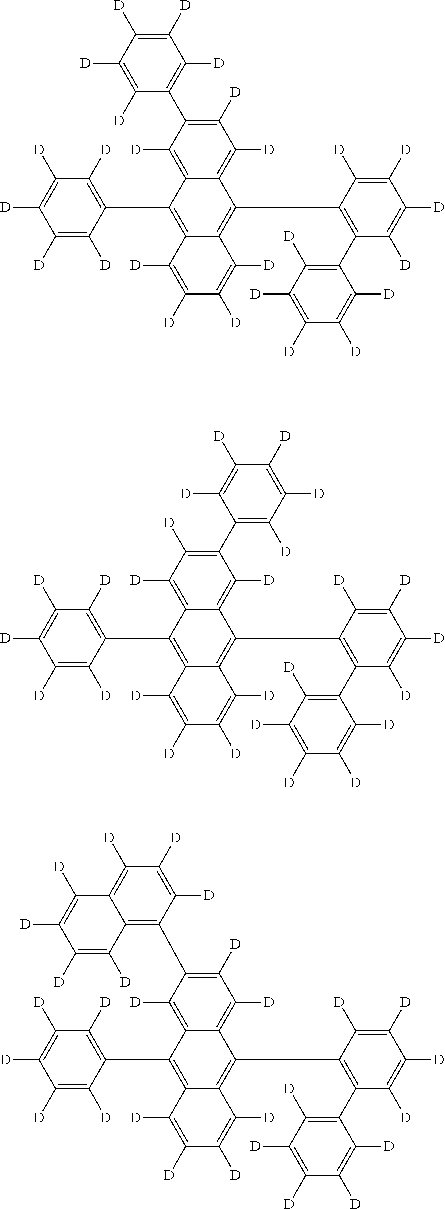

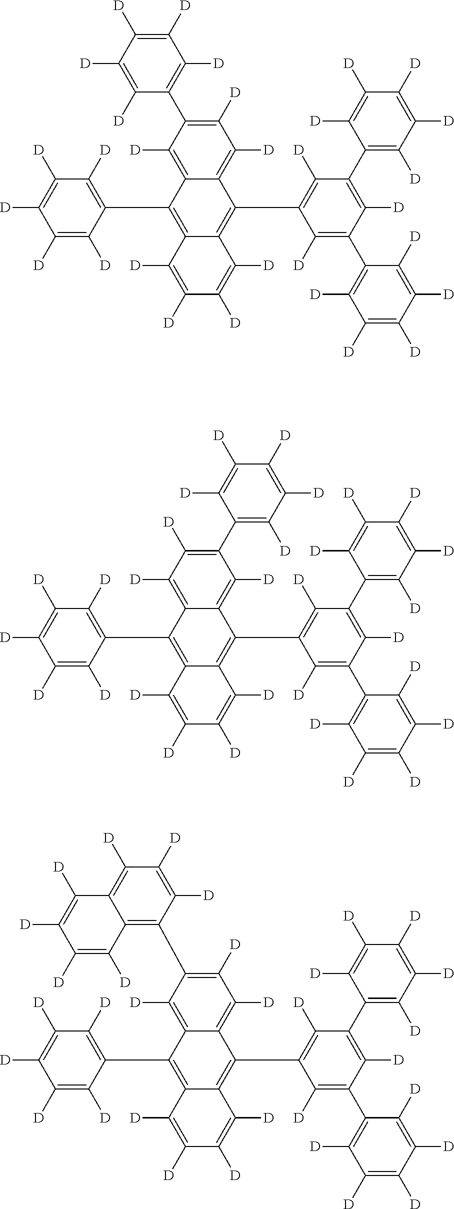

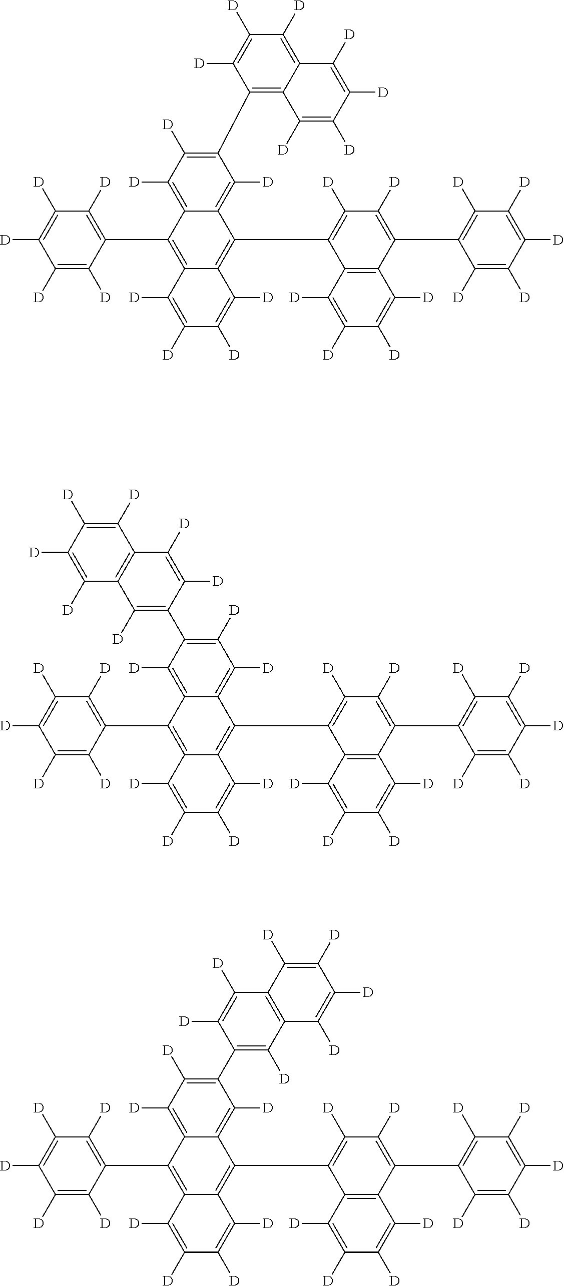

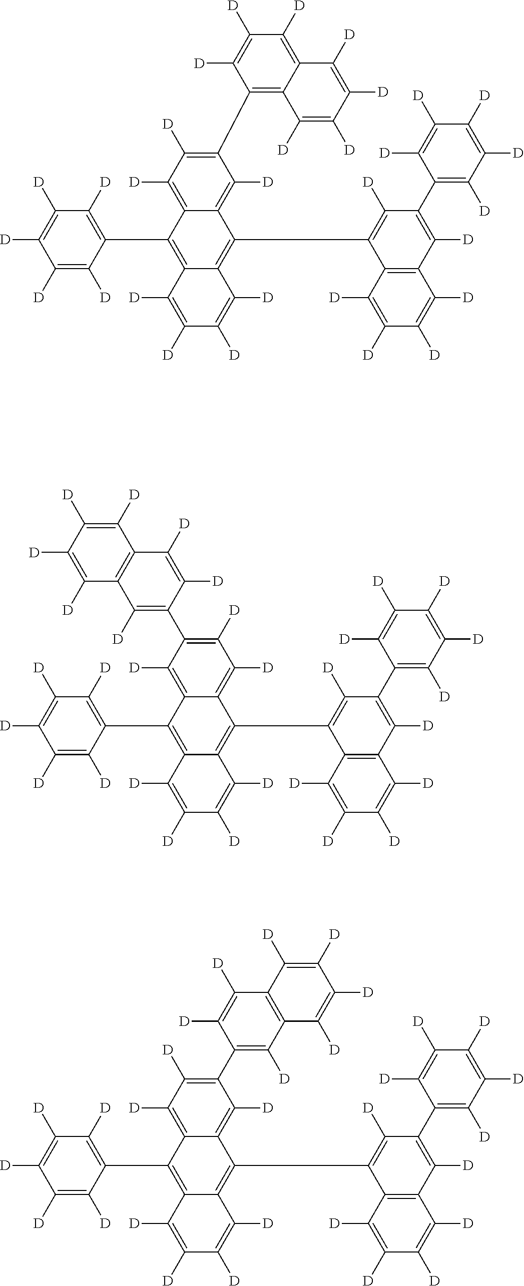

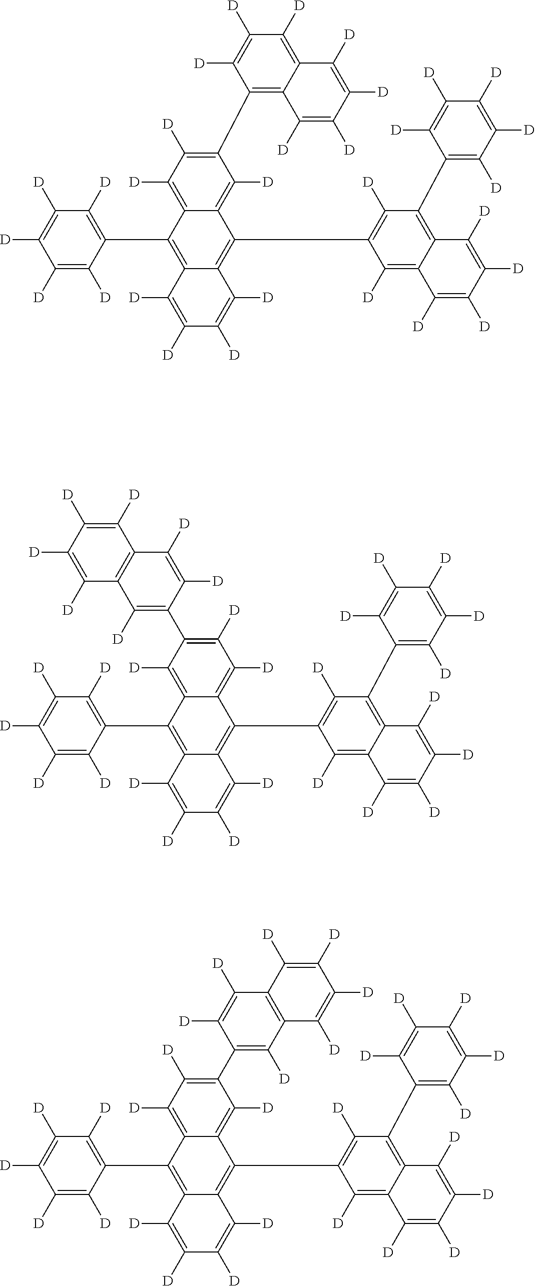

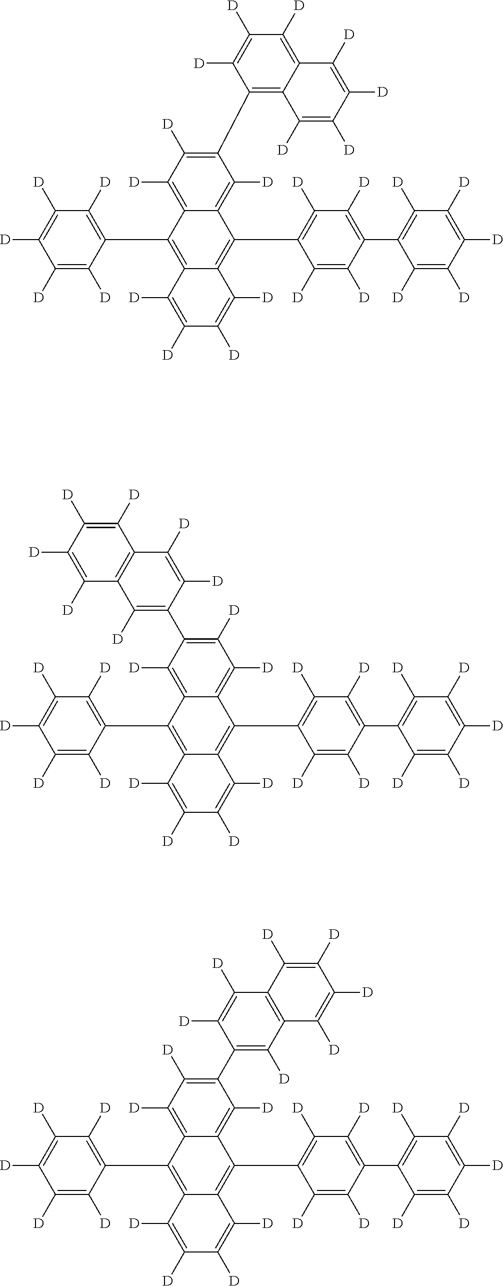

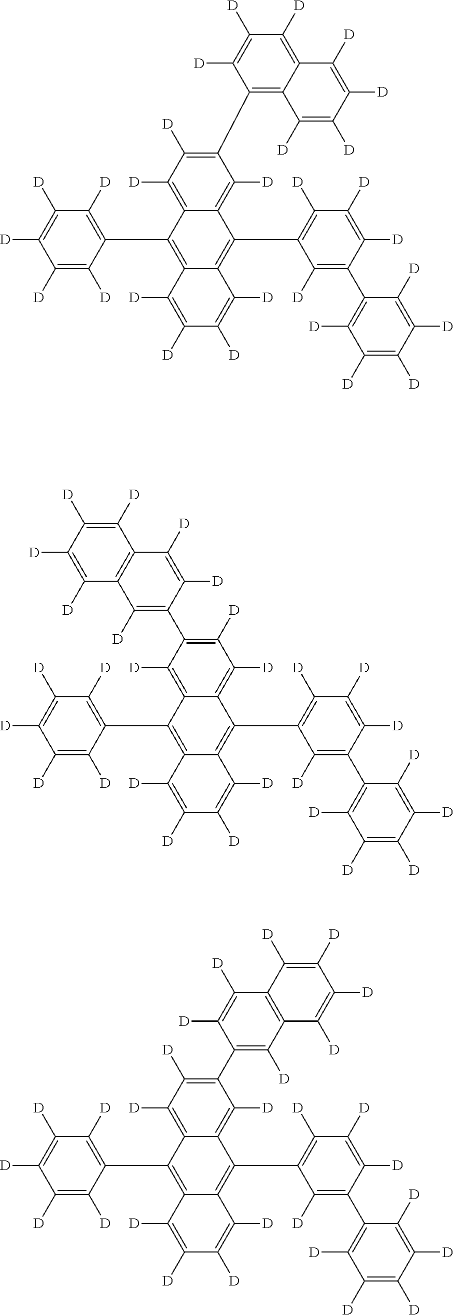

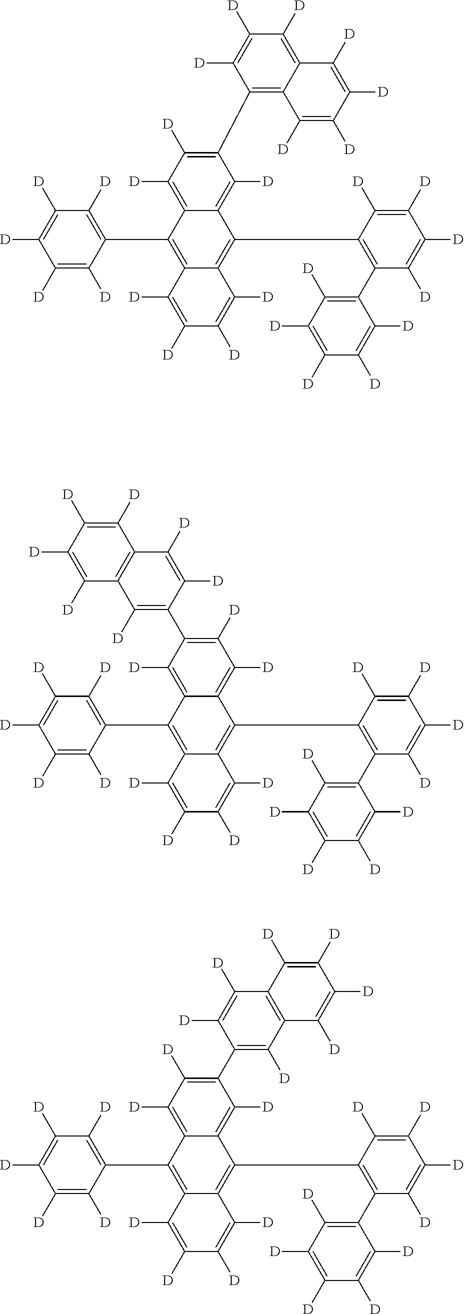

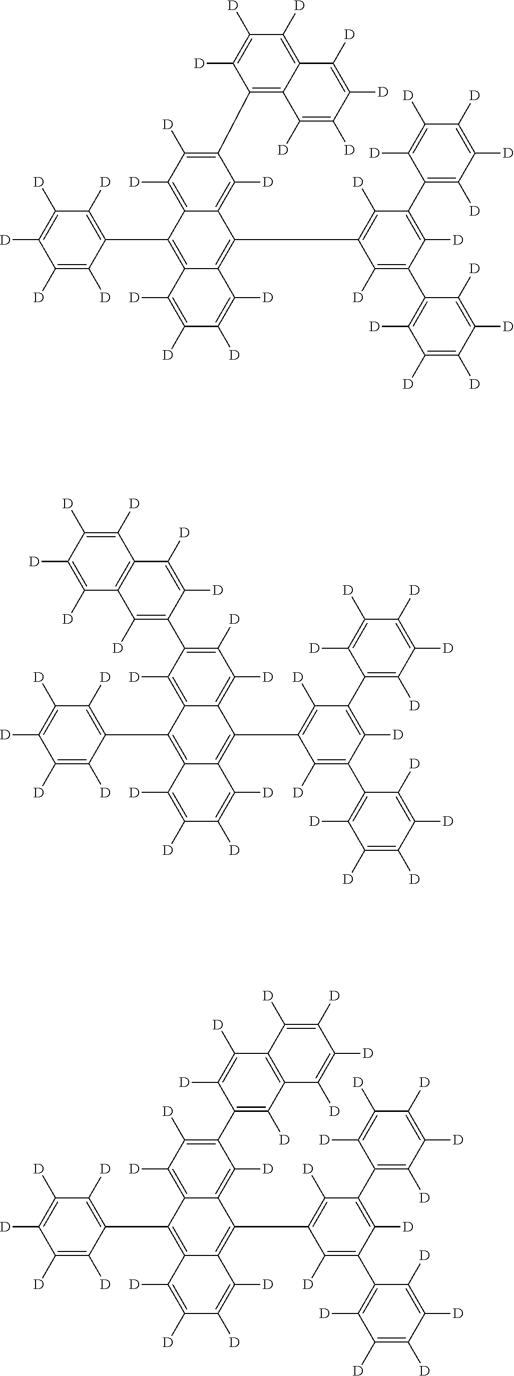

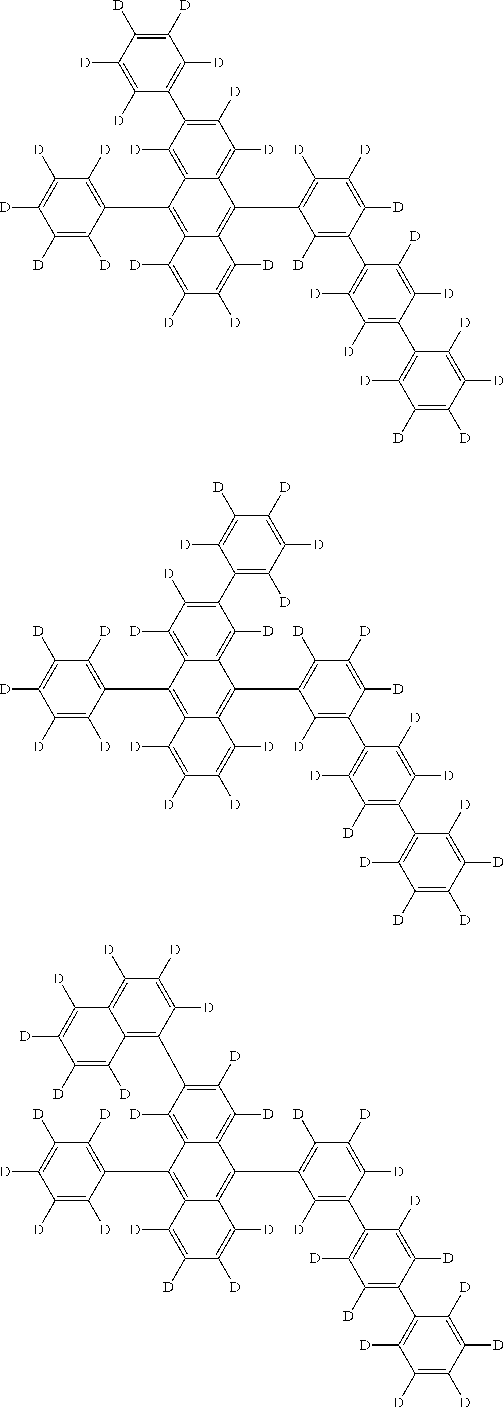

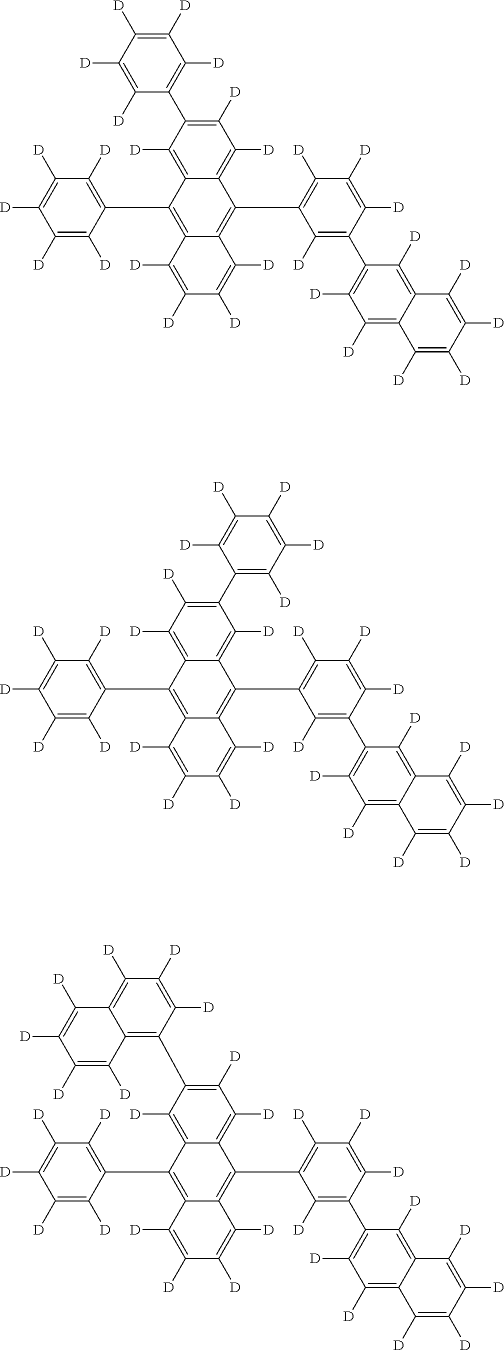

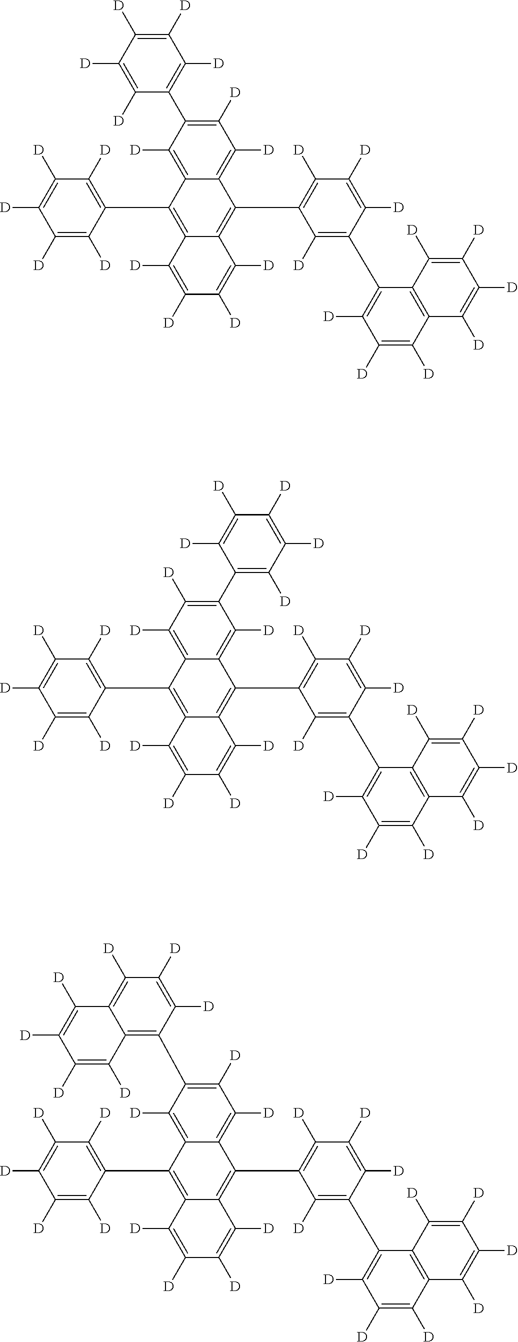

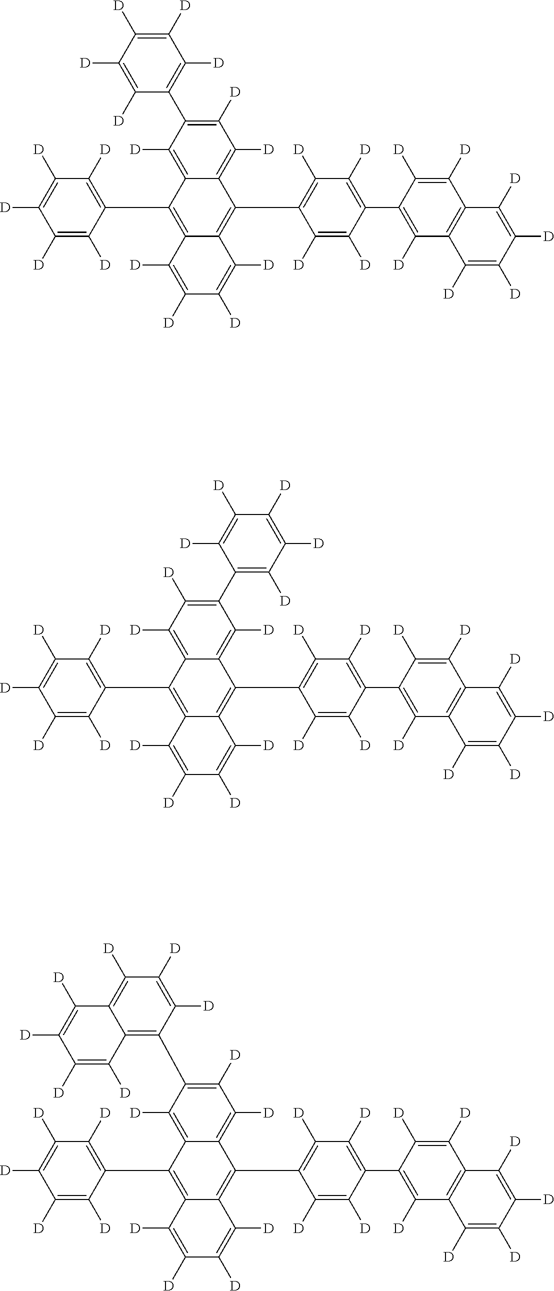

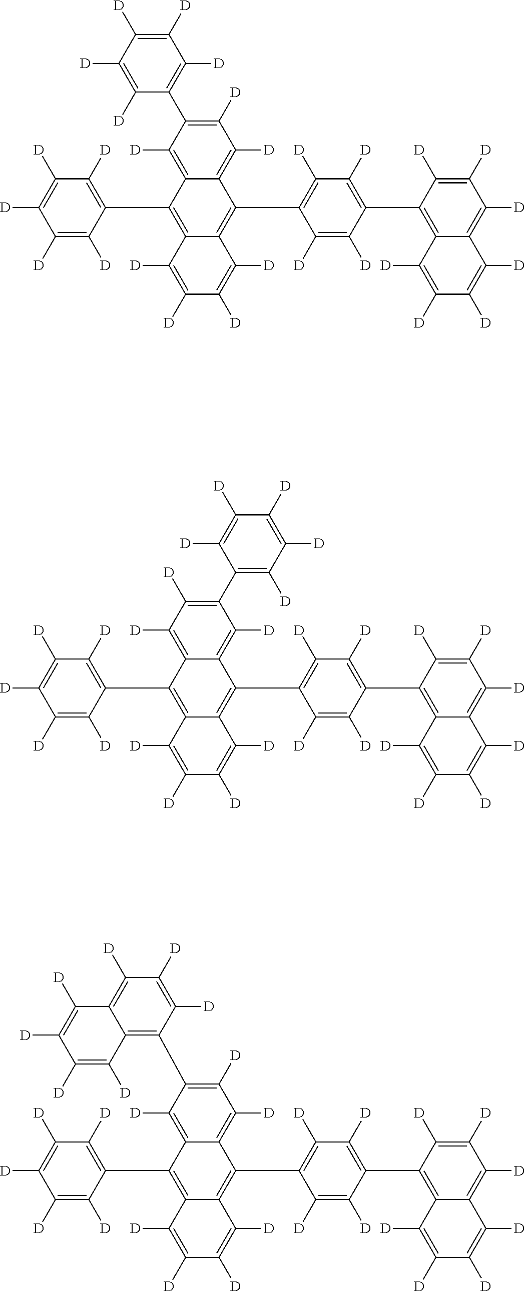

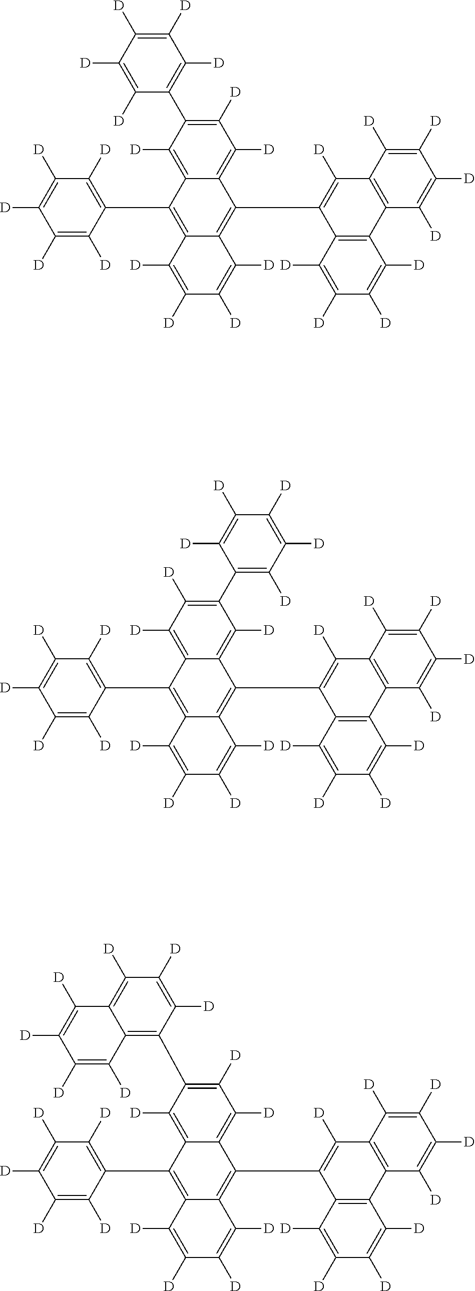

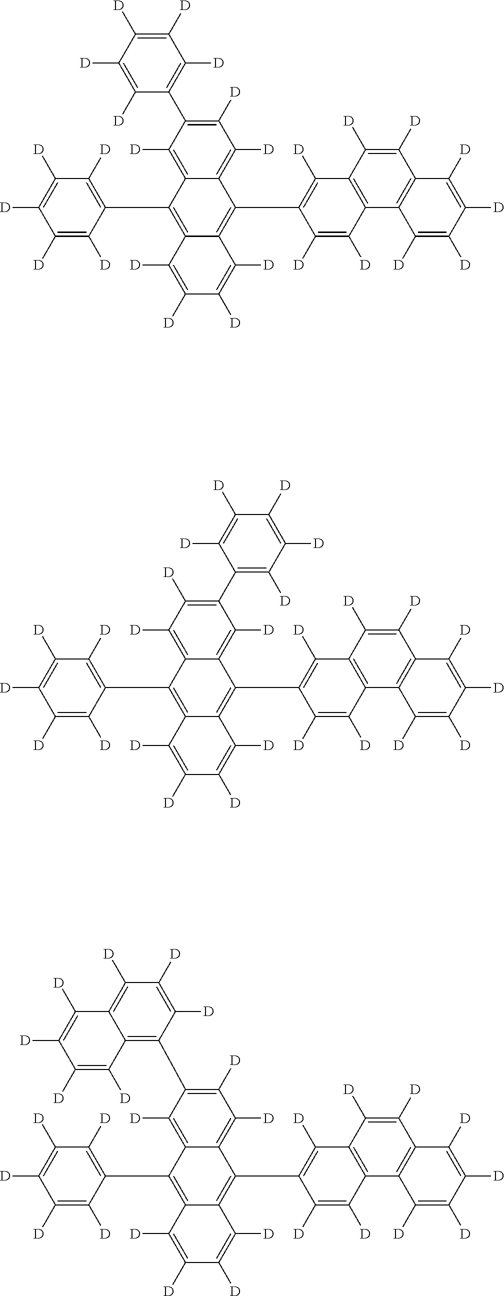

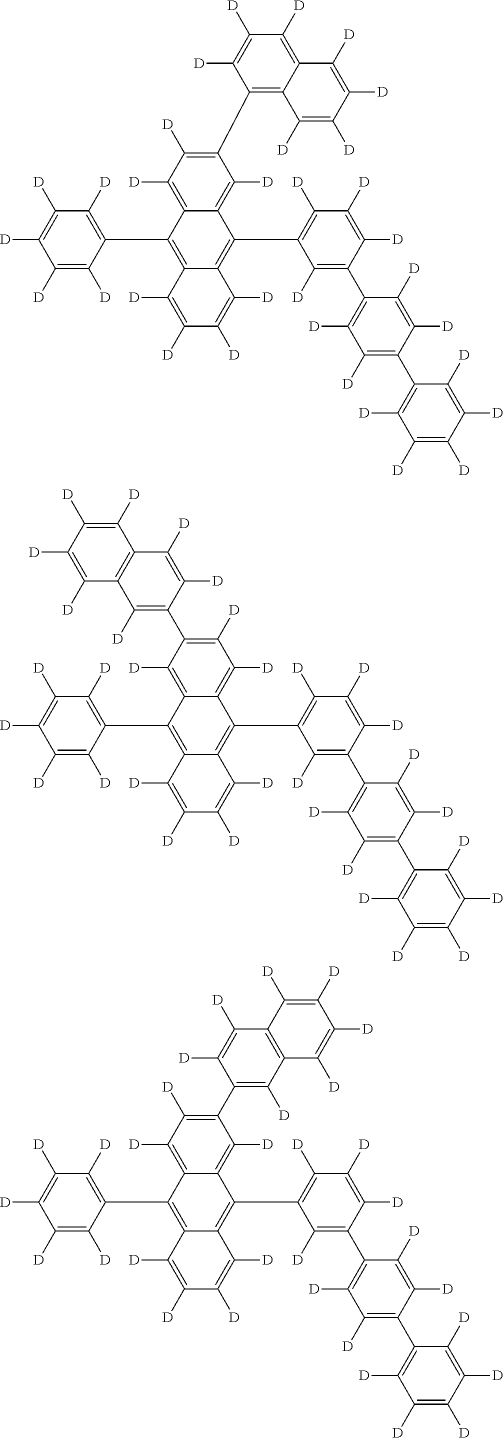

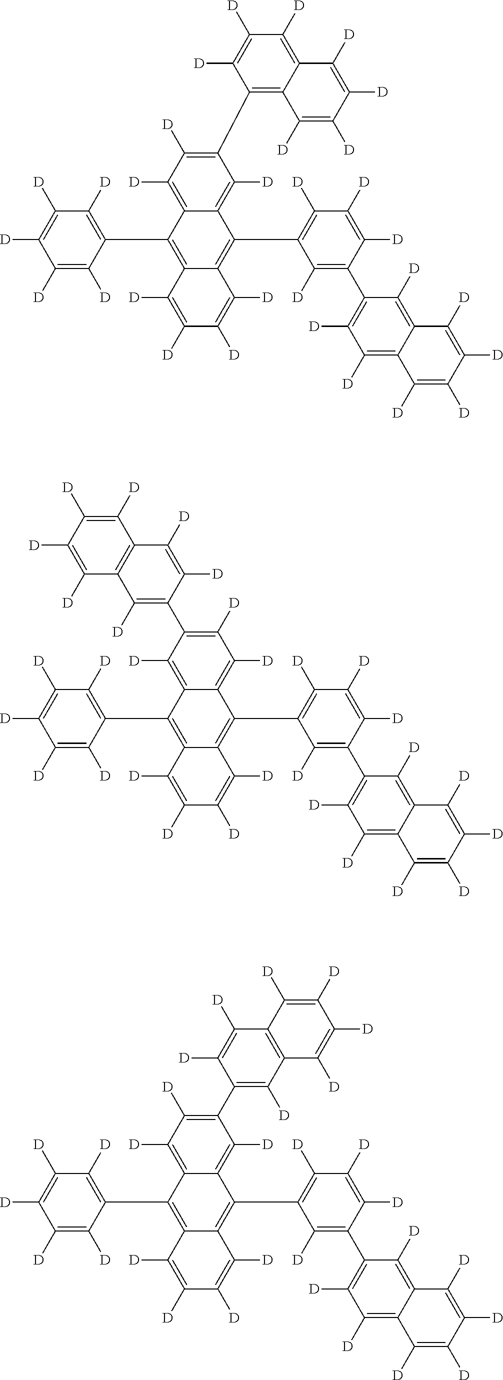

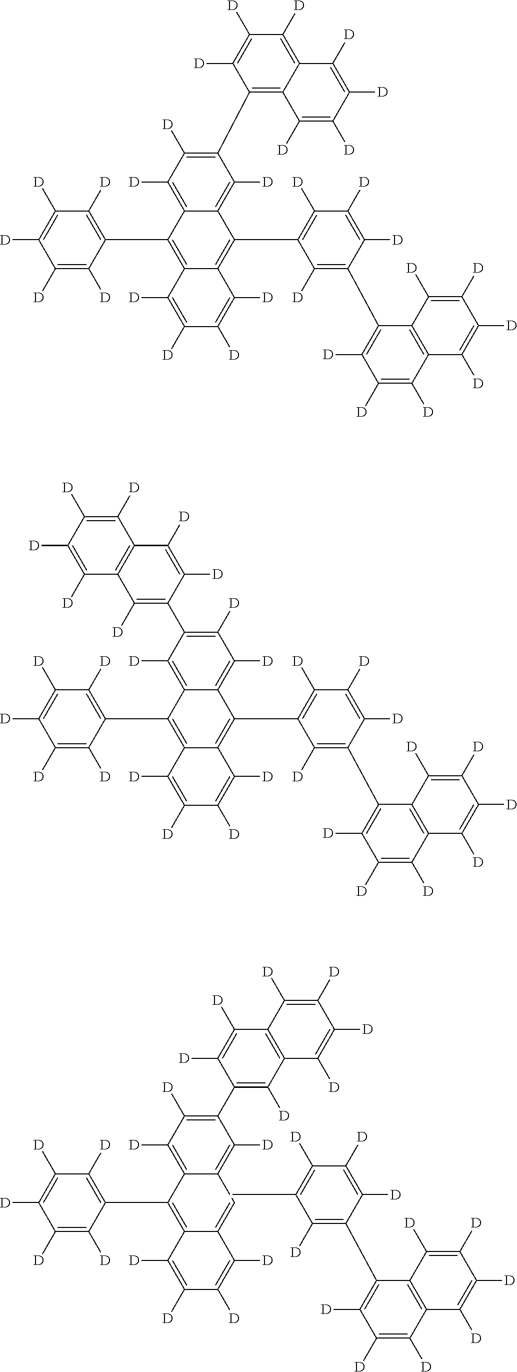

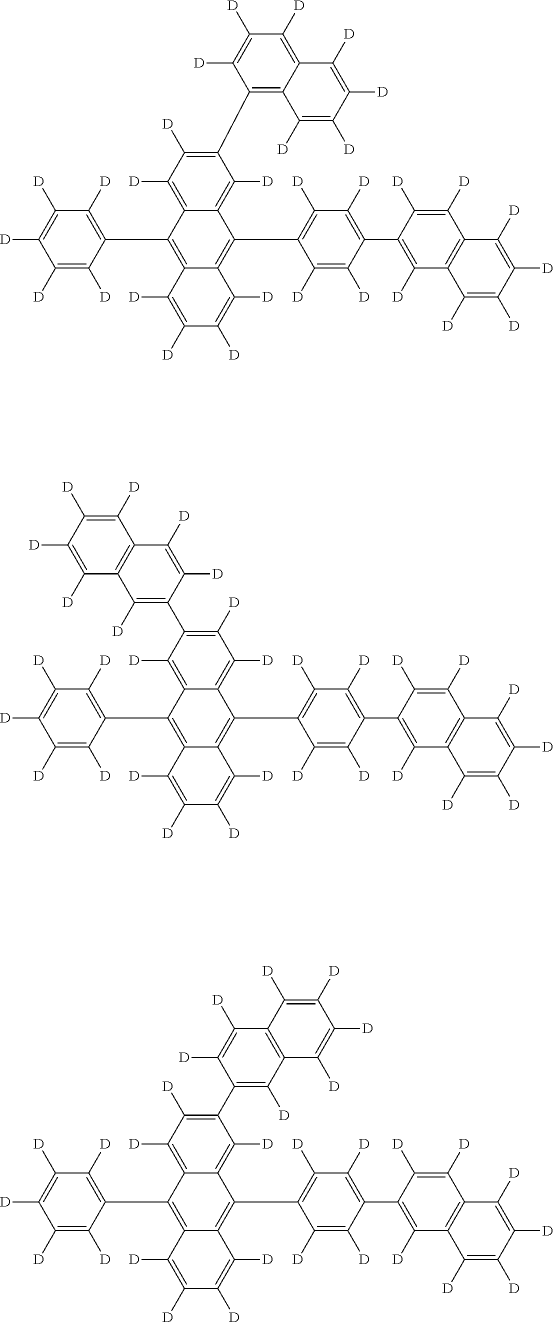

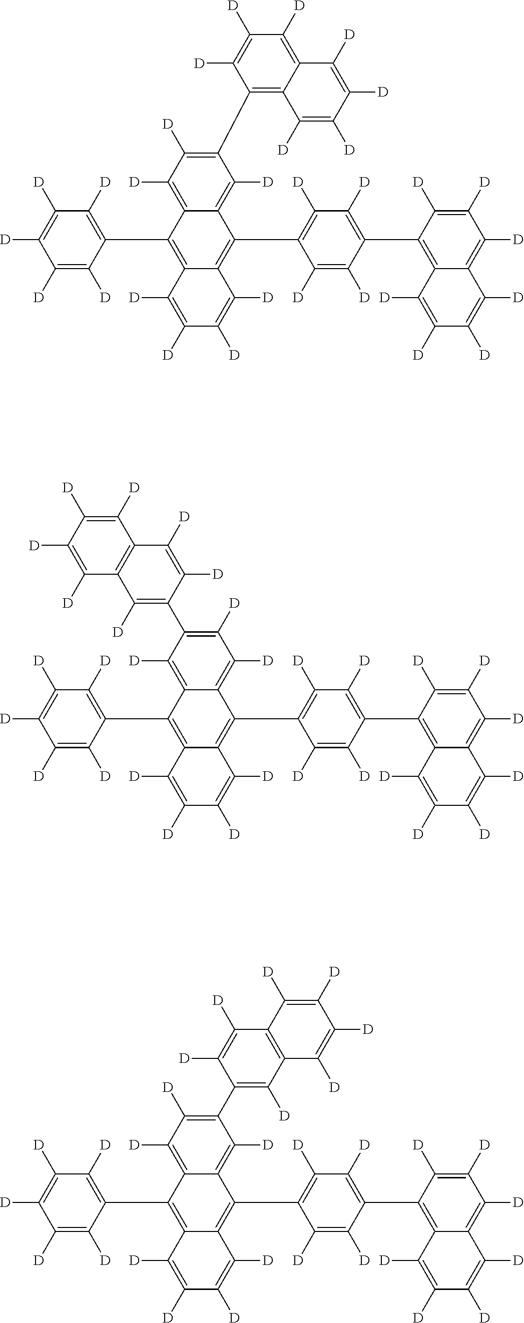

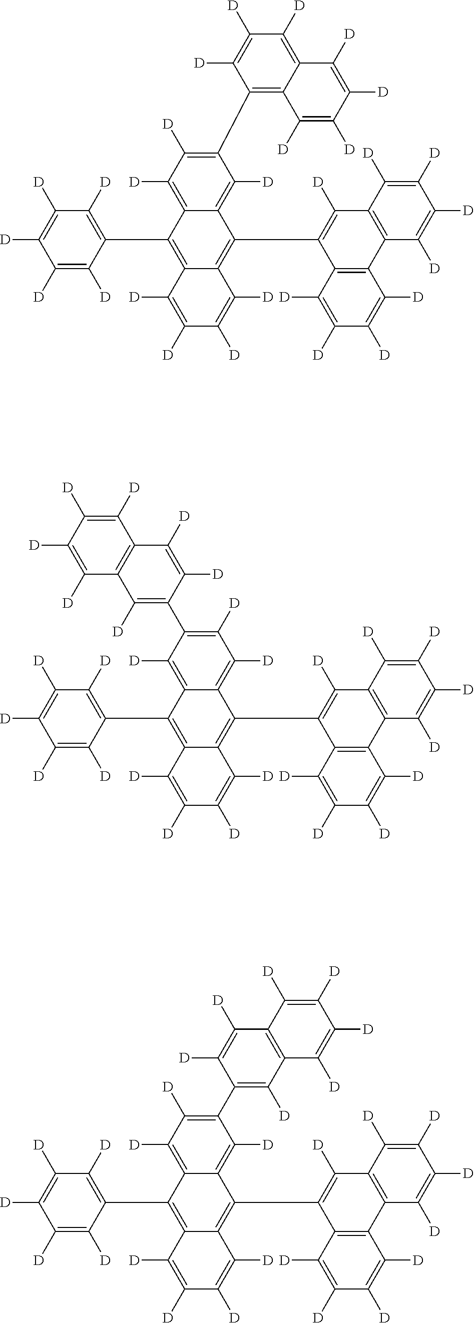

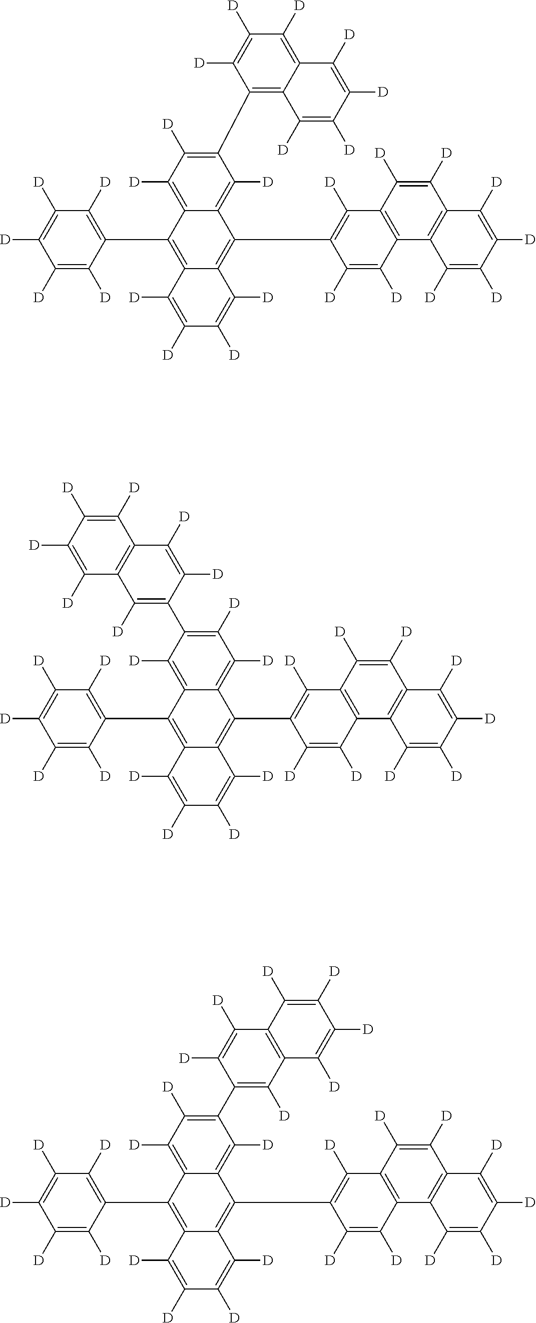

### <Compound Represented by the Formula (A1)>

The emitting layer of the organic EL device of an aspect of the invention comprises a compound represented by the following formula (A1).

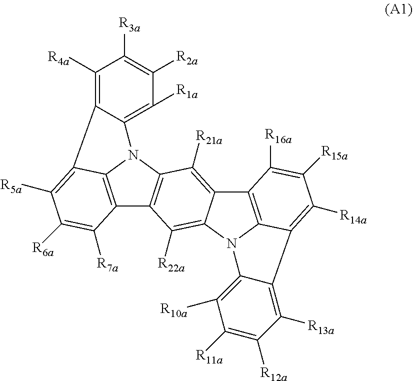

In the formula (A1),

one or more sets of adjacent two or more among R₁ₐ to R₇ₐ and R₁₀ₐ to R₁₆ₐ form a substituted or unsubstituted, saturated or unsaturated ring by bonding with each other, or do not form a substituted or unsubstituted, saturated or unsaturated ring;

R₁ₐ to R₇ₐ and R₁₀ₐ to R₁₆ₐ which do not form the substituted or unsubstituted, saturated or unsaturated ring, and R₂₁ₐ and R₂₂ₐ are independently a hydrogen atom or a substituent.

The substituent is

a substituted or unsubstituted alkyl group including 1 to 50 carbon atoms,

a substituted or unsubstituted alkenyl group including 2 to 50 carbon atoms,

a substituted or unsubstituted alkynyl group including 2 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄),

—S—(R₉₀₅),

—N(R₉₀₆)(R₉₀₇),

a halogen atom, a cyano group, a nitro group,

a substituted or unsubstituted aryl group including 6 to 50 ring carbon atoms, or

a substituted or unsubstituted monovalent heterocyclic group including 5 to 50 ring atoms.

R₉₀₁ to R₉₀₇ are independently

a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

when two or more of each of R₉₀₁ to R₉₀₇ are present, the two or more of each of R₉₀₁ to R₉₀₇ may be the same or different.

In one embodiment, in the formula (A1), one or more among R₁ₐ to R₇ₐ and R₁₀ₐ to R₁₆ₐ are —N(R₉₀₆)(R₉₇) (where R₉₀₆ and R₉₀₇ are as defined in the formula (A1)).

In one embodiment, in the formula (A1), two or more among R₁ₐ to R₇ₐ and R₁₀ₐ to R₁₆ₐ are —N (R₉₀₆)(R₉₀₇) (where R₉₀₆ and R₉₀₇ are as defined in the formula (A1)).

In one embodiment, the compound represented by the formula (A1) is a compound represented by the following formula (A10).

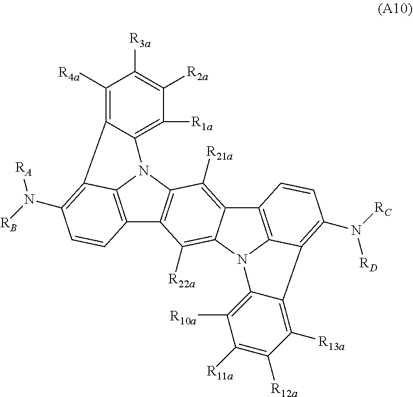

In the formula (A10),

R₁ₐ to R₄ₐ, R₁₀ₐ to R₁₃ₐ, R₂₁ₐ and R₂₂ₐ are as defined in the formula (A1);

R_{A}, R_{B}, R_{C} and R_{D} are independently a substituted or unsubstituted aryl group including 6 to 18 ring carbon atoms, or a substituted or unsubstituted monovalent heterocyclic group including 5 to 18 ring atoms.

In one embodiment, the compound represented by the formula (A10) is a compound represented by the following formula (A11).

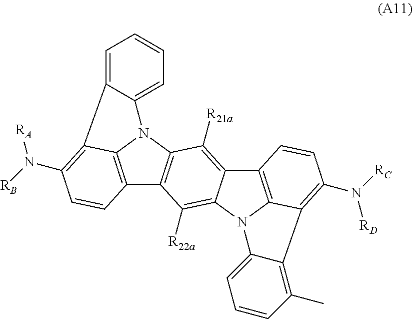

In the formula (A11),

R₂₁ₐ, R₂₂ₐ, R_{A}, R_{B}, R_{C} and R_{D} are as defined in the formula (A10).

In one embodiment, in the formulas (A10) and (A11), R_{A}, R_{B}, R_{C} and R_{D} are independently a substituted or unsubstituted aryl group including 6 to 18 ring carbon atoms.

In one embodiment, in the formulas (A10) and (A11), R_{A}, R_{B}, R_{C} and R_{D} are independently a substituted or unsubstituted phenyl group.

In one embodiment, in the formula (A1), one or more pairs selected from the group consisting of R₁ₐ and R₂ₐ, R₂ₐ and R₃ₐ, R₃ₐ and R₄ₐ, R₁₀ₐ and R₁₁ₐ, R₁₁ₐ and R₁₂ₐ, and R₁₂ₐ and R₁₃ₐ form a ring represented by the following formula (X).

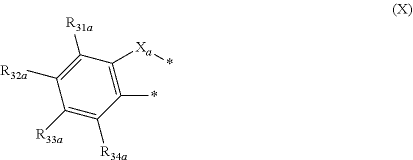

In the formula (X),

two “*” s are respectively bonded with R₁ₐ and R₂ₐ, R₂ₐ and R₃ₐ, R₃ₐ and R₄ₐ, R₁₀ₐ and R₁₁ₐ; R₁₁ₐ and R₁₂ₐ, and R₁₂ₐ and R₁₃ₐ in the formula (A1);

R₃₁ₐ to R₃₄ₐ are independently a hydrogen atom, a substituted or unsubstituted alkyl group including 1 to 50 carbon atoms, or a substituted or unsubstituted aryl group including 6 to 50 ring carbon atoms;

when two or more of each of R₃₁ₐ to R₃₄ₐ are present, the two or more of each of R₃₁ₐ to R₃₄ₐ may be the same or different;

Xₐ is selected from O, S and N(R₃₅ₐ);

when two or more Xₐ's are present, the two or more Xₐ's may be the same or different;

R₃₅ₐ forms a substituted or unsubstituted, saturated or unsaturated ring by bonding with R₃₁, or does not form a ring;

R₃₅ₐ which does not form a ring is a hydrogen atom,

a substituted or unsubstituted alkyl group including 1 to 50 carbon atoms,

a substituted or unsubstituted cycloalkyl group including 3 to 50 ring carbon atoms,

a substituted or unsubstituted aryl group including 6 to 50 ring carbon atoms, or

a substituted or unsubstituted monovalent heterocyclic group including 5 to 50 ring atoms.

In one embodiment, the compound represented by the formula (A1) is a compound represented by the following formula (A12).

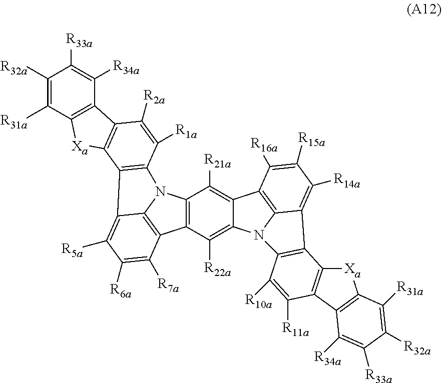

In the formula (A12),

R₁ₐ, R₂ₐ, R₅ₐ to R₇ₐ, R₁₀ₐ, R₁₁ₐ, R₁₄ₐ to R₁₆ₐ, R₂₁ₐ, R₂₂ₐ, R₃₁ₐ to R₃₄ₐ and Xₐ are as defined in the formulas (A1) and (X).

In one embodiment, the compound represented by the formula (A1) is a compound represented by the following formula (A13).

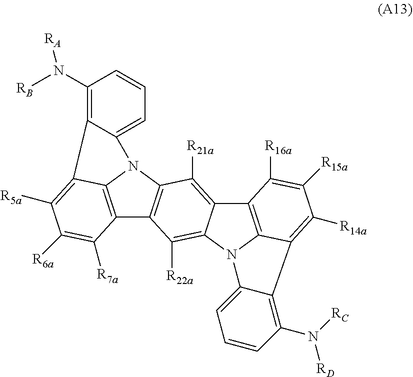

In the formula (A13), R₅ₐ to R₇ₐ, R₁₄ₐ to R₁₆ₐ, R₂₁ₐ, R₂₂ₐ, R_{A}, R_{B}, R_{C} and R_{D} are as defined in the formulas (A1) and (A10).

In one embodiment, the compound represented by the formula (A13) is a compound represented by the following formula (A14).

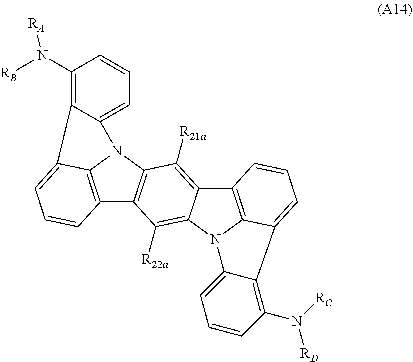

In the formula (A14), R₂₁ₐ, R₂₂ₐ, R_{A}, R_{B}, R_{C} and R_{D} are as defined in the formulas (A1) and (A10).

In one embodiment, the compound represented by the formula (A1) is a compound represented by the following formula (A15).

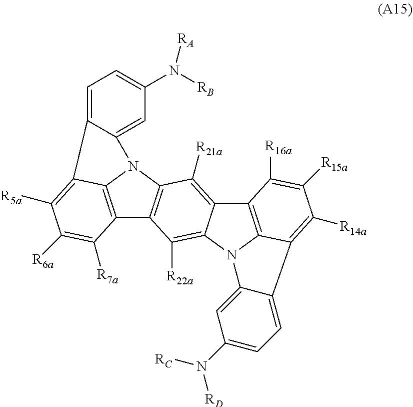

In the formula (A15), R₅ₐ to R₇ₐ, R₁₄ₐ to R₁₆ₐ, R₂₁ₐ, R₂₂ₐ, R_{A}, R_{B}, R_{C} and R_{D} are as defined in the formulas (A1) and (A10).

In one embodiment, the compound represented by the formula (A15) is a compound represented by the following formula (A16).

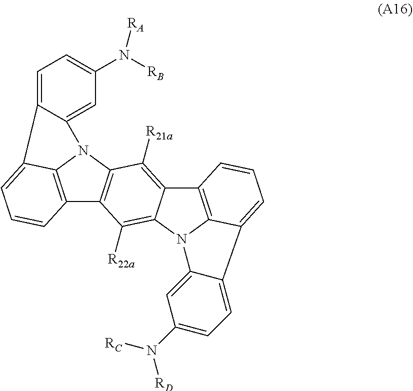

In the formula (A16), R₂₁ₐ, R₂₂ₐ, R_{A}, R_{B}, R_{C} and R_{D} are as defined in the formulas (A1) and (A10).

In one embodiment, in the formula (A1), R₂₁ₐ and R₂₂ₐ are hydrogen atoms.

Specific examples of the compound represented by the formula (A1) include the following compounds. In the following specific examples, “Ph” represents a phenyl group, and “D” represents a deuterium atom.

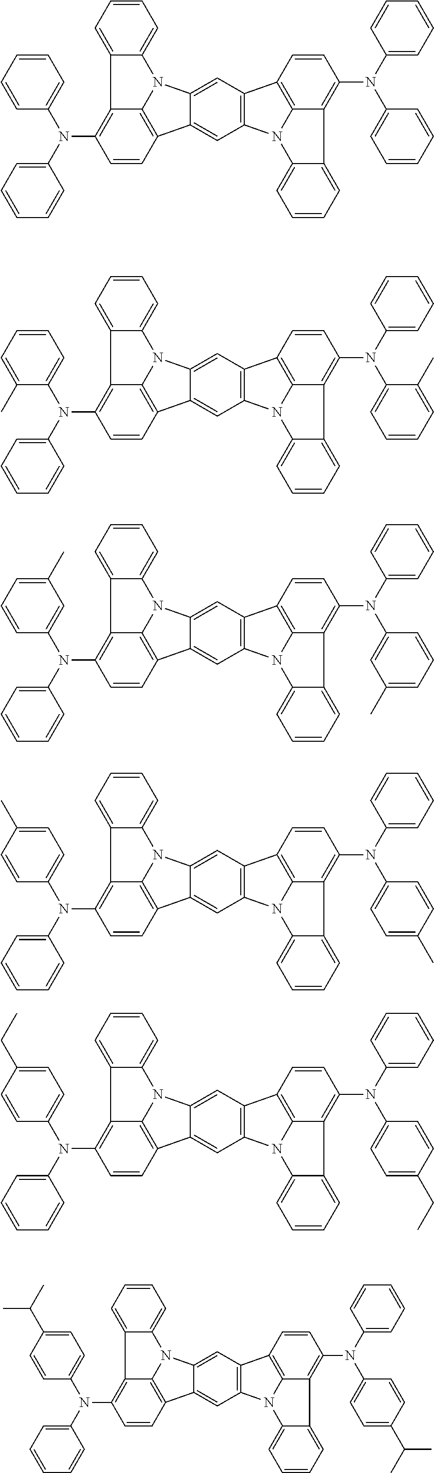

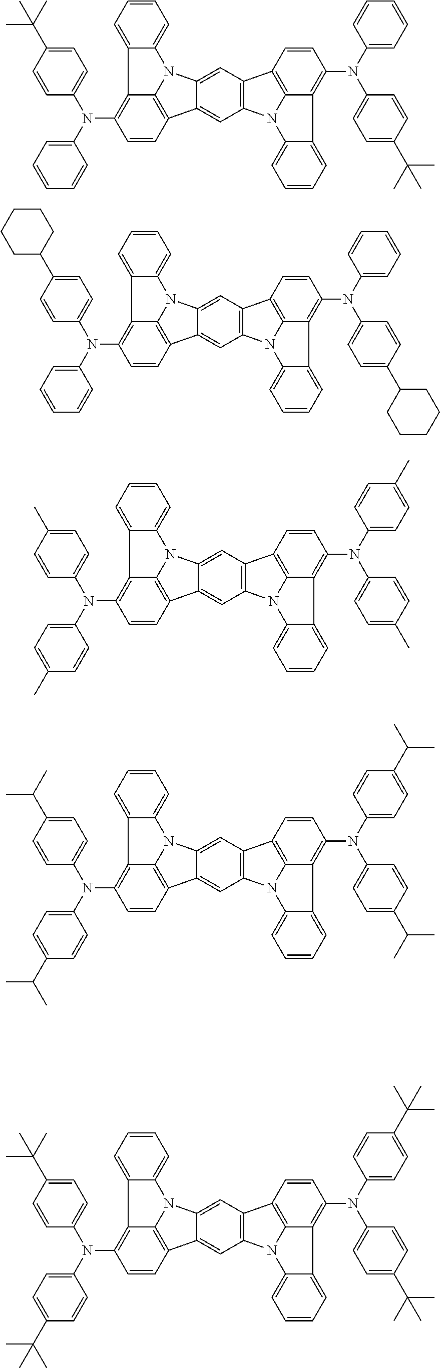

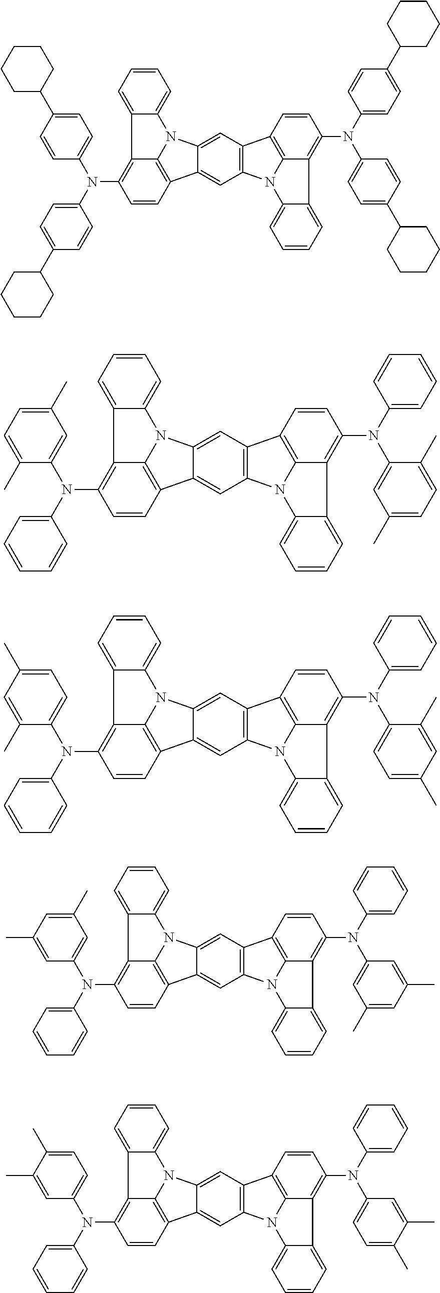

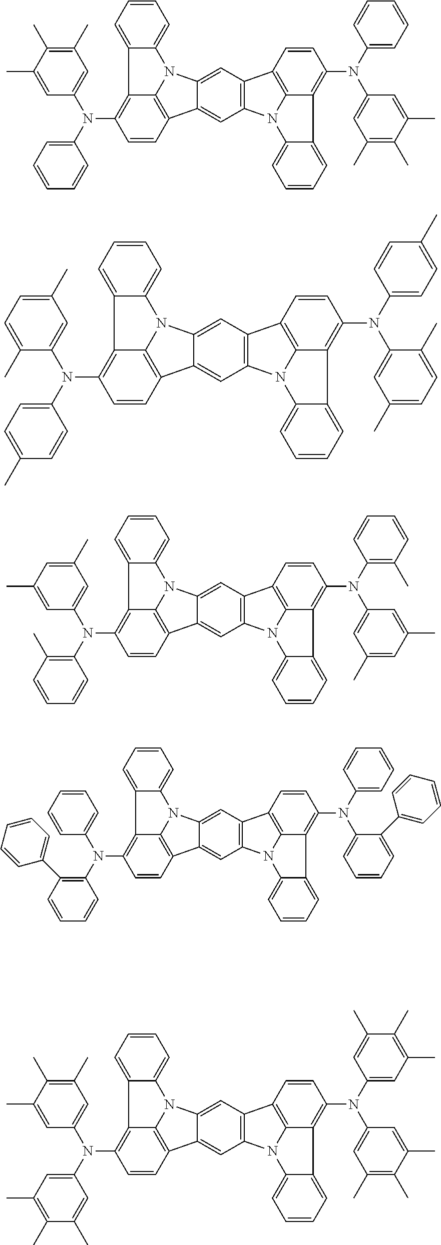

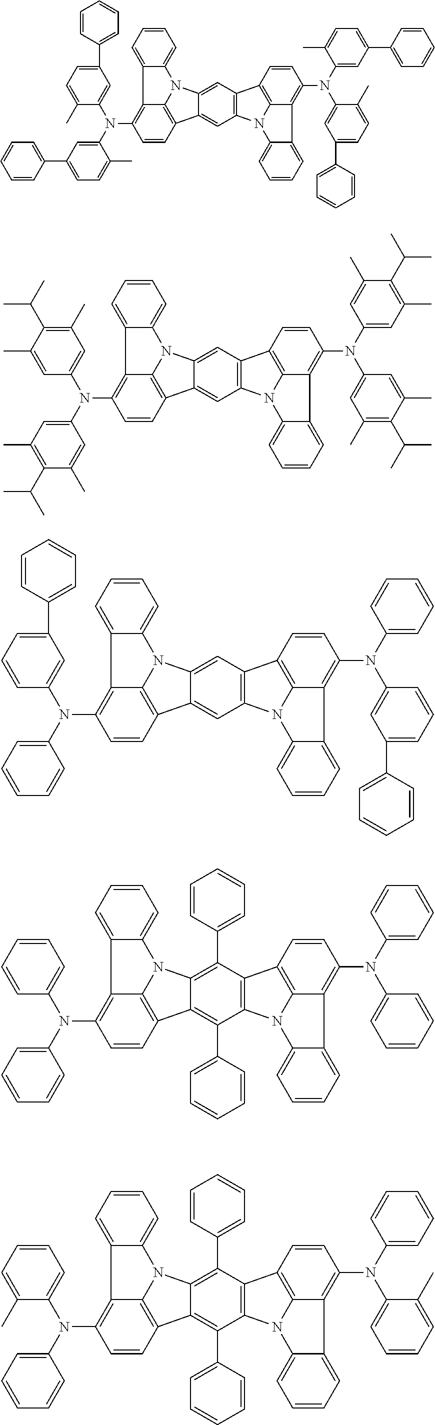

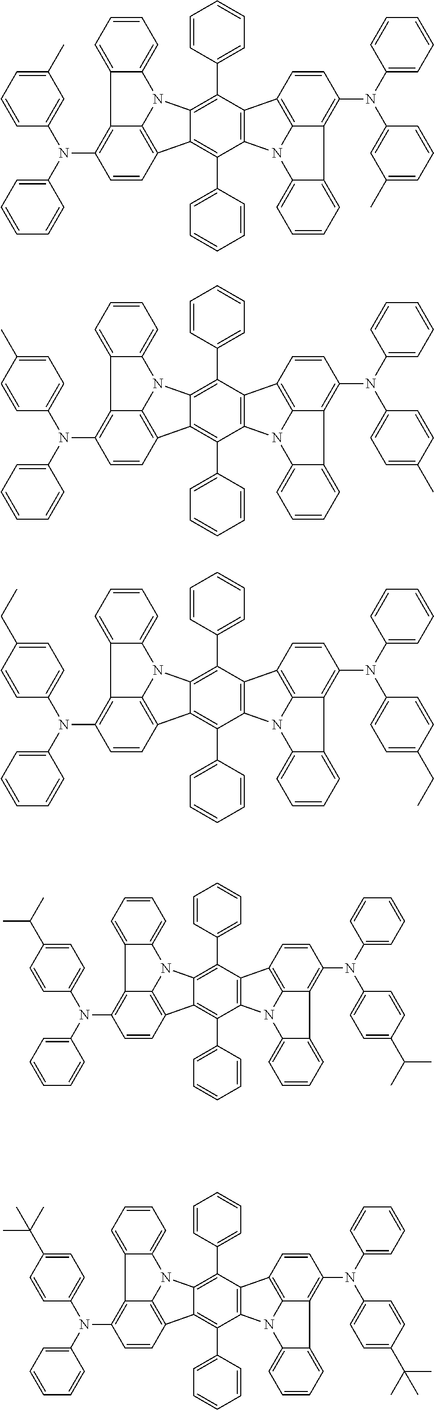

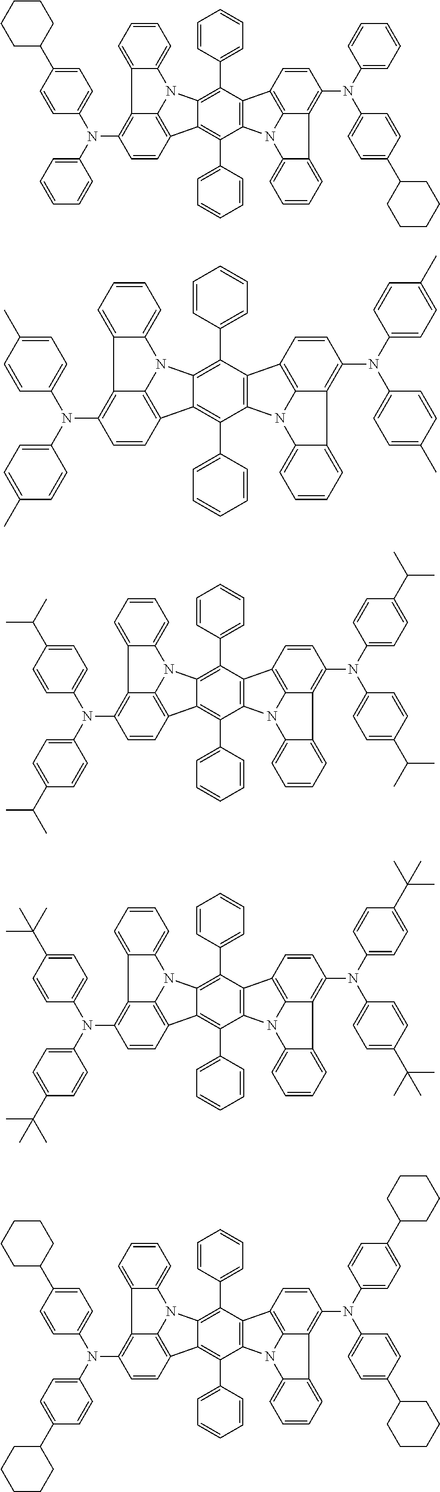

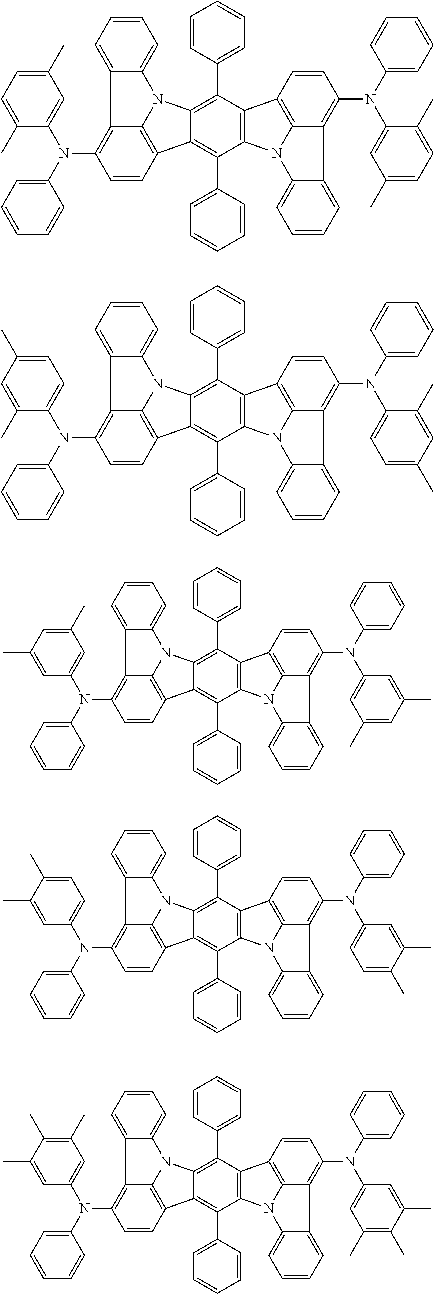

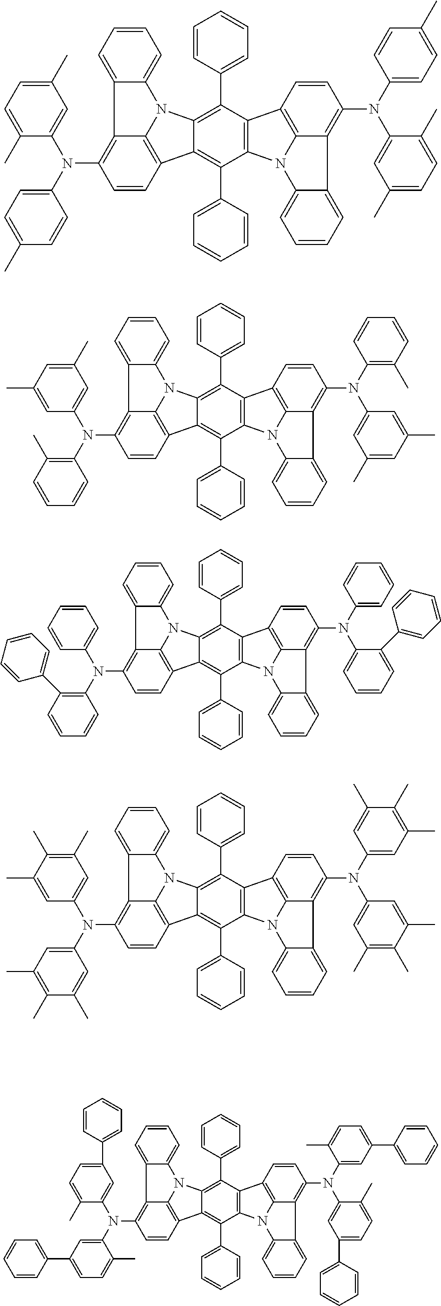

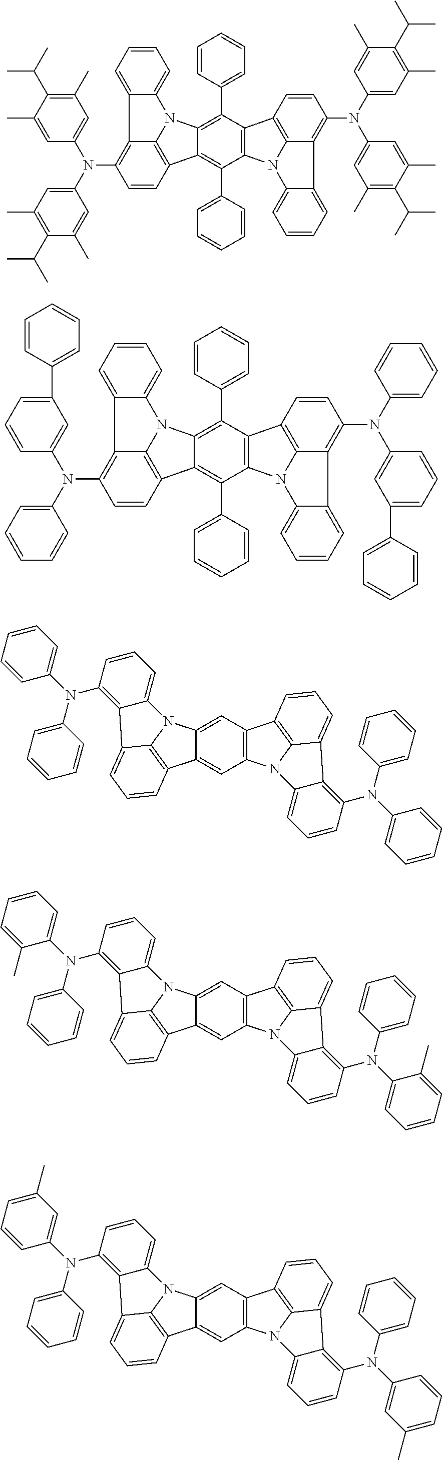

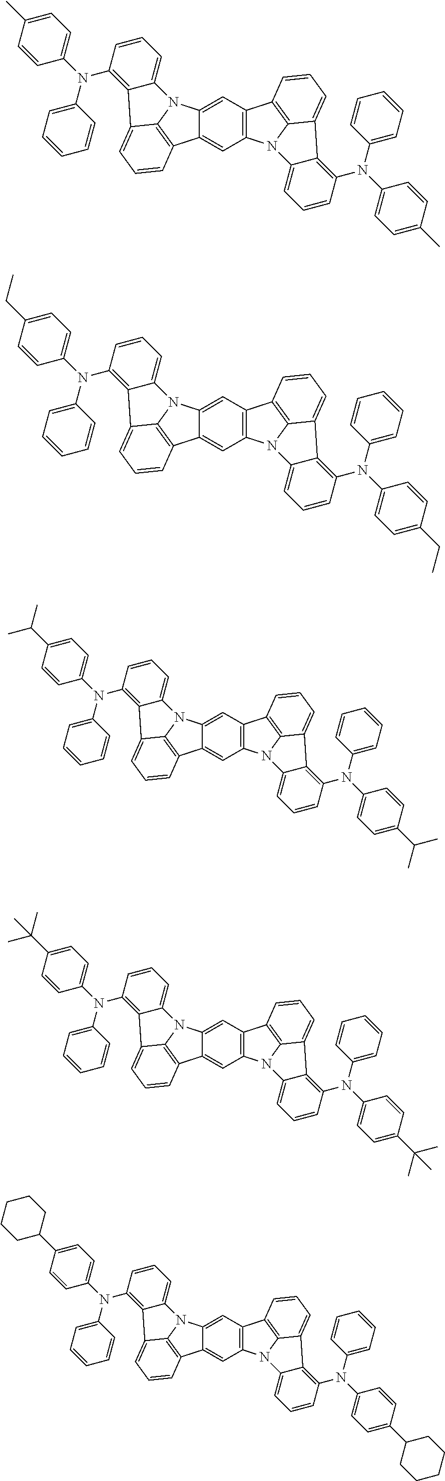

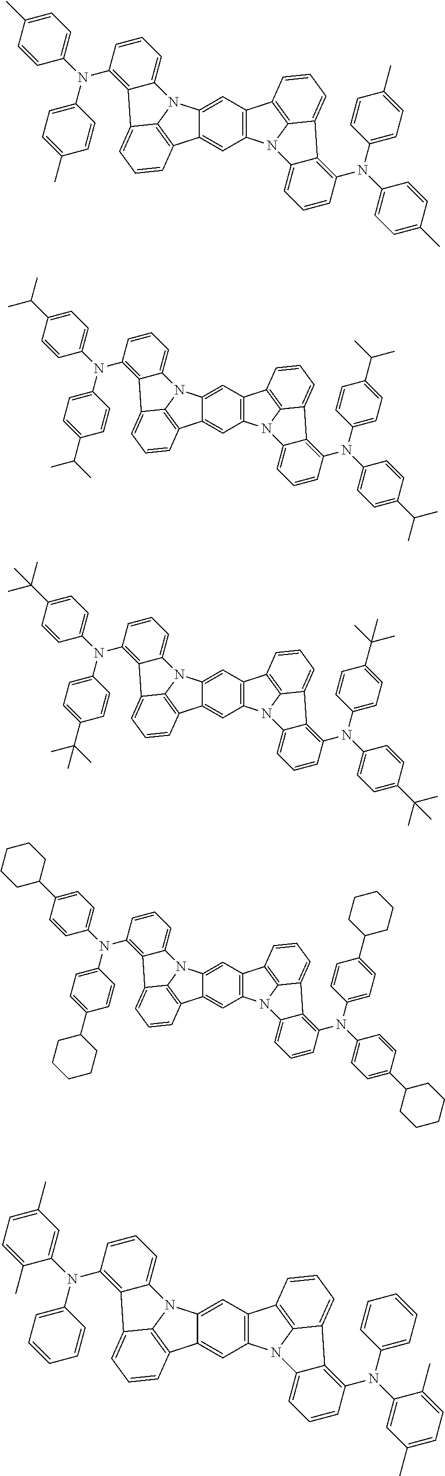

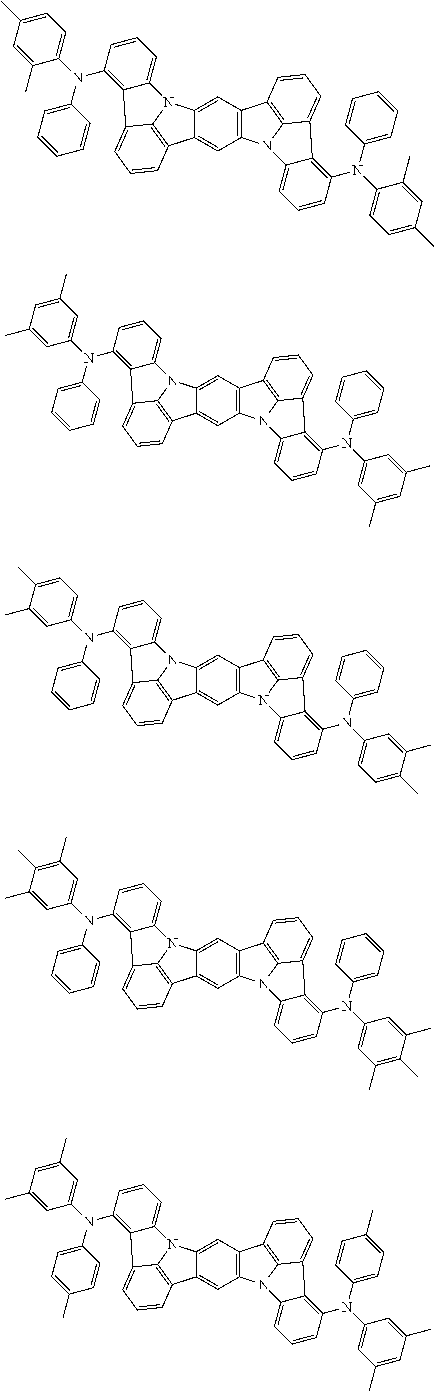

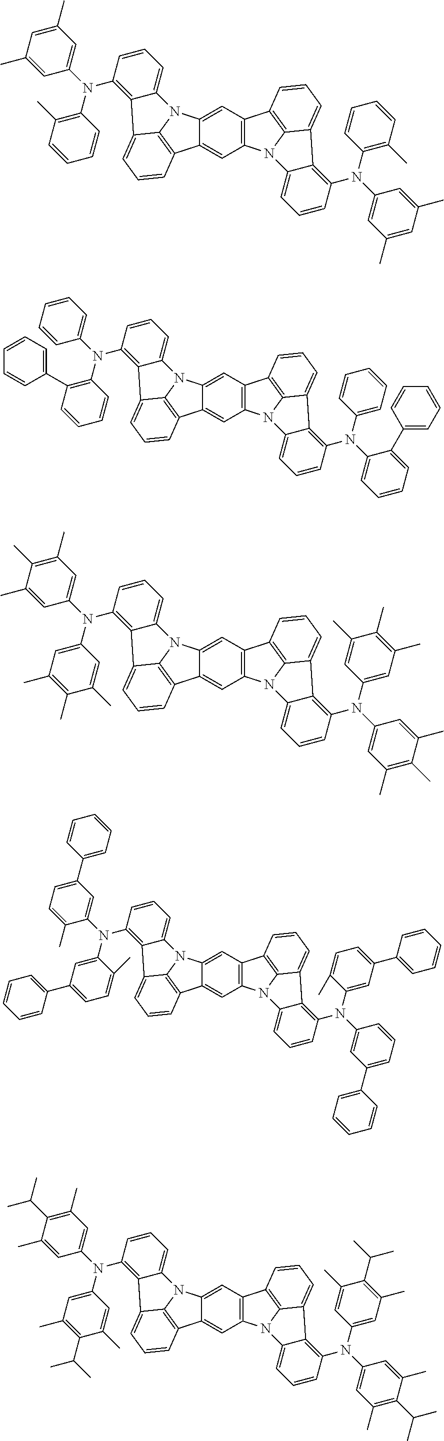

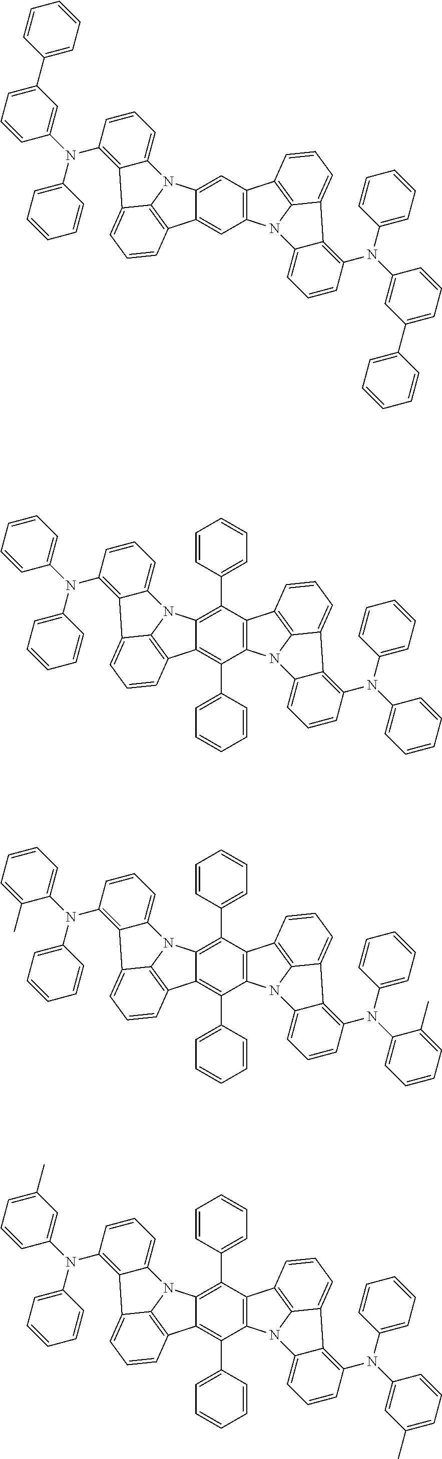 [SMILES: c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1 ; c1ccc(-c2c3c4cccc5c6c(N(c7ccccc7)c7ccccc7)cccc6n(c3c(-c3ccccc3)c3c6cccc7c8c(N(c9ccccc9)c9ccccc9)cccc8n(c23)c76)c54)cc1 ; Cc1ccccc1N(c1ccccc1)c1cccc2c1c1cccc3c4c(-c5ccccc5)c5c(c(-c6ccccc6)c4n2c13)c1cccc2c3c(N(c4ccccc4)c4ccccc4C)cccc3n5c21 ; Cc1cccc(N(c2ccccc2)c2cccc3c2c2cccc4c5c(-c6ccccc6)c6c(c(-c7ccccc7)c5n3c24)c2cccc3c4c(N(c5ccccc5)c5cccc(C)c5)cccc4n6c32)c1]

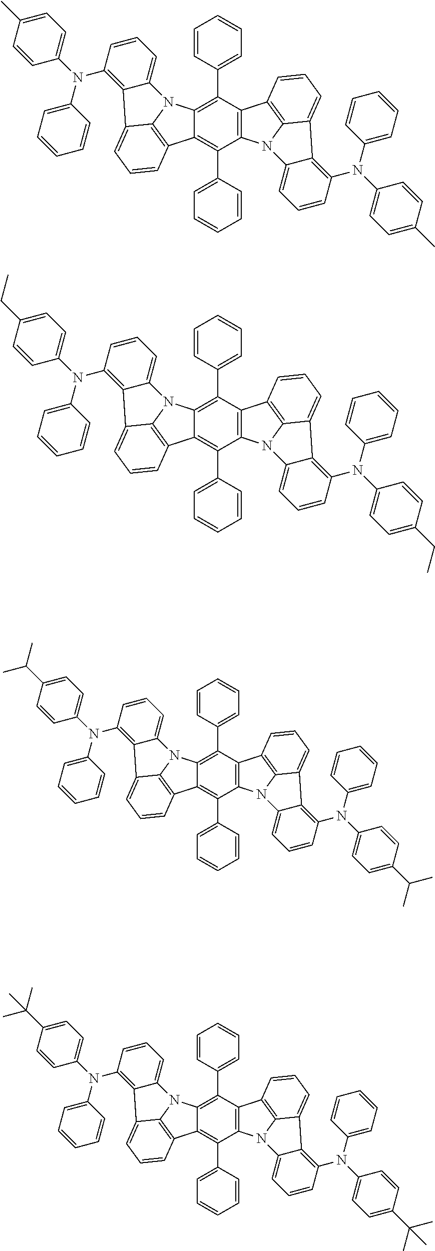

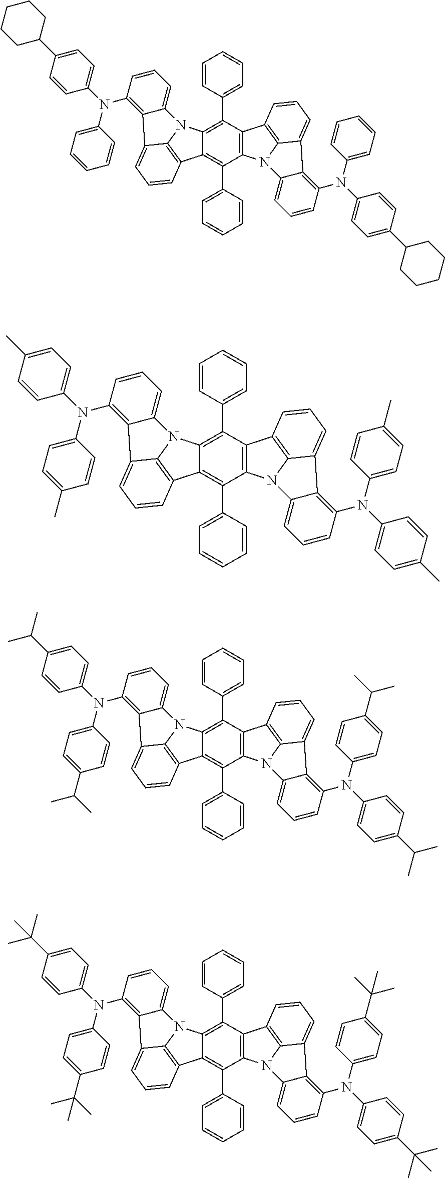

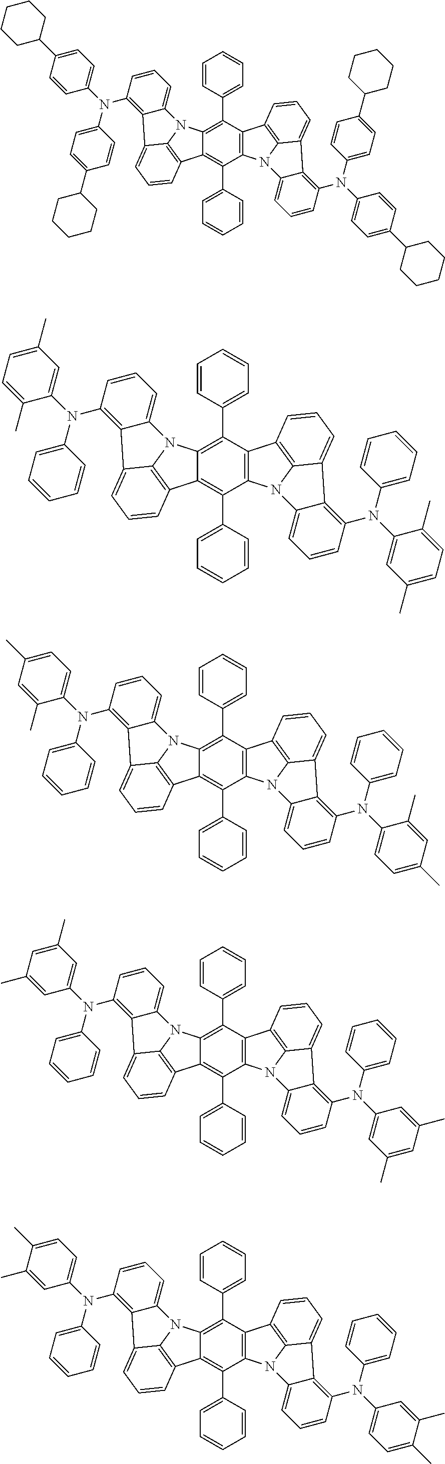

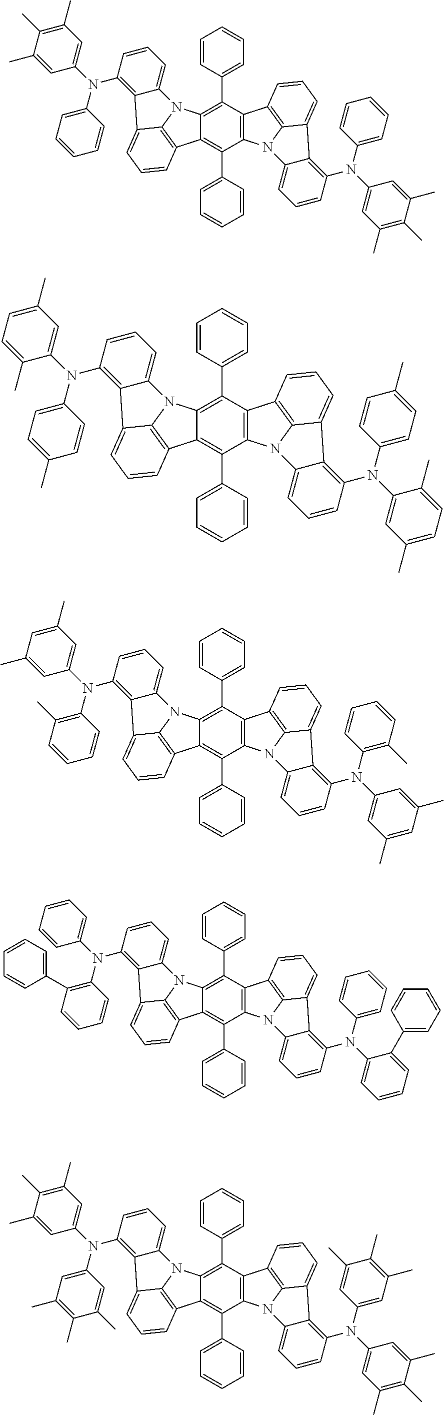

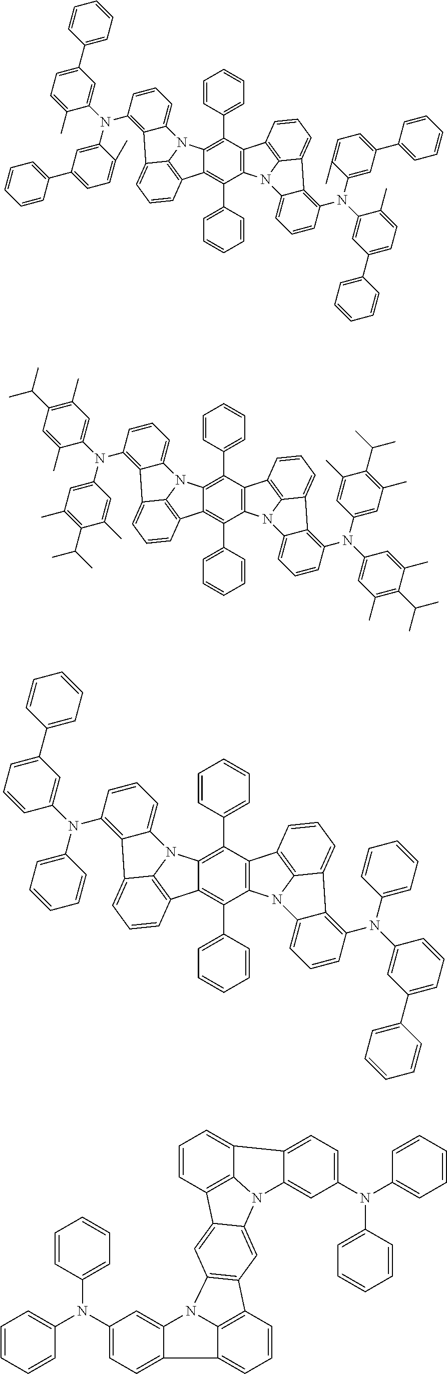

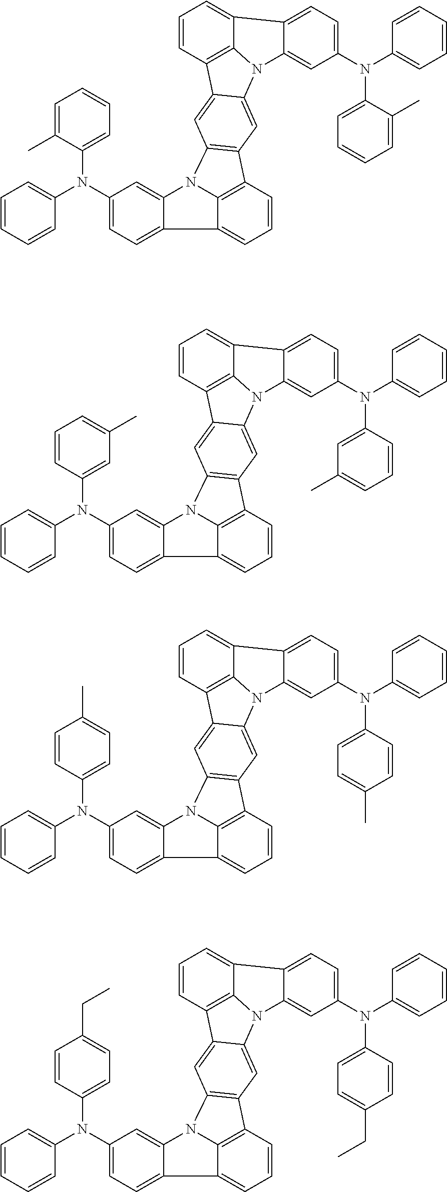 [SMILES: Cc1ccccc1N(c1ccccc1)c1ccc2c3cccc4c5cc6c(cc5n(c2c1)c34)c1cccc2c3ccc(N(c4ccccc4)c4ccccc4C)cc3n6c21 ; Cc1cccc(N(c2ccccc2)c2ccc3c4cccc5c6cc7c(cc6n(c3c2)c45)c2cccc3c4ccc(N(c5ccccc5)c5cccc(C)c5)cc4n7c32)c1 ; Cc1ccc(N(c2ccccc2)c2ccc3c4cccc5c6cc7c(cc6n(c3c2)c45)c2cccc3c4ccc(N(c5ccccc5)c5ccc(C)cc5)cc4n7c32)cc1 ; CCc1ccc(N(c2ccccc2)c2ccc3c4cccc5c6cc7c(cc6n(c3c2)c45)c2cccc3c4ccc(N(c5ccccc5)c5ccc(CC)cc5)cc4n7c32)cc1]

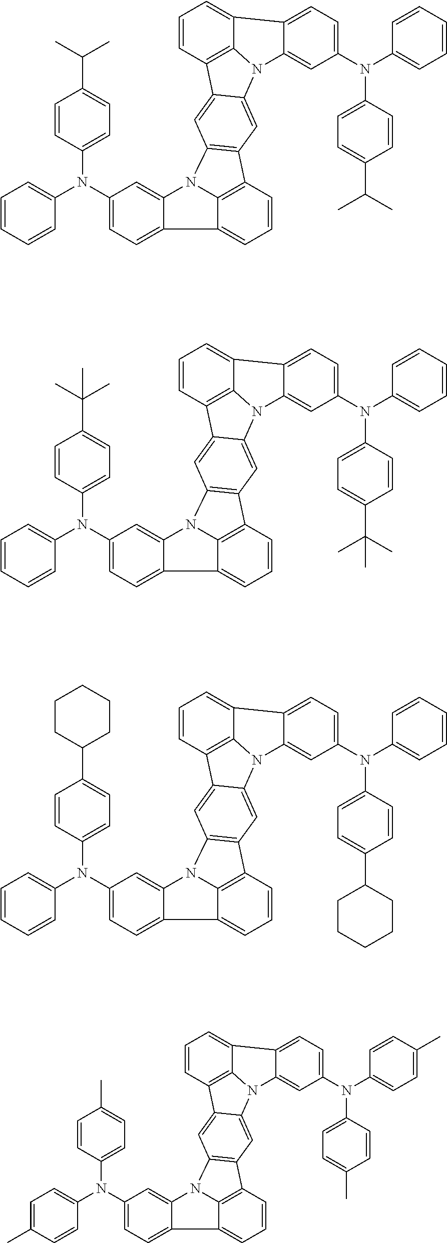

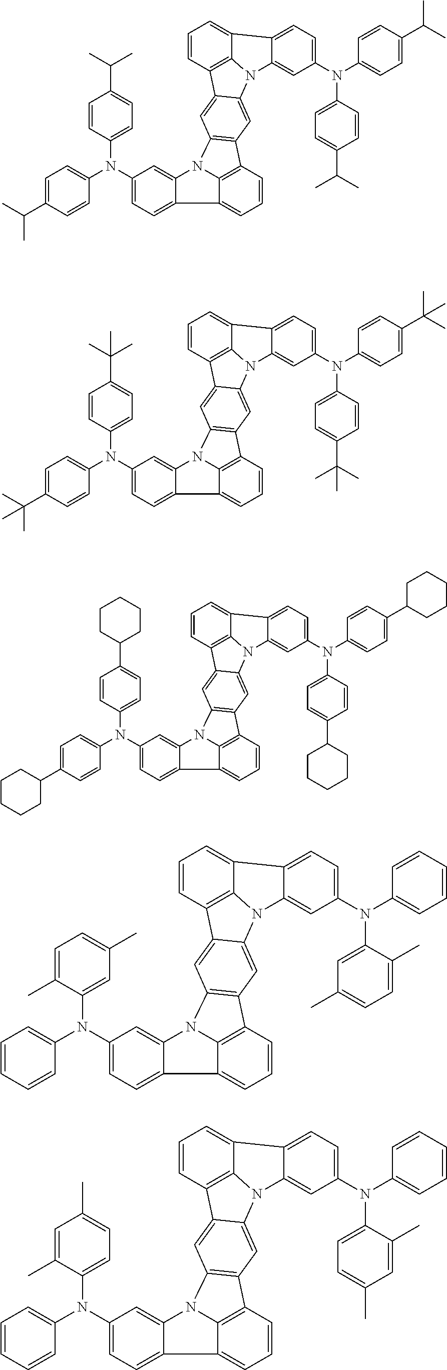

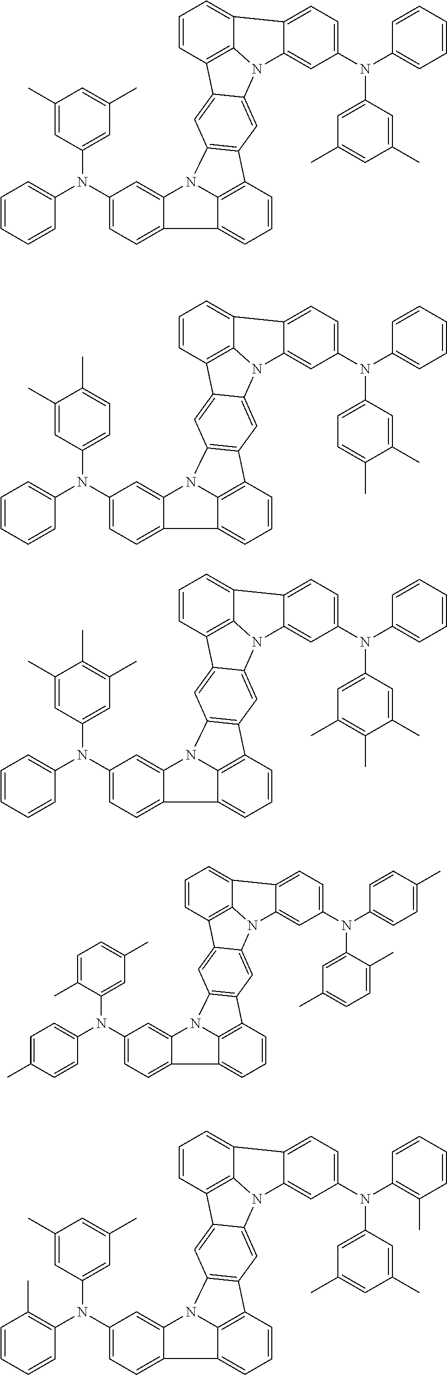

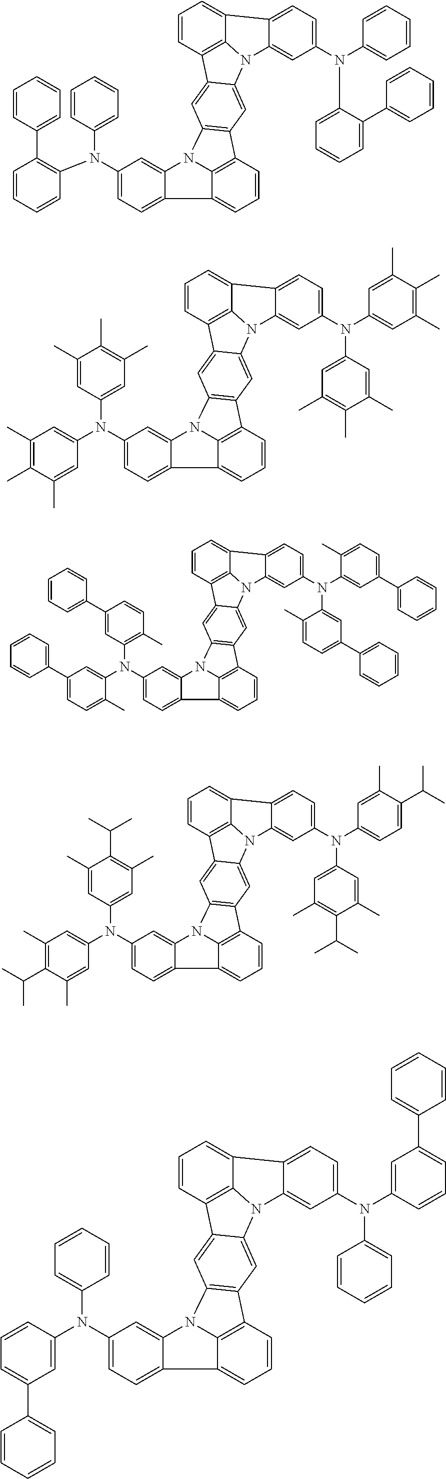

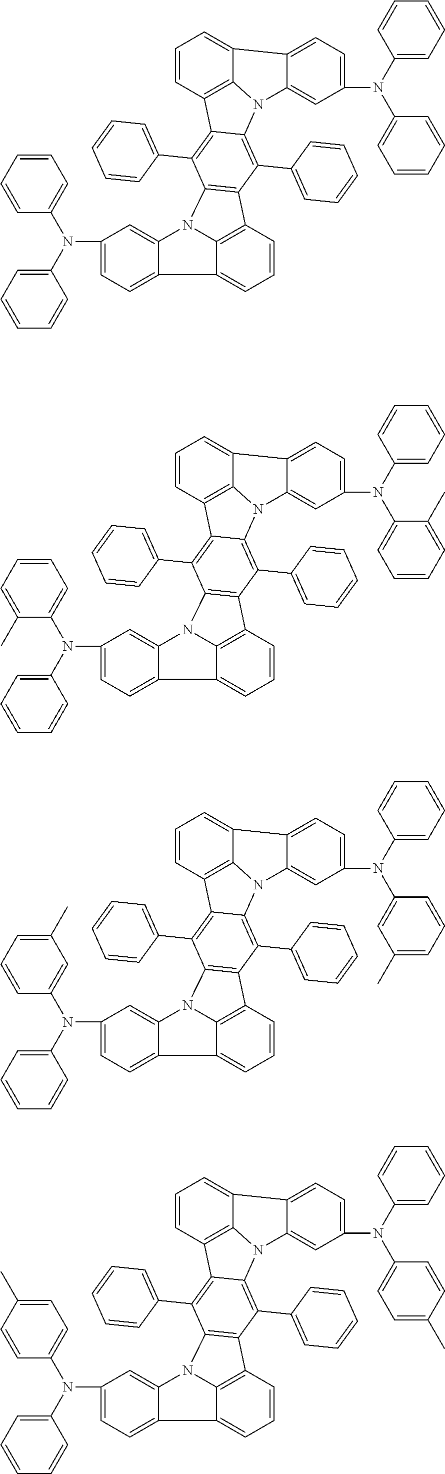 [SMILES: c1ccc(-c2c3c4cccc5c6ccc(N(c7ccccc7)c7ccccc7)cc6n(c3c(-c3ccccc3)c3c6cccc7c8ccc(N(c9ccccc9)c9ccccc9)cc8n(c23)c76)c54)cc1 ; Cc1ccccc1N(c1ccccc1)c1ccc2c3cccc4c5c(-c6ccccc6)c6c(c(-c7ccccc7)c5n(c2c1)c34)c1cccc2c3ccc(N(c4ccccc4)c4ccccc4C)cc3n6c21 ; Cc1cccc(N(c2ccccc2)c2ccc3c4cccc5c6c(-c7ccccc7)c7c(c(-c8ccccc8)c6n(c3c2)c45)c2cccc3c4ccc(N(c5ccccc5)c5cccc(C)c5)cc4n7c32)c1 ; Cc1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C)cc5)cc4n7c32)cc1]

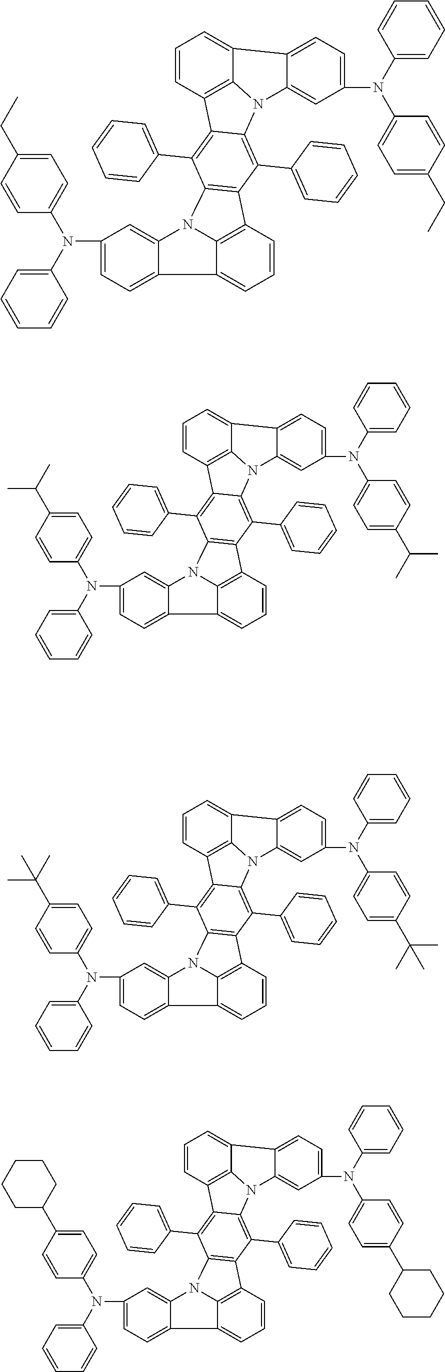

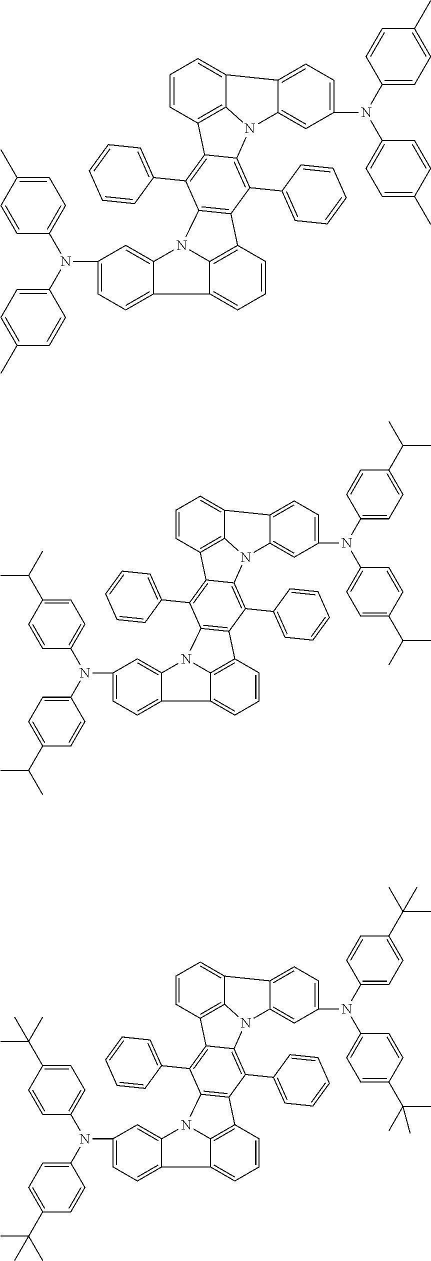

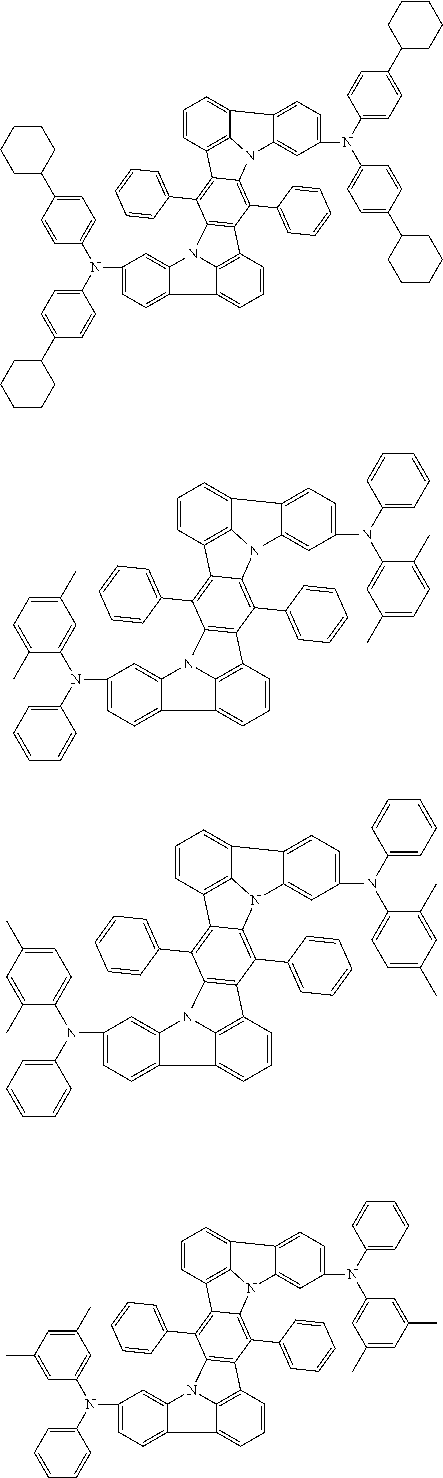

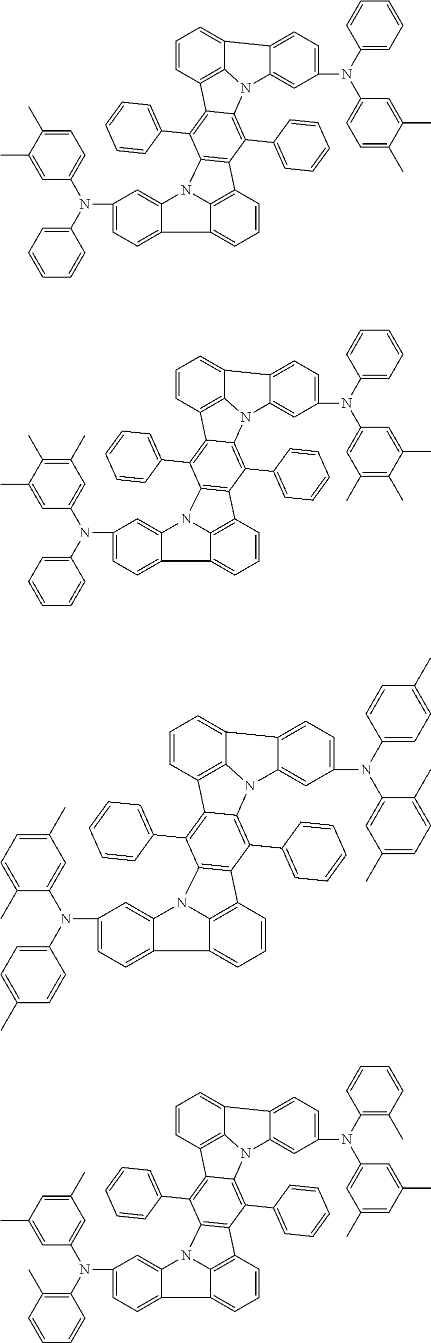

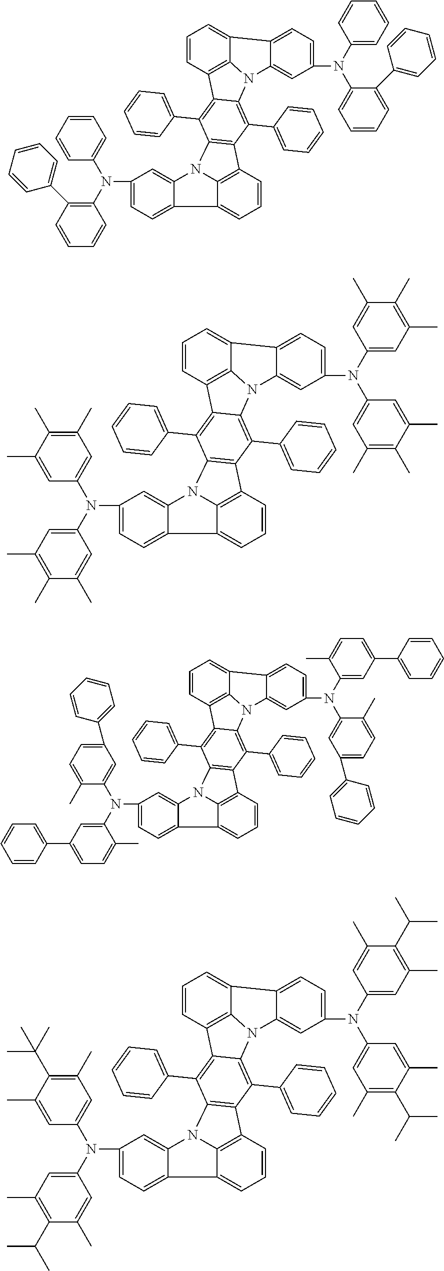

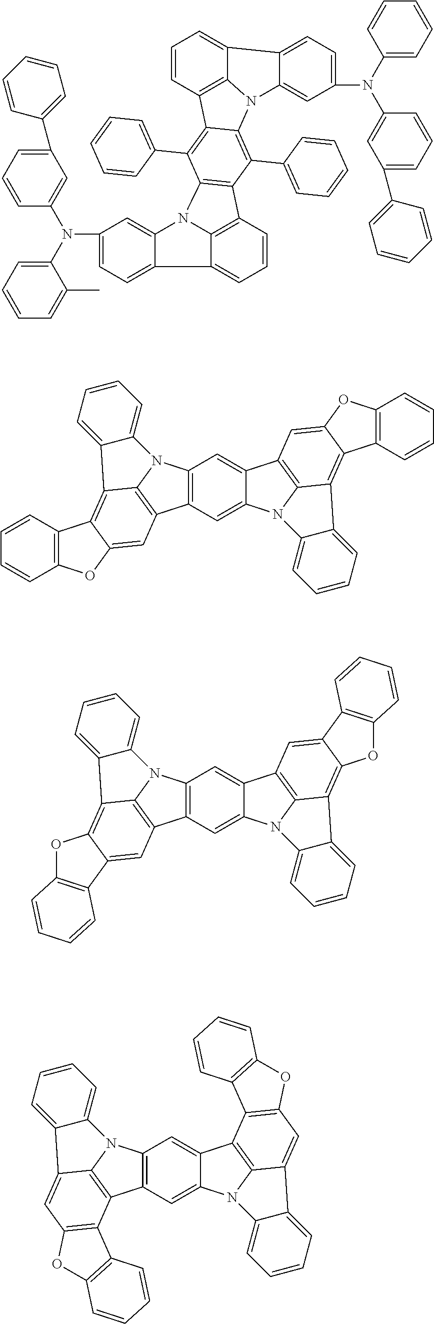 [SMILES: Cc1ccccc1N(c1cccc(-c2ccccc2)c1)c1ccc2c3cccc4c5c(-c6ccccc6)c6c(c(-c7ccccc7)c5n(c2c1)c34)c1cccc2c3ccc(N(c4ccccc4)c4cccc(-c5ccccc5)c4)cc3n6c21 ; c1ccc2c(c1)oc1cc3c4cc5c(cc4n4c6ccccc6c(c12)c34)c1cc2oc3ccccc3c2c2c3ccccc3n5c12 ; c1ccc2c(c1)oc1c2cc2c3cc4c(cc3n3c5ccccc5c1c23)c1cc2c3ccccc3oc2c2c3ccccc3n4c12 ; c1ccc2c(c1)oc1cc3c4ccccc4n4c5cc6c7c8c(cc9c%10ccccc%10n(c6cc5c(c12)c34)c97)oc1ccccc18]

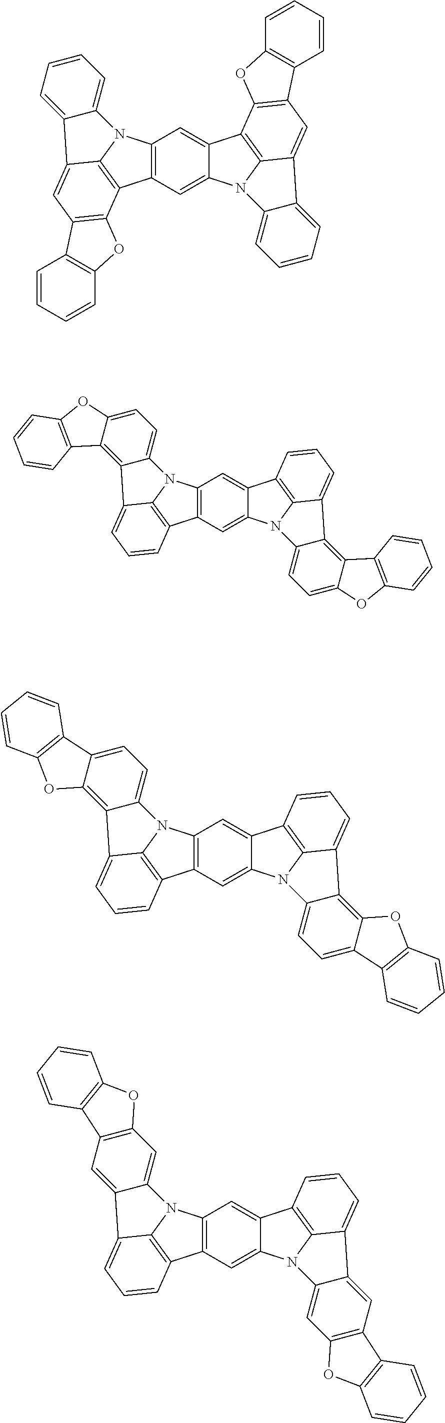 [SMILES: c1ccc2c(c1)oc1c2cc2c3ccccc3n3c4cc5c6c7oc8ccccc8c7cc7c8ccccc8n(c5cc4c1c23)c76 ; c1ccc2c(c1)oc1ccc3c(c4cccc5c6cc7c(cc6n3c54)c3cccc4c5c6c(ccc5n7c34)oc3ccccc36)c12 ; c1ccc2c(c1)oc1c2ccc2c1c1cccc3c4cc5c(cc4n2c31)c1cccc2c3c4oc6ccccc6c4ccc3n5c12 ; c1ccc2c(c1)oc1cc3c(cc12)c1cccc2c4cc5c(cc4n3c12)c1cccc2c3cc4c(cc3n5c21)oc1ccccc14]

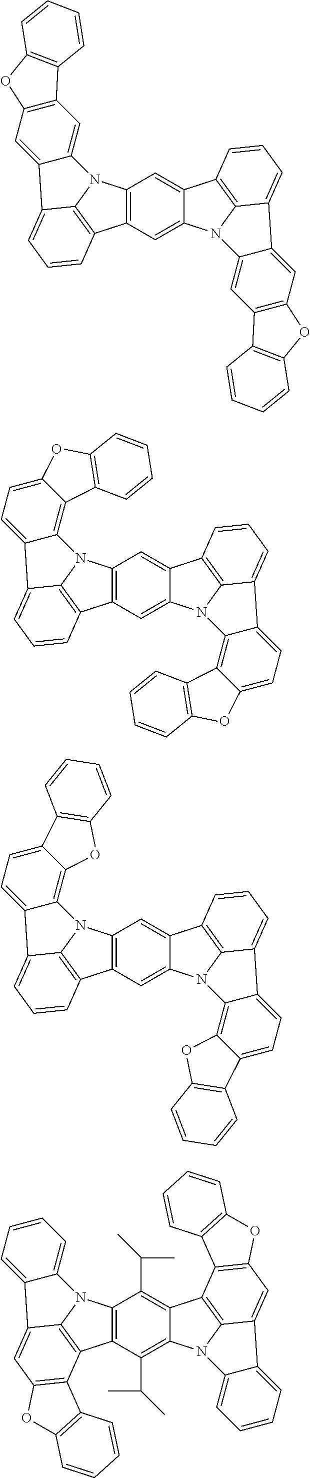

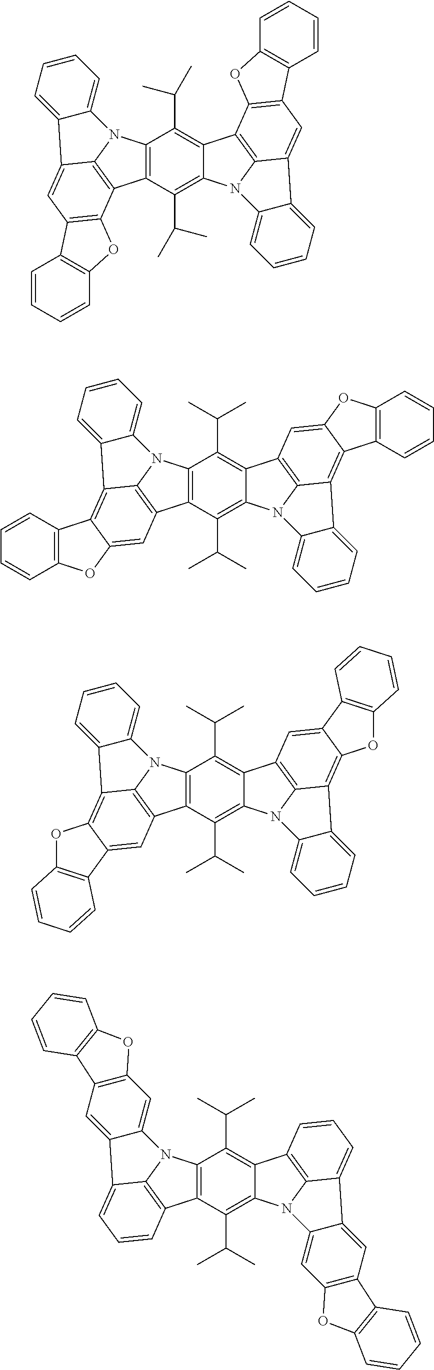 [SMILES: CC(C)c1c2c3c4oc5ccccc5c4cc4c5ccccc5n(c2c(C(C)C)c2c5c6oc7ccccc7c6cc6c7ccccc7n(c12)c65)c43 ; CC(C)c1c2c3cc4oc5ccccc5c4c4c5ccccc5n(c2c(C(C)C)c2c5cc6oc7ccccc7c6c6c7ccccc7n(c12)c56)c34 ; CC(C)c1c2c3cc4c5ccccc5oc4c4c5ccccc5n(c2c(C(C)C)c2c5cc6c7ccccc7oc6c6c7ccccc7n(c12)c56)c34 ; CC(C)c1c2c3cccc4c5cc6c(cc5n(c2c(C(C)C)c2c5cccc7c8cc9c(cc8n(c12)c75)oc1ccccc19)c43)oc1ccccc16]

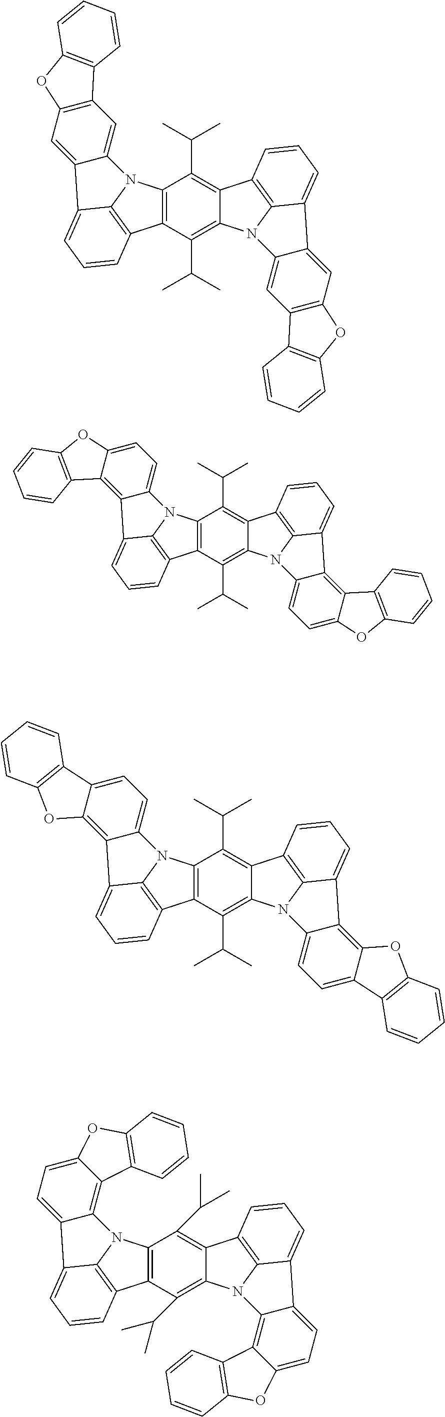 [SMILES: CC(C)c1c2c3cccc4c5cc6oc7ccccc7c6cc5n(c2c(C(C)C)c2c5cccc6c7cc8oc9ccccc9c8cc7n(c12)c65)c43 ; CC(C)c1c2c3cccc4c5c6c(ccc5n(c2c(C(C)C)c2c5cccc7c8c9c(ccc8n(c12)c57)oc1ccccc19)c34)oc1ccccc16 ; CC(C)c1c2c3cccc4c5c6oc7ccccc7c6ccc5n(c2c(C(C)C)c2c5cccc6c7c8oc9ccccc9c8ccc7n(c12)c56)c34 ; CC(C)c1c2c3cccc4c5ccc6oc7ccccc7c6c5n(c2c(C(C)C)c2c5cccc6c7ccc8oc9ccccc9c8c7n(c12)c65)c43]

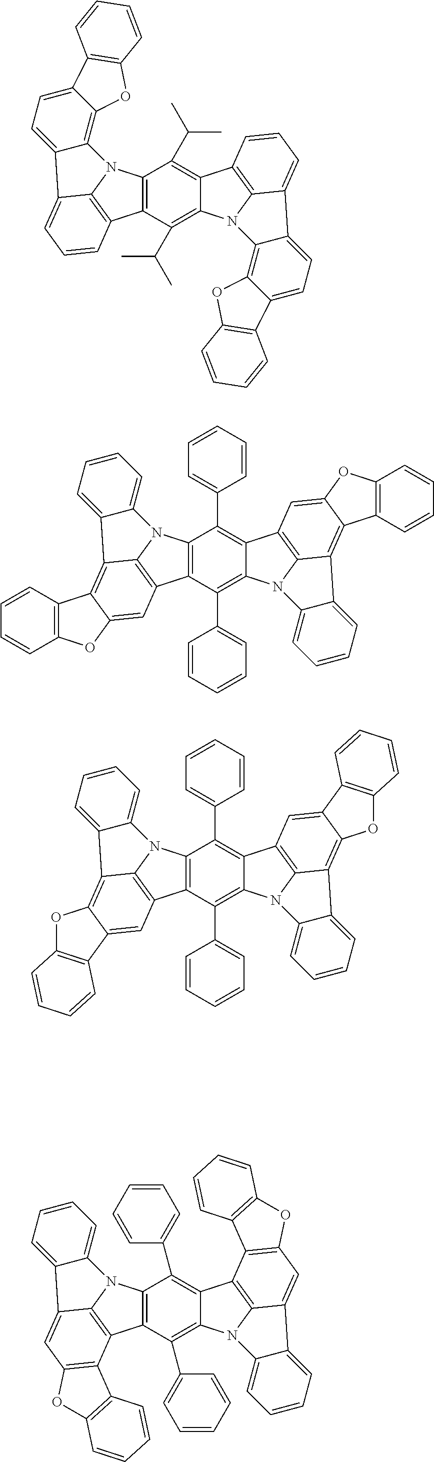

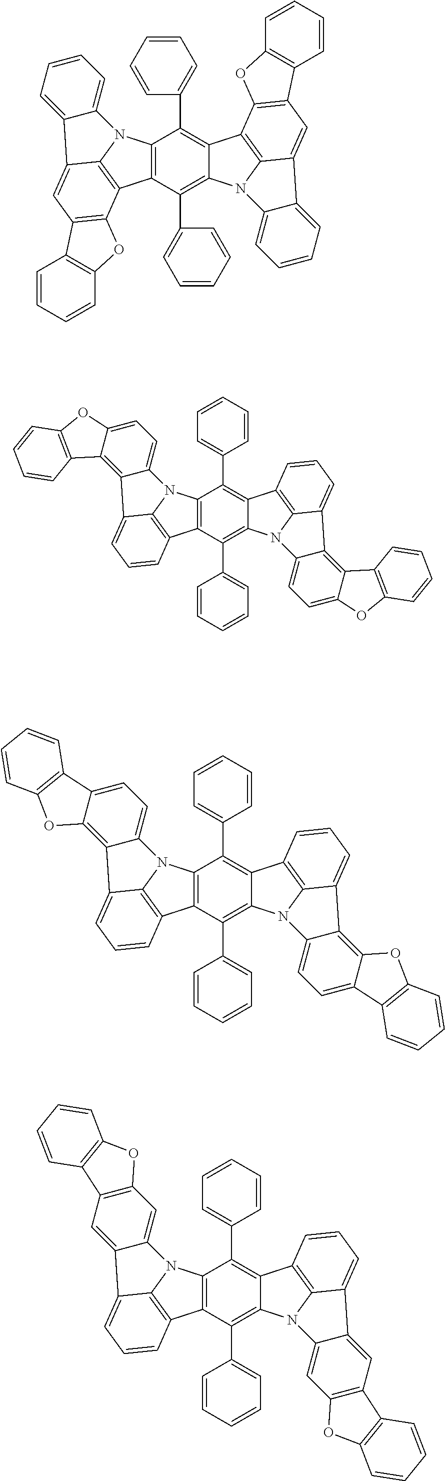 [SMILES: c1ccc(-c2c3c4c5oc6ccccc6c5cc5c6ccccc6n(c3c(-c3ccccc3)c3c6c7oc8ccccc8c7cc7c8ccccc8n(c23)c76)c54)cc1 ; c1ccc(-c2c3c4cccc5c6c7c(ccc6n(c3c(-c3ccccc3)c3c6cccc8c9c%10c(ccc9n(c23)c68)oc2ccccc2%10)c45)oc2ccccc27)cc1 ; c1ccc(-c2c3c4cccc5c6c7oc8ccccc8c7ccc6n(c3c(-c3ccccc3)c3c6cccc7c8c9oc%10ccccc%10c9ccc8n(c23)c67)c45)cc1 ; c1ccc(-c2c3c4cccc5c6cc7c(cc6n(c3c(-c3ccccc3)c3c6cccc8c9cc%10c(cc9n(c23)c86)oc2ccccc2%10)c54)oc2ccccc27)cc1]

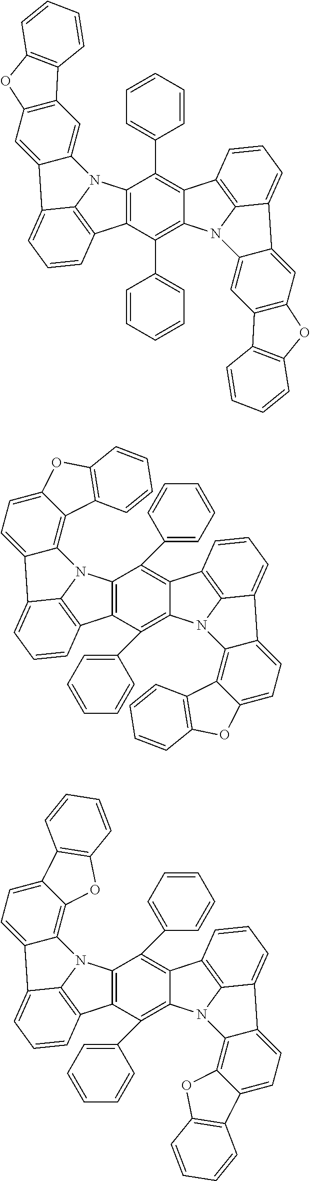 [SMILES: c1ccc(-c2c3c4cccc5c6cc7oc8ccccc8c7cc6n(c3c(-c3ccccc3)c3c6cccc7c8cc9oc%10ccccc%10c9cc8n(c23)c76)c54)cc1 ; c1ccc(-c2c3c4cccc5c6ccc7oc8ccccc8c7c6n(c3c(-c3ccccc3)c3c6cccc7c8ccc9oc%10ccccc%10c9c8n(c23)c76)c54)cc1 ; c1ccc(-c2c3c4cccc5c6ccc7c8ccccc8oc7c6n(c3c(-c3ccccc3)c3c6cccc7c8ccc9c%10ccccc%10oc9c8n(c23)c76)c54)cc1]

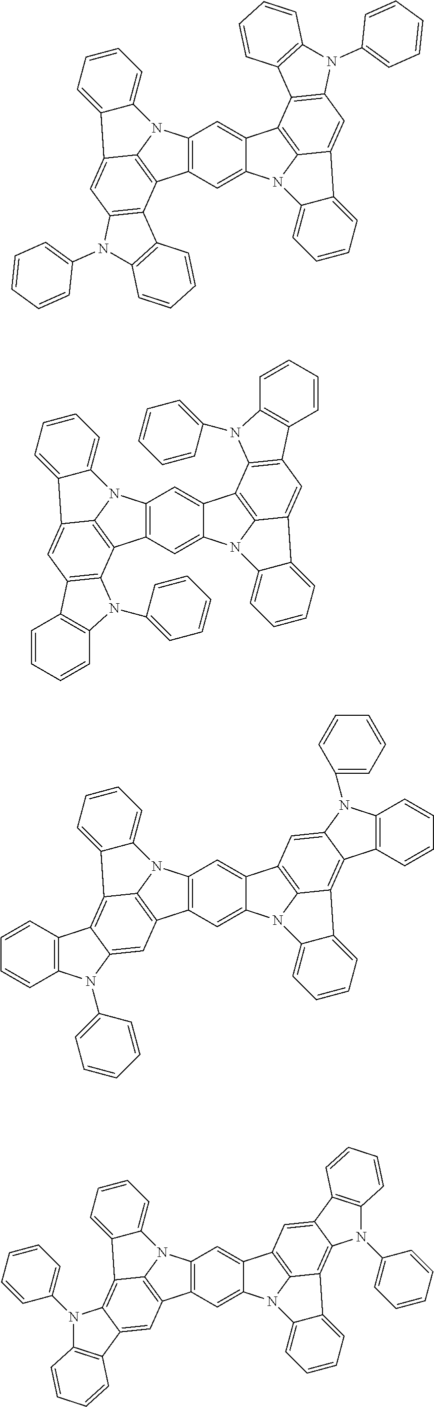 [SMILES: c1ccc(-n2c3ccccc3c3c4c5cc6c(cc5n5c7ccccc7c(cc32)c45)c2c3c4ccccc4n(-c4ccccc4)c3cc3c4ccccc4n6c32)cc1 ; c1ccc(-n2c3ccccc3c3cc4c5ccccc5n5c6cc7c8c9c(cc%10c%11ccccc%11n(c7cc6c(c32)c45)c%108)c2ccccc2n9-c2ccccc2)cc1 ; c1ccc(-n2c3ccccc3c3c4c5ccccc5n5c6cc7c8cc9c(c%10ccccc%10n9-c9ccccc9)c9c%10ccccc%10n(c7cc6c(cc32)c45)c89)cc1 ; c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1]

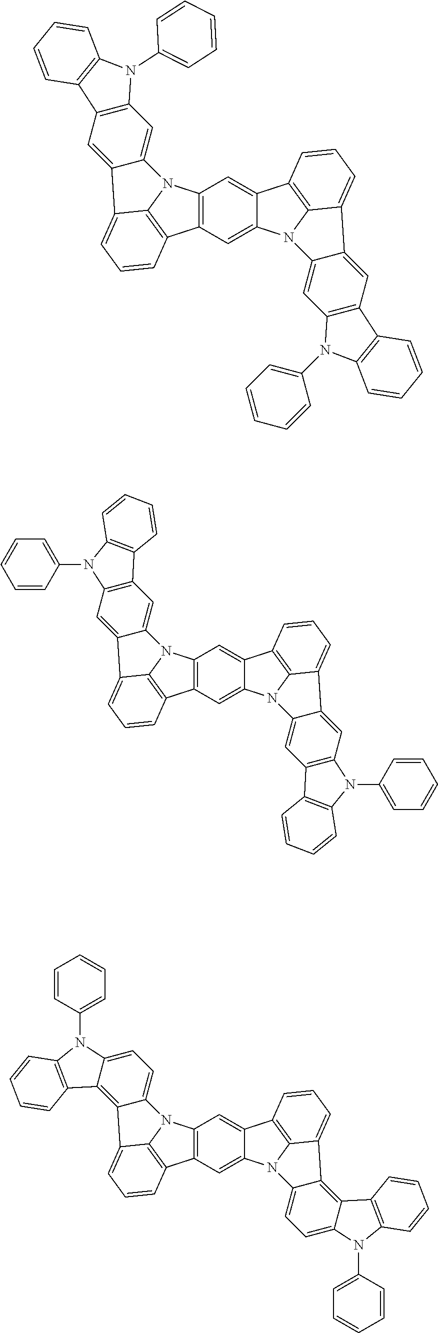 [SMILES: c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1 ; c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1 ; c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1]

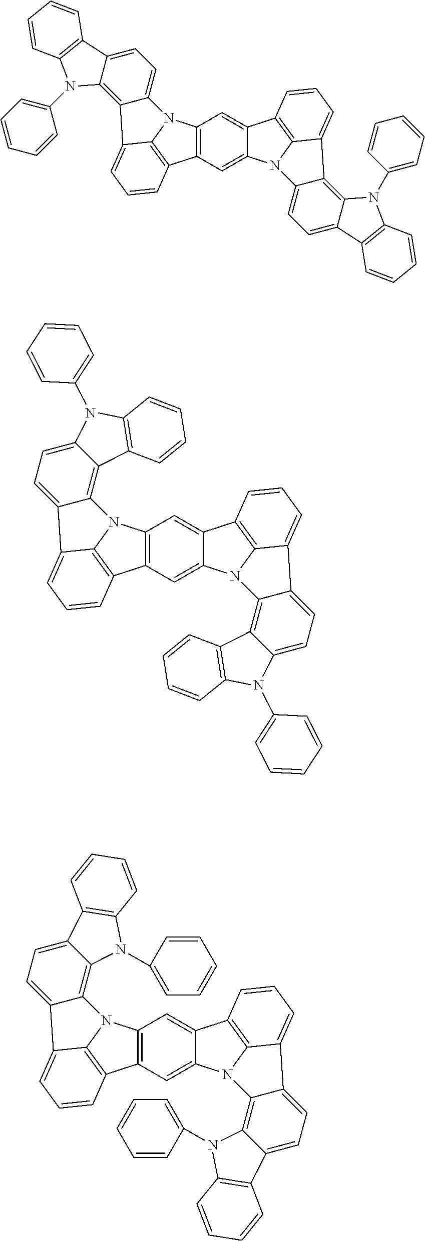 [SMILES: c1ccc(-n2c3ccccc3c3ccc4c(c5cccc6c7cc8c(cc7n4c65)c4cccc5c6c7c(ccc6n8c45)c4ccccc4n7-c4ccccc4)c32)cc1 ; c1ccc(-n2c3ccccc3c3c2ccc2c4cccc5c6cc7c(cc6n(c54)c23)c2cccc3c4ccc5c(c6ccccc6n5-c5ccccc5)c4n7c23)cc1 ; c1ccc(-n2c3ccccc3c3ccc4c5cccc6c7cc8c(cc7n(c65)c4c32)c2cccc3c4ccc5c6ccccc6n(-c6ccccc6)c5c4n8c23)cc1]

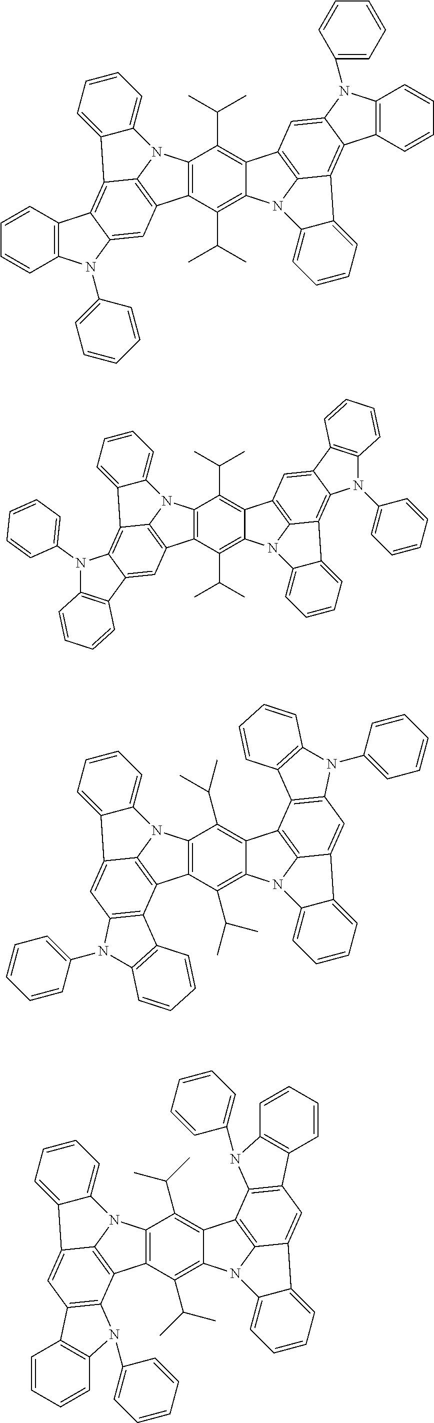 [SMILES: CC(C)c1c2c3cc4c(c5ccccc5n4-c4ccccc4)c4c5ccccc5n(c2c(C(C)C)c2c5cc6c(c7ccccc7n6-c6ccccc6)c6c7ccccc7n(c12)c56)c34 ; CC(C)c1c2c3cc4c5ccccc5n(-c5ccccc5)c4c4c5ccccc5n(c2c(C(C)C)c2c5cc6c7ccccc7n(-c7ccccc7)c6c6c7ccccc7n(c12)c56)c34 ; CC(C)c1c2c3c4c5ccccc5n(-c5ccccc5)c4cc4c5ccccc5n(c2c(C(C)C)c2c5c6c7ccccc7n(-c7ccccc7)c6cc6c7ccccc7n(c12)c65)c43 ; CC(C)c1c2c3c4c(cc5c6ccccc6n(c2c(C(C)C)c2c6c7c(cc8c9ccccc9n(c12)c86)c1ccccc1n7-c1ccccc1)c53)c1ccccc1n4-c1ccccc1]

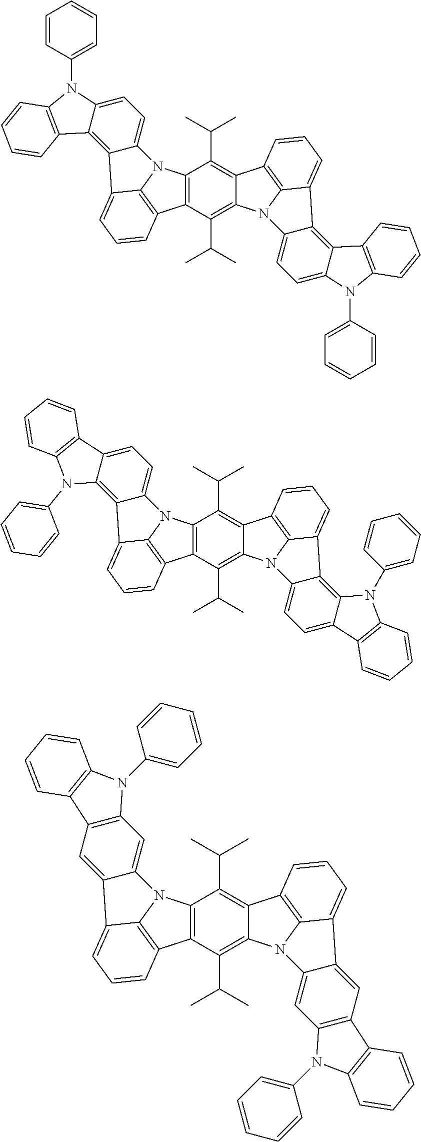 [SMILES: CC(C)c1c2c3cccc4c5c6c7ccccc7n(-c7ccccc7)c6ccc5n(c2c(C(C)C)c2c5cccc6c7c8c9ccccc9n(-c9ccccc9)c8ccc7n(c12)c56)c34 ; CC(C)c1c2c3cccc4c5c6c(ccc5n(c2c(C(C)C)c2c5cccc7c8c9c(ccc8n(c12)c57)c1ccccc1n9-c1ccccc1)c34)c1ccccc1n6-c1ccccc1 ; CC(C)c1c2c3cccc4c5cc6c7ccccc7n(-c7ccccc7)c6cc5n(c2c(C(C)C)c2c5cccc6c7cc8c9ccccc9n(-c9ccccc9)c8cc7n(c12)c65)c43]

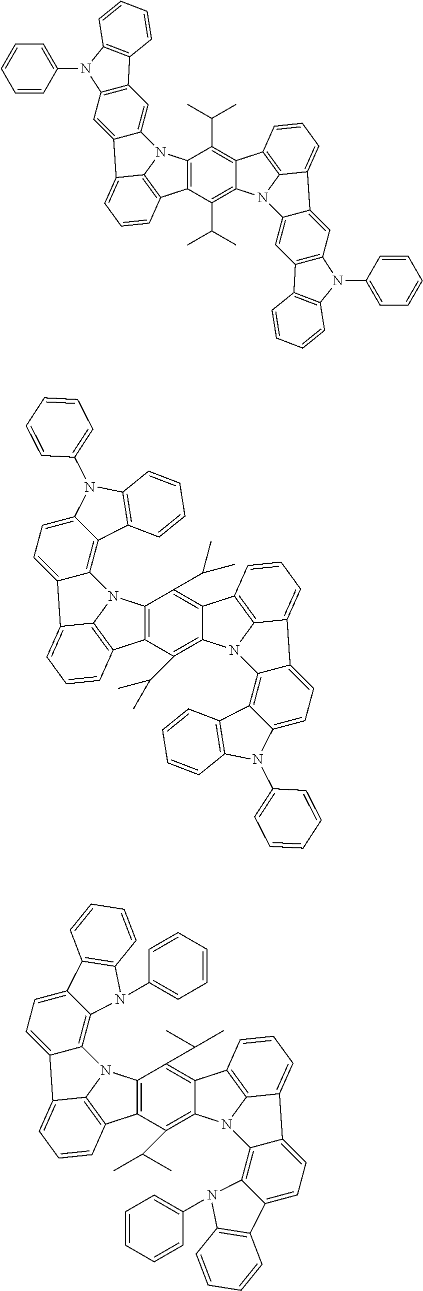 [SMILES: CC(C)c1c2c3cccc4c5cc6c(cc5n(c2c(C(C)C)c2c5cccc7c8cc9c(cc8n(c12)c75)c1ccccc1n9-c1ccccc1)c43)c1ccccc1n6-c1ccccc1 ; CC(C)c1c2c3cccc4c5ccc6c(c7ccccc7n6-c6ccccc6)c5n(c2c(C(C)C)c2c5cccc6c7ccc8c(c9ccccc9n8-c8ccccc8)c7n(c12)c65)c43 ; CC(C)c1c2c3cccc4c5ccc6c7ccccc7n(-c7ccccc7)c6c5n(c2c(C(C)C)c2c5cccc6c7ccc8c9ccccc9n(-c9ccccc9)c8c7n(c12)c65)c43]

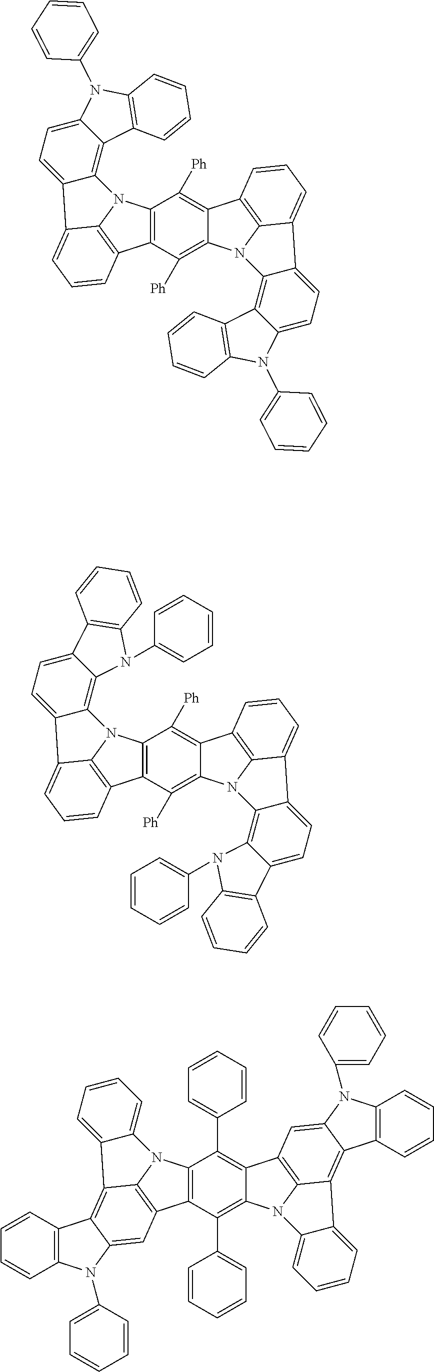 [SMILES: c1ccc(-c2c3c4cccc5c6ccc7c(c8ccccc8n7-c7ccccc7)c6n(c3c(-c3ccccc3)c3c6cccc7c8ccc9c(c%10ccccc%10n9-c9ccccc9)c8n(c23)c76)c54)cc1 ; c1ccc(-c2c3c4cccc5c6ccc7c8ccccc8n(-c8ccccc8)c7c6n(c3c(-c3ccccc3)c3c6cccc7c8ccc9c%10ccccc%10n(-c%10ccccc%10)c9c8n(c23)c76)c54)cc1 ; c1ccc(-c2c3c4cc5c(c6ccccc6n5-c5ccccc5)c5c6ccccc6n(c3c(-c3ccccc3)c3c6cc7c(c8ccccc8n7-c7ccccc7)c7c8ccccc8n(c23)c67)c45)cc1]

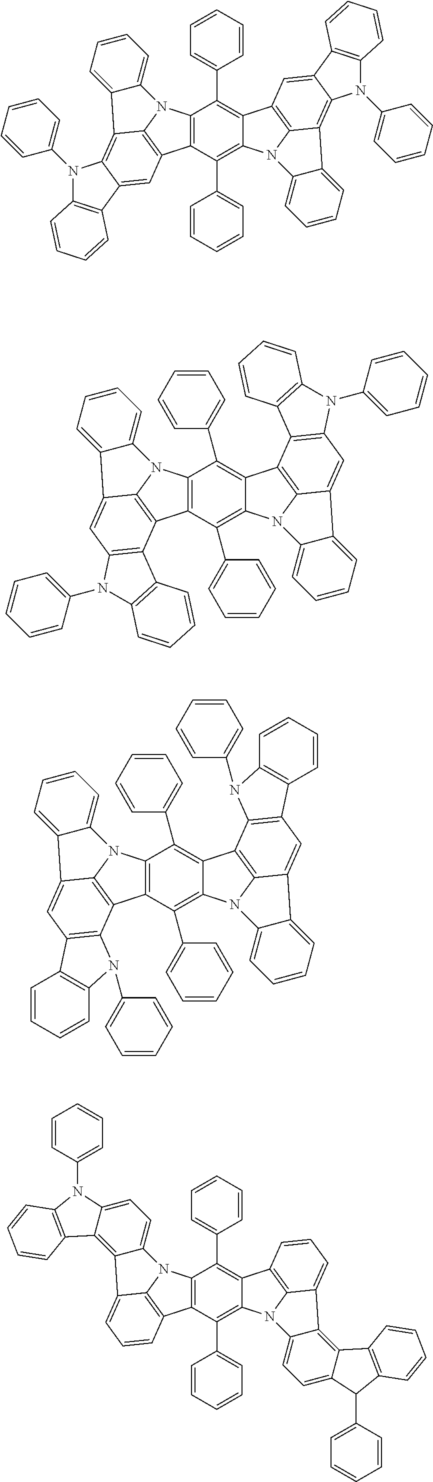 [SMILES: c1ccc(-c2c3c4cc5c6ccccc6n(-c6ccccc6)c5c5c6ccccc6n(c3c(-c3ccccc3)c3c6cc7c8ccccc8n(-c8ccccc8)c7c7c8ccccc8n(c23)c67)c45)cc1 ; c1ccc(-c2c3c4c5c6ccccc6n(-c6ccccc6)c5cc5c6ccccc6n(c3c(-c3ccccc3)c3c6c7c8ccccc8n(-c8ccccc8)c7cc7c8ccccc8n(c23)c76)c54)cc1 ; c1ccc(-c2c3c4c5c(cc6c7ccccc7n(c3c(-c3ccccc3)c3c7c8c(cc9c%10ccccc%10n(c23)c97)c2ccccc2n8-c2ccccc2)c64)c2ccccc2n5-c2ccccc2)cc1 ; c1ccc(-c2c3c4cccc5c6c7c8ccccc8n(-c8ccccc8)c7ccc6n(c3c(-c3ccccc3)c3c6cccc7c8c9c(ccc8n(c23)c76)C(c2ccccc2)c2ccccc2-9)c45)cc1]

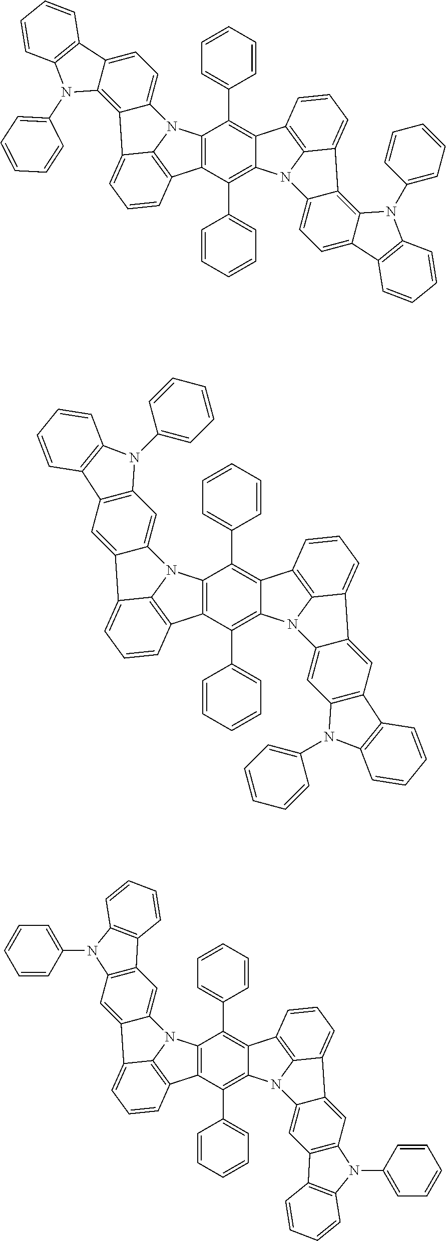 [SMILES: c1ccc(-c2c3c4cccc5c6c7c(ccc6n(c3c(-c3ccccc3)c3c6cccc8c9c%10c(ccc9n(c23)c68)c2ccccc2n%10-c2ccccc2)c45)c2ccccc2n7-c2ccccc2)cc1 ; c1ccc(-c2c3c4cccc5c6cc7c8ccccc8n(-c8ccccc8)c7cc6n(c3c(-c3ccccc3)c3c6cccc7c8cc9c%10ccccc%10n(-c%10ccccc%10)c9cc8n(c23)c76)c54)cc1 ; c1ccc(-c2c3c4cccc5c6cc7c(cc6n(c3c(-c3ccccc3)c3c6cccc8c9cc%10c(cc9n(c23)c86)c2ccccc2n%10-c2ccccc2)c54)c2ccccc2n7-c2ccccc2)cc1]

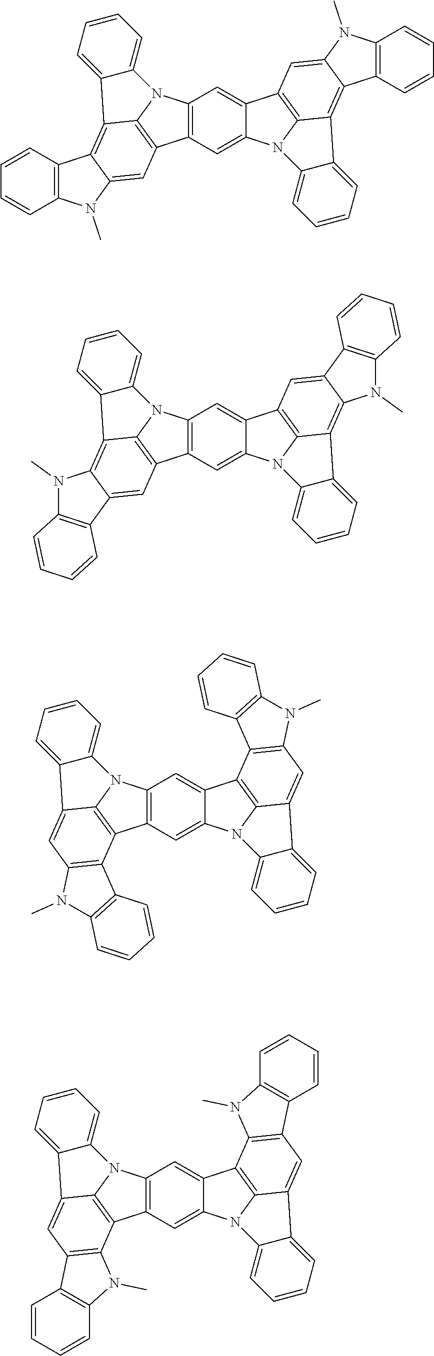

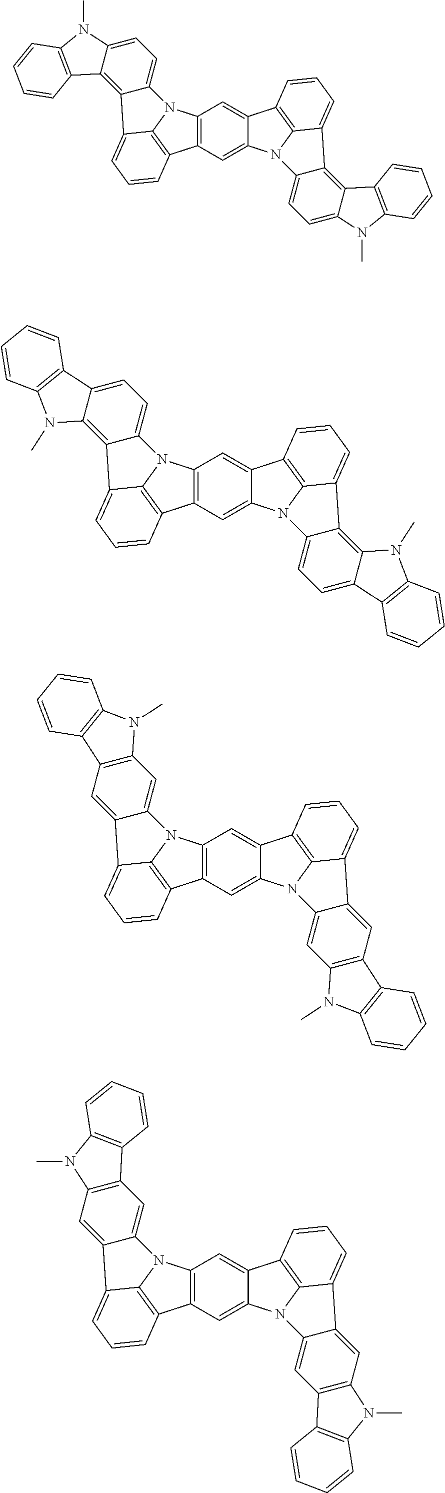

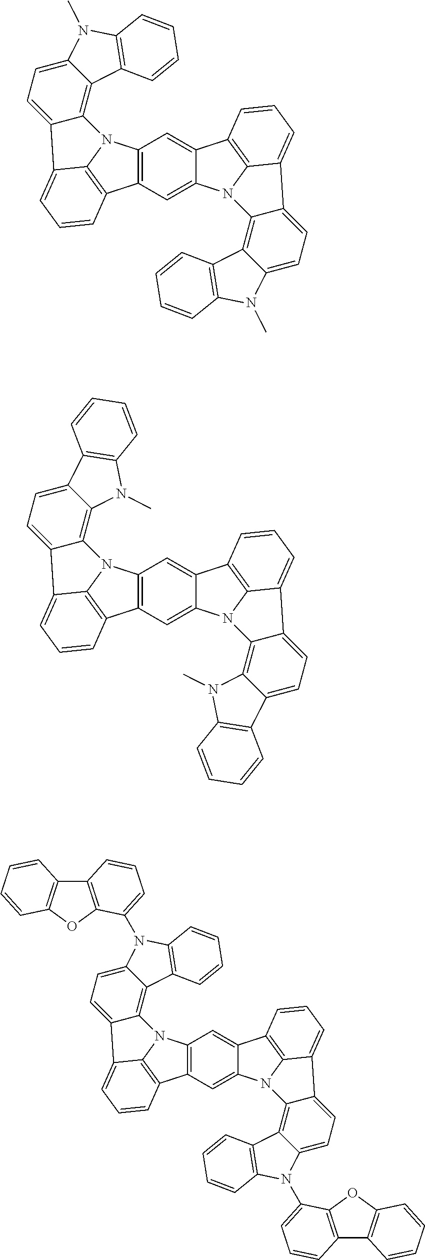 [SMILES: Cn1c2ccccc2c2c1ccc1c3cccc4c5cc6c(cc5n(c43)c12)c1cccc2c3ccc4c(c5ccccc5n4C)c3n6c12 ; Cn1c2ccccc2c2ccc3c4cccc5c6cc7c(cc6n(c54)c3c21)c1cccc2c3ccc4c5ccccc5n(C)c4c3n7c12 ; c1ccc2c(c1)oc1c(-n3c4ccccc4c4c3ccc3c5cccc6c7cc8c(cc7n(c65)c34)c3cccc4c5ccc6c(c7ccccc7n6-c6cccc7c6oc6ccccc67)c5n8c34)cccc12]

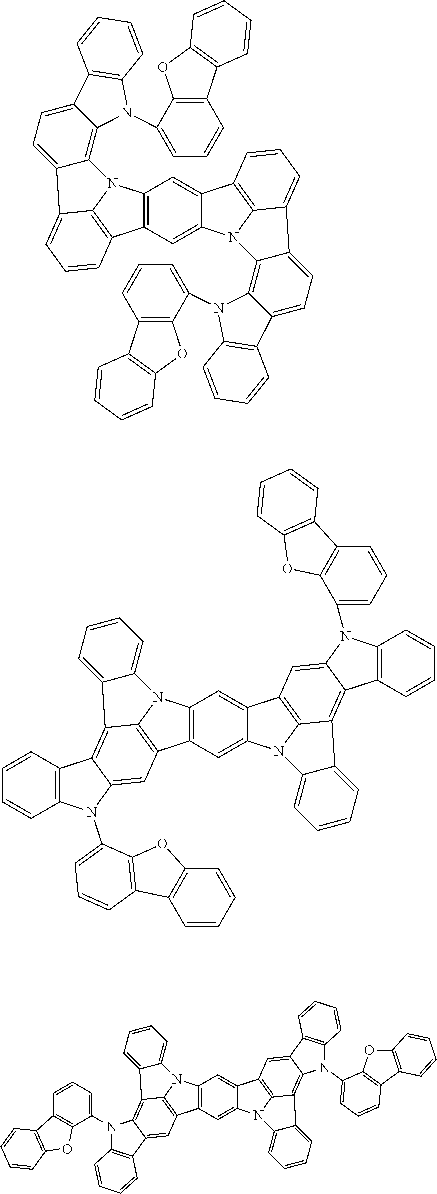 [SMILES: c1ccc2c(c1)oc1c(-n3c4ccccc4c4ccc5c6cccc7c8cc9c(cc8n(c76)c5c43)c3cccc4c5ccc6c7ccccc7n(-c7cccc8c7oc7ccccc78)c6c5n9c34)cccc12 ; c1ccc2c(c1)oc1c(-n3c4ccccc4c4c5c6ccccc6n6c7cc8c9cc%10c(c%11ccccc%11n%10-c%10cccc%11c%10oc%10ccccc%10%11)c%10c%11ccccc%11n(c8cc7c(cc43)c56)c9%10)cccc12 ; c1ccc2c(c1)oc1c(-n3c4ccccc4c4cc5c6cc7c(cc6n6c8ccccc8c(c43)c56)c3cc4c5ccccc5n(-c5cccc6c5oc5ccccc56)c4c4c5ccccc5n7c34)cccc12]

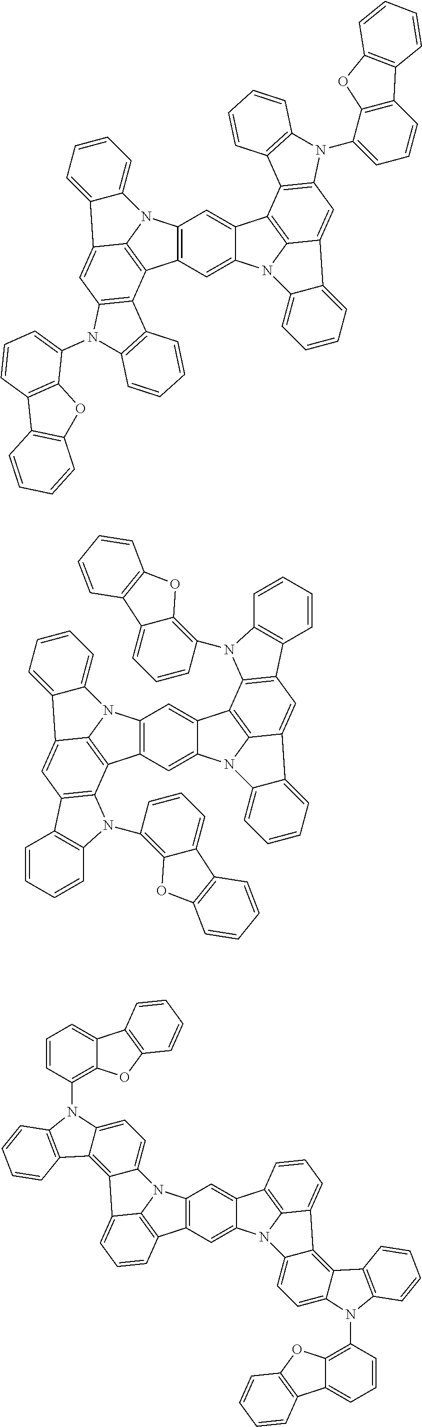 [SMILES: c1ccc2c(c1)oc1c(-n3c4ccccc4c4c5c6cc7c(cc6n6c8ccccc8c(cc43)c56)c3c4c5ccccc5n(-c5cccc6c5oc5ccccc56)c4cc4c5ccccc5n7c43)cccc12 ; c1ccc2c(c1)oc1c(-n3c4ccccc4c4cc5c6ccccc6n6c7cc8c9c%10c(cc%11c%12ccccc%12n(c8cc7c(c43)c56)c%119)c3ccccc3n%10-c3cccc4c3oc3ccccc34)cccc12 ; c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12]

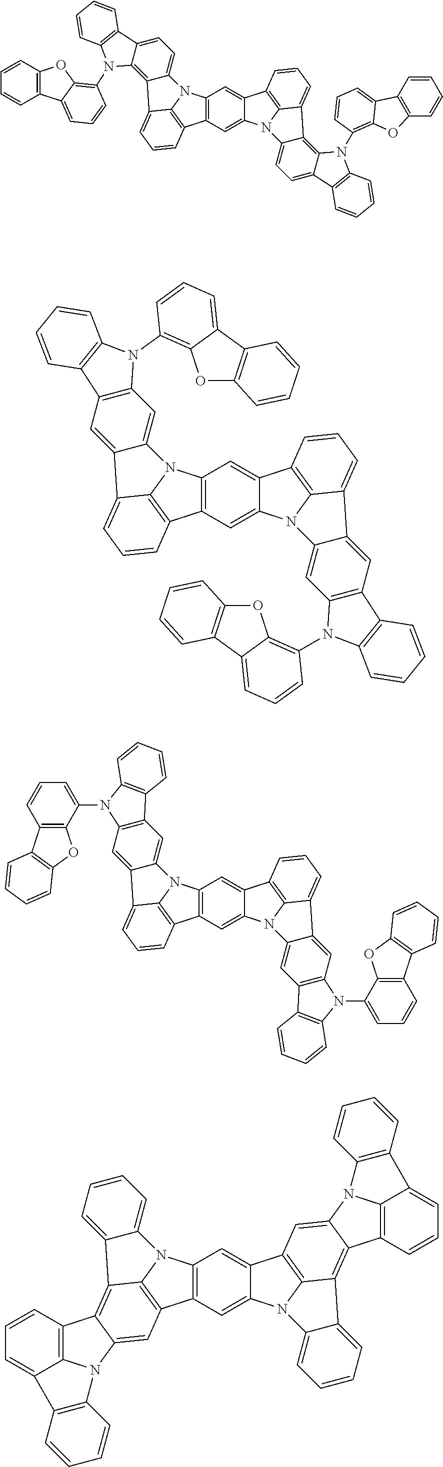

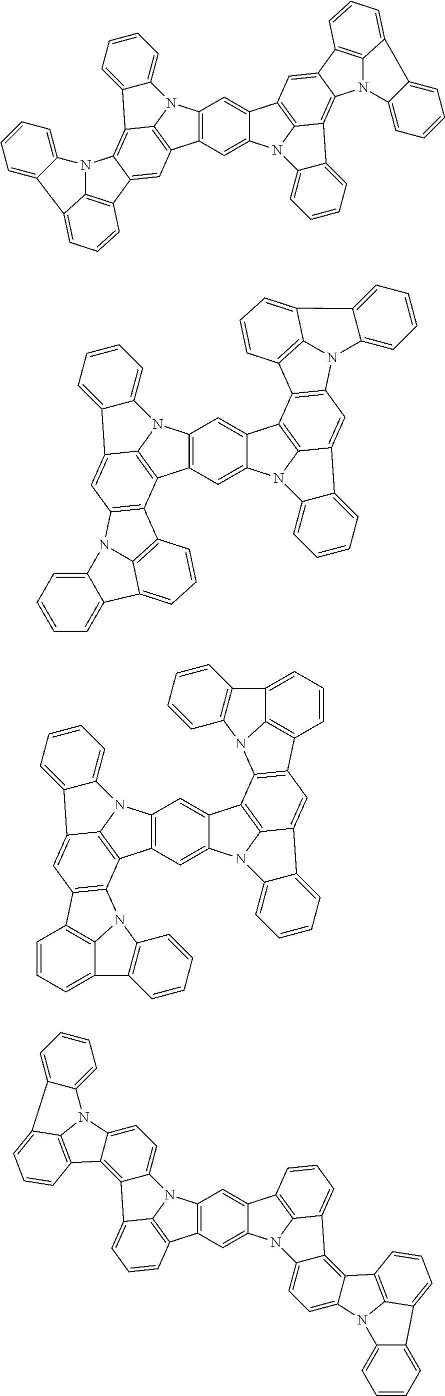

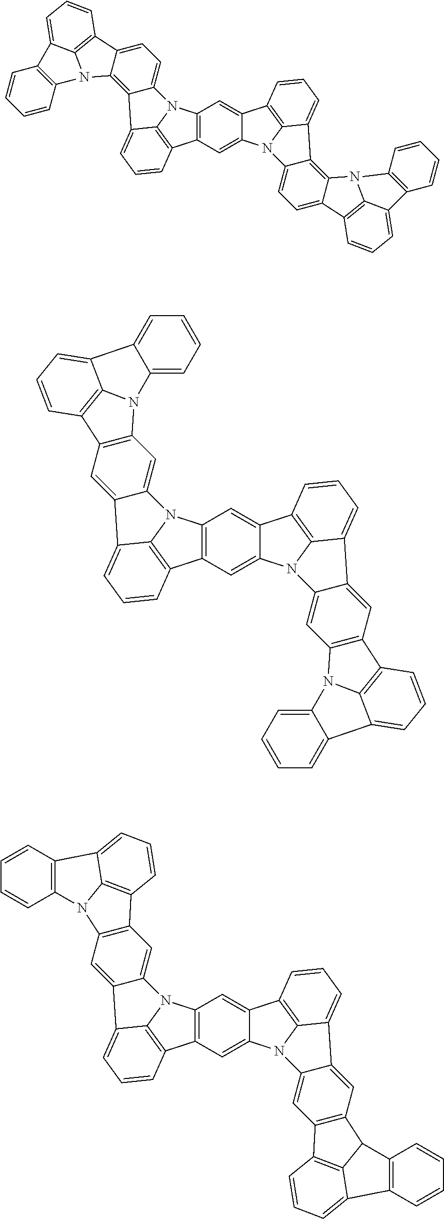 [SMILES: c1ccc2c(c1)c1cccc3c4ccc5c(c6cccc7c8cc9c(cc8n5c76)c5cccc6c7c(ccc8c%10cccc%11c%12ccccc%12n(c%11%10)c87)n9c56)c4n2c13 ; c1ccc2c(c1)c1cccc3c4cc5c6cccc7c8cc9c(cc8n(c5cc4n2c13)c76)c1cccc2c3cc4c5cccc6c7ccccc7n(c4cc3n9c12)c65 ; c1ccc2c(c1)-c1cccc3c1C2c1cc2c4cccc5c6cc7c(cc6n(c2cc1-3)c45)c1cccc2c3cc4c(cc3n7c21)c1cccc2c3ccccc3n4c21]

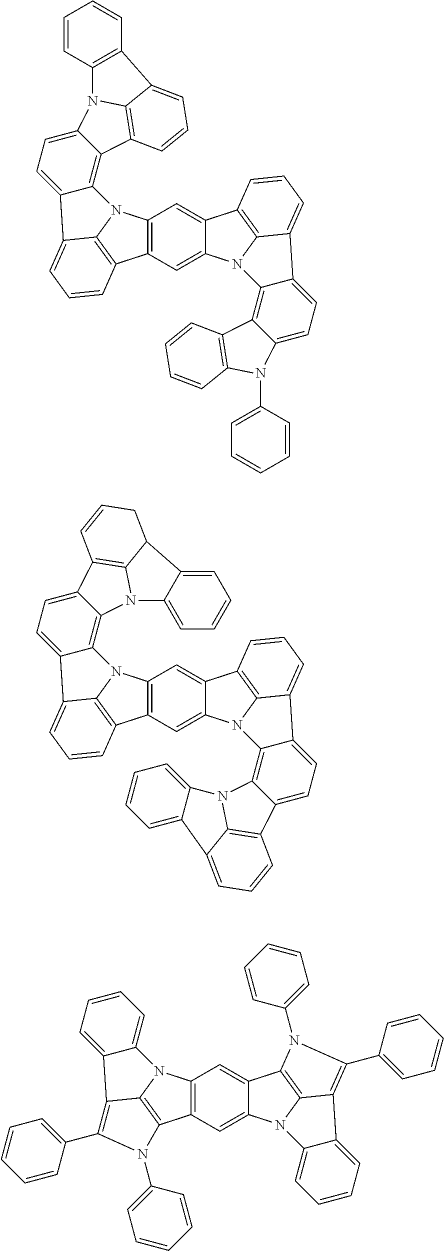 [SMILES: c1ccc(-n2c3ccccc3c3c2ccc2c4cccc5c6cc7c(cc6n(c54)c23)c2cccc3c4ccc5c(c6cccc8c9ccccc9n5c86)c4n7c23)cc1 ; C1=Cc2c3n(c4c2ccc2c5cccc6c7cc8c(cc7n(c65)c24)c2cccc4c5ccc6c7cccc9c%10ccccc%10n(c97)c6c5n8c24)-c2ccccc2C3C1 ; c1ccc(-c2c3c4ccccc4n4c5cc6c7c8c(c(-c9ccccc9)n7-c7ccccc7)c7ccccc7n8c6cc5c(c34)n2-c2ccccc2)cc1]

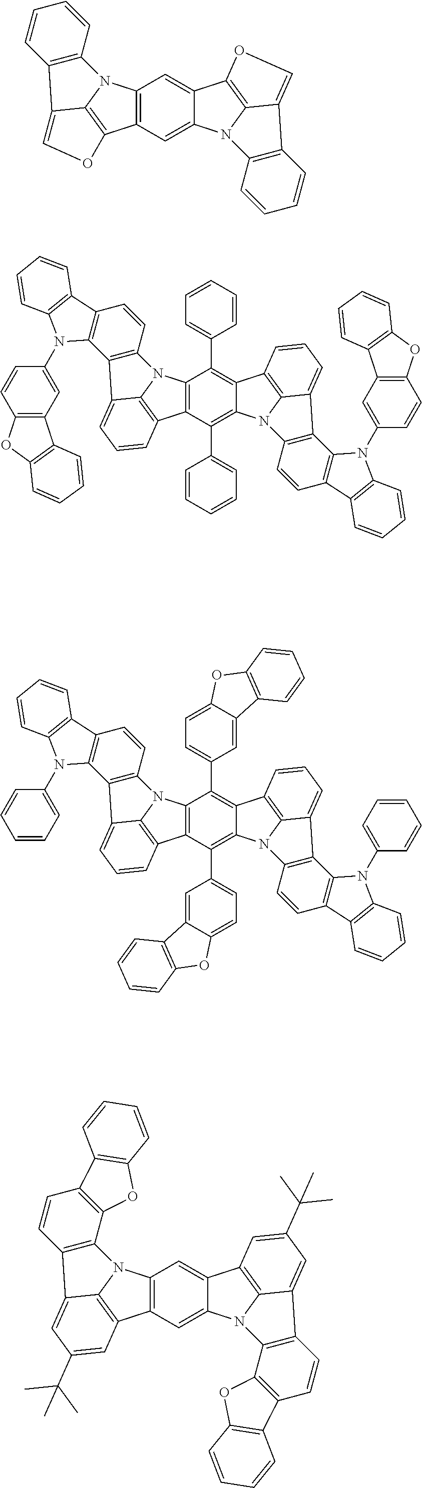 [SMILES: c1ccc2c(c1)c1coc3c4cc5c(cc4n2c13)c1occ2c3ccccc3n5c21 ; c1ccc(-c2c3c4cccc5c6c7c(ccc6n(c3c(-c3ccccc3)c3c6cccc8c9c%10c(ccc9n(c23)c68)c2ccccc2n%10-c2ccc3oc6ccccc6c3c2)c45)c2ccccc2n7-c2ccc3oc4ccccc4c3c2)cc1 ; c1ccc(-n2c3ccccc3c3ccc4c(c5cccc6c7c(-c8ccc9oc%10ccccc%10c9c8)c8c(c(-c9ccc%10oc%11ccccc%11c%10c9)c7n4c65)c4cccc5c6c7c(ccc6n8c45)c4ccccc4n7-c4ccccc4)c32)cc1 ; CC(C)(C)c1cc2c3cc4c(cc3n3c2c(c1)c1ccc2c5ccccc5oc2c13)c1cc(C(C)(C)C)cc2c3ccc5c6ccccc6oc5c3n4c12]

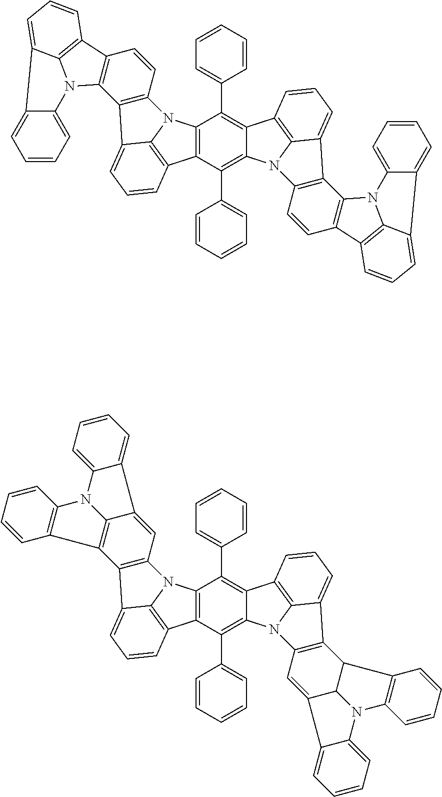 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; C1=C2c3ccccc3N3c4ccccc4C(c4c1n1c5c(-c6ccccc6)c6c7cccc8c9c%10c%11ccccc%11n%11c%12ccccc%12c(cc9n(c6c(-c6ccccc6)c5c5cccc4c51)c78)c%10%11)C23]

In one embodiment, in the compound represented by the formulas (1) and (A1), the substituent in the case of “substituted or unsubstituted” is a group selected from the group consisting of:

an alkyl group including 1 to 50 carbon atoms,

an aryl group including 6 to 50 ring carbon atoms, and

a monovalent heterocyclic group including 5 to 50 ring atoms.

In one embodiment, in the compound represented by the formulas (1) and (A1), the substituent in the case of “substituted or unsubstituted” is a group selected from the group consisting of:

an alkyl group including 1 to 18 carbon atoms,

an aryl group including 6 to 18 ring carbon atoms, and

a monovalent heterocyclic group including 5 to 18 ring atoms.

Specific examples of the groups in the formulas (1) and (A1) are as described in the section of [Definitions] of this specification.

As described above, known materials and device configurations may be applied to the organic EL device according to the first aspect of the invention, as long as the device comprises a cathode, an anode, and an emitting layer between the cathode and the anode, wherein the light-emitting layer comprises the compound represented by the formula (1) and the compound represented by the formula (1A), and the effect of the invention is not impaired.

Parts which can be used in the organic EL device according to the first aspect, materials for forming respective layers, other than the above-mentioned compounds, and the like, will be described later.

[Compound of the Second Aspect]

The compound according to the second aspect is represented by the following formula (1).

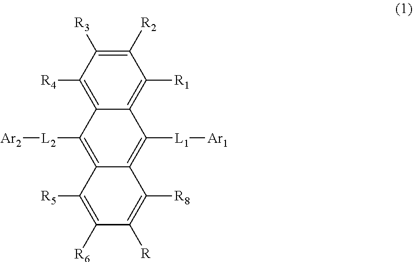

In the formula (1),

at least one of R₁ to R is a group represented by the following formula (1-2);

-L₃-Ar₃ (1-2)

L₁ to L₃ are independently

a single bond,

a substituted or unsubstituted phenylene group,

a substituted or unsubstituted naphthylene group,

a substituted or unsubstituted biphenylene group,

a substituted or unsubstituted terphenylene group,

a substituted or unsubstituted anthrylene group, or

a substituted or unsubstituted phenanthrylene group;

Ar₁ to Ar₃ are independently

a substituted or unsubstituted phenyl group,

a substituted or unsubstituted naphthyl group,

a substituted or unsubstituted biphenyl group,

a substituted or unsubstituted terphenyl group,

a substituted or unsubstituted anthryl group, or

a substituted or unsubstituted phenanthryl group;

when each of L₁, L₂, L₃, Ar₁, Ar₂ and Ar₃ has a substituent, the substituent is

an alkyl group including 1 to 50 carbon atoms,

a cycloalkyl group including 3 to 50 ring carbon atoms, or

an alkylsilyl group including 1 to 50 carbon atoms;

R₁ to R₈ which are not the group represented by the formula (1-2) are independently

a hydrogen atom,

a substituted or unsubstituted alkyl group including 1 to 50 carbon atoms,

a substituted or unsubstituted cycloalkyl group including 3 to 50 ring carbon atoms,

—Si(R₉₀₁)(R₉₀₂)(R₉₀₃),

—O—(R₉₀₄), or

—S—(R₉₀₅);

R₉₀₁ to R₉₀₅ are independently

a hydrogen atom,

a substituted or unsubstituted alkyl group including 1 to 50 carbon atoms,

a substituted or unsubstituted cycloalkyl group including 3 to 50 ring carbon atoms, or

a substituted or unsubstituted aryl group including 6 to 50 ring carbon atoms;

when two or more of each of R₉₀₁ to R₉₀₅ are present, the two or more of each of R₉₀₁ to R₉₀₅ may be the same or different;

at least one of R₁ to R₈ which are not the group represented by the formula (1-2) is a deuterium atom;

adjacent two or more among R₁ to R₄ do not form a ring by bonding with each other, and adjacent two or more among R₅ to R₈ do not form a ring by bonding with each other.

The compound represented by the formula (1) is used as a material for an organic EL device, whereby an organic electroluminescence device having a low CIEy value and a long lifetime can be provided.

In one embodiment, in the formula (1), at least two of R₁ to R₈ which are not the group represented by the formula (1-2) are deuterium atoms.

In one embodiment, in the formula (1), all of R₁ to R₈ which are not the group represented by the formula (1-2) are deuterium atoms.

In one embodiment, in the formula (1), at least one hydrogen atom which one or more selected from the group consisting of L₁ to L₃ have is a deuterium atom.

In one embodiment, in the formula (1), at least one hydrogen atom which one or more selected from the group consisting of Ar₁ to Ar₃ have is a deuterium atom.

In one embodiment, in the formula (1), Ar₁ to Ar₃ are independently

a substituted or unsubstituted phenyl group,

a substituted or unsubstituted naphthyl group, or

a substituted or unsubstituted biphenyl group.

In one embodiment, in the formula (1), L₁ to L₃ are independently

a single bond,

a substituted or unsubstituted phenylene group,

a substituted or unsubstituted naphthylene group, or

a substituted or unsubstituted biphenylene group.

In one embodiment, in the formula (1), Ar₁ to Ar₃ are independently

an unsubstituted phenyl group, or

an unsubstituted naphthyl group.

In one embodiment, in the formula (1), L₁ to L₃ are independently

a single bond, or

an unsubstituted phenylene group.

In one embodiment, the compound represented by the formula (1) is a compound represented by the following formula (2).

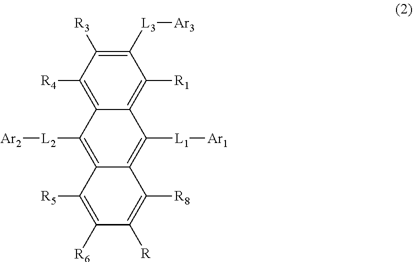

In the formula (2),

R₁, R₃ to R₈, L₁ to L₃ and Ar₁ to Ar₃ are as defined in the formula (1).

In one embodiment, the compound represented by the formula (1) is a compound represented by the following formula (3).

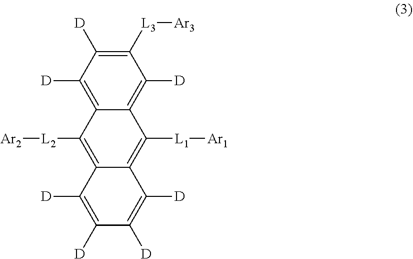

In the formula (3),

D is a deuterium atom; and

L₁ to L₃ and Ar₁ to Ar₃ are as defined in the formula (1).

In one embodiment, the compound represented by the formula (1) is a compound represented by the following formula (4).

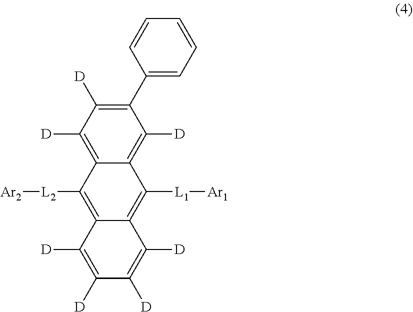

In the formula (4),

D is a deuterium atom; and

L₁, L₂, Ar₁ and Ar₂ are as defined in the formula (1).

In one embodiment, the compound represented by the formula (1) is a compound represented by the following formula (5).

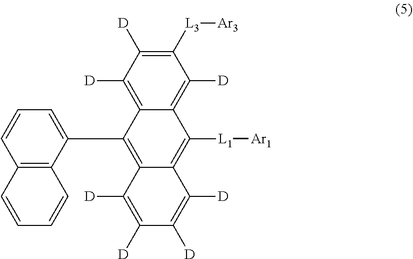

In the formula (5),

D is a deuterium atom; and

L₁, L₃, Ar₁ and Ar₃ are as defined in the formula (1).

In one embodiment, the compound represented by the formula (1) is a compound represented by the following formula (6).

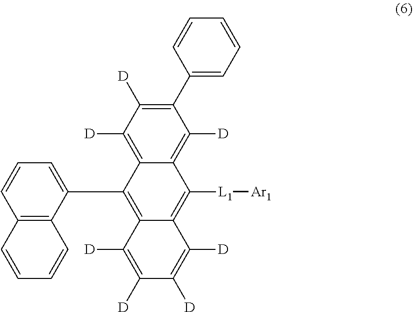

In the formula (6),

D is a deuterium atom; and

L₁ and Ar₁ are as defined in the formula (1).

Specific examples of the compound represented by the formula (1) of the second aspect include the same compounds as the specific examples of the compound represented by the formula (1) used in the organic EL device of the first aspect.

### [Material for an Organic Electroluminescence Device of the Second Aspect]

The material for an organic electroluminescence device according to the second aspect of the invention contains a compound represented by the formula (1).

The compound represented by the formula (1) is used as a material for an organic EL device, whereby an organic electroluminescence device having a low CIEy value and a long lifetime can be provided.

In one embodiment, the material for the organic EL device comprises the compound represented by the formula (1), and a compound having the same structures as the compound represented by the formula (1) except that the compound has only protium atoms as hydrogen atoms (hereinafter also referred to as “protium form”), and the content ratio of the former to the sum thereof is 30 mol % or more.

### [Organic Electroluminescence Device of the Second Aspect]

The organic electroluminescence device according to the second aspect of the invention comprises:

a cathode;

an anode; and

at least one organic layer disposed between the cathode and the anode,

wherein at least one of the at least one organic layer comprises a compound represented by the following formula (1).

In one embodiment, the organic layer includes an emitting layer, and the emitting layer contains the compound represented by the formula (1).

As described above, known materials and device configurations may be applied to the organic EL device according to the second aspect of the invention, as long as the device comprises a cathode, an anode, and at least one organic layer between the cathode and the anode, wherein at least one layer of the at least one organic layer comprises the compound represented by the formula (1), and the effect of the invention is not impaired.

Parts which can be used in the organic EL device according to the first and second aspects of the invention, materials for forming respective layers, other than the above-mentioned compounds, and the like, will be described later.

### (Substrate)

A substrate is used as a support of an emitting device. As the substrate, glass, quartz, plastic or the like can be used, for example. Further, a flexible substrate may be used. The “flexible substrate” means a bendable (flexible) substrate, and specific examples thereof include a plastic substrate formed of polycarbonate, polyvinyl chloride, or the like.

### (Anode)

For the anode formed on the substrate, metals, alloys, electrically conductive compounds, mixtures thereof, and the like, which have a large work function (specifically 4.0 eV or more) are preferably used. Specific examples include indium oxide-tin oxide (ITO: Indium Tin Oxide), indium oxide-zinc oxide, tungsten oxide, graphene, and the like. These electrodes may further contain other elements. For example, such other elements include silicon, iron, copper, chromium, nickel, and the like. In addition thereto, specific examples thereof include gold (Au), platinum (Pt), a nitride of a metallic material (for example, titanium nitride), and the like.

### (Hole-Injecting Layer)

The hole-injecting layer is a layer containing a substance having high hole-injecting property. As such a substance having high hole-injecting property molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, or a polymer compound (oligomers, dendrimers, polymers, etc.) can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. However, a substance other than the above-described substances may be used as long as the substance has higher hole-transporting property in comparison with an electron-transporting property. It should be noted that the layer containing the substance having high hole-transporting property may be not only a single layer, but also a layer in which two or more layers formed of the above-described substances are stacked.

### (Guest Material for Emitting Layer)

The emitting layer is a layer containing a substance having a high emitting property, and various materials can be used for forming it. For example, as the substance having a high emitting property, a fluorescent compound which emits fluorescence or a phosphorescent compound which emits phosphorescence can be used in addition to the compound represented by the formula (A1). The fluorescent compound is a compound which can emit from a singlet excited state, and the phosphorescent compound is a compound which can emit from a triplet excited state.

As a blue fluorescent emitting material which can be used for an emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for an emitting layer, aromatic amine derivatives and the like can be used. As a red fluorescent emitting material which can be used for an emitting layer, tetracene derivatives, diamine derivatives and the like can be used.

As a blue phosphorescent emitting material which can be used for an emitting layer, metal complexes such as iridium complexes, osmium complexes, platinum complexes and the like are used. As a green phosphorescent emitting material which can be used for an emitting layer, iridium complexes and the like are used. As a red phosphorescent emitting material which can be used for an emitting layer, metal complexes such as iridium complexes, platinum complexes, terbium complexes, europium complexes and the like are used.

(Host Material for Emitting Layer) The emitting layer may have a constitution in which the substance having a high emitting property (guest material) is dispersed in another substance (host material). As a substance for dispersing the substance having a high emitting property, a variety of substances can be used in addition to the compound represented by the formula (1), and it is preferable to use a substance having a higher lowest unoccupied orbital level (LUMO level) and a lower highest occupied orbital level (HOMO level) than the substance having a high emitting property.

As a substance for dispersing the substance having a high emitting property (host material), 1) metal complexes such as aluminum complexes, beryllium complexes, zinc complexes, or the like; 2) heterocyclic compounds such as oxadiazole derivatives, benzimidazole derivatives, phenanthroline derivatives, or the like; 3) fused aromatic compounds such as carbazole derivatives, anthracene derivatives, phenanthrene derivatives, pyrene derivatives, chrysene derivatives, or the like; and 3) aromatic amine compound such as triarylamine derivatives, aromatic amine derivatives, or the like are used.

### (Electron-Transporting Layer)

An electron-transporting layer is a layer which contains a substance having high electron-transporting property. For the electron-transporting layer, 1) metal complexes such as aluminum complexes, beryllium complexes, zinc complexes, or the like; 2) heteroaromatic complexes such as imidazole derivatives, benzimidazole derivatives, azine derivatives, carbazole derivatives, phenanthroline derivatives, or the like; and 3) polymer compounds can be used.

### (Electron-Injecting Layer)

An electron-injecting layer is a layer which contains a substance having a high electron-injecting property. For the electron-injecting layer, metal complex compounds such as lithium (Li), ytterbium (Yb), lithium fluoride (LiF), cesium fluoride (CsF), calcium fluoride (CaF₂), 8-hydroxyquinolinolato-lithium (Liq); alkali metals such as lithium oxide (LiOₓ); alkaline earth metals; or a compound thereof can be used.

### (Cathode)

For the cathode, metals, alloys, electrically conductive compounds, mixtures thereof, and the like having a small work function (specifically, 3.8 eV or less) are preferably used. Specific examples of such a cathode material include elements belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AlLi); rare earth metals such as europium (Eu) and ytterbium (Yb), and alloys containing these metals.

In the organic EL device according to an aspect of the invention, the methods for forming the respective layers are not particularly limited. A conventionally-known method for forming each layer according to a vacuum deposition process, a spin coating process or the like can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

In the organic EL device according to an aspect of the invention, the thickness of each layer is not particularly limited, but is generally preferable that the thickness be in the range of several nm to 1 μm in order to suppress defects such as pinholes, to suppress applied voltages to be low, and to improve luminous efficiency.

### [Electronic Apparatus of the First and Second Aspects]

The electronic apparatus according to the first aspect of the invention is characterized in that the organic EL device according to the first aspect of the invention is provided.

The electronic apparatus according to the second aspect of the invention is characterized in that the organic EL device according to the second aspect of the invention is provided.

Specific examples of the electronic apparatus include a display component such as an organic EL panel module, and the like; a display device such as a television, a cellular phone, a personal computer, and the like; and an emitting device such as a light, a vehicular lamp, and the like.

### EXAMPLES

Next, the invention will be explained in more detail with reference to the following Examples and Comparative Examples. However, it should be noted that the invention be not limited due to the description of Examples at all.

### (Compound)

Compounds used in the following Examples and Comparative Examples are as follows.

The compounds represented by the formula (1) used for fabricating the organic EL device of Examples 1 to 24 are as follows:

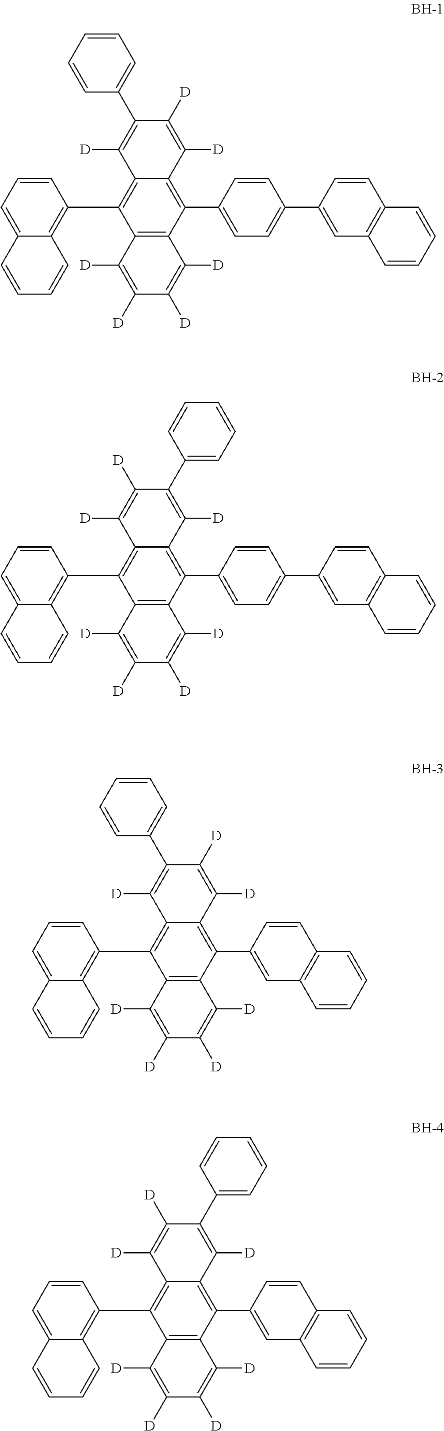 [SMILES: [2H]c1c([2H])c([2H])c2c(-c3cccc4ccccc34)c3c([2H])c(-c4ccccc4)c([2H])c([2H])c3c(-c3ccc(-c4ccc5ccccc5c4)cc3)c2c1[2H] ; [2H]c1c([2H])c([2H])c2c(-c3cccc4ccccc34)c3c([2H])c([2H])c(-c4ccccc4)c([2H])c3c(-c3ccc(-c4ccc5ccccc5c4)cc3)c2c1[2H] ; [2H]c1c([2H])c([2H])c2c(-c3cccc4ccccc34)c3c([2H])c(-c4ccccc4)c([2H])c([2H])c3c(-c3ccc4ccccc4c3)c2c1[2H] ; [2H]c1c([2H])c([2H])c2c(-c3cccc4ccccc34)c3c([2H])c([2H])c(-c4ccccc4)c([2H])c3c(-c3ccc4ccccc4c3)c2c1[2H]]

The compounds represented by the formula (1A) used for fabricating the organic EL devices of Examples 1 to 24 are as follows:

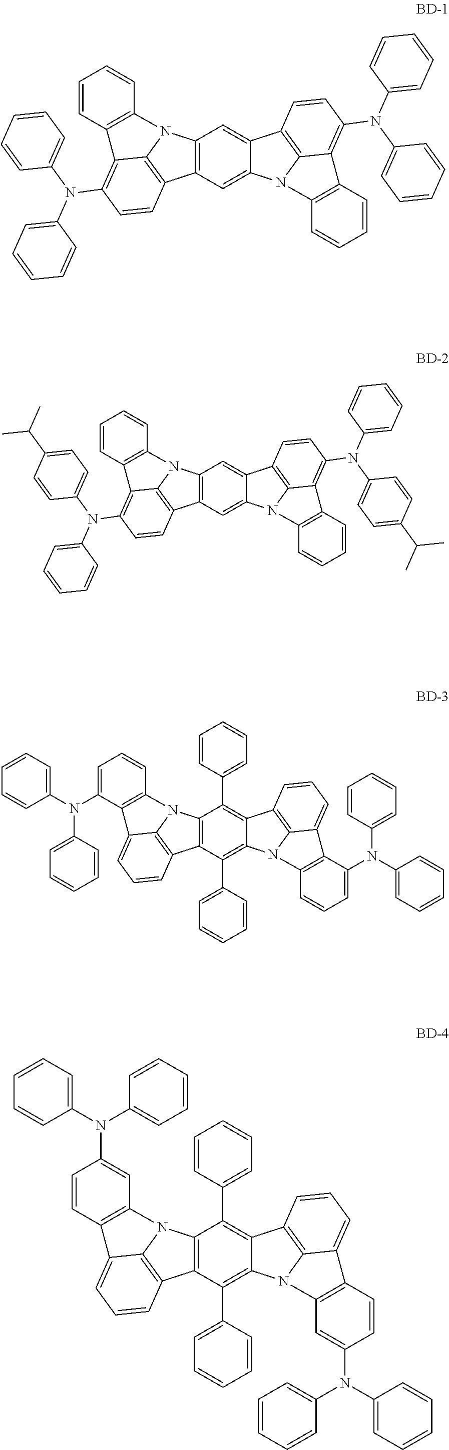 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; c1ccc(-c2c3c4cccc5c6c(N(c7ccccc7)c7ccccc7)cccc6n(c3c(-c3ccccc3)c3c6cccc7c8c(N(c9ccccc9)c9ccccc9)cccc8n(c23)c76)c54)cc1 ; c1ccc(-c2c3c4cccc5c6ccc(N(c7ccccc7)c7ccccc7)cc6n(c3c(-c3ccccc3)c3c6cccc7c8ccc(N(c9ccccc9)c9ccccc9)cc8n(c23)c76)c54)cc1]

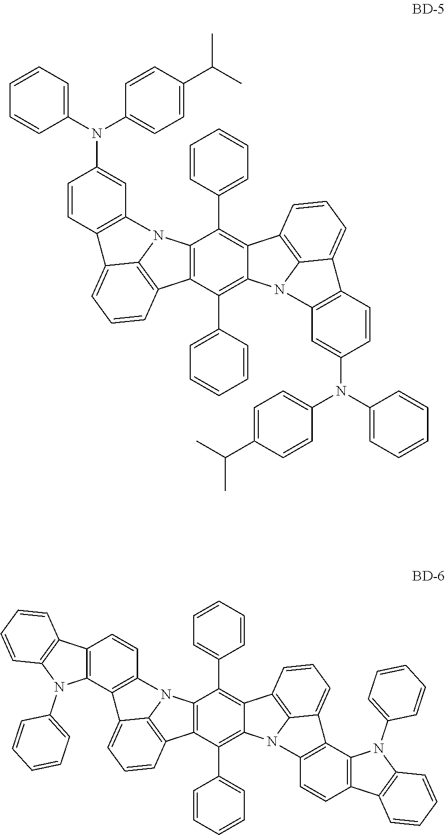 [SMILES: CC(C)c1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C(C)C)cc5)cc4n7c32)cc1 ; c1ccc(-c2c3c4cccc5c6c7c(ccc6n(c3c(-c3ccccc3)c3c6cccc8c9c%10c(ccc9n(c23)c68)c2ccccc2n%10-c2ccccc2)c45)c2ccccc2n7-c2ccccc2)cc1]

The structures of the compounds used for fabricating the organic EL devices of Comparative Examples 1 to 24 are as follows:

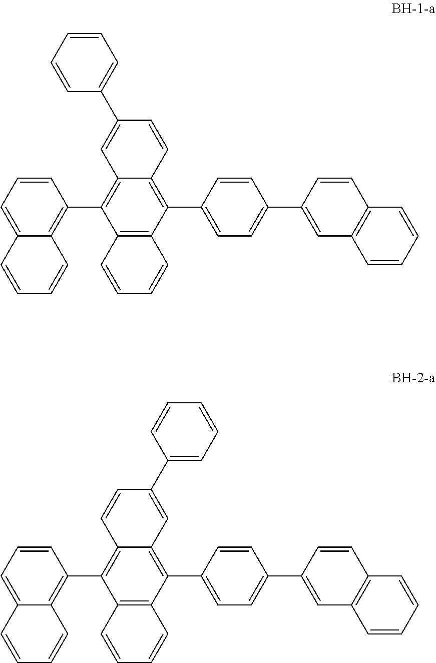

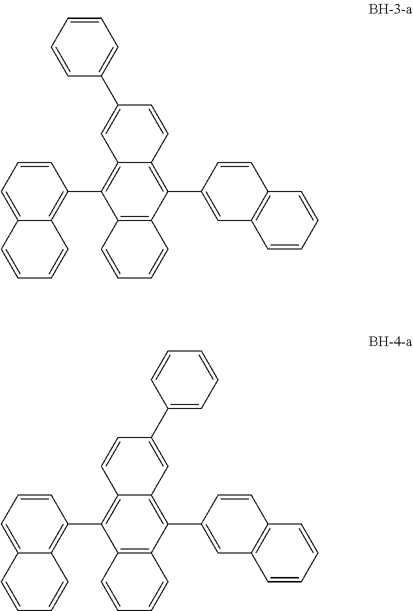

The structure of the other compounds used for fabricating the organic EL devices of Examples 1 to 24 and Comparative Examples 1 to 24 are as follows:

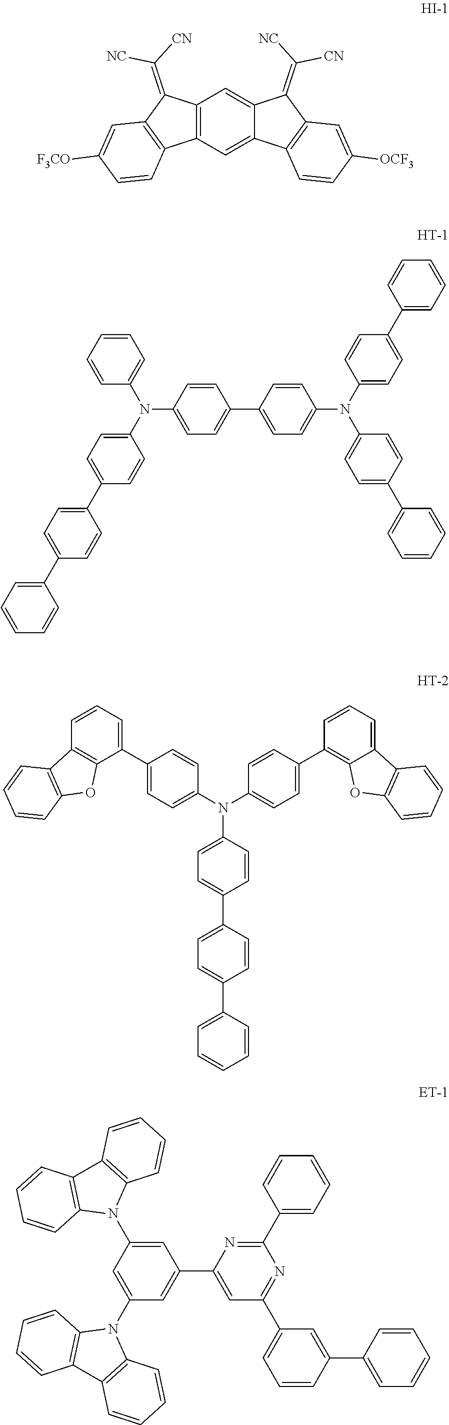 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

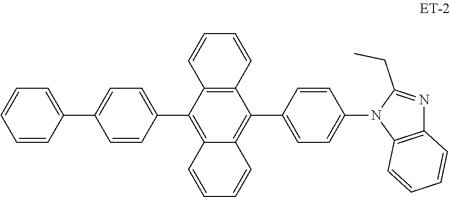 [SMILES: CCc1nc2ccccc2n1-c1ccc(-c2c3ccccc3c(-c3ccc(-c4ccccc4)cc3)c3ccccc23)cc1]

### Example 1

### (Fabrication of Organic EL Devices)

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HI-1 was deposited on a surface on the side on which the transparent electrode was formed so as to cover the transparent electrode to form a HI-1 film having a thickness of 5 nm. The HI-1 film functions as a hole-injecting layer.

Subsequent to the formation of the HI-1 film, a compound HT-1 was deposited thereon to form an HT-1 film having a thickness of 80 nm on the HI-1 film. The HT-1 film functions as a hole-transporting layer (first hole-transporting layer).

Subsequent to the formation of the HT-1 film, a compound HT-2 was deposited thereon to form an HT-2 film having a thickness of 10 nm on the HT-1 film. The HT-2 film functions as an electron-blocking layer (second hole-transporting layer).

A compound BH-1 (host material) and a compound BD-1 (dopant material) were co-deposited on the HT-2 film such that the proportion of the compound BD-1 became 2 mass %, and a BH-1:BD-1 film having a thickness of 25 nm was formed. The BH-1:BD-1 film functions as an emitting layer.

A compound ET-1 was deposited on the emitting layer to form an ET-1 film having a thickness of 10 nm. The ET-1 film functions as a hole barrier layer.

A compound ET-2 was deposited on the ET-1 film to form an ET-2 film having a thickness of 15 nm. The ET-2 film functions as an electron-transporting layer. LiF was deposited on the ET-2 film to form a LiF film having a thickness of 1 nm. A1 metal was deposited on the LiF film to form a metal cathode having a thickness of 80 nm to obtain an organic EL device.

The layer configuration of the obtained organic EL device is as follows: ITO(130)/HI-1(5)/HT-1(80)/HT-2(10)/BH-1:BD-1(25:2 mass %)/ET-1(10)/ET-2(15)/LiF(1)/Al(80)

The numerical values in parentheses indicate the film thickness (unit: nm). The numerical value represented in percentages in parentheses indicates the proportion (mass %) of the host material and the dopant material in the emitting layer.

### (Evaluation of Organic EL Device)

A voltage was applied to the obtained organic EL device so that the current density became 50 mA/cm², and the time until the luminance became 95% of the initial luminance (LT95 (unit: hours)) was measured. The results are shown in Table 1.

Further, a voltage was applied to the obtained organic EL device so that the current density became 10 mA/cm², where CIE1931 chromaticity coordinates (x, y) were measured by obtaining a spectral radiance using a spectroradiometer CS-1000 (manufactured by Konica Minolta, Inc.). The results are shown in Table 1.

### Example 2 and Comparative Examples 1 to 2

The organic EL device was fabricated and evaluated in the same manner as in Example 1 except that the compounds shown in Table 1 were used as the host material (BH) and the dopant material (BD), respectively. The results are shown in Table 1.

TABLE 1

| | |
|---|---|
| Emitting layer | Chromaticity |

| | BH | BD | LT95 (hr) | CIEx | CIEy |
|---|---|---|---|---|---|
| Example 1 | BH-1 | BD-1 | 135 | 0.140 | 0.081 |
| Comparative | BH-1-a | BD-1 | 90 | 0.140 | 0.081 |
| Example 1 | | | | | |
| Example 2 | BH-2 | BD-1 | 136 | 0.140 | 0.080 |
| Comparative | BH-2-a | BD-1 | 91 | 0.140 | 0.080 |
| Example 2 | | | | | |

### Examples 3 to 4 and Comparative Examples 3 to 4

The organic EL device was fabricated and evaluated in the same manner as in Example 1 except that the compounds shown in Table 2 were used as the host material (BH) and the dopant material (BD), respectively. The results are shown in Table 2.

TABLE 2

| | |
|---|---|
| Emitting layer | Chromaticity |

| | BH | BD | LT95 (hr) | CIEx | CIEy |
|---|---|---|---|---|---|
| Example 3 | BH-1 | BD-2 | 140 | 0.135 | 0.098 |
| Comparative | BH-1-a | BD-2 | 108 | 0.135 | 0.098 |
| Example 3 | | | | | |
| Example 4 | BH-2 | BD-2 | 142 | 0.135 | 0.098 |
| Comparative | BH-2-a | BD-2 | 110 | 0.135 | 0.098 |
| Example 4 | | | | | |

### Examples 5 to 6 and Comparative Examples 5 to 6

The organic EL device was fabricated and evaluated in the same manner as in Example 1 except that the compounds shown in Table 3 were used as the host material (BH) and the dopant material (BD), respectively. The results are shown in Table 3.

TABLE 3

| | |
|---|---|
| Emitting layer | Chromaticity |

| | BH | BD | LT95 (hr) | CIEx | CIEy |
|---|---|---|---|---|---|
| Example 5 | BH-1 | BD-3 | 144 | 0.135 | 0.086 |
| Comparative | BH-1-a | BD-3 | 110 | 0.135 | 0.085 |
| Example 5 | | | | | |
| Example 6 | BH-2 | BD-3 | 145 | 0.135 | 0.085 |
| Comparative | BH-2-a | BD-3 | 114 | 0.135 | 0.086 |
| Example 6 | | | | | |

### Examples 7 to 8 and Comparative Examples 7 to 8

The organic EL device was fabricated and evaluated in the same manner as in Example 1 except that the compounds shown in Table 4 were used as the host material (BH) and the dopant material (BD), respectively. The results are shown in Table 4.

TABLE 4

| | |
|---|---|
| Emitting layer | Chromaticity |

| | BH | BD | LT95 (hr) | CIEx | CIEy |
|---|---|---|---|---|---|
| Example 7 | BH-1 | BD-4 | 198 | 0.135 | 0.080 |
| Comparative | BH-1-a | BD-4 | 133 | 0.135 | 0.080 |
| Example 7 | | | | | |
| Example 8 | BH-2 | BD-4 | 200 | 0.135 | 0.080 |
| Comparative | BH-2-a | BD-4 | 132 | 0.135 | 0.080 |
| Example 8 | | | | | |

### Examples 9 to 10 and Comparative Examples 9 to 10

The organic EL device was fabricated and evaluated in the same manner as in Example 1 except that the compounds shown in Table 5 were used as the host material (BH) and the dopant material (BD), respectively. The results are shown in Table 5.

TABLE 5

| | |
|---|---|
| Emitting layer | Chromaticity |

| | BH | BD | LT95 (hr) | CIEx | CIEy |
|---|---|---|---|---|---|
| Example 9 | BH-1 | BD-5 | 228 | 0.136 | 0.090 |
| Comparative | BH-1-a | BD-5 | 157 | 0.136 | 0.090 |
| Example 9 | | | | | |
| Example 10 | BH-2 | BD-5 | 237 | 0.136 | 0.090 |
| Comparative | BH-2-a | BD-5 | 158 | 0.136 | 0.090 |
| Example 10 | | | | | |

### Examples 11 to 12 and Comparative Examples 11 to 12

The organic EL device was fabricated and evaluated in the same manner as in Example 1 except that the compounds shown in Table 6 were used as the host material (BH) and the dopant material (BD), respectively. The results are shown in Table 6.

TABLE 6

| | |
|---|---|
| Emitting layer | Chromaticity |

| | BH | BD | LT95 (hr) | CIEx | CIEy |
|---|---|---|---|---|---|
| Example 11 | BH-1 | BD-6 | 132 | 0.144 | 0.060 |
| Comparative | BH-1-a | BD-6 | 88 | 0.144 | 0.060 |
| Example 11 | | | | | |
| Example 12 | BH-2 | BD-6 | 133 | 0.144 | 0.061 |
| Comparative | BH-2-a | BD-6 | 90 | 0.144 | 0.061 |
| Example 12 | | | | | |

From the results in Tables 1 to 6, it can be seen that the organic EL devices of Examples 1 to 12 in which a specific host material and a specific dopant material are used in combination have a low CIEy value and a long lifetime, and that both of these characteristics are satisfied at the same time. In particular, it can be seen from the comparison between the Examples and the Comparative Examples using the same dopant material that the lifetime is significantly improved.

### Examples 13 to 14 and Comparative Examples 13 to 14

The organic EL device was fabricated and evaluated in the same manner as in Example 1 except that the compounds shown in Table 7 were used as the host material (BH) and the dopant material (BD), respectively. The results are shown in Table 7.

TABLE 7

| | |
|---|---|
| Emitting layer | Chromaticity |

| | BH | BD | LT95 (hr) | CIEx | CIEy |
|---|---|---|---|---|---|
| Example 13 | BH-3 | BD-1 | 154 | 0.140 | 0.080 |
| Comparative | BH-3-a | BD-1 | 97 | 0.140 | 0.081 |
| Example 13 | | | | | |
| Example 14 | BH-4 | BD-1 | 171 | 0.140 | 0.080 |
| Comparative | BH-4-a | BD-1 | 104 | 0.140 | 0.080 |
| Example 14 | | | | | |

### Examples 15 to 16 and Comparative Examples 15 to 16

The organic EL device was fabricated and evaluated in the same manner as in Example 1 except that the compounds shown in Table 8 were used as the host material (BH) and the dopant material (BD), respectively. The results are shown in Table 8.

TABLE 8

| | |
|---|---|
| Emitting layer | Chromaticity |

| | BH | BD | LT95 (hr) | CIEx | CIEy |
|---|---|---|---|---|---|
| Example 15 | BH-3 | BD-2 | 185 | 0.135 | 0.098 |
| Comparative | BH-3-a | BD-2 | 117 | 0.135 | 0.098 |
| Example 15 | | | | | |
| Example 16 | BH-4 | BD-2 | 162 | 0.135 | 0.098 |
| Comparative | BH-4-a | BD-2 | 119 | 0.135 | 0.098 |
| Example 16 | | | | | |

### Examples 17 to 18 and Comparative Examples 17 to 18

The organic EL device was fabricated and evaluated in the same manner as in Example 1 except that the compounds shown in Table 9 were used as the host material (BH) and the dopant material (BD), respectively. The results are shown in Table 9.

TABLE 9

| | |
|---|---|
| Emitting layer | Chromaticity |

| | BH | BD | LT95 (hr) | CIEx | CIEy |
|---|---|---|---|---|---|
| Example 17 | BH-3 | BD-3 | 190 | 0.135 | 0.086 |
| Comparative | BH-3-a | BD-3 | 139 | 0.135 | 0.086 |
| Example 17 | | | | | |
| Example 18 | BH-4 | BD-3 | 157 | 0.135 | 0.085 |
| Comparative | BH-4-a | BD-3 | 137 | 0.135 | 0.085 |
| Example 18 | | | | | |

### Examples 19 to 20 and Comparative Examples 19 to 20

The organic EL device was fabricated and evaluated in the same manner as in Example 1 except that the compounds shown in Table 10 were used as the host material (BH) and the dopant material (BD), respectively. The results are shown in Table 10.

TABLE 10

| | |
|---|---|
| Emitting layer | Chromaticity |

| | BH | BD | LT95 (hr) | CIEx | CIEy |
|---|---|---|---|---|---|
| Example 19 | BH-3 | BD-4 | 249 | 0.135 | 0.080 |
| Comparative | BH-3-a | BD-4 | 160 | 0.135 | 0.080 |
| Example 19 | | | | | |
| Example 20 | BH-4 | BD-4 | 228 | 0.135 | 0.080 |
| Comparative | BH-4-a | BD-4 | 166 | 0.135 | 0.080 |
| Example 20 | | | | | |

### Examples 21 to 22 and Comparative Examples 21 to 22

The organic EL device was fabricated and evaluated in the same manner as in Example 1 except that the compounds shown in Table 11 were used as the host material (BH) and the dopant material (BD), respectively. The results are shown in Table 11.

TABLE 11

| | |
|---|---|
| Emitting layer | Chromaticity |

| | BH | BD | LT95 (hr) | CIEx | CIEy |
|---|---|---|---|---|---|
| Example 21 | BH-3 | BD-5 | 260 | 0.136 | 0.090 |
| Comparative | BH-3-a | BD-5 | 179 | 0.136 | 0.090 |
| Example 21 | | | | | |
| Example 22 | BH-4 | BD-5 | 270 | 0.136 | 0.090 |
| Comparative | BH-4-a | BD-5 | 209 | 0.136 | 0.090 |
| Example 22 | | | | | |

### Examples 23 to 24 and Comparative Examples 23 to 24

The organic EL device was fabricated and evaluated in the same manner as in Example 1 except that the compounds shown in Table 12 were used as the host material (BH) and the dopant material (BD), respectively. The results are shown in Table 12.

TABLE 12

| | |
|---|---|
| Emitting layer | Chromaticity |

| | BH | BD | LT95 (hr) | CIEx | CIEy |
|---|---|---|---|---|---|
| Example 23 | BH-3 | BD-6 | 143 | 0.144 | 0.061 |
| Comparative | BH-3-a | BD-6 | 106 | 0.144 | 0.061 |
| Example 23 | | | | | |
| Example 24 | BH-4 | BD-6 | 160 | 0.144 | 0.060 |
| Comparative | BH-4-a | BD-6 | 113 | 0.144 | 0.060 |
| Example 24 | | | | | |

From the results in Tables 7 to 12, it can be seen that the organic EL devices of Examples 13 to 24 in which a specific host material and a specific dopant material are used in combination have a low CIEy value and a long lifetime, and that both of these characteristics are satisfied at the same time.

### Synthesis Example 1 [Synthesis of Compound BH-1]

Synthetic scheme of BH-1 is shown below.

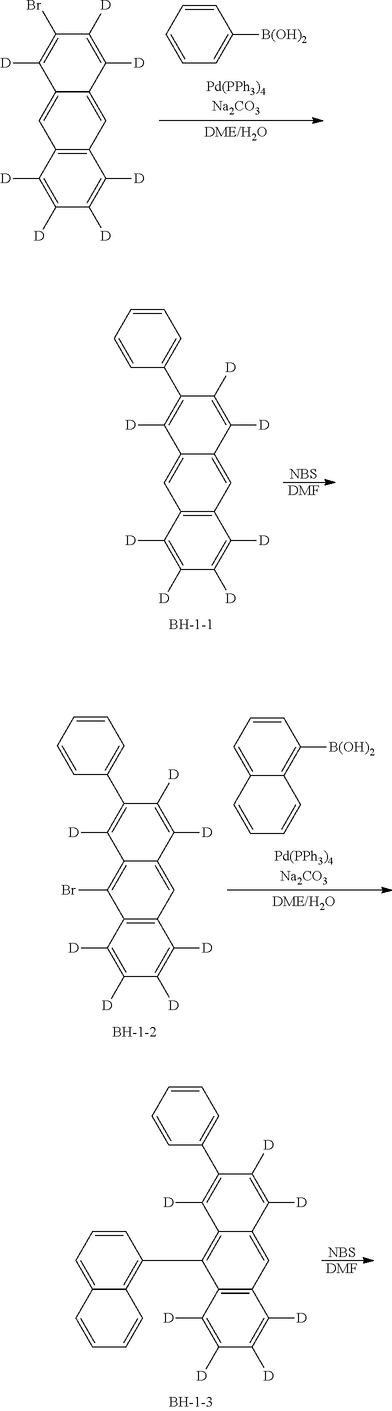

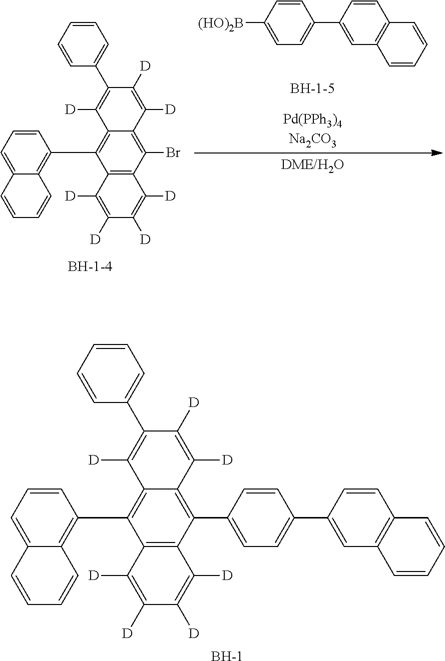

### (1-1) Synthesis of BH-1-1

Under an argon atmosphere, 376 mL of dimethoxyethane (DME) and 61 mL of water were added to a flask containing 23.1 g of 2-bromo-1,3,4,5,6,7,8-d7-anthracene (87.5 mmol), 11.7 g of phenylboronic acid (96.3 mmol), and 2.02 g of Pd(PPh₃)₄ (1.75 mmol), and 18.6 g of Na₂CO₃ (175.0 mmol)), and the mixture was heated at about 80° C. for 7 hours under stirring.

After completion of the reaction, the reaction mixture was cooled to room temperature, and water was added thereto to precipitate a solid, and the solid was collected by filtration, and the solid was purified by silica gel column chromatography to obtain a white solid of 15.6 g. The obtained compound was subjected to field desorption mass spectrometry (FD-MS analysis; Field Desorption Mass Spectrometry) and identified as BH-1-1 (yield: 68%).

### (1-2) Synthesis of BH-1-2

A solution of 15.4 g of BH-1-1 (59.0 mmol) dissolved in 680 mL of dimethylformamide (DMF) was heated at about 80° C. under stirring. 10.5 g of N-bromosuccinimide (NBS) (59.0 mmol) dissolved in 1180 mL of DMF was added dropwise thereto.

After completion of the reaction, the reaction mixture was cooled to room temperature, water was added thereto to precipitate a solid, and the solid was collected by filtration and purified by silica gel column chromatography. The resulting solid was purified by recrystallization with toluene to obtain 11.3 g of a white solid. The resulting compound was analyzed by FD-MS and identified as BH-1-2 (yield: 56%).

### (1-3) Synthesis of BH-1-3

Under an argon atmosphere, 55 mL of DME and 9 mL of water were added to a 300 mL-flask containing 4.39 g of BH-1-2 (12.9 mmol), 2.44 g of 1-naphthylboronic acid (14.2 mmol), 0.30 g of Pd(PPh₃)₄ (0.26 mmol), and 2.74 g of Na₂CO₃ (25.8 mmol), and the mixture was heated at about 80° C. for 24 hours under stirring.

After completion of the reaction, the mixture was cooled to room temperature, and the reaction solution was transferred to a separatory funnel and extracted with toluene. The organic phase was dried over anhydrous MgSO₄, filtered and concentrated. The obtained solid was purified by silica gel column chromatography to obtain 3.60 g of a solid. The resulting compound was analyzed by FD-MS and identified as BH-1-3 (yield: 72%).

### (1-4) Synthesis of BH-1-4

To 3.29 g of BH-1-3 (8.49 mmol), 170 mL of DMF was added, and heated at about 50° C. to dissolve BH-1-3, and then the temperature of the solution was lowered to room temperature. A DMF solution of 1.51 g of N-bromosuccinimide (NBS) (8.49 mmol) was added dropwise thereto and the obtained solution was stirred at room temperature.

After completion of the reaction, the reaction mixture was cooled to room temperature, water was added thereto to precipitate a solid, and the solid was collected by filtration and purified by silica gel column chromatography. The resulting solid was purified by recrystallization with toluene to obtain 1.90 g of a white solid. The resulting compound was analyzed by FD-MS and identified as BH-1-4 (yield: 48%).

### (1-5) Synthesis of BH-1

Under an argon atmosphere, 17 mL of DME and 2.8 mL of water were added to a flask containing 1.84 g of BH-1-4 (3.95 mmol), 1.08 g of BH-1-5 (4.34 mmol), 0.090 g of Pd(PPh₃)₄ (0.08 mmol), and 0.84 g of Na₂CO₃ (7.89 mmol), and the mixture was heated at about 80° C. for 7 hours under stirring.

After completion of the reaction, the reaction mixture was cooled to room temperature, water was added thereto to precipitate a solid, and the solid was collected by filtration and purified by silica gel column chromatography. Recrystallization with toluene was carried out repeatedly to obtain 1.21 g of a light yellow solid. The resulting compound was analyzed by FD-MS and identified as BH-1 (yield: 52%).

### Synthesis Example 2 [Synthesis of Compound BH-2]

Synthetic scheme of BH-2 is shown below.

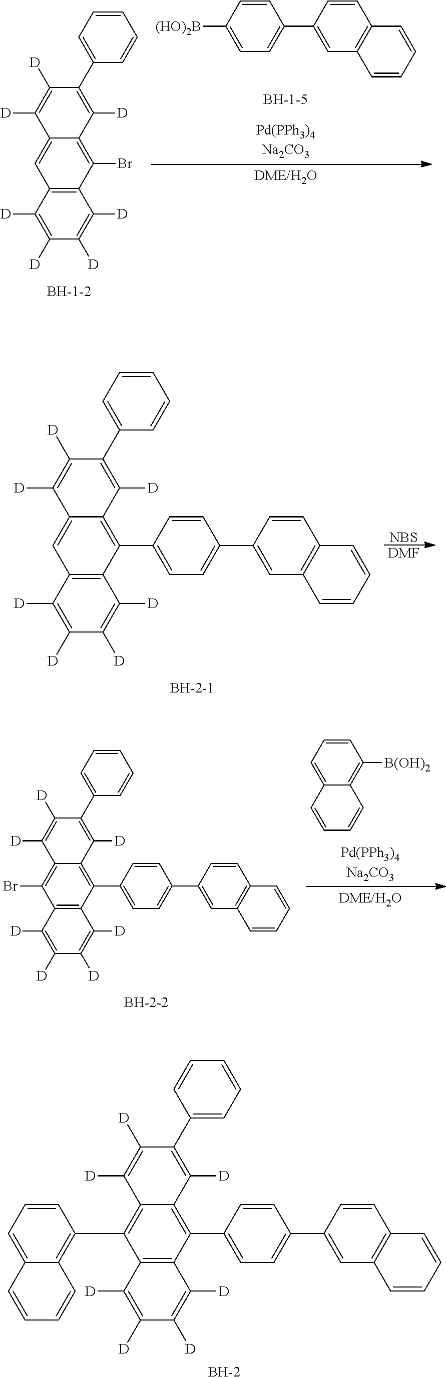

### (2-1) Synthesis of BH-2-1

Under an argon atmosphere, 63 mL of DME and 10 mL of water were added to a flask containing 5.00 g of BH-1-2 (14.7 mmol), 4.01 g of BH-1-5 (16.2 mmol), 0.34 g of Pd(PPh₃)₄ (0.29 mmol), and 3.12 g of Na₂CO₃ (29.4 mmol), and the mixture was heated at about 80° C. for 24 hours under stirring.

After completion of the reaction, the reaction solution was cooled to room temperature, and transferred to a separatory funnel and extracted with toluene. The organic phase was dried over anhydrous MgSO₄, filtered and concentrated. The obtained solid was purified by silica gel column chromatography to obtain 4.29 g of a solid. The resulting compound was analyzed by FD-MS and identified as BH-2-1 (yield: 63%).

### (2-2) Synthesis of BH-2-2

To 4.2 g of BH-2-1 (9.06 mmol), 180 mL of DMF was added, and heated at about 50° C. to dissolve BH-2-1, and then the temperature of the solution was lowered to room temperature. A DMF solution of 1.61 g of N-bromosuccinimide (NBS) (9.06 mmol) was added dropwise thereto and the obtained solution was stirred at room temperature.

After completion of the reaction, the reaction mixture was cooled to room temperature, water was added thereto to precipitate a solid, and the solid was collected by filtration and purified by silica gel column chromatography. The resulting solid was purified by recrystallization with toluene to obtain 2.02 g of a white solid. The resulting compound was analyzed by FD-MS and identified as BH-2-2 (yield: 41%).

### (2-3) Synthesis of BH-2

Under an argon atmosphere, 16 mL of DME and 2.6 mL of water were added to a flask containing 2.00 g of BH-2-2 (3.68 mmol), 0.70 g of 1-naphthylboronic acid (4.05 mmol), 0.09 g of Pd(PPh₃)₄ (0.07 mmol), and 0.78 g of Na₂CO₃ (7.36 mmol), and the mixture was heated at about 80° C. for 24 hours under stirring.

After completion of the reaction, the reaction mixture was cooled to room temperature, water was added thereto to precipitate a solid, the solid was collected by filtration and purified by silica gel column chromatography. Recrystallization with toluene was carried out repeatedly to obtain 1.09 g of a light yellow solid. The resulting compound was analyzed by FD-MS and identified as BH-2 (yield: 50%).

### Synthesis Example 3 [Synthesis of Compound BH-3]

Synthetic scheme of BH-3 is shown below.

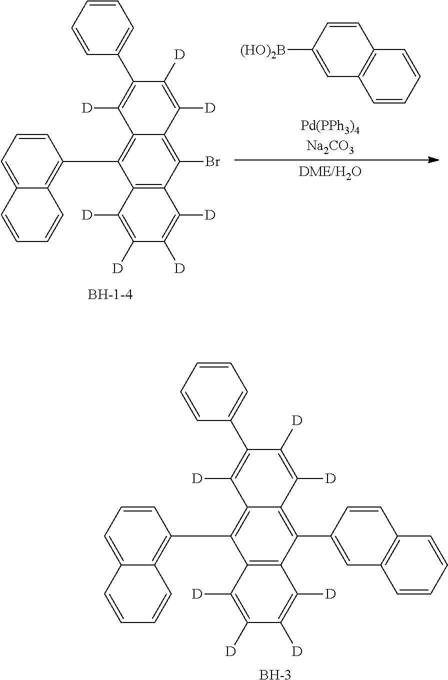

### Synthesis of BH-3

Under an argon atmosphere, 17 mL of DME and 2.8 mL of water were added to a flask containing 1.84 g of BH-1-4 (3.95 mmol), 0.75 g of 2-naphthylboronic acid (4.34 mmol) and 0.090 g of Pd(PPh₃)₄ (0.08 mmol), and 0.84 g of Na₂CO₃ (7.89 mmol), and the mixture was heated at about 80° C. for 7 hours under stirring.

After completion of the reaction, the reaction mixture was cooled to room temperature, water was added thereto to precipitate a solid, and the solid was collected by filtration and purified by silica gel column chromatography. Recrystallization with toluene was carried out repeatedly to obtain 1.26 g of a light yellow solid. The resulting compound was analyzed by FD-MS and identified as BH-3 (yield: 62%).

### Synthesis Example 4 [Synthesis of Compound BH-4]

Synthetic scheme of BH-4 is shown below.

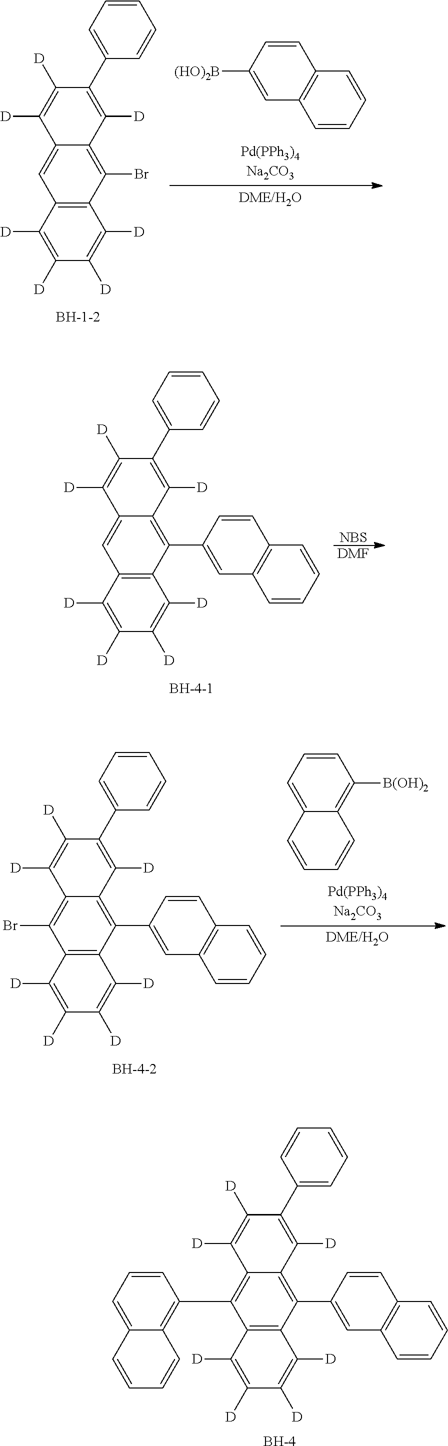

### (4-1) Synthesis of BH-4-1

Under an argon atmosphere, 63 mL of DME and 10 mL of water were added to a flask containing 5.00 g of BH-1-2 (14.7 mmol), 2.79 g of 2-naphthylboronic acid (16.2 mmol), 0.34 g of Pd(PPh₃)₄ (0.29 mmol), and 3.12 g of Na₂CO₃ (29.4 mmol), and the mixture was heated at about 80° C. for 24 hours under stirring.

After completion of the reaction, the reaction solution was cooled to room temperature and transferred to a separatory funnel and extracted with toluene. The organic phase was dried over anhydrous MgSO₄, filtered and concentrated. The solid was purified by silica gel column chromatography to obtain 3.42 g of a solid. The resulting compound was analyzed by FD-MS and identified as BH-4-1 (yield: 60%).

### (4-2) Synthesis of BH-4-2

To 3.4 g of BH-4-1 (8.77 mmol), 175 mL of DMF was added, and the mixture was heated at about 50° C. to dissolve BH-4-1, and then the temperature of the solution was lowered to room temperature. A DMF solution of 1.56 g of N-bromosuccinimide (NBS) (8.77 mmol) was added dropwise thereto and the obtained solution was stirred at room temperature.

After completion of the reaction, the reaction solution was cooled to room temperature, water was added thereto to precipitate a solid, and the solid was collected by filtration and purified by silica gel column chromatography. The resulting solid was subjected to recrystallization with toluene to obtain 1.64 g of a white solid. The resulting compound was analyzed by FD-MS and identified as BH-4-2 (yield: 40%).

### (4-3) Synthesis of BH-4

Under an argon atmosphere, 13 mL of DME and 2.1 mL of water were added to a flask containing 1.60 g of BH-4-2 (2.94 mmol), 0.56 g of 1-naphthylboronic acid (3.24 mmol), 0.07 g of Pd(PPh₃)₄ (0.06 mmol), and 0.62 g of Na₂CO₃ (5.89 mmol), and the mixture was heated at about 80° C. for 24 hours under stirring.

After completion of the reaction, the reaction solution was cooled to room temperature, water was added thereto to precipitate a solid, and the solid was collected by filtration and purified by silica gel column chromatography. Recrystallization with toluene was carried out repeatedly to obtain 0.80 g of a light yellow solid. The resulting compound was analyzed by FD-MS and identified as BH-4 (yield: 53%).

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, all such modifications are intended to be included within the scope of this invention.

The documents described in the specification and the entire contents of the application which is the basis of the priority under the Paris Convention of this application are incorporated herein by reference in its entirety.

1. An organic electroluminescence device, comprising:
a cathode;
an anode; and
an emitting layer disposed between the cathode and the anode,
wherein the emitting layer comprises:
a compound represented by the following formula (1) and
a compound represented by the following formula (A1): 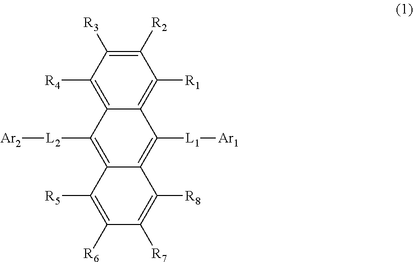
wherein in the formula (1),
at least one of R₁ to R₈ is a group represented by the following formula (1-2);
-L₃-Ar₃ (1-2)
L₁ to L₃ are independently
a single bond,
a substituted or unsubstituted phenylene group,
a substituted or unsubstituted naphthylene group,
a substituted or unsubstituted biphenylene group,
a substituted or unsubstituted terphenylene group,
a substituted or unsubstituted anthrylene group, or
a substituted or unsubstituted phenanthrylene group;
Ar₁ to Ar₃ are independently
a substituted or unsubstituted phenyl group,
a substituted or unsubstituted naphthyl group,
a substituted or unsubstituted biphenyl group,
a substituted or unsubstituted terphenyl group,
a substituted or unsubstituted anthryl group, or
a substituted or unsubstituted phenanthryl group;
when each of L₁, L₂, L₃, Ar₁, Ar₂ and Ar₃ has a substituent, the substituent is
an alkyl group including 1 to 50 carbon atoms,
a cycloalkyl group including 3 to 50 ring carbon atoms, or
an alkylsilyl group including 1 to 50 carbon atoms;
R₁ to R₈ which are not the group represented by the formula (1-2) are independently
a hydrogen atom,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄), or
—S—(R₉₀₅);
R₉₀₁ to R₉₀₅ are independently
a hydrogen atom,
a substituted or unsubstituted alkyl group including 1 to 50 carbon atoms,
a substituted or unsubstituted cycloalkyl group including 3 to 50 ring carbon atoms, or
a substituted or unsubstituted aryl group including 6 to 50 ring carbon atoms;
when two or more of each of R₉₀₁ to R₉₀₅ are present, the two or more of each of R₉₀₁ to R₉₀₅ may be the same or different;
at least one of R₁ to R₈ which are not the group represented by the formula (1-2) is a deuterium atom;
adjacent two or more among R₁ to R₄ do not form a ring by bonding with each other, and adjacent two or more among R₅ to R₈ do not form a ring by bonding with each other; 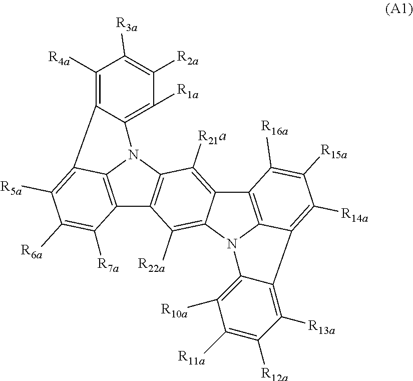
wherein in the formula (A1),
one or more sets of adjacent two or more among R₁ₐ to R₇ₐ and R₁₀ₐ to R₁₆ₐ form a substituted or unsubstituted, saturated or unsaturated ring by bonding with each other, or do not form a substituted or unsubstituted, saturated or unsaturated ring;
R₁ₐ to R₇ₐ and R₁₀ₐ to R₁₆ₐ which do not form the substituted or unsubstituted, saturated or unsaturated ring and R₂₁ₐ and R₂₂ₐ are independently a hydrogen atom or a substituent;
the substituent is
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
when two or more of each of R₉₀₁ to R₉₀₇ are present, the two or more of each of R₉₀₁ to R₉₀₇ may be the same or different.

2. The organic electroluminescence device according to claim 1, wherein in the formula (1), at least two among R₁ to R₈ in the formula (1) which are not the group represented by the formula (1-2) are deuterium atoms.

3. The organic electroluminescence device according to claim 1, wherein in the formula (1), all of R₁ to R₈ which are not the group represented by the formula (1-2) are deuterium atoms.

4. The organic electroluminescence device according to claim 1, wherein in the formula (1), at least one hydrogen atom which one or more selected from the group consisting of L₁ and L₂ have is a deuterium atom.

5. The organic electroluminescence device according to claim 1, wherein in the formula (1), L₁ and L₂ are independently
a single bond,
a substituted or unsubstituted phenylene group, or
a substituted or unsubstituted naphthylene group.

6. The organic electroluminescence device according to claim 1, wherein in formula (1), at least one hydrogen atom which one or more selected from the group consisting of Ar₁ and Ar₂ have is a deuterium atom.

7. The organic electroluminescence device according to claim 1, wherein in the formula (1), Ar₁ and Ar₂ are independently
a substituted or unsubstituted phenyl group,
a substituted or unsubstituted naphthyl group, or
a substituted or unsubstituted phenanthryl group.

8. The organic electroluminescence device according to claim 1, wherein the emitting layer comprises a compound represented by the formula (1) and a compound having the same structure as the compound represented by the formula (1) except that the compound comprises only protium atoms as hydrogen atoms, and the content ratio of the latter to the sum thereof is 99 mol % or less.

9. The organic electroluminescence device according to claim 1, wherein in the formula (A1), one or more among R₁ₐ to R₇ₐ and R₁₀ₐ to R₁₆ₐ are —N(R₉₀₆)(R₉₀₇) (where R₉₀₆ and R₉₀₇ are as defined in the formula (A1)).

10. The organic electroluminescence device according to claim 1, wherein in the formula (A1), two or more among R₁ₐ to R₇ₐ and R₁₀ₐ to R₁₆ₐ are —N(R₉₀₆)(R₉₀₇) (where R₉₀₆ and R₉₀₇ are as defined in the formula (A1)).

11. The organic electroluminescence device according to claim 1, wherein the compound represented by the formula (A1) is a compound represented by the following formula (A10): 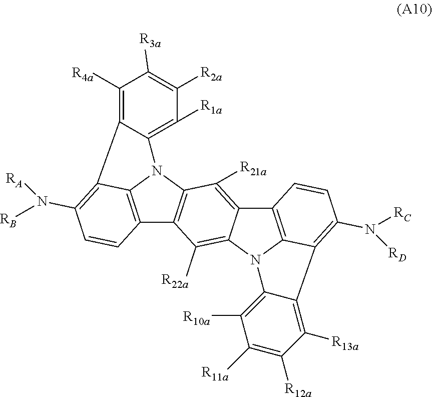
wherein in the formula (A10),
R₁ₐ to R₄ₐ, R₁₀ₐ to R₁₃ₐ, R₂₁ₐ and R₂₂ₐ are as defined in the formula (A1);
R_{A}, R_{B}, R_{C} and R_{D} are independently a substituted or unsubstituted aryl group including 6 to 18 ring carbon atoms, or a substituted or unsubstituted monovalent heterocyclic group including 5 to 18 ring atoms.

12. The organic electroluminescence device according to claim 11, wherein the compound represented by the formula (A10) is a compound represented by the following formula (A11): 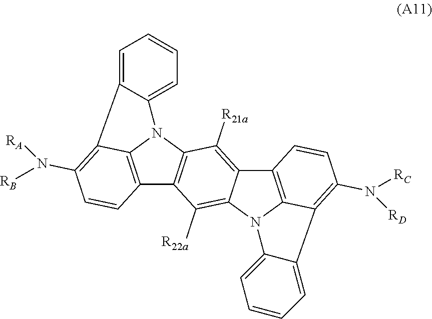
wherein in the formula (A11),
R₂₁ₐ, R₂₂ₐ, R_{A}, R_{B}, R_{C} and R_{D} are as defined in the formula (A10).

13. The organic electroluminescence device according to claim 11, wherein in the formula (A10), R_{A}, R_{B}, R_{C} and R_{D} are independently a substituted or unsubstituted aryl group including 6 to 18 ring carbon atoms.

14. The organic electroluminescence device according to claim 11, wherein in the formula (A10), R_{A}, R_{B}, R_{C} and R_{D} are independently a substituted or unsubstituted phenyl group.

15. The organic electroluminescence device according to claim 1, wherein in the formula (A1), one or more pairs selected from the group consisting of R₁ₐ and R₂ₐ, R₂ₐ and R₃ₐ, R₃ₐ and R₄ₐ, R₁₀ₐ and R₁₁ₐ, R₁₁ₐ and R₁₂ₐ, and R₁₂ₐ and R₁₃ₐ form a ring represented by the following formula (X): 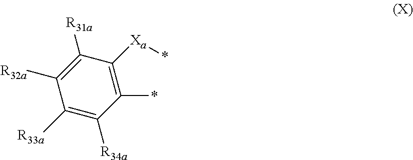
wherein in the formula (X),
two “*”s are respectively bonded with R₁ₐ and R₂ₐ, R₂ₐ and R₃ₐ, R₃ₐ and R₄ₐ, R₁₀ₐ and R₁₁ₐ, R₁₁ₐ and R₁₂ₐ, or R₁₂ₐ and R₁₃ₐ in the formula (A1);
R₃₁ₐ to R₃₄ₐ are independently a hydrogen atom, a substituted or unsubstituted alkyl group including 1 to 50 carbon atoms, or a substituted or unsubstituted aryl group including 6 to 50 ring carbon atoms;
when two or more of each of R₃₁ₐ to R₃₄ₐ are present, the two or more of each of R₃₁ₐ to R₃₄ₐ may be the same as or different to each other;
Xₐ is selected from O, S and N(R₃₅ₐ); when two or more Xₐ's are present, the two or more Xₐ's may be the same as or different to each other;
R₃₅ₐ forms a substituted or unsubstituted, saturated or unsaturated ring by bonding with R₃₁, or does not form a ring;
R₃₅ₐ which does not form a ring is a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms.

16. The organic electroluminescence device according to claim 15, wherein the compound represented by the formula (A1) is a compound represented by the following formula (A12): 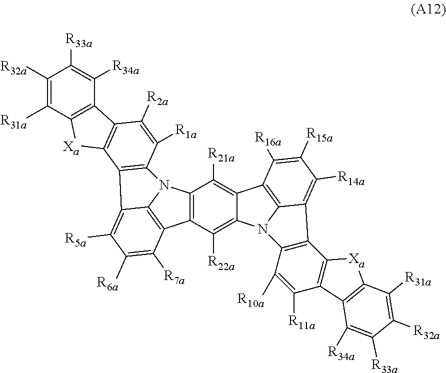
wherein in the formula (A12),
R₁ₐ, R₂ₐ, R₅ₐ to R₇ₐ, R₁₀ₐ, R₁₁ₐ, R₁₄ₐ to R₁₆ₐ, R₂₁ₐ, R₂₂ₐ, R₃₁ₐ to R₃₄ₐ and Xₐ are as defined in the formulas (A1) and (X).

17. The organic electroluminescence device according to claim 1, wherein R₂₁ₐ and R₂₂ₐ are hydrogen atoms.

18. The organic electroluminescence device according to claim 1, which further comprises a hole-transporting layer between the anode and the emitting layer.

19. The organic electroluminescence device according to claim 1, which further comprises an electron-transporting layer between the cathode and the emitting layer.

20. An electronic apparatus, comprising the organic electroluminescence device according to claim 1.

21. A compound represented by the following formula (1): 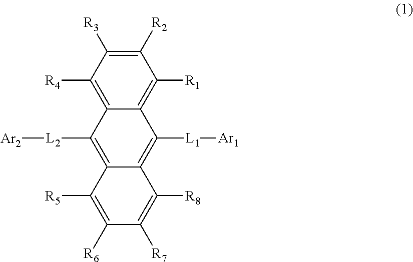
wherein in the formula (1),
at least one of R₁ to R₈ is a group represented by the following formula (1-2);
-L₃-Ar₃ (1-2)
L₁ to L₃ are independently
a single bond,
a substituted or unsubstituted phenylene group,
a substituted or unsubstituted naphthylene group,
a substituted or unsubstituted biphenylene group,
a substituted or unsubstituted terphenylene group,
a substituted or unsubstituted anthrylene group, or
a substituted or unsubstituted phenanthrylene group;
Ar₁ to Ar₃ are independently
a substituted or unsubstituted phenyl group,
a substituted or unsubstituted naphthyl group,
a substituted or unsubstituted biphenyl group,
a substituted or unsubstituted terphenyl group,
a substituted or unsubstituted anthryl group, or
a substituted or unsubstituted phenanthryl group;
when each of L₁, L₂, L₃, Ar₁, Ar₂ and Ar₃ has a substituent, the substituent is
an alkyl group including 1 to 50 carbon atoms,
a cycloalkyl group including 3 to 50 ring carbon atoms,
or an alkylsilyl group including 1 to 50 carbon atoms;
R₁ to R₈ which are not the group represented by the formula (1-2) are independently
a hydrogen atom,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄), or
—S—(R₉₀₅);
R₉₀₁ to R₉₀₅ are independently
a hydrogen atom,
a substituted or unsubstituted alkyl group including 1 to 50 carbon atoms,
a substituted or unsubstituted cycloalkyl group including 3 to 50 ring carbon atoms,
Or a substituted or unsubstituted aryl group including 6 to 50 ring carbon atoms;
when two or more of each of R₉₀₁ to R₉₀₅ are present, the two or more of each of R₉₀₁ to R₉₀₅ may be the same or different;
at least one of R₁ to R which are not the group represented by the formula (1-2) is a deuterium atom;
adjacent two or more among R₁ to R₄ do not form a ring by bonding with each other, and adjacent two or more among R₅ to R₈ do not form a ring by bonding with each other.

22. The compound according to claim 21, wherein in the formula (1), at least two of R₁ to R which are not the group represented by the formula (1-2) are deuterium atoms.

23. The compound according to claim 21, wherein in the formula (1), all of R₁ to R which are not the group represented by the formula (1-2) are deuterium atoms.

24. The compound according to claim 21, wherein in the formula (1), at least one hydrogen atom which one or more selected from the group consisting of L₁ to L₃ have is a deuterium atom.

25. The compound according to claim 21, wherein in the formula (1), at least one hydrogen atom which one or more selected from the group consisting of Ar₁ to Ar₃ have is a deuterium atom.

26. The compound according to claim 21, wherein in the formula (1), Ar₁ to Ar₃ are independently
a substituted or unsubstituted phenyl group,
a substituted or unsubstituted naphthyl group, or
a substituted or unsubstituted biphenyl group.

27. The compound according to claim 21, wherein in the formula (1), L₁ to L₃ are independently
a single bond,
a substituted or unsubstituted phenylene group,
a substituted or unsubstituted naphthylene group, or
a substituted or unsubstituted biphenylene group.

28. The compound according to claim 21, wherein in the formula (1), Ar₁ to Ar₃ are independently
an unsubstituted phenyl group, or
an unsubstituted naphthyl group.

29. The compound according to claim 21, wherein in the formula (1), L₁ to L₃ are independently
a single bond, or
an unsubstituted phenylene group.

30. The compound according to claim 21, wherein the compound represented by the formula (1) is a compound represented by the following formula (2): 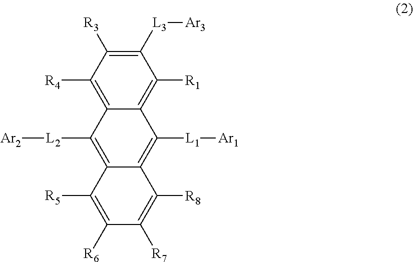
wherein in the formula (2),
R₁, R₃ to R₈, L₁ to L₃ and Ar₁ to Ar₃ are as defined in the formula (1).

31. The compound according to claim 21, wherein the compound represented by the formula (1) is a compound represented by the following formula (3): 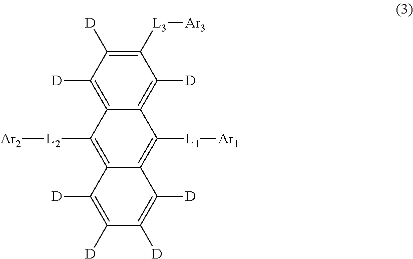
wherein in the formula (3),
D is a deuterium atom; and
L₁ to L₃ and Ar₁ to Ar₃ are as defined in the formula (1).

32. The compound according to claim 21, wherein the compound represented by the formula (1) is a compound represented by the following formula (4): 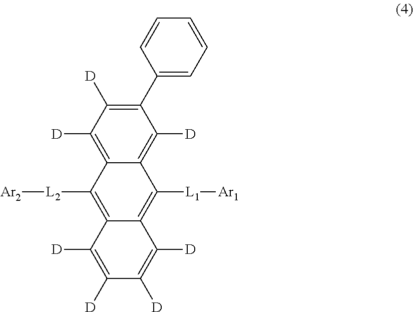
wherein in the formula (4),
D is a deuterium atom; and
L₁, L₂, Ar₁ and Ar₂ are as defined in the formula (1).

33. The compound according to claim 21, wherein the compound represented by the formula (1) is a compound represented by the following formula (5): 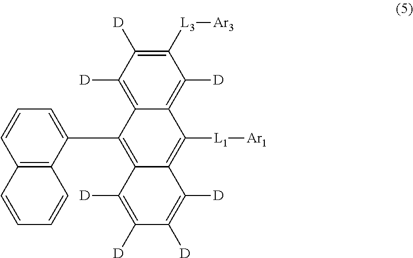
wherein in the formula (5),
D is a deuterium atom; and
L₁, L₃, Ar₁ and Ar₃ are as defined in the formula (1).

34. The compound according to claim 21, wherein the compound represented by the formula (1) is a compound represented by the following formula (6): 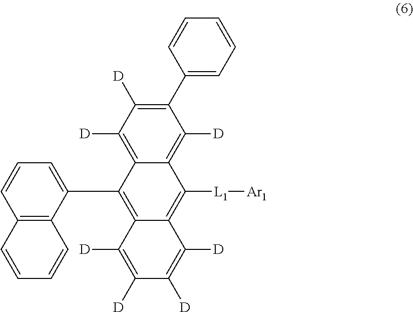
wherein in the formula (6),
D is a deuterium atom; and
L₁ and Ar₁ are as defined in the formula (1).

35. A material for an organic electroluminescence device, comprising the compound represented by the formula (1) according to claim 21.

36. The material for an organic electroluminescence device according to claim 35, wherein the material comprises a compound represented by the formula (1), and a compound having the same structures as the compound represented by the formula (1) except that the compound comprises only protium atoms as hydrogen atoms, and the content ratio of the former to the sum thereof is 30 mol % or more.

37. An organic electroluminescence device, comprising:
a cathode;
an anode; and
at least one organic layer disposed between the cathode and the anode,
wherein at least one layer of the at least one organic layer comprises the compound according to claim 21.

38. The organic electroluminescence device according to claim 36, wherein the organic layer comprises an emitting layer, and the emitting layer comprises the compound.

39. An electronic apparatus, comprising the organic electroluminescence device according to claim 37.
